(19) United States
(12) Patent Application Publication
(10) Pub. No.: US 2008/0305169 A1
(43) Pub. Date: Dec. 11, 2008

(54) Pharmaceutical Compositions Comprising Nitrogen-Containing Fused Ring Coumpounds

(75) Inventors: Kazuki Miki, Takatsuki (JP); Fuyuki Arimoto, Takatsuki (JP); Masaki Sunami, Takatsuki (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Japan Tobacco Inc., Tokyo (JP)

(21) Appl. No.: 11/753,247

(22) Filed: May 24, 2007

Related U.S. Application Data
(60) Provisional application No. 60/810,008, filed on Jun. 1, 2006.

(30) Foreign Application Priority Data
May 26, 2006 (JP) 2006-147440

(51) Int. Cl. A61K 9/36; A61K 31/538; A61K 31/5415; A61K 31/55; A61K 31/498; A61K 31/553; A61K 9/28

(52) U.S. Cl. 424/480; 514/230.5; 514/224.2; 514/213.01; 514/249; 514/211.09; 424/474

(57) ABSTRACT

[Problems] The present invention provides pharmaceutical composition which is effective for the prophylaxis or treatment of pathology showing involvement of uric acid (hyperuricemia, gouty tophus, acute gout arthritis, chronic gout arthritis, gouty kidney, urolithiasis, renal function disorder, coronary arterial disease, ischemic heart disease and the like) and the like, and is superior in the time-course stability and dissolution property (disintegration property).
[Solving Means] The pharmaceutical composition of the present invention is a pharmaceutical composition comprising a nitrogen-containing fused ring compound represented by the following formula [1] or a pharmaceutically acceptable salt thereof, and one or more pharmaceutically acceptable additives, wherein the nitrogen-containing fused ring compound or a pharmaceutically acceptable salt thereof is not in contact with a basic additive:

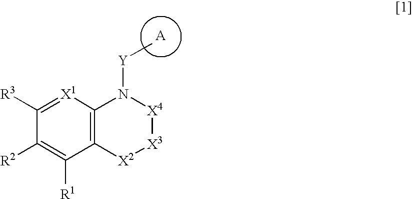

wherein each symbol is as described in the specification.

### TECHNICAL FIELD

The present invention relates to a pharmaceutical composition comprising a nitrogen-containing fused ring compound having an inhibitory action on URAT1 activity or a pharmaceutically acceptable salt.

### BACKGROUND ART

Uric acid is a substance having a molecular weight of 168 and a dissociation constant (pKa value) of 5.75, which is present in the form of uric acid or a conjugate base (urate) thereof in the body fluid depending on the pH of the body fluid. In human, since the function of urate oxidase (uricase) of the liver is lack by mutation, uric acid is the final metabolite of purine form. To be specific, dietarily or endogenously produced purine form becomes inosine from adenosine, then hypoxanthine, and then xanthine, or becomes guanine from guanosine, and then xanthine, and this xanthine is subject to oxidization by xanthine oxidase or xanthine dehydrogenase to become uric acid. Uric acid is mainly excreted from the kidney.

Hyperuricemia becomes severe, and when the blood uric acid level exceeds the upper limit of solubility, sodium urate crystal forms in the cartilage tissues and joints and then sediments called gouty tophus (tophi) are produced. This gouty tophus causes acute gouty arthritis, which progresses into chronic gouty arthritis. Besides these, it has been clarified that nephropathy (gouty kidney) and urolithiasis occur as complications of sodium urate crystal deposition due to hyperuricemia, and that hyperuricemia itself induces renal function disorder.

Many hyperuricemia patients have complications such as hyperlipidemia, diabetes, hypertension, obesity and the like. While these complications are each a risk factor for coronary artery disease and death rates, hyperuricemia patients have long been known to show significantly high complication rate of coronary artery diseases and short survival, as compared to patient groups having normal blood uric acid level. Fang et al. conducted a large-scale investigation on the death rates of coronary artery disease in 5926 cases ranging from 25 to 74 years old whose blood uric acid level was measured during the period of 22 years from 1971 to 1992, and clarified that increased blood uric acid level alone can be a risk for ischemic heart diseases. It has been also reported that a treatment aiming at decreasing the blood uric acid level itself, along with the treatment of complications, is useful for preventing the onset of and decreasing the death rates of coronary artery disease, and a treatment to decrease the blood uric acid level should be positively employed for asymptomatic hyperuricemia, too. Recently, it has become determinative that hyperuricemia associated with hypertension is an independent risk factor for cardiovascular diseases. Moreover, it has been also known that 1) nephropathy (also referred to as gouty kidney) and urolithiasis, which are complications associated with sodium urate crystal deposition, highly frequently occur, 2) control of blood uric acid level is important from the aspect of prevention of a recurrence of cerebral or cardiovascular incidents, 3) patients with hyperuricemia or gout show frequent complication of hyperlipidemia, 4) obesity should not be ignored as etiology or exacerbation factor of hyperuricemia, 5) uricosuric agents are basically used for decreased uric acid excretion hyperuricemia, 6) hyperuricemia associated with hypertension is highly likely an independent risk factor of cardiovascular incidents, and the like. Therefore, it is almost certain that decreasing the blood uric acid level is not the only effective measure for the prophylaxis or treatment of the above-mentioned diseases, but so is combined use of a pharmaceutical agent that decreases the blood uric acid level with therapeutic or prophylactic agents for these above-mentioned diseases.

While uric acid is mainly excreted from the kidney, uric acid in blood is once filtered off almost completely by renal glomerulus, after which uric acid is mostly reabsorbed by proximal renal tubule. Therefore, only a small amount of uric acid is excreted into urine. The proximal reabsorption of uric acid has been clarified to be a transport via a transporter by an experiment using membrane vesicle prepared from renal cortex, and its substrate selectivity, inhibitors thereof and the like have also been elucidated.

In recent years, a gene encoding a human kidney uric acid transporter (SLC22A12) has been identified. The transporter (urate transporter 1, URAT1) encoded by this gene is a 12-spanning transmembrane molecule belonging to the organic anion transporter (OAT) family, and Northern blot using the full-length cDNA thereof as a probe has revealed that it specifically expresses in adult and embryo kidneys. It has been also confirmed by immunostaining of human renal tissue section conducted using a polyclonal antibody specific to C-terminal peptide thereof that it is localized on the lumen of the proximal renal tubule in the cortex. In an experiment using a Xenopus laevis oocyte expression system, uric acid uptake via URAT1 increased in a time-dependent manner, and the uric acid uptake showed saturation at high uric acid concentration, which is characteristic of carrier transport. Moreover, it has been clarified that the uptake is based on the exchange with organic anion such as lactic acid, pyrazine carboxylic acid, nicotinic acid and the like, and that the uptake is inhibited by uricosuric agents such as probenecid, benzbromarone and the like, and URAT1 has been demonstrated to be the transporter being elucidated by experiments using the above-mentioned membrane vesicle. In other words, URAT1 has been clarified to be a main transporter responsible for reabsorption of uric acid in the kidney.

Furthermore, gene mutations of URAT1 have been identified by gene analysis of idiopathic renal hypouricemia patients, and when these mutant URAT1 were expressed in Xenopus laevis oocyte, the uric acid transport activity had been lost. These facts also clarify that URAT1 is involved in the control of blood uric acid level.

With regard to the relationship between URAT1 and diseases, moreover, many reports have documented that probenecid and benzbromarone that inhibit the uric acid transport activity of URAT1 are therapeutic agents for hyperuricemia, and useful as agents for the prophylaxis or treatment of pathology exhibiting high blood uric acid level, such as hyperuricemia, gouty tophus, gout arthritis, gouty kidney, urolithiasis and renal function disorder.

Furthermore, since some of the drugs of nucleic acid metabolic antagonists, hypotensive diuretics, antituberculosis, anti-inflammatory analgesic drugs, hyperlipidemic drugs, therapeutic agents for asthma, immunosuppressants and the like increase blood uric acid level, thus creating the problems of progress into or exacerbation of pathology caused by increase in the above-mentioned blood uric acid level.

Therefore, a pharmaceutical composition comprising a substance having an inhibitory action on URAT1 activity would be useful as an agent for the prophylaxis or treatment of pathology suggesting the involvement of uric acid, such as pathology suggesting the involvement of high blood uric acid level, specifically, hyperuricemia, gouty tophus, gout arthritis, gouty kidney, urolithiasis, renal function disorder and the like, and further as an agent for the prophylaxis or treatment of hyperlipidemia, diabetes, obesity or cardiovascular diseases (e.g., hypertension, coronary arterial disease, vascular endothelial disorder, ischemic heart disease etc.) because it decreases the blood uric acid level. A concurrent use of these other prophylactic or therapeutic agents with the pharmaceutical composition comprising the substance having an inhibitory action on URAT1 activity would be useful for more effective prophylaxis or treatment of these diseases.

A pharmaceutical composition comprising a substance having an inhibitory action on URAT1 activity can be said to be useful because it can prevent increase in the blood uric acid level when concurrently used together with a pharmaceutical agent that prevents increase in the blood uric acid level, such as nucleic acid metabolic antagonist, hypotensive diuretic, anti-tuberculosis, anti-inflammatory analgesic drugs, hyperlipidemic drugs, therapeutic agents for asthma, immunosuppressants and the like.

### DISCLOSURE OF THE INVENTION

### Problems to be Solved by the Invention

At present, as an agent for the prophylaxis or treatment of hyperuricemia, benzbromarone, which is a uricosuric agent having an inhibitory action on URAT1 activity is used. However, the inhibitory action on URAT1 activity of benzbromarone is not sufficient. Moreover, a possibility of inducing a pharmacokinetic drug interaction has been suggested in view of its CYP inhibitory action. Therefore, there is a strong demand for the development of an agent for the prophylaxis or treatment of hyperuricemia, which has more potent inhibitory action on URAT1 activity and has no or very weak CYP inhibitory action.

The present inventors have already found that a compound represented by the following formula [1], which is an active ingredient of the pharmaceutical composition of the present invention, inhibits a URAT1 activity and decreases the blood uric acid level, therefore, it is effective for the prophylaxis or treatment of pathology showing involvement of uric acid (hyperuricemia, gouty tophus, acute gout arthritis, chronic gout arthritis, gouty kidney, urolithiasis, renal function disorder, coronary arterial disease, ischemic heart disease and the like) and the like, and it has no or very weak CYP inhibitory action.

However, it has been found that the compound represented by the formula [1] shows good stability by itself but, depending on the combination with a conventionally-used additive, sometimes shows poor stability and insufficient dissolution property (disintegratability), and the like. Accordingly, the development of a pharmaceutical composition wherein the time-course stability of the compound during manufacturing process or storage thereof has been improved, or a pharmaceutical composition showing rapid dissolution property (disintegratability), or a pharmaceutical composition simultaneously having these characteristics is considered to be important.

The present invention aims at provision of a novel pharmaceutical composition having more potent inhibitory action on URAT1 activity as compared to conventional URAT1 activity inhibitors and having no or very weak CYP inhibitory action, wherein the time-course stability of the active ingredient during manufacturing process or storage thereof has been improved, or a pharmaceutical composition showing rapid dissolution property (disintegratability), or a pharmaceutical composition simultaneously having these characteristics.

### Means of Solving the Problems

The present inventors have conducted intensive studies in an attempt to develop a novel agent for the prophylaxis or treatment of hyperuricemia that replaces the conventional agents for the prophylaxis or treatment of hyperuricemia, and found that a pharmaceutical composition superior in the time-course stability and dissolution property (disintegratability) can be obtained by adding a particular component to the compound represented by the following formula [1], which resulted in the completion of the present invention.

Accordingly, the present invention provides the following.

<1> A pharmaceutical composition comprising a nitrogen-containing fused ring compound represented by the following formula [1] or a pharmaceutically acceptable salt thereof (hereinafter to be referred to as compound [1]), and one or more pharmaceutically acceptable additives, wherein the nitrogen-containing fused ring compound or a pharmaceutically acceptable salt thereof is not in contact with a basic additive:

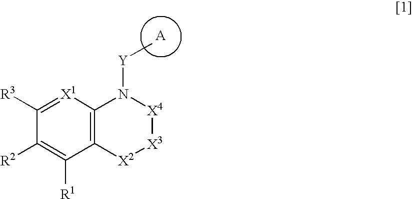

wherein
R¹, R² and R³ are the same or different and each is
1) a hydrogen atom, or
2) a group selected from group A below, or
3) R¹ and R² may form, together with the carbon atoms they are bonded to, a saturated or unsaturated carbon ring having 3 to 14 carbon atoms optionally substituted by one or more, the same or different substituents selected from group A below, or
4) R² and R³ may form, together with the carbon atoms they are bonded to, a saturated or unsaturated carbon ring having 3 to 14 carbon atoms optionally substituted by one or more, the same or different substituents selected from group A below;

### Y is

1) —CO—,
2) —CS—, or
3) —S(═O)₂—;

### X¹ is

1) a nitrogen atom, or
2) CR⁴ wherein R⁴ is
   (a) a hydrogen atom, or
   (b) a group selected from group A below, or
   (c) R³ and R⁴ may form, together with the carbon atoms they are bonded to, a saturated or unsaturated carbon ring having 3 to 14 carbon atoms optionally substituted by one or more, the same or different substituents selected from group A below;

### X² is

1) an oxygen atom,
2) —N(R⁵)— wherein R⁵ is
   (a) a hydrogen atom, or
   (b) a C₁₋₆ alkyl group optionally substituted by one or more, the same or different substituents selected from group B below,
3) —N(COR⁶)— wherein R⁶ is
   (a) a hydroxyl group,
   (b) a C₁₋₆ alkyl group optionally substituted by one or more, the same or different substituents selected from group B below,
   (c) a C₁₋₆ alkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   (d) a saturated or unsaturated carbon ring group having 3 to 14 carbon atoms optionally substituted by one or more, the same or different substituents selected from group A below,
   (e) a cycloalkylalkoxy group optionally substituted by one or more, the same or different substituents selected from group A below,
   (f) an aralkyl group optionally substituted by one or more, the same or different substituents selected from group A below, or
   (g) an aralkoxy group optionally substituted by one or more, the same or different substituents selected from group A below,
4) —N(S(═O)₂R⁶)— wherein R⁶ is as defined above,
5) —N(CONR⁷R⁸)— wherein R⁷ and R⁸ are the same or different and each is
   (a) a hydrogen atom, or
   (b) a C₁₋₆ alkyl group optionally substituted by one or more, the same or different substituents selected from group B below, or
   (c) R⁷ and R⁸ may form, together with the nitrogen atom they are bonded to, a monocyclic nitrogen-containing saturated heterocycle optionally substituted by one or more, the same or different substituents selected from group A below,
6) a sulfur atom,
7) —S(═O)—,
8) —S(═O)₂—, or
9) —CR⁹R¹⁰— wherein R⁹ and R¹⁰ are the same or different and each is
   (a) a hydrogen atom, or
   (b) a C₁₋₆ alkyl group optionally substituted by one or more, the same or different substituents selected from group B below, or
   (c) R⁹ and R¹⁰ may in combination form an oxo group;

### —X³—X⁴— is

—(CR¹¹R¹²)n- wherein n is an integer of 1 to 3, and R¹¹ and R¹² each in the number of n are the same or different and each is
   (a) a hydrogen atom, or
   (b) a C₁₋₆ alkyl group optionally substituted by one or more, the same or different substituents selected from group B below, or
   (c) R¹¹ and R¹² bonded to a single carbon atom may in combination form an oxo group, or
   (d) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms optionally substituted by one or more, the same or different substituents selected from group A below; and
      ring A is
1) a saturated or unsaturated carbon ring group having 3 to 14 carbon atoms optionally substituted by one or more, the same or different substituents selected from group A below, or
2) a saturated or unsaturated heterocyclic group containing at least one heteroatom selected from a nitrogen atom, an oxygen atom and a sulfur atom, optionally substituted by one or more, the same or different substituents selected from group A below,
   [group A]
   1) a halogen atom,
   2) —OR¹³ wherein R¹³ is
(a) a hydrogen atom,
(b) a C₁₋₆ alkyl group optionally substituted by one or more, the same or different substituents selected from group B below, or
(c) —COR¹⁴ wherein R¹⁴ is
   a) a hydrogen atom,
   b) a hydroxyl group,
   c) a C₁₋₆ alkyl group optionally substituted by one or more, the same or different substituents selected from group B below,
   d) a C₁₋₆ alkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   e) a saturated or unsaturated carbon ring group having 3 to 14 carbon atoms optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   f) a cycloalkylalkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   g) an aralkyl group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below, or
   h) an aralkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
         3) a C₁₋₆ alkyl group optionally substituted by one or more, the same or different substituents selected from group B below,
         4) a cycloalkylalkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   5) an aralkyl group optionally substituted by one or more, the same or different substituents selected from (a) and (b):
(a) a substituent selected from group B below,
(b) a C₁₋₆ alkyl group optionally substituted by one or more, the same or different substituents selected from group B below,
   6) an aralkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   7) —COR¹⁴ wherein R¹⁴ is as defined above,
   8) —NR¹⁵R¹⁶ wherein R¹⁵ and R¹⁶ are the same or different and each is
(a) a hydrogen atom, or
(b) a C₁₋₆ alkyl group optionally substituted by one or more, the same or different substituents selected from group B below, or
(c) R¹⁵ and R¹⁶ may form, together with the nitrogen atom they are bonded to, a monocyclic nitrogen-containing saturated heterocycle optionally substituted by one or more, the same or different substituents selected from (i) and (ii):
   (i) a substituent selected from group B below,
   (ii) a C₁₋₆ alkyl group optionally substituted by one or more, the same or different substituents selected from group B below,
      9) —CONR¹⁵R¹⁶ wherein R¹⁵ and R¹⁶ are as defined above,
      10) —NR¹⁷COR¹⁴ wherein R¹⁴ is as defined above, and R¹⁷ is
(a) a hydrogen atom, or
(b) a C₁₋₆ alkyl group optionally substituted by one or more, the same or different substituents selected from group B below,
   11) —NR¹⁷S(═O)₂R¹⁴ wherein R¹⁴ and R¹⁷ are as defined above,
   12) —NR¹⁷CONR¹⁵R¹⁶ wherein R¹⁵, R¹⁶ and R¹⁷ are as defined above,
   13) —SR¹³ wherein R¹³ is as defined above,
   14) —S(═O)R¹⁴ wherein R¹⁴ is as defined above,
   15) —S(═O)₂R¹⁴ wherein R¹⁴ is as defined above,
   16) —S(═O)₂NR¹⁵R¹⁶ wherein R¹⁵ and R¹⁶ are as defined above,
   17) a saturated or unsaturated carbon ring group having 3 to 14 carbon atoms optionally substituted by one or more, the same or different substituents selected from (a) and (b):
(a) a substituent selected from group B below,
(b) a C₁₋₆ alkyl group optionally substituted by one or more, the same or different substituents selected from group B below,
   18) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B below,
(b) a C₁₋₆ alkyl group optionally substituted by one or more, the same or different substituents selected from group B below,
   19) an aryloxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   20) a cyano group, and
   21) a nitro group,
   [group B]
   1) a halogen atom,
   2) a hydroxyl group,
   3) a C₁₋₆ alkoxy group,
   4) —NR¹⁸R¹⁹ wherein R¹⁸ and R¹⁹ are the same or different and each is
(a) a hydrogen atom, or
(b) a C₁₋₆ alkyl group, or
(c) R¹⁸ and R¹⁹ may form, together with the nitrogen atom they are bonded to, a monocyclic nitrogen-containing saturated heterocycle,
   5) —CONR¹⁸R¹⁹ wherein R¹⁸ and R¹⁹ are as defined above,
   6) —COR²⁰ wherein R²⁰ is
(a) a hydrogen atom,
(b) a hydroxyl group,
(c) a C₁₋₆ alkyl group, or
(d) a C₁₋₆ alkoxy group,
   7) —NR²¹COR²⁰ wherein R²⁰ is as defined above, and R²¹ is
(a) a hydrogen atom, or
(b) a C₁₋₆ alkyl group,
   8) —NR²¹CONR¹⁸R¹⁹ wherein R¹⁸, R¹⁹ and R²¹ are as defined above,
   9) —NR²¹S(═O)₂R²² wherein R²¹ is as defined above, and R²² is a C₁₋₆ alkyl group, and
   10) —S(═O)₂R²² wherein R²² is as defined above,
   wherein the C₁₋₆ alkyl group and C₁₋₆ alkoxy group in 3) to 10) above are optionally further substituted by one or more, the same or different substituents selected from
   1′) a halogen atom,
   2′) a hydroxyl group,
   3′) a C₁₋₆ alkoxy group,
   4′) —NR¹⁸′R¹⁹′ wherein R¹⁸′ and R¹⁹′ are the same or different and each is
(a) a hydrogen atom, or
(b) a C₁₋₆ alkyl group, or
(c) R¹⁸′ and R¹⁹′ may form, together with the nitrogen atom they are bonded to, a monocyclic nitrogen-containing saturated heterocycle,
   5′) —CONR¹⁸′R¹⁹′ wherein R¹⁸′ and R¹⁹′ are as defined above,
   6′) —COR²⁰′ wherein R²⁰′ is
(a) a hydrogen atom,
(b) a hydroxyl group,
(c) a C₁₋₆ alkyl group, or
(d) a C₁₋₆ alkoxy group,
   7′) —NR²¹′COR²⁰′ wherein R²⁰′ is as defined above, and R²¹′ is
(a) a hydrogen atom, or
(b) a C₁₋₆ alkyl group,
   8′) —NR²¹′CONR¹⁸′R¹⁹′ wherein R¹⁸′, R¹⁹′ and R²¹′ are as defined above,
   9′) —NR²¹′S(═O)₂R²²′ wherein R²¹′ is as defined above, and R²²′ is a C₁₋₆ alkyl group, and
   10′) —S(═O)₂R²²′ wherein R²²′ is as defined above, and the monocyclic nitrogen-containing saturated heterocycle in 4), 5) and 8) above are optionally further substituted by one or more substituents selected from a C₁₋₆ alkyl group and 1′) to 10′) above.
   <2> A pharmaceutical composition comprising a nitrogen-containing fused ring compound represented by the following formula [1] or a pharmaceutically acceptable salt thereof, and one or more pharmaceutically acceptable additives, wherein the additives are free of a basic additive, or comprise less than 1 part by weight of a basic additive per 1 part by weight of the nitrogen-containing fused ring compound represented by the following formula [1] or a pharmaceutically acceptable salt thereof:

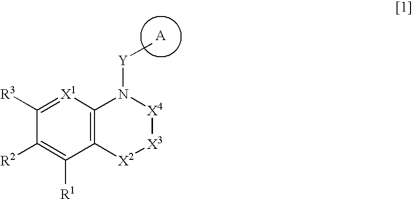

wherein each symbol is as defined in the above-mentioned <1>.
<3> The pharmaceutical composition of the above-mentioned <1>, wherein all of the additives are not basic additives.
<4> The pharmaceutical composition of any one of the above-mentioned <1> to <3>, wherein the basic additive is selected from an alkali metal hydroxide, an alkaline earth metal hydroxide, an alkali metal carbonate, an alkaline earth metal carbonate, an alkali metal hydrogencarbonate, an alkaline earth metal hydrogencarbonate, an alkali metal silicate and an alkaline earth metal silicate.
<5> The pharmaceutical composition of any one of the above-mentioned <1> to <3>, wherein the basic additive is selected from an alkali metal silicate and an alkaline earth metal silicate.
<6> The pharmaceutical composition of any one of the above-mentioned <1> to <3>, wherein the basic additive is calcium silicate.
<7> A pharmaceutical composition comprising a nitrogen-containing fused ring compound represented by the following formula [1] or a pharmaceutically acceptable salt thereof, and one or more pharmaceutically acceptable additives selected from a group consisting of an acidic additive and a neutral additive, which dose not comprise a basic additive or comprises less than 1 part by weight of a basic additive per 1 part by weight of the nitrogen-containing fused ring compound represented by the following formula [1] or a pharmaceutically acceptable salt thereof:

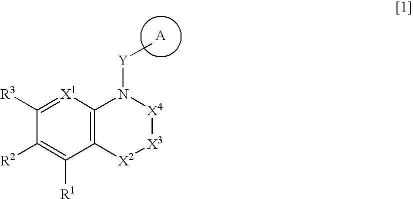

wherein each symbol is as defined in the above-mentioned <1>.
<8> A pharmaceutical composition comprising a nitrogen-containing fused ring compound represented by the following formula [1] or a pharmaceutically acceptable salt thereof, and one or more pharmaceutically acceptable additives, wherein all of the additives are selected from an acidic additive and a neutral additive:

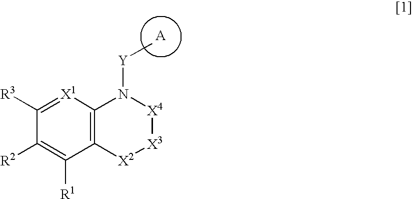

wherein each symbol is as defined in the above-mentioned <1>.
<9> The pharmaceutical composition of any one of the above-mentioned <1> to <8>, wherein the additive to be contained is one or more selected from a group consisting of D-mannitol, crystalline cellulose, low-substituted hydroxypropylcellulose, light anhydrous silicic acid, crospovidone, magnesium stearate, lactose, corn starch, sodium croscarmellose, carmellose, sodium carmellose, calcium carmellose, sodium carboxymethylstarch, hydroxypropylcellulose, hydroxypropylmethylcellulose, povidone, titanium oxide and macrogol.
<10> The pharmaceutical composition of any one of the above-mentioned <1> to <9>, which comprises low-substituted hydroxypropylcellulose.
<11> The pharmaceutical composition of any one of the above-mentioned <1> to <9>, which comprises crospovidone.
<12> The pharmaceutical composition of any one of the above-mentioned <1> to <9>, which comprises low-substituted hydroxypropylcellulose and crospovidone.
<13> The pharmaceutical composition of any one of the above-mentioned <1> to <9>, which comprises D-mannitol, crystalline cellulose, low-substituted hydroxypropylcellulose, light anhydrous silicic acid, hydroxypropylmethylcellulose, crospovidone and magnesium stearate.
<14> A pharmaceutical composition comprising a nitrogen-containing fused ring compound represented by the following formula [1] or a pharmaceutically acceptable salt thereof, and low-substituted hydroxypropylcellulose:

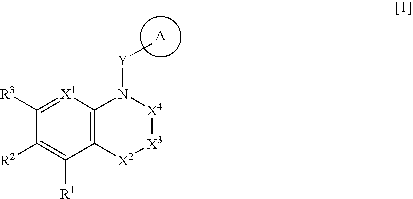

wherein each symbol is as defined in the above-mentioned <1>.
<15> A pharmaceutical composition comprising a nitrogen-containing fused ring compound represented by the following formula [1] or a pharmaceutically acceptable salt thereof, and crospovidone:

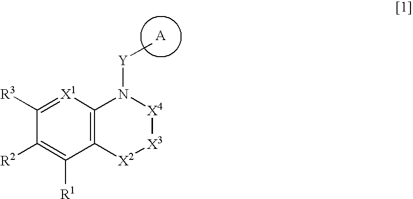

wherein each symbol is as defined in the above-mentioned <1>.
<16> A pharmaceutical composition comprising a nitrogen-containing fused ring compound represented by the following formula [1] or a pharmaceutically acceptable salt thereof, and low-substituted hydroxypropylcellulose and crospovidone:

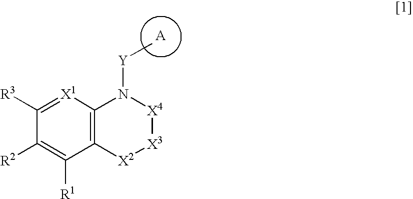

wherein each symbol is as defined in the above-mentioned <1>.
<17> The pharmaceutical composition of any one of the above-mentioned <1> to <16>, which is in the form of a tablet.
<18> The pharmaceutical composition of the above-mentioned <17>, which is coated with a coating agent.
<19> The pharmaceutical composition of the above-mentioned <18>, wherein the coating agent comprises hydroxypropylmethylcellulose, titanium oxide and macrogol.
<20> The pharmaceutical composition of any one of the above-mentioned <1> to <19>, which is a URAT1 activity inhibitor.
<21> The pharmaceutical composition of any one of the above-mentioned <1> to <19>, which is an agent for decreasing a blood uric acid value.
<22> The pharmaceutical composition of any one of the above-mentioned <1> to <19>, which is an agent for the prophylaxis or treatment of pathology with involvement of uric acid.
<23> The pharmaceutical composition of the above-mentioned <22>, wherein the pathology with involvement of uric acid is hyperuricemia, gouty tophus, acute gouty arthritis, chronic gouty arthritis, gouty kidney, urolithiasis, renal function disorder, coronary artery disease or ischemic heart disease.
<24> The pharmaceutical composition of any one of the above-mentioned <1> to <19>, which does not substantially inhibit CYP.
<25> The pharmaceutical composition of any of the above-mentioned <1> to <24>, wherein the nitrogen-containing fused ring compound represented by the formula [1] is a nitrogen-containing fused ring compound represented by the following formula [2] (hereinafter to be referred to as compound [2]):

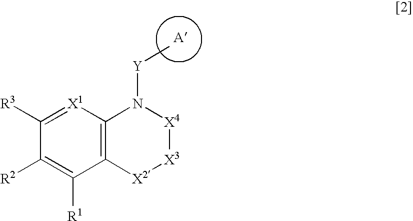

wherein R¹, R², R³, Y, X¹, X³, and X⁴ are as defined in the above-mentioned <1>,
ring A′ is
1) a saturated or unsaturated carbon ring group having 3 to 14 carbon atoms optionally substituted by one or more, the same or different substituents selected from the group C below, or
2) a saturated or unsaturated heterocyclic group containing at least one heteroatom selected from a nitrogen atom, an oxygen atom and a sulfur atom, optionally substituted by one or more, the same or different substituents selected from the group C below,
   the ring A′ is substituted by at least one —OR¹³′ wherein R¹³′ is as defined in the group C below;

### X²′ is

1) an oxygen atom,
2) —N(R⁵)— wherein R⁵ is
   (a) a hydrogen atom, or
   (b) a C₁₋₆ alkyl group optionally substituted by one or more, the same or different substituents selected from group B below,
3) —N(COR⁶)— wherein R⁶ is
   (a) a hydroxyl group,
   (b) a C₁₋₆ alkyl group optionally substituted by one or more, the same or different substituents selected from group B below,
   (c) a C₁₋₆ alkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   (d) a saturated or unsaturated carbon ring group having 3 to 14 carbon atoms optionally substituted by one or more, the same or different substituents selected from group A below,
   (e) a cycloalkylalkoxy group optionally substituted by one or more, the same or different substituents selected from group A below,
   (f) an aralkyl group optionally substituted by one or more, the same or different substituents selected from group A below, or
   (g) an aralkoxy group optionally substituted by one or more, the same or different substituents selected from group A below,
4) —N(S(═O)₂R⁶)— wherein R⁶ is as defined above,
5) —N(CONR⁷R⁸)— wherein R⁷ and R⁸ are the same or different and each is
   (a) a hydrogen atom, or
   (b) a C₁₋₆ alkyl group optionally substituted by one or more, the same or different substituents selected from group B below, or
   (c) R⁷ and R⁸ may form, together with the nitrogen atom they are bonded to, a monocyclic nitrogen-containing saturated heterocycle optionally substituted by one or more, the same or different substituents selected from group A below,
6) a sulfur atom,
7) —S(═O)—,
8) —S(═O)₂—, or
9) —CH₂—,
   (provided that when X²′ is —CH₂—,
   then —X³—X⁴— should be
—(CR¹¹R¹²)n- wherein n is an integer of 1 to 3, and R¹¹ and R¹² each in the number of n are the same or different and each is
   (a) a hydrogen atom, or
   (b) R¹¹ and R¹² bonded to a single carbon atom may in combination form an oxo group, or
   (c) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms optionally substituted by one or more, the same or different substituents selected from group A below;
      R¹³′ should be a hydrogen atom; and
      ring A′ should be further substituted by at least one a halogen atom;
provided that when both R¹¹ and R¹² are hydrogen atoms, and n is 2, then all of R¹, R² and R³ should be hydrogen atoms),
   [group A]
   1) a halogen atom,
   2) —OR¹³ wherein R¹³ is
(a) a hydrogen atom,
(b) a C₁₋₆ alkyl group optionally substituted by one or more, the same or different substituents selected from group B below, or
(c) —COR¹⁴ wherein R¹⁴ is
   a) a hydrogen atom,
   b) a hydroxyl group,
   c) a C₁₋₆ alkyl group optionally substituted by one or more, the same or different substituents selected from group B below,
   d) a C₁₋₆ alkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   e) a saturated or unsaturated carbon ring group having 3 to 14 carbon atoms optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   f) a cycloalkylalkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
   g) an aralkyl group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below, or
   h) an aralkoxy group optionally substituted by one or more, the same or different substituents selected from (i) and (ii):
      (i) a substituent selected from group B below,
      (ii) a C₁₋₆ alkyl group optionally substituted by one or more, the same or different substituents selected from group B below,
         3) a C₁₋₆ alkyl group optionally substituted by one or more, the same or different substituents selected from group B below,
         4) a cycloalkylalkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   5) an aralkyl group optionally substituted by one or more, the same or different substituents selected from (a) and (b):
(a) a substituent selected from group B below,
(b) a C₁₋₆ alkyl group optionally substituted by one or more, the same or different substituents selected from group B below,
   6) an aralkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   7) —COR¹⁴ wherein R¹⁴ is as defined above,
   8) —NR¹⁵R¹⁶ wherein R¹⁵ and R¹⁶ are the same or different and each is
(a) a hydrogen atom, or
(b) a C₁₋₆ alkyl group optionally substituted by one or more, the same or different substituents selected from group B below, or
(c) R¹⁵ and R¹⁶ may form, together with the nitrogen atom they are bonded to, a monocyclic nitrogen-containing saturated heterocycle optionally substituted by one or more, the same or different substituents selected from (i) and (ii):
   (i) a substituent selected from group B below,
   (ii) a C₁₋₆ alkyl group optionally substituted by one or more, the same or different substituents selected from group B below,
      9) —CONR¹⁵R¹⁶ wherein R¹⁵ and R¹⁶ are as defined above,
      10) —NR¹⁷COR¹⁴ wherein R¹⁴ is as defined above, and R¹⁷ is
(a) a hydrogen atom, or
(b) a C₁₋₆ alkyl group optionally substituted by one or more, the same or different substituents selected from group B below,
   11) —NR¹⁷S(═O)₂R¹⁴ wherein R¹⁴ and R¹⁷ are as defined above,
   12) —NR¹⁷CONR¹⁵R¹⁶ wherein R¹⁵, R¹⁶ and R¹⁷ are as defined above,
   13) —SR¹³ wherein R¹³ is as defined above,
   14) —S(═O)R¹⁴ wherein R¹⁴ is as defined above,
   15) —S(═O)₂R¹⁴ wherein R¹⁴ is as defined above,
   16) —S(═O)₂NR¹⁵R¹⁶ wherein R¹⁵ and R¹⁶ are as defined above,
   17) a saturated or unsaturated carbon ring group having 3 to 14 carbon atoms optionally substituted by one or more, the same or different substituents selected from (a) and (b):
(a) a substituent selected from group B below,
(b) a C₁₋₆ alkyl group optionally substituted by one or more, the same or different substituents selected from group B below,
   18) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B below,
(b) a C₁₋₆ alkyl group optionally substituted by one or more, the same or different substituents selected from group B below,
   19) an aryloxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
   20) a cyano group, and
   21) a nitro group,
   [group B]
   1) a halogen atom,
   2) a hydroxyl group,
   3) a C₁₋₆ alkoxy group,
   4) —NR¹⁸R¹⁹ wherein R¹⁸ and R¹⁹ are the same or different and each is
(a) a hydrogen atom, or
(b) a C₁₋₆ alkyl group, or
(c) R¹⁸ and R¹⁹ may form, together with the nitrogen atom they are bonded to, a monocyclic nitrogen-containing saturated heterocycle,
   5) —CONR¹⁸R¹⁹ wherein R¹⁸ and R¹⁹ are as defined above,
   6) —COR²⁰ wherein R²⁰ is
(a) a hydrogen atom,
(b) a hydroxyl group,
(c) a C₁₋₆ alkyl group, or
(d) a C₁₋₆ alkoxy group,
   7) —NR²¹COR²⁰ wherein R²⁰ is as defined above, and R²¹ is
(a) a hydrogen atom, or
(b) a C₁₋₆ alkyl group,
   8) —NR²¹CONR¹⁸R¹⁹ wherein R¹⁸, R¹⁹ and R²¹ are as defined above,
   9) —NR²¹S(═O)₂R²² wherein R²¹ is as defined above, and R²² is a C₁₋₆ alkyl group, and
   10) —S(═O)₂R²² wherein R²² is as defined above, wherein the C₁₋₆ alkyl group and C₁₋₆ alkoxy group in 3) to 10) above are optionally further substituted by one or more, the same or different substituents selected from
   1′) a halogen atom,
   2′) a hydroxyl group,
   3′) a C₁₋₆ alkoxy group,
   4′) —NR¹⁸′R¹⁹′ wherein R¹⁸′ and R¹⁹′ are the same or different and each is
(a) a hydrogen atom, or
(b) a C₁₋₆ alkyl group, or
(c) R¹⁸′ and R¹⁹′ may form, together with the nitrogen atom they are bonded to, a monocyclic nitrogen-containing saturated heterocycle,
   5′) —CONR¹⁸′R¹⁹′ wherein R¹⁸′ and R¹⁹′ are as defined above,
   6′) —COR²⁰′ wherein R²⁰′ is
(a) a hydrogen atom,
(b) a hydroxyl group,
(c) a C₁₋₆ alkyl group, or
(d) a C₁₋₆ alkoxy group,
   7′) —NR²¹′COR²⁰′ wherein R²⁰′ is as defined above, and R²¹′ is
(a) a hydrogen atom, or
(b) a C₁₋₆ alkyl group,
   8′) —NR²¹′CONR¹⁸′R¹⁹′ wherein R¹⁶′, R¹⁹′ and R²¹′ are as defined above,
   9′) —NR²¹′S(═O)₂R²²′ wherein R²¹′ is as defined above, and R²²′ is a C₁₋₆ alkyl group, and
   10′) —S(═O)₂R²²′ wherein R²²′ is as defined above, and the monocyclic nitrogen-containing saturated heterocycle in 4), 5) and 8) above are optionally further substituted by one or more substituents selected from a C₁₋₆ alkyl group and 1′) to 10′) above.
   [group C]
   1) a halogen atom,
   2) —OR¹³′ wherein R¹³′ is
(a) a hydrogen atom,
(b) a C₁₋₆ alkyl group optionally substituted by one or more, the same or different substituents selected from group B above, or
(c) —COR¹⁴′ wherein R¹⁴′ is
   a) a hydrogen atom,
   b) a hydroxyl group,
   c) a C₁₋₆ alkyl group optionally substituted by one or more, the same or different substituents selected from group B above,
   d) a C₁₋₆ alkoxy group optionally substituted by one or more, the same or different substituents selected from (i) and (ii):
      (i) a substituent selected from group B above,
      (ii) a C₁₋₆ alkyl group optionally substituted by one or more, the same or different substituents selected from group B above,
   e) a saturated or unsaturated carbon ring group having 3 to 14 carbon atoms optionally substituted by one or more, the same or different substituents selected from (i) and (ii):
      (i) a substituent selected from group B above,
      (ii) a C₁₋₆ alkyl group optionally substituted by one or more, the same or different substituents selected from group B above,
   f) a cycloalkylalkoxy group optionally substituted by one or more, the same or different substituents selected from (i) and (ii):
      (i) a substituent selected from group B above,
      (ii) a C₁₋₆ alkyl group optionally substituted by one or more, the same or different substituents selected from group B above,
   g) an aralkyl group optionally substituted by one or more, the same or different substituents selected from (i) and (ii):
      (i) a substituent selected from group B above,
      (ii) a C₁₋₆ alkyl group optionally substituted by one or more, the same or different substituents selected from group B above, or
   h) an aralkoxy group optionally substituted by one or more, the same or different substituents selected from (i) and (ii):
      (i) a substituent selected from group B above,
      (ii) a C₁₋₆ alkyl group optionally substituted by one or more, the same or different substituents selected from group B above,
         3) a C₁₋₆ alkyl group optionally substituted by one or more, the same or different substituents selected from group B above,
         4) a cycloalkylalkoxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
   5) an aralkyl group optionally substituted by one or more, the same or different substituents selected from (a) and (b):
(a) a substituent selected from group B above,
(b) a C₁₋₆ alkyl group optionally substituted by one or more, the same or different substituents selected from group B above,
   6) an aralkoxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
   7) —COR¹⁴′ wherein R¹⁴′ is as defined above,
   8) —NR¹⁵′R¹⁶′ wherein R¹⁵′ and R¹⁶′ are the same or different and each is
(a) a hydrogen atom, or
(b) a C₁₋₆ alkyl group optionally substituted by one or more, the same or different substituents selected from group B above, or
(c) R¹⁵′ and R¹⁶′ may form, together with the nitrogen atom they are bonded to, a monocyclic nitrogen-containing saturated heterocycle optionally substituted by one or more, the same or different substituents selected from (i) and (ii):
   (i) a substituent selected from group B above,
   (ii) a C₁₋₆ alkyl group optionally substituted by one or more, the same or different substituents selected from group B above,
      9) —NR¹⁷′COR¹⁴′ wherein R¹⁴′ is as defined above, and R¹⁷′ is
(a) a hydrogen atom, or
(b) a C₁₋₆ alkyl group optionally substituted by one or more, the same or different substituents selected from group B above,
   10) —NR¹⁷′S(═O)₂R¹⁴′ wherein R¹⁴′ and R¹⁷′ are as defined above,
   11) —NR¹⁷′CONR¹⁵′R¹⁶′ wherein R¹⁵′, R¹⁶′ and R¹⁷′ are as defined above,
   12) —SR¹³′ wherein R¹³′ is as defined above,
   13) —S(═O)R¹⁴′ wherein R¹⁴′ is as defined above,
   14) —S(═O)₂R¹⁴′ wherein R¹⁴′ is as defined above,
   15) —S(═O)₂NR¹⁵′R¹⁶′ wherein R¹⁵′ and R¹⁶′ are as defined above,
   16) a saturated or unsaturated carbon ring group having 3 to 14 carbon atoms optionally substituted by one or more, the same or different substituents selected from (a) and (b):
(a) a substituent selected from group B above,
(b) a C₁₋₆ alkyl group optionally substituted by one or more, the same or different substituents selected from group B above,
   17) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B above,
(b) a C₁₋₆ alkyl group optionally substituted by one or more, the same or different substituents selected from group B above,
   18) an aryloxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
   19) a cyano group, and
   20) a nitro group.
   <26> The pharmaceutical composition of any one of the above-mentioned <1> to <25>, wherein the nitrogen-containing fused ring compound represented by the formula [1] is (3-chloro-4-hydroxyphenyl)-(2,3-dihydrobenzo[1,4]oxazin-4-yl)-methanone.
   <27> The pharmaceutical composition of any one of the above-mentioned <1> to <25>, wherein the nitrogen-containing fused ring compound represented by the formula [1] is (3-bromo-4-hydroxyphenyl)-(2,3-dihydrobenzo[1,4]oxazin-4-yl)-methanone.
   <28> The pharmaceutical composition of any one of the above-mentioned <1> to <25>, wherein the nitrogen-containing fused ring compound represented by the formula [1] is (3,5-dichloro-4-hydroxyphenyl)-(2,3-dihydrobenzo[1,4]oxazin-4-yl)-methanone.
   <29> A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the nitrogen-containing fused ring compound or a pharmaceutically acceptable salt thereof is not in contact with a basic additive:

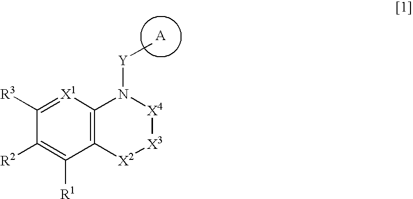

wherein each symbol is as defined in the above-mentioned <1>.
<30> A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof,
wherein the additives are free of a basic additive, or comprise less than 1 part by weight of a basic additive per 1 part by weight of the nitrogen-containing fused ring compound or a pharmaceutically acceptable salt thereof:

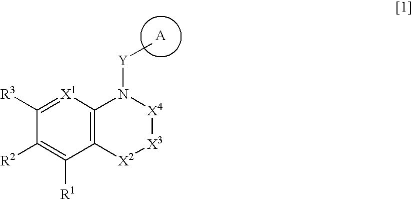

wherein each symbol is as defined in the above-mentioned <1>.
<31> A method of stabilizing a nitrogen-containing fused ring compound represented by the following formula [1] or a pharmaceutically acceptable salt thereof, comprising adding one or more pharmaceutically acceptable additives selected from a group consisting of an acidic additive and a neutral additive to the nitrogen-containing fused ring compound or a pharmaceutically acceptable salt thereof,
wherein the additives are free of a basic additive, or comprise less than 1 part by weight of a basic additive per 1 part by weight of the nitrogen-containing fused ring compound or a pharmaceutically acceptable salt thereof:

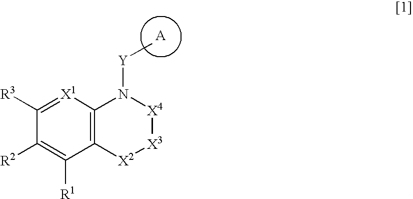

wherein each symbol is as defined in the above-mentioned <1>.
<32> A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein all of the additives are selected from a group consisting of an acidic additive and a neutral additive:

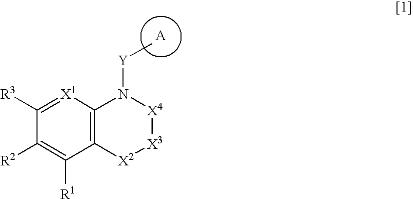

wherein each symbol is as defined in the above-mentioned <1>.
<33> A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the nitrogen-containing fused ring compound or a pharmaceutically acceptable salt thereof is not in contact with a basic additive:

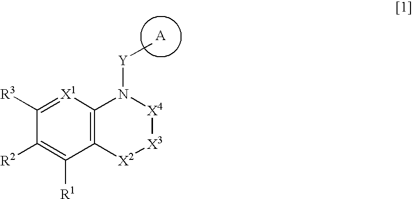

wherein each symbol is as defined in the above-mentioned <1>.
<34> A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof,
wherein the additives are free of a basic additive, or comprise less than 1 part by weight of a basic additive per 1 part by weight of the nitrogen-containing fused ring compound or a pharmaceutically acceptable salt thereof:

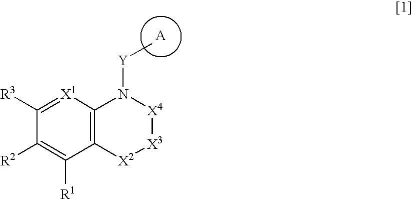

wherein each symbol is as defined in the above-mentioned <1>.
<35> A method of preserving a nitrogen-containing fused ring compound represented by the following formula [1] or a pharmaceutically acceptable salt thereof, comprising adding one or more pharmaceutically acceptable additives selected from a group consisting of an acidic additive and a neutral additive to the nitrogen-containing fused ring compound or a pharmaceutically acceptable salt thereof,
wherein the additives are free of a basic additive, or comprise less than 1 part by weight of a basic additive per 1 part by weight of the nitrogen-containing fused ring compound or a pharmaceutically acceptable salt thereof:

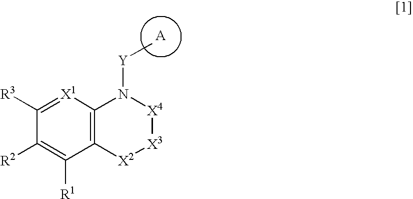

wherein each symbol is as defined in the above-mentioned <1>.
<36> A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein all of the additives are selected from a group consisting of an acidic additive and a neutral additive:

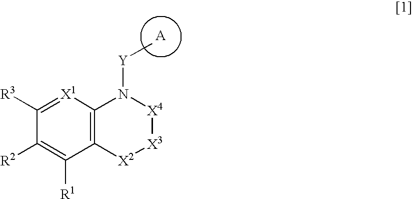

wherein each symbol is as defined in the above-mentioned <1>.

### EFFECT OF THE INVENTION

Since compound [1], which is an active ingredient of the pharmaceutical composition of the present invention, inhibits a URAT1 activity and decreases the blood uric acid level, it is useful as an agent for the prophylaxis or treatment of pathology showing involvement of uric acid, such as hyperuricemia, gouty tophus, acute gout arthritis, chronic gout arthritis, gouty kidney, urolithiasis, renal function disorder, coronary arterial disease, ischemic heart disease and the like, and the like. In addition, unlike conventional agents for the prophylaxis or treatment of hyperuricemia, since it does not substantially inhibit CYP, the possibility of causing pharmacokinetic drug interaction is extremely low, and therefore, the effect of reduced side effects can also be expected.

Moreover, the pharmaceutical composition of the present invention is superior in the time-course stability of compound [1] during manufacturing process or storage thereof and provides an effect of enabling a long-term preservation. In addition, the pharmaceutical composition of the present invention realizes rapid dissolution property (disintegratability) of compound [1], and provides an effect of rapidly increasing the drug concentration in blood.

### BRIEF DESCRIPTION OF THE DRAWINGS

FIG. 1 is a powder X-ray diffraction pattern of (3-chloro-4-hydroxyphenyl)-(2,3-dihydrobenzo[1,4]oxazin-4-yl)-methanone (Reference Example 1).

FIG. 2 is a powder X-ray diffraction pattern of (3-bromo-4-hydroxyphenyl)-(2,3-dihydrobenzo[1,4]oxazin-4-yl)-methanone (Reference Example 2).

FIG. 3 is a powder X-ray diffraction pattern of (3,5-dichloro-4-hydroxyphenyl)-(2,3-dihydrobenzo[1,4]oxazin-4-yl)-methanone (Reference Example 3).

FIG. 4 is a powder X-ray diffraction pattern of (3,5-dibromo-4-hydroxyphenyl)-(2,3-dihydrobenzo[1,4]oxazin-4-yl)-methanone (Reference Example 4).

FIG. 5 is a powder X-ray diffraction pattern of (2,3-dihydrobenzo[1,4]oxazin-4-yl)-(4-hydroxy-3,5-diiodophenyl)-methanone (Reference Example 5).

FIG. 6 is a powder X-ray diffraction pattern of (3,5-difluoro-4-hydroxyphenyl)-(2,3-dihydrobenzo[1,4]oxazin-4-yl)-methanone (Reference Example 6).

FIG. 7 is a powder X-ray diffraction pattern of (2,3-dihydrobenzo[1,4]oxazin-4-yl)-(4-hydroxy-3,5-dimethylphenyl)-methanone (Reference Example 7).

FIG. 8 is a powder X-ray diffraction pattern of (3,5-dichloro-4-hydroxyphenyl)-(1,1-dioxo-2,3-dihydro-1H-1λ⁶-benzo[1,4]thiazin-4-yl)-methanone (Reference Example 10).

FIG. 9 is a powder X-ray diffraction pattern of (3,5-dichloro-4-hydroxyphenyl)-(6-methyl-2,3-dihydrobenzo[1,4]oxazin-4-yl)-methanone (Reference Example 12).

FIG. 10 is a powder X-ray diffraction pattern of (3,5-dichloro-4-hydroxyphenyl)-(5-methyl-2,3-dihydrobenzo[1,4]oxazin-4-yl)-methanone (Reference Example 14).

FIG. 11 is a powder X-ray diffraction pattern of (3,5-dichloro-4-hydroxyphenyl)-(8-methyl-2,3-dihydrobenzo[1,4]oxazin-4-yl)-methanone (Reference Example 15).

FIG. 12 is a powder X-ray diffraction pattern of (3,5-dichloro-4-hydroxyphenyl)-(2,3-dihydronaphtho[2,1-b][1,4]oxazin-1-yl)-methanone (Reference Example 16).

FIG. 13 is a powder X-ray diffraction pattern of (3,5-dichloro-4-hydroxyphenyl)-(6-methoxy-2,3-dihydrobenzo[1,4]oxazin-4-yl)-methanone (Reference Example 17).

FIG. 14 is a powder X-ray diffraction pattern of (3,5-dichloro-4-hydroxyphenyl)-(7-methoxy-2,3-dihydrobenzo[1,4]oxazin-4-yl)-methanone (Reference Example 18).

FIG. 15 is a powder X-ray diffraction pattern of (3,5-dichloro-4-hydroxyphenyl)-(6-hydroxy-2,3-dihydrobenzo[1,4]oxazin-4-yl)-methanone (Reference Example 19).

FIG. 16 is a powder X-ray diffraction pattern of (3,5-dichloro-4-hydroxyphenyl)-(7-hydroxy-2,3-dihydrobenzo[1,4]oxazin-4-yl)-methanone (Reference Example 20).

FIG. 17 is a powder X-ray diffraction pattern of 4-(3,5-dichloro-4-hydroxybenzoyl)-3,4-dihydro-2H-benzo[1,4]oxazine-6-sulfonic acid diethylamide (Reference Example 21).

FIG. 18 is a powder X-ray diffraction pattern of (3,5-dichloro-4-hydroxyphenyl)-(3,4-dihydro-2H-quinolin-1-yl)-methanone (Reference Example 26).

FIG. 19 is a powder X-ray diffraction pattern of (3,5-dichloro-4-hydroxyphenyl)-(2,3,4,5-tetrahydrobenzo[b]azepin-1-yl)-methanone (Reference Example 27).

FIG. 20 is a powder X-ray diffraction pattern of (3-chloro-4-hydroxy-5-nitrophenyl)-(2,3-dihydrobenzo[1,4]oxazin-4-yl)-methanone (Reference Example 31).

FIG. 21 is a powder X-ray diffraction pattern of (3,5-dichloro-4-hydroxyphenyl)-(2,3-dihydropyrido[3,2-b][1,4]oxazin-4-yl)-methanone (Reference Example 36).

FIG. 22 is a powder X-ray diffraction pattern of (3,5-dichloro-2-hydroxyphenyl)-(2,3-dihydrobenzo[1,4]oxazin-4-yl)-methanone (Reference Example 38).

FIG. 23 is a powder X-ray diffraction pattern of (2,3-dihydrobenzo[1,4]oxazin-4-yl)-(4-hydroxy-3-trifluoromethylphenyl)-methanone (Reference Example 39).

FIG. 24 is a powder X-ray diffraction pattern of (3-chloro-4-hydroxy-5-methoxyphenyl)-(2,3-dihydrobenzo[1,4]oxazin-4-yl)-methanone (Reference Example 40).

FIG. 25 is a powder X-ray diffraction pattern of (4-chloro-3-hydroxyphenyl)-(2,3-dihydrobenzo[1,4]oxazin-4-yl)-methanone (Reference Example 41).

FIG. 26 is a powder X-ray diffraction pattern of (3,5-dichloro-4-hydroxyphenyl)-(6-fluoro-2,3-dihydrobenzo[1,4]oxazin-4-yl)-methanone (Reference Example 44).

FIG. 27 is a powder X-ray diffraction pattern of (3,5-dichloro-2,4-dihydroxyphenyl)-(2,3-dihydrobenzo[1,4]oxazin-4-yl)-methanone (Reference Example 50).

FIG. 28 is a powder X-ray diffraction pattern of (6-chloro-2,3-dihydrobenzo[1,4]oxazin-4-yl)-(3,5-dichloro-4-hydroxyphenyl)-methanone (Reference Example 51).

FIG. 29 is a powder X-ray diffraction pattern of (3,5-dichloro-4-hydroxyphenyl)-(6-hydroxymethyl-2,3-dihydrobenzo[1,4]oxazin-4-yl)-methanone (Reference Example 56).

FIG. 30 is a powder X-ray diffraction pattern of (3,5-dichloro-4-hydroxyphenyl)-(6,8-dimethyl-2,3-dihydrobenzo[1,4]oxazin-4-yl)-methanone (Reference Example 66).

FIG. 31 is a powder X-ray diffraction pattern of (3,5-dichloro-4-hydroxyphenyl)-(6-nitro-2,3-dihydrobenzo[1,4]oxazin-4-yl)-methanone (Reference Example 67).

FIG. 32 is a powder X-ray diffraction pattern of (3,5-dibromo-4-hydroxyphenyl)-(6-nitro-2,3-dihydrobenzo[1,4]oxazin-4-yl)-methanone (Reference Example 69).

FIG. 33 is a powder X-ray diffraction pattern of 1-[4-(3,5-dichloro-4-hydroxybenzoyl)-3,4-dihydro-2H-quinoxalin-1-yl]-ethanone (Reference Example 73).

FIG. 34 is a powder X-ray diffraction pattern of (3,5-dichloro-4-hydroxyphenyl)-(2-methyl-2,3-dihydroindol-1-yl)-methanone (Reference Example 76).

FIG. 35 is a powder X-ray diffraction pattern of (3,5-dichloro-4-hydroxyphenyl)-(2,3-dihydroindol-1-yl)-methanone (Reference Example 77).

FIG. 36 is a powder X-ray diffraction pattern of (3,5-dibromo-4-hydroxyphenyl)-(6-fluoro-2,3-dihydrobenzo[1,4]oxazin-4-yl)-methanone (Reference Example 79).

FIG. 37 is a powder X-ray diffraction pattern of (3,5-dibromo-4-hydroxyphenyl)-(2,3-dihydronaphtho[2,1-b][1,4]oxazin-1-yl)-methanone (Reference Example 80).

FIG. 38 is a powder X-ray diffraction pattern of (3,5-dibromo-4-hydroxyphenyl)-(6-methyl-2,3-dihydrobenzo[1,4]oxazin-4-yl)-methanone (Reference Example 81).

FIG. 39 is a powder X-ray diffraction pattern of (6-chloro-2,3-dihydrobenzo[1,4]oxazin-4-yl)-(3,5-dibromo-4-hydroxyphenyl)-methanone (Reference Example 82).

FIG. 40 is a powder X-ray diffraction pattern of (3,5-dichloro-4-hydroxyphenyl)-(6-trifluoromethyl-2,3-dihydrobenzo[1,4]oxazin-4-yl)-methanone (Reference Example 85).

FIG. 41 is a powder X-ray diffraction pattern of 2,6-dichloro-4-(2,3-dihydrobenzo[1,4]oxazine-4-carbonyl)phenyl acetate (Reference Example 87).

### BEST MODE FOR CARRYING OUT THE INVENTION

Each substituent used in the present specification is defined in the following.

The “C₁₋₆ alkyl group” is a straight chain or branched chain alkyl group having 1 to 6 carbon atoms and, for example, methyl group, ethyl group, propyl group, isopropyl group, butyl group, isobutyl group, sec-butyl group, tert-butyl group, pentyl group, isopentyl group, neopentyl group, tert-pentyl group, hexyl group and the like can be mentioned. Preferred is a C₁₋₄ alkyl group and particularly preferred are methyl group, ethyl group, isopropyl group and tert-butyl group.

The “C₁₋₄ alkyl group” is a straight chain or branched chain alkyl group having 1 to 4 carbon atoms and, for example, methyl group, ethyl group, propyl group, isopropyl group, butyl group, isobutyl group, sec-butyl group, tert-butyl group and the like can be mentioned. Preferred are methyl group, ethyl group, isopropyl group and tert-butyl group.

The “C₂₋₆ alkenyl group” is a straight chain or branched chain alkenyl group having 2 to 6 carbon atoms and, for example, vinyl group, n-propenyl group, isopropenyl group, n-butenyl group, isobutenyl group, sec-butenyl group, n-pentenyl group, isopentenyl group, 1-methylpropenyl group, n-hexenyl group, isohexenyl group, 1,1-dimethylbutenyl group, 2,2-dimethylbutenyl group, 3,3-dimethylbutenyl group, 3,3-dimethylpropenyl group, 2-ethylbutenyl group and the like can be mentioned. Preferred is a straight chain or branched chain alkenyl group having 2 to 4 carbon atoms and particularly preferred are vinyl group, n-propenyl group and isopropenyl group.

The “halogen atom” is fluorine atom, chlorine atom, bromine atom or iodine atom.

The “C₁₋₆ alkoxy group” is an alkoxy group wherein the alkyl moiety is the “C₁₋₆ alkyl group” defined above and, for example, methoxy group, ethoxy group, propoxy group, isopropoxy group, butoxy group, isobutoxy group, sec-butoxy group, tert-butoxy group, pentyloxy group, hexyloxy group and the like can be mentioned. Preferred is an alkoxy group wherein the alkyl moiety is the “C₁₋₄ alkyl group” defined above and particularly preferred are methoxy group and ethoxy group.

The “C₁₋₄ alkoxy group” is an alkoxy group wherein the alkyl moiety is the “C₁₋₄ alkyl group” defined above and, for example, methoxy group, ethoxy group, propoxy group, isopropoxy group, butoxy group, isobutoxy group, sec-butoxy group, tert-butoxy group and the like can be mentioned. Particularly preferred are methoxy group and ethoxy group.

The “saturated or unsaturated carbon ring group having 3 to 14 carbon atoms” is a saturated or unsaturated cyclic hydrocarbon group having 3 to 14 carbon atoms, which is specifically an aryl group, a cycloalkyl group, a cycloalkenyl group, a group derived from a fused carbon ring, wherein two or more of rings constituting them are condensed, and the like.

The “aryl group” is an aromatic hydrocarbon group having 6 to 14 carbon atoms and, for example, phenyl group, naphthyl group, biphenyl group, anthryl group, azulenyl group, phenanthryl group, pentalenyl group and the like can be mentioned. Preferred is phenyl group.

The “cycloalkyl group” is a cycloalkyl group having 3 to 8 carbon atoms and, for example, cyclopropyl group, cyclobutyl group, cyclopentyl group, cyclohexyl group, cycloheptyl group, cyclooctyl group and the like can be mentioned. Preferred is a cycloalkyl group having 3 to 6 carbon atoms, which is specifically cyclopropyl group, cyclobutyl group, cyclopentyl group and cyclohexyl group. Particularly preferred are cyclopropyl group and cyclohexyl group.

The “cycloalkenyl group” is a cycloalkenyl group having 3 to 8 carbon atoms, and contains at least one, preferably 1 or 2 double bonds. For example, cyclopropenyl group, cyclobutenyl group, cyclopentenyl group, cyclopentadienyl group, cyclohexenyl group, cyclohexadienyl group (2,4-cyclohexadien-1-yl group, 2,5-cyclohexadien-1-yl group etc.), cycloheptenyl group, cyclooctenyl group and the like can be mentioned. Preferred is a cycloalkenyl group having 3 to 6 carbon atoms, and particularly preferred is cyclohexenyl group.

As the group derived from a fused carbon ring, wherein two or more rings constituting these “aryl group”, “cycloalkyl group” and “cycloalkenyl group” are condensed, for example, indenyl group, indanyl group, fluorenyl group, 1,4-dihydronaphthyl group, 1,2,3,4-tetrahydronaphthyl group (1,2,3,4-tetrahydro-2-naphthyl group, 5,6,7,8-tetrahydro-2-naphthyl group etc.), perhydronaphthyl group and the like can be mentioned.

The “saturated or unsaturated carbon ring having 3 to 14 carbon atoms” is a ring constituting the “saturated or unsaturated carbon ring group having 3 to 14 carbon atoms” defined above.

The “aralkyl group” is an arylalkyl group wherein the aryl moiety is the “aryl group” defined above and the alkyl moiety is the “C₁₋₆ alkyl group” defined above and, for example, benzyl group, phenethyl group, 3-phenylpropyl group, 4-phenylbutyl group, 6-phenylhexyl group and the like can be mentioned. Preferred is an aralkyl group having 7 to 14 carbon atoms and particularly preferred is benzyl group.

The “aralkoxy group” is an arylalkoxy group wherein the aryl moiety is the “aryl group” defined above and the alkoxy moiety is the “C₁₋₆ alkoxy group” defined above and, for example, benzyloxy group, 3-phenylpropyloxy group, 4-phenylbutyloxy group, 6-phenylhexyloxy group and the like can be mentioned. Preferred is an aralkoxy group having 7 to 14 carbon atoms and particularly preferred is benzyloxy group.

The “cycloalkylalkoxy group” is a cycloalkylalkoxy group wherein the cycloalkyl moiety is the “cycloalkyl group” defined above and the alkoxy moiety is the “C₁₋₆ alkoxy group” defined above and, for example, cyclopropylmethoxy group, cyclobutylmethoxy group, cyclopentylmethoxy group, cyclohexylmethoxy group and the like can be mentioned. Preferred is a cycloalkylalkoxy group having 4 to 8 carbon atoms and particularly Preferred are cyclopropylmethoxy group and cyclohexylmethoxy group.

The “aryloxy group” is an aryloxy group wherein the aryl moiety is the “aryl group” defined above and, for example, phenoxy group, naphthyloxy group, biphenyloxy group and the like can be mentioned. Preferred is phenoxy group.

The “saturated or unsaturated heterocyclic group containing at least one heteroatom selected from a nitrogen atom, an oxygen atom and a sulfur atom” is a saturated or unsaturated (including partially unsaturated and completely unsaturated) monocyclic 5-membered or 6-membered heterocyclic group, containing, besides carbon atoms, at least one, preferably 1 to 4, heteroatoms selected from a nitrogen atom, an oxygen atom and a sulfur atom; a fused ring group wherein two or more of these heterocycles are condensed; or a fused ring group wherein one of the heterocycles and a carbon ring selected from benzene, cyclopentane and cyclohexane are condensed.

As the “saturated monocyclic 5-membered or 6-membered heterocyclic group”, for example, pyrrolidinyl group, tetrahydrofuryl group, tetrahydrothienyl group, imidazolidinyl group, pyrazolidinyl group, 1,3-dioxolanyl group, 1,3-oxathiolanyl group, oxazolidinyl group, thiazolidinyl group, piperidinyl group, piperazinyl group, tetrahydropyranyl group, tetrahydrothiopyranyl group, dioxanyl group, morpholinyl group, thiomorpholinyl group, 2-oxopyrrolidinyl group, 2-oxopiperidinyl group, 4-oxopiperidinyl group, 2,6-dioxopiperidinyl group and the like can be mentioned.

As the “unsaturated monocyclic 5-membered or 6-membered heterocyclic group”, for example, pyrrolyl group, furyl group, thienyl group, imidazolyl group, 1,2-dihydro-2-oxoimidazolyl group, pyrazolyl group, oxazolyl group, isoxazolyl group, thiazolyl group, isothiazolyl group, triazolyl group (e.g., 1,2,4-triazolyl group, 1,2,3-triazolyl group etc.), tetrazolyl group, 1,3,4-oxadiazolyl group, 1,2,4-oxadiazolyl group, 1,3,4-thiadiazolyl group, 1,2,4-thiadiazolyl group, furazanyl group, pyridyl group, pyrimidinyl group, 3,4-dihydro-4-oxopyrimidinyl group, pyridazinyl group, pyrazinyl group, 1,3,5-triazinyl group, imidazolinyl group, pyrazolinyl group, oxazolinyl group (e.g., 2-oxazolinyl group, 3-oxazolinyl group, 4-oxazolinyl group etc.), isoxazolinyl group, thiazolinyl group, isothiazolinyl group, pyranyl group, 2-oxopyranyl group, 2-oxo-2,5-dihydrofuranyl group, 1,1-dioxo-1H-isothiazolyl group and the like can be mentioned.

As the “fused heterocyclic group”, for example, indolyl group (e.g., 4-indolyl group, 7-indolyl group etc.), isoindolyl group, 1,3-dihydro-1,3-dioxoisoindolyl group, benzofuranyl group (e.g., 4-benzofuranyl group, 7-benzofuranyl group etc.), indazolyl group, isobenzofuranyl group, benzothiophenyl group (e.g., 4-benzothiophenyl group, 7-benzothiophenyl group etc.), benzoxazolyl group (e.g., 4-benzoxazolyl group, 7-benzoxazolyl group etc.), benzimidazolyl group (e.g., 4-benzimidazolyl group, 7-benzimidazolyl group etc.), benzothiazolyl group (e.g., 4-benzothiazolyl group, 7-benzothiazolyl group etc.), indolizinyl group, quinolyl group, isoquinolyl group, 1,2-dihydro-2-oxoquinolyl group, quinazolinyl group, quinoxalinyl group, cinnolinyl group, phthalazinyl group, quinolizinyl group, purinyl group, pteridinyl group, indolinyl group, isoindolinyl group, 5,6,7,8-tetrahydroquinolyl group, 1,2,3,4-tetrahydroquinolyl group, 2-oxo-1,2,3,4-tetrahydroquinolyl group, benzo[1,3]dioxolyl group, 3,4-methylenedioxypyridyl group, 4,5-ethylenedioxypyrimidinyl group, chromenyl group, chromanyl group, isochromanyl group and the like can be mentioned.

The “monocyclic nitrogen-containing saturated heterocycle” formed together with the adjacent nitrogen atom is a saturated 5-membered or 6-membered monocyclic heterocycle containing at least one nitrogen atom, such as piperidine, morpholine, piperazine, pyrrolidine and the like.

Being “optionally substituted by one or more, the same or different substituents” means being unsubstituted or being substituted by at least one to the acceptable maximum number of substituents. In the case of a methyl group, for example, it means being optionally substituted by 1 to 3 substituents, and in the case of an ethyl group, it means being optionally substituted by 1 to 5 substituents. When substituted by 2 or more substituents, the substituents may be the same or different and the position of the substituents may be any, without any particular limitation. Preferred is being “optionally substituted by the same or different, 1 to 3 substituents”.

Preferable examples of each group are as follows.

R¹, R² and R³ are preferably the same or different and each is

1) a hydrogen atom,
2) a halogen atom,
3) —OR¹³ (R¹³ is as defined above),
4) a C₁₋₆ alkyl group optionally substituted by one or more, the same or different substituents selected from the aforementioned group B
5) —COR¹⁴ (R¹⁴ is as defined above),
6) —NR¹⁵R¹⁶ (R¹⁵ and R¹⁶ are as defined above),
7) —NR¹⁷S(═O)₂R¹⁴ (R¹⁴ and R¹⁷ are as defined above),
8) —S(═O)₂R¹⁴ (R¹⁴ is as defined above),
9) —S(═O)₂NR¹⁵R¹⁶ (R¹⁵ and R¹⁶ are as defined above),
10) a saturated or unsaturated carbon ring group having 3 to 14 carbon atoms optionally substituted by one or more, the same or different substituents selected from (a) and (b):
(a) a substituent selected from the aforementioned group B,
(b) a C₁₋₆ alkyl group optionally substituted by one or more, the same or different substituents selected from the aforementioned group B, or
   11) a nitro group, or
   12) R¹ and R², or R² and R³ may form, together with the carbon atoms they are bonded to, a saturated or unsaturated carbon ring having 3 to 14 carbon atoms optionally substituted by one or more, preferably 2 or 3, the same or different substituents selected from the aforementioned group A.

R¹, R² and R³ are more preferably the same or different and each is

1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group),
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, isopropyl group, tert-butyl group), trifluoromethyl group, hydroxymethyl group),

### 6) —COOH,

7) —CO—C₁₋₆ alkoxy group (preferably —CO—C₁₋₄ alkoxy group, more preferably —CO-methoxy group),
8) an amino group,
9) —NHS(═O)₂—C₁₋₆ alkyl group (preferably —NHS(═O)₂—C₁₋₄ alkyl group, more preferably —NHS(═O)₂-methyl group),
10) —S(═O)₂—C₁₋₆ alkyl group (preferably —S(═O)₂—C₁₋₄ alkyl group, more preferably —S(═O)₂-ethyl group),
11) —S(═O)₂—NR¹⁵R¹⁶ (R¹⁵ and R¹⁶ are the same or different and each is
(a) a hydrogen atom, or
(b) a C₁₋₆ alkyl group (preferably a C₁₋₄ alkyl group, more preferably methyl group, ethyl group), or
(c) R¹⁵ and R¹⁶ may form, together with the nitrogen atom they are bonded to, a monocyclic nitrogen-containing saturated heterocycle (for example, pyrrolidine, piperidine, imidazolidine, piperazine and the like, preferably pyrrolidine)),
   12) a saturated or unsaturated carbon ring group having 3 to 14 carbon atoms (preferably an aryl group, more preferably phenyl group), or
   13) a nitro group.

For each of R¹, R² and R³, the following are more preferable.

R¹ is more preferably

1) a hydrogen atom,
2) a hydroxyl group,
3) a C₁₋₆ alkyl group (preferably a C₁₋₄ alkyl group, more preferably methyl group, isopropyl group),

### 4) —COOH, or

5) —CO—C₁₋₆ alkoxy group (preferably —CO—C₁₋₄ alkoxy group, more preferably —CO-methoxy group).

R² is more preferably

1) a hydrogen atom,
2) a halogen atom (preferably chlorine atom),
3) a hydroxyl group,
4) a C₁₋₆ alkoxy group (preferably a C₁₋₄ alkoxy group, more preferably methoxy group),
5) a C₁₋₆ alkyl group (preferably a C₁₋₄ alkyl group, more preferably methyl group),

### 6) —COOH,

7) —CO—C₁₋₆ alkoxy group (preferably —CO—C₁₋₄ alkoxy group, more preferably —CO-methoxy group),
8) an amino group,
9) —NHS(═O)₂—C₁₋₆ alkyl group (preferably —NHS(═O)₂—C₁₋₄ alkyl group, more preferably —NHS(═O)₂-methyl group), or
10) a nitro group.

R³ is more preferably

1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group),
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, tert-butyl group), trifluoromethyl group, hydroxymethyl group),

### 6) —COOH,

7) —CO—C₁₋₆ alkoxy group (preferably —CO—C₁₋₄ alkoxy group, more preferably —CO-methoxy group),
8) an amino group,
9) —S(═O)₂—C₁₋₆ alkyl group (preferably —S(═O)₂—C₁₋₄ alkyl group, more preferably —S(═O)₂-ethyl group),
10) —S(═O)₂—NR¹⁵R¹⁶ (R¹⁵ and R¹⁶ are the same or different and each is
(a) a hydrogen atom, or
(b) a C₁₋₆ alkyl group (preferably a C₁₋₄ alkyl group, more preferably methyl group, ethyl group), or
(c) R¹⁵ and R¹⁶ may form, together with the nitrogen atom they are bonded to, a monocyclic nitrogen-containing saturated heterocycle (for example, pyrrolidine, piperidine, imidazolidine, piperazine and the like, preferably pyrrolidine)),
   11) a saturated or unsaturated carbon ring group having 3 to 14 carbon atoms (preferably an aryl group, more preferably phenyl group), or
   12) a nitro group.

R¹, R² and R³ are particularly preferably the same or different and each is

1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group),
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, isopropyl group, tert-butyl group), trifluoromethyl group, hydroxymethyl group),
6) —CO—C₁₋₆ alkoxy group (preferably —CO—C₁₋₄ alkoxy group, more preferably —CO-methoxy group),
7) —S(═O)₂—NR¹⁵R¹⁶ (R¹⁵ and R¹⁶ are the same or different and each is
(a) a C₁₋₆ alkyl group (preferably a C₁₋₄ alkyl group, more preferably ethyl group), or
(b) R¹⁵ and R¹⁶ may form, together with the nitrogen atom they are bonded to, a monocyclic nitrogen-containing saturated heterocycle (preferably pyrrolidine)), or
   8) a nitro group.

For each of R¹, R² and R³, the following are particularly preferable.

R¹ is particularly preferably

1) a hydrogen atom,
2) a hydroxyl group,
3) a C₁₋₆ alkyl group (preferably a C₁₋₄ alkyl group, more preferably methyl group, isopropyl group), or
4) —CO—C₁₋₆ alkoxy group (preferably —CO—C₁₋₄ alkoxy group, more preferably —CO-methoxy group).

R² is particularly preferably

1) a hydrogen atom,
2) a halogen atom (preferably chlorine atom),
3) a hydroxyl group,
4) a C₁₋₆ alkoxy group (preferably a C₁₋₄ alkoxy group, more preferably methoxy group),
5) a C₁₋₆ alkyl group (preferably a C₁₋₄ alkyl group, more preferably methyl group),
6) —CO—C₁₋₆ alkoxy group (preferably —CO—C₁₋₄ alkoxy group, more preferably —CO-methoxy group), or
7) a nitro group.

R³ is particularly preferably

1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group),
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, tert-butyl group), trifluoromethyl group, hydroxymethyl group),
6) —CO—C₁₋₆ alkoxy group (preferably —CO—C₁₋₄ alkoxy group, more preferably —CO-methoxy group),
7) —S(═O)₂—NR¹⁵R¹⁶ (R¹⁵ and R¹⁶ are the same or different and each is,
(a) a C₁₋₆ alkyl group (preferably a C₁₋₄ alkyl group, more preferably ethyl group), or
(b) R¹⁵ and R¹⁶ may form, together with the nitrogen atom they are bonded to, a monocyclic nitrogen-containing saturated heterocycle (preferably pyrrolidine)), or
   8) a nitro group.

Y is preferably —CO— or —CS—.

X¹ is preferably

1) a nitrogen atom, or
2) CR⁴ wherein R⁴ is
(a) a hydrogen atom,
(b) a hydroxyl group,
(c) a C₁₋₆ alkyl group (preferably a C₁₋₄ alkyl group, more preferably methyl group), or
(d) —CO—C₁₋₆ alkoxy group (preferably —CO—C₁₋₄ alkoxy group, more preferably —CO-methoxy group), or
(e) R³ and R⁴ (R³ is as defined above) may form, together with the carbon atoms they are bonded to, a saturated or unsaturated carbon ring having 3 to 14 carbon atoms (preferably an aromatic hydrocarbon, more preferably benzene ring).

X¹ is more preferably

CR⁴ wherein R⁴ is
(a) a hydrogen atom,
(b) a hydroxyl group,
(c) a C₁₋₆ alkyl group (preferably a C₁₋₄ alkyl group, more preferably methyl group), or
(d) —CO—C₁₋₆ alkoxy group (preferably —CO—C₁₋₄ alkoxy group, more preferably —CO-methoxy group), or
(e) R³ and R⁴ (R³ is as defined above) may form, together with the carbon atoms they are bonded to, a saturated or unsaturated carbon ring having 3 to 14 carbon atoms (preferably an aromatic hydrocarbon, more preferably benzene ring).

X² is preferably

1) an oxygen atom,
2) —N(R⁵)— wherein R⁵ is
(a) a hydrogen atom, or
(b) a C₁₋₆ alkyl group (preferably a C₁₋₄ alkyl group, more preferably methyl group),
   3) —N(COR⁶)— wherein R⁶ is
(a) a C₁₋₆ alkyl group (preferably a C₁₋₄ alkyl group, more preferably methyl group), or
(b) a saturated or unsaturated carbon ring group having 3 to 14 carbon atoms (preferably an aryl group, more preferably phenyl group) optionally substituted by one or more, the same or different substituents selected from a halogen atom (preferably chlorine atom) and a hydroxyl group),
   4) —N(S(═O)₂—C₁₋₆ alkyl group)- (preferably —N(—S(═O)₂—C₁₋₄ alkyl group)-, more preferably —N(—S(═O)₂-methyl group)-),
   5) a sulfur atom,

### 6) —S(═O)—,

### 7) —S(═O)₂—, or

### 8) —CH₂—.

X²′ is preferably

1) an oxygen atom,
2) —N(R⁵)— wherein R⁵ is
(a) a hydrogen atom, or
(b) a C₁₋₆ alkyl group (preferably a C₁₋₄ alkyl group, more preferably methyl group),
   3) —N(COR⁶) wherein R⁶ is
(a) a C₁₋₆ alkyl group (preferably a C₁₋₄ alkyl group, more preferably methyl group), or
(b) a saturated or unsaturated carbon ring group having 3 to 14 carbon atoms (preferably an aryl group, more preferably phenyl group) optionally substituted by one or more, the same or different substituents selected from a halogen atom (preferably chlorine atom) and a hydroxyl group),
   4) —N(S(═O)₂—C₁₋₆ alkyl group)- (preferably —N(—S(═O)₂—C₁₋₄ alkyl group)-, more preferably —N(—S(═O)₂-methyl group)-),
   5) a sulfur atom,

### 6) —S(═O)—,

### 7) —S(═O)₂—, or

### 8) —CH₂—.

X²′ is more preferably an oxygen atom.

—X³—X⁴— is preferably

—(CR¹¹R¹²)n- wherein n is an integer of 1 to 3, and R¹¹ and R¹² each in the number of n are the same or different and each is
(a) a hydrogen atom, or
(b) R¹¹ and R¹² bonded to a single carbon atom may in combination form an oxo group, or
(c) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms (preferably an aromatic hydrocarbon, more preferably benzene ring).
   “n” of —(CR¹¹R¹²)n- for —X³—X⁴— is preferably 2 or 3, more preferably 2.

As —(CR¹¹R¹²)n-,

### 1) —CH₂—,

### 2) —CH(CH₃)—

### 3) —CH(CH₂CH₃)—

### 4) —CH(CH(CH₃)₂)—

### 5) —CH₂—CH₂—,

### 6) —CH(CH₃)—CH₂—,

### 7) —CH(CH₂CH₃)—CH₂—,

### 8) —CH(CH(CH₃)₂) —CH₂—,

### 9) —CH₂—CH(CH₃)—,

### 10) —CH₂—CH(CH₂CH₃)—,

### 11) —CH₂—CH(CH(CH₃)₂)

### 12) —CH₂—CH₂—CH₂—,

### 13) —CH₂—CH(CH₃)—CH₂—,

### 14) —CH₂—CH₂—CH(CH₃)—,

### 15) —CO—,

### 16) —CO—CH₂—,

### 17) —CH₂—CO—,

### 18) —CO—CH₂—,

### 19) —CO—CH₂—CH₂—,

### 20) —CH₂—CO—CH₂—,

### 21) —CH₂—CH₂—CO—,

22)

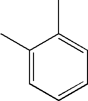

and the like can be specifically mentioned.

—X³—X⁴— is more preferably —CH₂—CH₂—.

Ring A is preferably an unsaturated carbon ring group having 3 to 14 carbon atoms optionally substituted by one or more, preferably 2 or 3, the same or different substituents selected from the aforementioned group A, or an unsaturated heterocyclic group containing at least one heteroatom selected from a nitrogen atom, an oxygen atom and a sulfur atom, optionally substituted by one or more, preferably 2 or 3, the same or different substituents selected from the aforementioned group A.

Ring A is more preferably

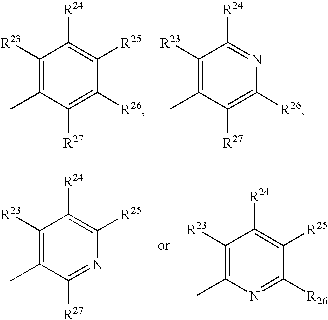

wherein R²³ to R²⁷ are the same or different and each is
1) a hydrogen atom, or
2) a group selected from the aforementioned group A.

R²³ to R²⁷ on ring A are preferably are the same or different and each is

1) a hydrogen atom,
2) a halogen atom,
3) —OR¹³ (R¹³ is as defined above),
4) a C₁₋₆ alkyl group optionally substituted by one or more, the same or different substituents selected from the aforementioned group B,
5) —COR¹⁴ (R¹⁴ is as defined above),
6) —NR¹⁵R¹⁶ (R¹⁵ and R¹⁶ are as defined above),
7) —NR¹⁷S(═O)₂R¹⁴ (R¹⁴ and R¹⁷ are as defined above), or
8) a nitro group.

R²³ to R²⁷ on ring A are more preferably the same or different and each is

1) a hydrogen atom,
2) a halogen atom,
3) a hydroxyl group,
4) a C₁₋₆ alkoxy group optionally substituted by one or more, the same or different substituents selected from —COOH and —CO—C₁₋₆ alkoxy group (preferably —CO—C₁₋₄ alkoxy group, more preferably —CO-ethoxy group)
(preferably a C₁₋₄ alkoxy group (preferably methoxy group), carboxymethoxy group, (ethoxycarbonyl)methoxy group),
5) —O—CO—C₁₋₆ alkyl group (preferably —O—CO—C₁₋₄ alkyl group, more preferably —O—CO-methyl group),
6) a C₁₋₆ alkyl group optionally substituted by one or more, the same or different halogen atoms (preferably fluorine atom)
(preferably a C₁₋₄ alkyl group (preferably methyl group), trifluoromethyl group),

### 7) —COOH,

8) —CO—C₁₋₆ alkoxy group (preferably —CO—C₁₋₄ alkoxy group, more preferably —CO-methoxy group),
9) an amino group,
10) —NHS(═O)₂—C₁₋₆ alkyl group (preferably —NHS(═O)₂—C₁₋₄ alkyl group, more preferably —NHS(═O)₂-methyl group), or
11) a nitro group.

For each of R²³ to R²⁷ on ring A, the following are more preferable.

R²³ on ring A is more preferably

1) a hydrogen atom,
2) a hydroxyl group, or

### 3) —COOH.

R²⁴ on ring A is more preferably

1) a hydrogen atom,
2) a halogen atom,
3) a hydroxyl group,
4) a C₁₋₆ alkoxy group (preferably a C₁₋₆ alkoxy group, more preferably methoxy group),
5) a C₁₋₆ alkyl group optionally substituted by one or more, the same or different halogen atoms (preferably fluorine atom)
(preferably a C₁₋₄ alkyl group (preferably methyl group), trifluoromethyl group),

### 6) —COOH,

7) —CO—C₁₋₆ alkoxy group (preferably —CO—C₁₋₄ alkoxy group, more preferably —CO-methoxy group), or
8) nitro group.

R²⁵ on ring A is more preferably

1) a hydrogen atom,
2) a halogen atom,
3) a hydroxyl group,
4) a C₁₋₆ alkoxy group optionally substituted by one or more, the same or different substituents selected from —COOH and —CO—C₁₋₆ alkoxy group (preferably —CO—C₁₋₄ alkoxy group, more preferably —CO-ethoxy group)
(preferably a C₁₋₄ alkoxy group (preferably methoxy group), carboxymethoxy group, (ethoxycarbonyl)methoxy group),
5) —O—CO—C₁₋₆ alkyl group (preferably —O—CO—C₁₋₄ alkyl group, more preferably —O—CO-methyl group),

### 6) —COOH,

7) —CO—C₁₋₆ alkoxy group (preferably —CO—C₁₋₄ alkoxy group, more preferably —CO-methoxy group),
8) an amino group,
9) —NHS(═O)₂—C₁₋₆ alkyl group (preferably —NHS(═O)₂—C₁₋₄ alkyl group, more preferably —NHS(═O)₂-methyl group), or
10) a nitro group.

R²⁶ on ring A is more preferably

1) a hydrogen atom,
2) a halogen atom,
3) a hydroxyl group,
4) a C₁₋₆ alkoxy group (preferably a C₁₋₆ alkoxy group, more preferably methoxy group),
5) a C₁₋₆ alkyl group optionally substituted by one or more, the same or different halogen atoms (preferably fluorine atom)
(preferably a C₁₋₄ alkyl group (preferably methyl group), trifluoromethyl group),

### 6) —COOH,

7) —CO—C₁₋₆ alkoxy group (preferably —CO—C₁₋₄ alkoxy group, more preferably —CO-methoxy group), or
8) nitro group.

R²⁷ on ring A is more preferably

1) a hydrogen atom,
2) a hydroxyl group, or

### 3) —COOH.

As such ring A,

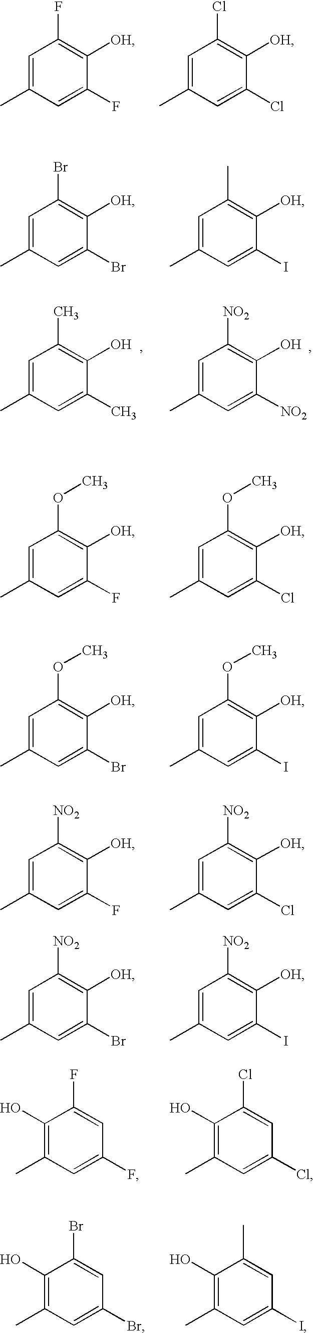

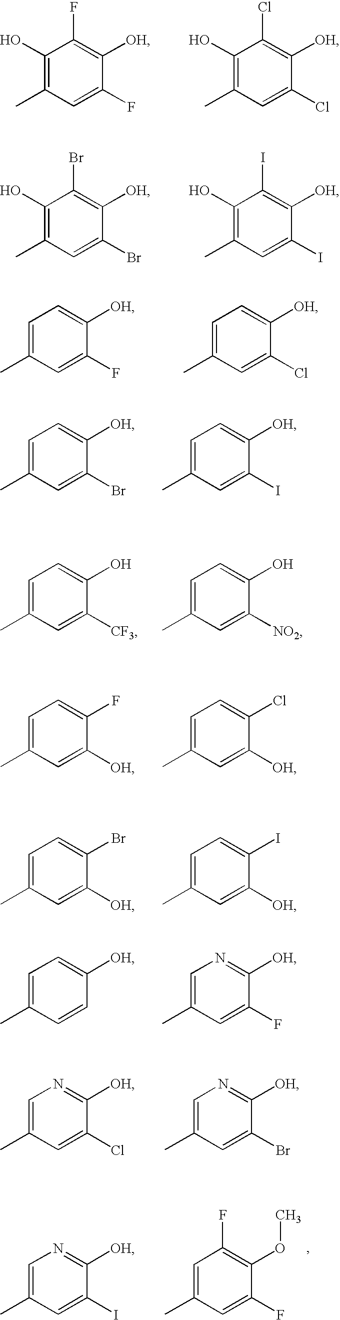

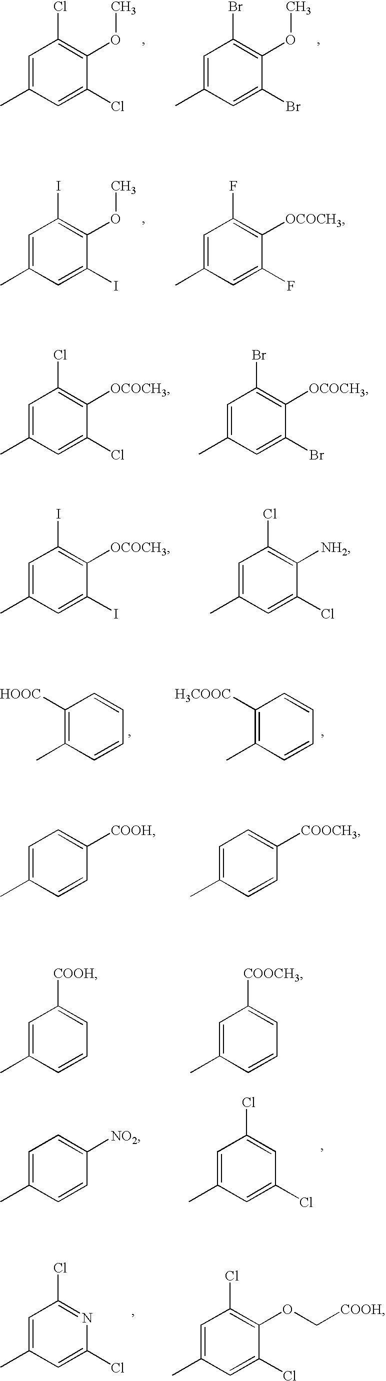

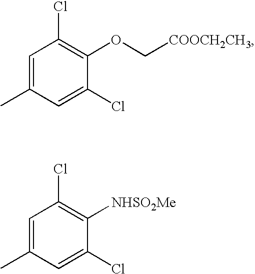

and the like can be mentioned.

R²³ to R²⁷ on ring A are particularly preferably the same or different and each is

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group.

For each of R²³ to R²⁷ on ring A, the following are particularly preferable.

R²³ on ring A is particularly preferably

1) a hydrogen atom, or
2) a hydroxyl group.

R²⁴ on ring A is particularly preferably

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group.

R²⁵ on ring A is particularly preferably

1) a hydrogen atom,
2) a halogen atom,
3) a hydroxyl group, or
4) nitro group.

R²⁶ on ring A is particularly preferably

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group.

R²⁷ on ring A is particularly preferably

1) a hydrogen atom, or
2) a hydroxyl group.

Ring A′ is preferably an unsaturated carbon ring group having 3 to 14 carbon atoms optionally substituted by one or more, preferably 2 or 3, the same or different substituents selected from the aforementioned group C, or an unsaturated heterocyclic group containing at least one heteroatom selected from a nitrogen atom, an oxygen atom and a sulfur atom, optionally substituted by one or more, preferably 2 or 3, the same or different substituents selected from the aforementioned group C. Ring A′ is substituted by at least one —OR¹³′ wherein R¹³′ is as defined above.

Ring A′ is preferably

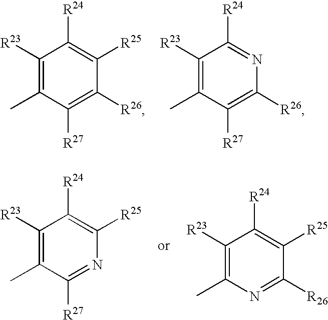

wherein
R²³ to R²⁷ are the same or different and each is
   1) a hydrogen atom, or
   2) a group selected from the aforementioned group C, and
   at least one of R²³ to R²⁷ is —OR¹³′ wherein R¹³ is as defined above.
R²³ to R²⁷ on ring A′ are preferably are the same or different and each is
   1) a hydrogen atom,
   2) a halogen atom,
   3) —OR¹³′ (R¹³′ is as defined above),
   4) a C₁₋₆ alkyl group optionally substituted by one or more, the same or different substituents selected from the aforementioned group B,
   5) —COR¹⁴′ (R¹⁴′ is as defined above),
   6) —NR¹⁵′R¹⁶′ (R¹⁵′ and R¹⁶′ are as defined above),
   7) —NR¹⁷′S(═O)₂R¹⁴′ (R¹⁴′ and R¹⁷′ are as defined above), or
   8) a nitro group, and
   at least one of R²³ to R²⁷ is —OR¹³′ wherein R¹³′ is as defined above.

R²³ to R²⁷ on ring A′ are more preferably the same or different and each is

1) a hydrogen atom,
2) a halogen atom,
3) a hydroxyl group,
4) a C₁₋₆ alkoxy group optionally substituted by one or more, the same or different substituents selected from —COOH and —CO—C₁₋₆ alkoxy group (preferably —CO—C₁₋₄ alkoxy group, more preferably —CO-ethoxy group)
(preferably a C₁₋₄ alkoxy group (preferably methoxy group), carboxymethoxy group, (ethoxycarbonyl)methoxy group),
5) —O—CO—C₁₋₆ alkyl group (preferably —O—CO—C₁₋₄ alkyl group, more preferably —O—CO-methyl group),
6) a C₁₋₆ alkyl group optionally substituted by one or more, the same or different halogen atoms (preferably fluorine atom)
(preferably a C₁₋₄ alkyl group (preferably methyl group), trifluoromethyl group),

### 7) —COOH,

8) —CO—C₁₋₆ alkoxy group (preferably —CO—C₁₋₄ alkoxy group, more preferably —CO-methoxy group),
9) an amino group,
10) —NHS(═O)₂—C₁₋₆ alkyl group (preferably —NHS(═O)₂—C₁₋₄ alkyl group, more preferably —NHS(═O)₂-methyl group), or
11) a nitro group, and
at least one of R²³ to R²⁷ is a group selected from hydroxyl group, C₁₋₆ alkoxy group (preferably C₁₋₄ alkoxy group, more preferably methoxy group) optionally substituted by one or more, the same or different substituents selected from —COOH or —CO—C₁₋₆ alkoxy group (preferably —CO—C₁₋₄ alkoxy group, more preferably —CO-ethoxy group), and —O—CO—C₁₋₆ alkyl group (preferably —O—CO—C₁₋₄ alkyl group, more preferably —O—CO-methyl group).

For each of R²³ to R²⁷ on ring A′, the following are more preferable.

R²³ on ring A′ is more preferably

1) a hydrogen atom,
2) a hydroxyl group, or

### 3) —COOH.

R²⁴ on ring A′ is more preferably

1) a hydrogen atom,
2) a halogen atom,
3) a hydroxyl group,
4) a C₁₋₆ alkoxy group (preferably a C₁₋₆ alkoxy group, more preferably methoxy group),
5) a C₁₋₆ alkyl group optionally substituted by one or more, the same or different halogen atoms (preferably fluorine atom)
(preferably a C₁₋₄ alkyl group (preferably methyl group), trifluoromethyl group),

### 6) —COOH,

7) —CO—C₁₋₆ alkoxy group (preferably —CO—C₁₋₄ alkoxy group, more preferably —CO-methoxy group), or
8) nitro group.

R²⁵ on ring A′ is more preferably

1) a hydrogen atom,
2) a halogen atom,
3) a hydroxyl group,
4) a C₁₋₆ alkoxy group optionally substituted by one or more, the same or different substituents selected from —COOH and —CO—C₁₋₆ alkoxy group (preferably —CO—C₁₋₄ alkoxy group, more preferably —CO-ethoxy group)
(preferably a C₁₋₄ alkoxy group (preferably methoxy group), carboxymethoxy group, (ethoxycarbonyl)methoxy group),
5) —O—CO—C₁₋₆ alkyl group (preferably —O—CO—C₁₋₄ alkyl group, more preferably —O—CO-methyl group),

### 6) —COOH,

7) —CO—C₁₋₆ alkoxy group (preferably —CO—C₁₋₄ alkoxy group, more preferably —CO-methoxy group),
8) an amino group,
9) —NHS(═O)₂—C₁₋₆ alkyl group (preferably —NHS(═O)₂—C₁₋₄ alkyl group, more preferably —NHS(═O)₂-methyl group), or
10) a nitro group.

R²⁶ on ring A′ is more preferably

1) a hydrogen atom,
2) a halogen atom,
3) a hydroxyl group,
4) a C₁₋₆ alkoxy group (preferably a C₁₋₆ alkoxy group, more preferably methoxy group),
5) a C₁₋₆ alkyl group optionally substituted by one or more, the same or different halogen atoms (preferably fluorine atom)
(preferably a C₁₋₄ alkyl group (preferably methyl group), trifluoromethyl group),

### 6) —COOH,

7) —CO—C₁₋₆ alkoxy group (preferably —CO—C₁₋₄ alkoxy group, more preferably —CO-methoxy group), or
8) nitro group.

R²⁷ on ring A′ is more preferably

1) a hydrogen atom,
2) a hydroxyl group, or

### 3) —COOH.

As such ring A′,

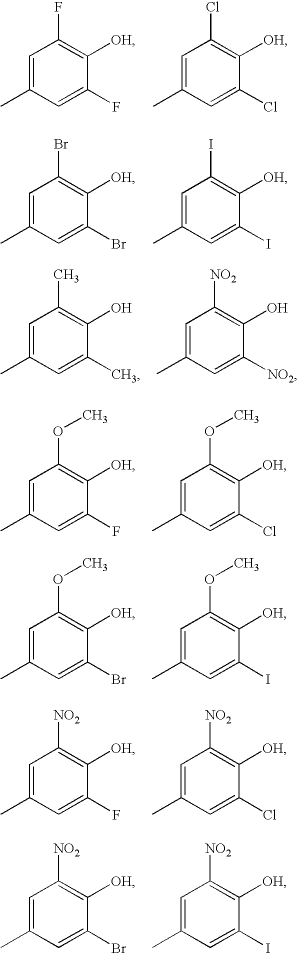

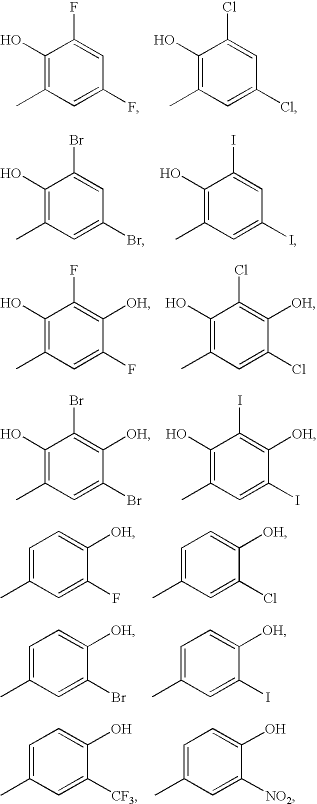

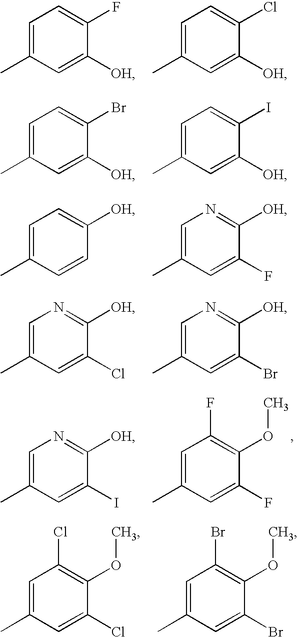

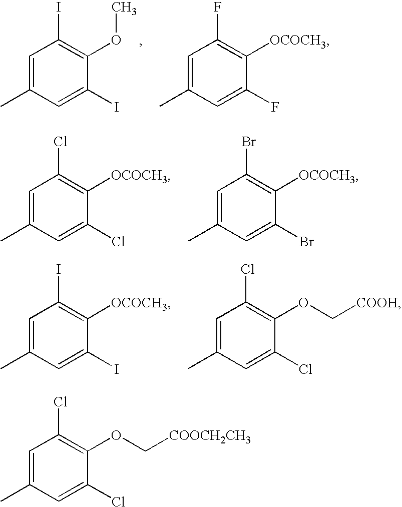

and the like can be mentioned.

R²³ to R²⁷ on ring A′ are particularly preferably the same or different and each is

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group, and
at least one of R²³ to R²⁷ is a hydroxyl group.

For each of R²³ to R²⁷ on ring A′, the following are particularly preferable.

R²³ on ring A′ is particularly preferably

1) a hydrogen atom, or
2) a hydroxyl group.

R²⁴ on ring A′ is particularly preferably

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group.

R²⁵ on ring A′ is particularly preferably

1) a hydrogen atom,
2) a halogen atom,
3) a hydroxyl group, or
4) nitro group.

R²⁶ on ring A′ is particularly preferably

1) a hydrogen atom,
2) a halogen atom,
3) a hydroxyl group,
4) a C₁₋₆ alkyl group optionally substituted by one or more, the same or different halogen atoms (preferably fluorine atom) (preferably a C₁₋₄ alkyl group (preferably methyl group), trifluoromethyl group), or
5) a nitro group.

R²⁷ on ring A′ is particularly preferably

1) a hydrogen atom, or
2) a hydroxyl group.

Of compound [1], compound [2] is preferable. A compound wherein

R¹, R² and R³ are the same or different and each is
1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group),
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, isopropyl group, tert-butyl group), trifluoromethyl group, hydroxymethyl group),
6) —COOH,
7) —CO—C₁₋₆ alkoxy group (preferably —CO—C₁₋₄ alkoxy group, more preferably —CO-methoxy group),
8) an amino group,
9) —NHS(═O)₂—C₁₋₆ alkyl group (preferably —NHS(═O)₂—C₁₋₄ alkyl group, more preferably —NHS(═O)₂-methyl group),
10) —S(═O)₂—C₁₋₆ alkyl group (preferably —S(═O)₂—C₁₋₄ alkyl group, more preferably —S(═O)₂-ethyl group),
11) —S(═O)₂—NR¹⁵R¹⁶ (R¹⁵ and R¹⁶ are the same or different and each is
   (a) a hydrogen atom, or
   (b) a C₁₋₆ alkyl group (preferably a C₁₋₄ alkyl group, more preferably methyl group, ethyl group), or
   (c) R¹⁵ and R¹⁶ may form, together with the nitrogen atom they are bonded to, a monocyclic nitrogen-containing saturated heterocycle (for example, pyrrolidine, piperidine, imidazolidine, piperazine and the like, preferably pyrrolidine)),
12) a saturated or unsaturated carbon ring group having 3 to 14 carbon atoms (preferably an aryl group, more preferably phenyl group), or
13) a nitro group;

### Y is

1) —CO—,
2) —CS—, or
3) —S(═O)₂—;

### X¹ is

1) a nitrogen atom, or
2) CR⁴ wherein R⁴ is
   (a) a hydrogen atom,
   (b) a hydroxyl group,
   (c) a C₁₋₆ alkyl group (preferably a C₁₋₄ alkyl group, more preferably methyl group), or
   (d) —CO—C₁₋₆ alkoxy group (preferably —CO—C₁₋₄ alkoxy group, more preferably —CO-methoxy group), or
   (e) R³ and R⁴ (R³ is as defined above) may form, together with the carbon atoms they are bonded to, a saturated or unsaturated carbon ring having 3 to 14 carbon atoms (preferably an aromatic hydrocarbon, more preferably benzene ring);

### X²′ is

1) an oxygen atom,
2) —N(R⁵)— wherein R⁵ is
   (a) a hydrogen atom, or
   (b) a C₁₋₆ alkyl group (preferably a C₁₋₄ alkyl group, more preferably methyl group),
3) —N(COR⁶)— wherein R⁶ is
   (a) a C₁₋₆ alkyl group (preferably a C₁₋₄ alkyl group, more preferably methyl group), or
   (b) a saturated or unsaturated carbon ring group having 3 to 14 carbon atoms (preferably an aryl group, more preferably phenyl group) optionally substituted by one or more, the same or different substituents selected from a halogen atom (preferably chlorine atom) and a hydroxyl group),
4) —N(S(═O)₂—C₁₋₆ alkyl group)- (preferably —N(—S(═O)₂—C₁₋₄ alkyl group)-, more preferably —N(—S(═O)₂-methyl group)-),
5) a sulfur atom,
6) —S(═O)—,
7) —S(═O)₂—, or
8) —CH₂—;

### —X³—X⁴— is

—(CR¹¹R¹²)n- wherein n is an integer of 1 to 3, and R¹¹ and R¹² each in the number of n are the same or different and each is
   (a) a hydrogen atom, or
   (b) R¹¹ and R¹² bonded to a single carbon atom may in combination form an oxo group, or
   (c) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms (preferably an aromatic hydrocarbon, more preferably benzene ring); and
      ring A′ is

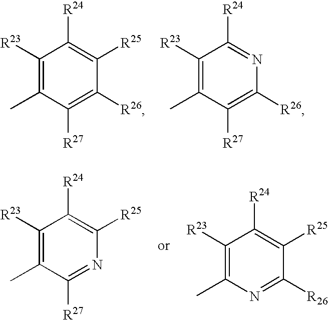

wherein
R²³ to R²⁷ are the same or different and each is
   1) a hydrogen atom,
   2) a halogen atom,
   3) a hydroxyl group,
   4) a C₁₋₆ alkoxy group optionally substituted by one or more, the same or different substituents selected from —COOH and —CO—C₁₋₆ alkoxy group (preferably —CO—C₁₋₄ alkoxy group, more preferably —CO-ethoxy group) (preferably a C₁₋₄ alkoxy group (preferably methoxy group), carboxymethoxy group, (ethoxycarbonyl)methoxy group),
   5) —O—CO—C₁₋₆ alkyl group (preferably —O—CO—C₁₋₄ alkyl group, more preferably —O—CO-methyl group),
   6) a C₁₋₆ alkyl group optionally substituted by one or more, the same or different halogen atoms
   (preferably fluorine atom)
   (preferably a C₁₋₄ alkyl group (preferably methyl group), trifluoromethyl group),
   7) —COOH,
   8) —CO—C₁₋₆ alkoxy group (preferably —CO—C₁₋₄ alkoxy group, more preferably —CO-methoxy group),
   9) an amino group,
   10) —NHS(═O)₂—C₁₋₆ alkyl group (preferably —NHS(═O)₂—C₁₋₄ alkyl group, more preferably —NHS(═O)₂-methyl group), or
   11) a nitro group, and
at least one of R²³ to R²⁷ is a group selected from a hydroxyl group, a C₁₋₆ alkoxy group (preferably a C₁₋₄ alkoxy group, more preferably methoxy group) optionally substituted by one or more, the same or different substituents selected from —COOH and —CO—C₁₋₆ alkoxy group (preferably —CO—C₁₋₄ alkoxy group, more preferably —CO-ethoxy group), and —O—CO—C₁₋₆ alkyl group (preferably —O—CO—C₁₋₄ alkyl group, more preferably —O—CO-methyl group);
   (provided that when X²′ is —CH₂—,
   then —X³—X⁴— should be
−(CR¹¹R¹²)n- wherein n is an integer of 1 to 3, and R¹¹ and R¹² each in the number of n are the same or different and each is
   (a) a hydrogen atom, or
   (b) R¹¹ and R¹² bonded to a single carbon atom may in combination form an oxo group, or
   (c) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms (preferably an aromatic hydrocarbon, more preferably benzene ring);
      R¹³′ should be a hydrogen atom; and
      ring A′ should be further substituted by at least one a halogen atom;
provided that when both R¹¹ and R¹² are hydrogen atoms, and n is 2, then all of each R¹, R² and R³ should be hydrogen atoms),
   is preferable.

Particularly, a compound wherein

R¹, R² and R³ are the same or different and each is
1) a hydrogen atom,
2) a halogen atom (preferably fluorine atom, chlorine atom),
3) a hydroxyl group,
4) a C₁₋₆ alkoxy group (preferably a C₁₋₄ alkoxy group, more preferably methoxy group)
5) a C₁₋₆ alkyl group optionally substituted by one or more, the same or different substituents selected from a halogen atom (preferably fluorine atom) and a hydroxyl group (preferably a C₁₋₄ alkyl group (preferably methyl group, isopropyl group, tert-butyl group), trifluoromethyl group, hydroxymethyl group),
6) —CO—C₁₋₆ alkoxy group (preferably —CO—C₁₋₄ alkoxy group, more preferably —CO-methoxy group),
7) —S(═O)₂—NR¹⁵R¹⁶ (R¹⁵ and R¹⁶ are the same or different and each is
   (a) a C₁₋₆ alkyl group (preferably a C₁₋₄ alkyl group, more preferably ethyl group), or
   (b) R¹⁵ and R¹⁶ may form, together with the nitrogen atom they are bonded to, a monocyclic nitrogen-containing saturated heterocycle (preferably pyrrolidine)), or
8) a nitro group;

### Y is

1) —CO—, or
2) —CS—;

### X¹ is

CR⁴ wherein R⁴ is
   (a) a hydrogen atom,
   (b) a hydroxyl group,
   (c) a C₁₋₆ alkyl group (preferably a C₁₋₄ alkyl group, more preferably methyl group), or
   (d) —CO—C₁₋₆ alkoxy group (preferably —CO—C₁₋₄ alkoxy group, more preferably —CO-methoxy group), or
   (e) R³ and R⁴ (R³ is as defined above) may form, together with the carbon atoms they are bonded to, a saturated or unsaturated carbon ring having 3 to 14 carbon atoms (preferably an aromatic hydrocarbon, more preferably benzene ring);
      X²′ is an oxygen atom;

### —X³—X⁴— is —CH₂—CH₂—; and

ring A′ is

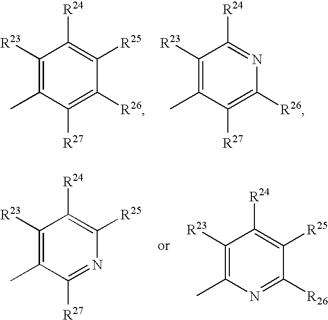

wherein
R²³ to R²⁷ are the same or different and each is
   1) a hydrogen atom,
   2) a halogen atom,
   3) a hydroxyl group,
   4) a C₁₋₆ alkyl group optionally substituted by one or more, the same or different halogen atoms (preferably fluorine atom)
   (preferably a C₁₋₄ alkyl group (preferably methyl group), trifluoromethyl group), or
   5) a nitro group, and
at least one of R²³ to R²⁷ is a hydroxyl group;
   is more preferable.

The compound selected from the following group or a pharmaceutically acceptable salt thereof is particularly preferable.
(1) (3-chloro-4-hydroxyphenyl)-(2,3-dihydrobenzo[1,4]oxazin-4-yl)-methanone,
(2) (3-bromo-4-hydroxyphenyl)-(2,3-dihydrobenzo[1,4]oxazin-4-yl)-methanone,
(3) (3,5-dichloro-4-hydroxyphenyl)-(2,3-dihydrobenzo[1,4]oxazin-4-yl)-methanone,
(4) (3,5-dibromo-4-hydroxyphenyl)-(2,3-dihydrobenzo[1,4]oxazin-4-yl)-methanone,
(5) (2,3-dihydrobenzo[1,4]oxazin-4-yl)-(4-hydroxy-3,5-diiodophenyl)-methanone,
(6) (3,5-difluoro-4-hydroxyphenyl)-(2,3-dihydrobenzo[1,4]oxazin-4-yl)-methanone,
(7) (2,3-dihydrobenzo[1,4]oxazin-4-yl)-(4-hydroxy-3,5-dimethylphenyl)-methanone,
(8) (3,5-dichloro-4-hydroxyphenyl)-(2,3-dihydrobenzo[1,4]thiazin-4-yl)-methanone,
(9) (3,5-dichloro-4-hydroxyphenyl)-(1-oxo-2,3-dihydro-1H-1λ⁴-benzo[1,4]thiazin-4-yl)-methanone,
(10) (3,5-dichloro-4-hydroxyphenyl)-(1,1-dioxo-2,3-dihydro-1H-1λ⁶-benzo[1,4]thiazin-4-yl)-methanone,
(11) (3,5-dichloro-4-hydroxyphenyl)-(2,3-dihydrobenzo[1,4]oxazin-4-yl)-methanethione,
(12) (3,5-dichloro-4-hydroxyphenyl)-(6-methyl-2,3-dihydrobenzo[1,4]oxazin-4-yl)-methanone,
(13) (3,5-dichloro-4-hydroxyphenyl)-(7-methyl-2,3-dihydrobenzo[1,4]oxazin-4-yl)-methanone,
(14) (3,5-dichloro-4-hydroxyphenyl)-(5-methyl-2,3-dihydrobenzo[1,4]oxazin-4-yl)-methanone,
(15) (3,5-dichloro-4-hydroxyphenyl)-(8-methyl-2,3-dihydrobenzo[1,4]oxazin-4-yl)-methanone,
(16) (3,5-dichloro-4-hydroxyphenyl)-(2,3-dihydronaphtho[2,1-b][1,4]oxazin-1-yl)-methanone,
(17) (3,5-dichloro-4-hydroxyphenyl)-(6-methoxy-2,3-dihydrobenzo[1,4]oxazin-4-yl)-methanone,
(18) (3,5-dichloro-4-hydroxyphenyl)-(7-methoxy-2,3-dihydrobenzo[1,4]oxazin-4-yl)-methanone,
(19) (3,5-dichloro-4-hydroxyphenyl)-(6-hydroxy-2,3-dihydrobenzo[1,4]oxazin-4-yl)-methanone,
(20) (3,5-dichloro-4-hydroxyphenyl)-(7-hydroxy-2,3-dihydrobenzo[1,4]oxazin-4-yl)-methanone,
(21) 4-(3,5-dichloro-4-hydroxybenzoyl)-3,4-dihydro-2H-benzo[1,4]oxazine-6-sulfonic acid diethylamide,
(22) 2,6-dichloro-4-(2,3-dihydrobenzo[1,4]oxazine-4-sulfonyl)phenol,
(23) (6-tert-butyl-2,3-dihydrobenzo[1,4]oxazin-4-yl)-(3,5-dichloro-4-hydroxyphenyl)-methanone,
(24) 4-(3,5-dichloro-4-hydroxybenzoyl)-4H-benzo[1,4]oxazin-3-one,
(25) 4-(3,5-dichloro-4-hydroxybenzoyl)-3,4-dihydro-2H-benzo[1,4]oxazine-6-sulfonamide,
(26) (3,5-dichloro-4-hydroxyphenyl)-(3,4-dihydro-2H-quinolin-1-yl)-methanone,
(27) (3,5-dichloro-4-hydroxyphenyl)-(2,3,4,5-tetrahydrobenzo[b]azepin-1-yl)-methanone,
(28) (4-amino-3,5-dichlorophenyl)-(2,3-dihydrobenzo[1,4]oxazin-4-yl)-methanone,
(29) (5-chloro-6-hydroxypyridin-3-yl)-(2,3-dihydrobenzo[1,4]oxazin-4-yl)-methanone,
(30) (2,3-dihydrobenzo[1,4]oxazin-4-yl)-(4-hydroxy-3,5-dinitrophenyl)-methanone,
(31) (3-chloro-4-hydroxy-5-nitrophenyl)-(2,3-dihydrobenzo[1,4]oxazin-4-yl)-methanone,
(32) (3,5-dichloro-4-hydroxyphenyl)-(2,8-diisopropyl-2,3-dihydrobenzo[1,4]oxazin-4-yl)-methanone,
(33) (3,5-dichloro-4-hydroxyphenyl)-[6-(pyrrolidine-1-sulfonyl)-2,3-dihydrobenzo[1,4]oxazin-4-yl]-methanone,
(34) 4-(3,5-dichloro-4-hydroxybenzoyl)-3,4-dihydro-2H-benzo[1,4]oxazine-6-sulfonic acid ethylamide,
(35) 4-(3,5-dichloro-4-hydroxybenzoyl)-3,4-dihydro-2H-benzo[1,4]oxazine-6-sulfonic acid dimethylamide,
(36) (3,5-dichloro-4-hydroxyphenyl)-(2,3-dihydropyrido[3,2-b][1,4]oxazin-4-yl)-methanone,
(37) 5-(3,5-dichloro-4-hydroxybenzoyl)-1,3,4,5-tetrahydrobenzo[b][1,4]diazepin-2-one,
(38) (3,5-dichloro-2-hydroxyphenyl)-(2,3-dihydrobenzo[1,4]oxazin-4-yl)-methanone,
(39) (2,3-dihydrobenzo[1,4]oxazin-4-yl)-(4-hydroxy-3-trifluoromethylphenyl)-methanone,
(40) (3-chloro-4-hydroxy-5-methoxyphenyl)-(2,3-dihydrobenzo[1,4]oxazin-4-yl)-methanone,
(41) (4-chloro-3-hydroxyphenyl)-(2,3-dihydrobenzo[1,4]oxazin-4-yl)-methanone,
(42) (2,6-dichloropyridin-4-yl)-(2,3-dihydrobenzo[1,4]oxazin-4-yl)-methanone,
(43) (2,3-dihydrobenzo[1,4]oxazin-4-yl)-(4-nitrophenyl)-methanone,
(44) (3,5-dichloro-4-hydroxyphenyl)-(6-fluoro-2,3-dihydrobenzo[1,4]oxazin-4-yl)-methanone,
(45) 2-(2,3-dihydrobenzo[1,4]oxazine-4-carbonyl)benzoic acid,
(46) methyl 4-(2,3-dihydrobenzo[1,4]oxazine-4-carbonyl)benzoate,
(47) 4-(2,3-dihydrobenzo[1,4]oxazine-4-carbonyl)benzoic acid,
(48) methyl 3-(2,3-dihydrobenzo[1,4]oxazine-4-carbonyl)benzoate,
(49) 3-(2,3-dihydrobenzo[1,4]oxazine-4-carbonyl)benzoic acid,
(50) (3,5-dichloro-2,4-dihydroxyphenyl)-(2,3-dihydrobenzo[1,4]oxazin-4-yl)-methanone,
(51) (6-chloro-2,3-dihydrobenzo[1,4]oxazin-4-yl)-(3,5-dichloro-4-hydroxyphenyl)-methanone,
(52) (7-chloro-2,3-dihydrobenzo[1,4]oxazin-4-yl)-(3,5-dichloro-4-hydroxyphenyl)-methanone,
(53) [4-(3,5-dichloro-4-hydroxybenzoyl)-3,4-dihydro-2H-quinoxalin-1-yl]-(3,5-dichloro-4-hydroxyphenyl)-methanone,
(54) (3,5-dichloro-4-hydroxyphenyl)-(3,4-dihydro-2H-quinoxalin-1-yl)-methanone,
(55) methyl 4-(3,5-dichloro-4-hydroxybenzoyl)-3,4-dihydro-2H-benzo[1,4]oxazine-6-carboxylate,
(56) (3,5-dichloro-4-hydroxyphenyl)-(6-hydroxymethyl-2,3-dihydrobenzo[1,4]oxazin-4-yl)-methanone,
(57) 4-(3,5-dichloro-4-hydroxybenzoyl)-3,4-dihydro-2H-benzo[1,4]oxazine-6-carboxylic acid,
(58) methyl 4-(3,5-dichloro-4-hydroxybenzoyl)-3,4-dihydro-2H-benzo[1,4]oxazine-5-carboxylate,
(59) methyl 4-(3,5-dichloro-4-hydroxybenzoyl)-3,4-dihydro-2H-benzo[1,4]oxazine-7-carboxylate,
(60) 4-(3,5-dichloro-4-hydroxybenzoyl)-3,4-dihydro-2H-benzo[1,4]oxazine-7-carboxylic acid,
(61) methyl 4-(3,5-dichloro-4-hydroxybenzoyl)-3,4-dihydro-2H-benzo[1,4]oxazine-8-carboxylate,
(62) 4-(3,5-dichloro-4-hydroxybenzoyl)-3,4-dihydro-2H-benzo[1,4]oxazine-8-carboxylic acid,
(63) (3,5-dichlorophenyl)-(2,3-dihydrobenzo[1,4]oxazin-4-yl)-methanone,
(64) (3,5-dichloro-4-hydroxyphenyl)-(phenoxazin-10-yl)-methanone,
(65) (3,5-dichloro-4-hydroxyphenyl)-(6-phenyl-2,3-dihydrobenzo[1,4]oxazin-4-yl)-methanone,
(66) (3,5-dichloro-4-hydroxyphenyl)-(6,8-dimethyl-2,3-dihydrobenzo[1,4]oxazin-4-yl)-methanone,
(67) (3,5-dichloro-4-hydroxyphenyl)-(6-nitro-2,3-dihydrobenzo[1,4]oxazin-4-yl)-methanone,
(68) (6-amino-2,3-dihydrobenzo[1,4]oxazin-4-yl)-(3,5-dichloro-4-hydroxyphenyl)-methanone,
(69) (3,5-dibromo-4-hydroxyphenyl)-(6-nitro-2,3-dihydrobenzo[1,4]oxazin-4-yl)-methanone,
(70) (3,5-dichloro-4-hydroxyphenyl)-(7-nitro-2,3-dihydrobenzo[1,4]oxazin-4-yl)-methanone,
(71) (7-amino-2,3-dihydrobenzo[1,4]oxazin-4-yl)-(3,5-dichloro-4-hydroxyphenyl)-methanone,
(72) N-[4-(3,5-dichloro-4-hydroxybenzoyl)-3,4-dihydro-2H-benzo[1,4]oxazin-7-yl]-methanesulfonamide,
(73) 1-[4-(3,5-dichloro-4-hydroxybenzoyl)-3,4-dihydro-2H-quinoxalin-1-yl]-ethanone,
(74) (3,5-dichloro-4-hydroxyphenyl)-(4-methyl-3,4-dihydro-2H-quinoxalin-1-yl)-methanone,
(75) (2,3-dihydrobenzo[1,4]oxazin-4-yl)-(4-hydroxy-3-nitrophenyl)-methanone,
(76) (3,5-dichloro-4-hydroxyphenyl)-(2-methyl-2,3-dihydroindol-1-yl)-methanone,
(77) (3,5-dichloro-4-hydroxyphenyl)-(2,3-dihydroindol-1-yl)-methanone,
(78) (5-amino-2,3-dihydroindol-1-yl)-(3,5-dichloro-4-hydroxyphenyl)-methanone,
(79) (3,5-dibromo-4-hydroxyphenyl)-(6-fluoro-2,3-dihydrobenzo[1,4]oxazin-4-yl)-methanone,
(80) (3,5-dibromo-4-hydroxyphenyl)-(2,3-dihydronaphtho[2,1-b][1,4]oxazin-1-yl)-methanone,
(81) (3,5-dibromo-4-hydroxyphenyl)-(6-methyl-2,3-dihydrobenzo[1,4]oxazin-4-yl)-methanone,
(82) (6-chloro-2,3-dihydrobenzo[1,4]oxazin-4-yl)-(3,5-dibromo-4-hydroxyphenyl)-methanone,
(83) (3,5-dichloro-4-hydroxyphenyl)-(4-methanesulfonyl-3,4-dihydro-2H-quinoxalin-1-yl)-methanone,
(84) (3,5-dichloro-4-hydroxyphenyl)-(6-ethanesulfonyl-2,3-dihydrobenzo[1,4]oxazin-4-yl)-methanone,
(85) (3,5-dichloro-4-hydroxyphenyl)-(6-trifluoromethyl-2,3-dihydrobenzo[1,4]oxazin-4-yl)-methanone,
(86) (3,5-dichloro-4-methoxyphenyl)-(2,3-dihydrobenzo[1,4]oxazin-4-yl)-methanone,
(87) 2,6-dichloro-4-(2,3-dihydrobenzo[1,4]oxazine-4-carbonyl)phenyl acetate,
(88) (2,3-dihydrobenzo[1,4]oxazin-4-yl)-(4-hydroxyphenyl)-methanone,
(89) (3,5-dichloro-4-hydroxyphenyl)-(5-hydroxy-2,3-dihydrobenzo[1,4]oxazin-4-yl)-methanone,
(90) (3,5-dichloro-4-hydroxyphenyl)-(8-hydroxy-2,3-dihydrobenzo[1,4]oxazin-4-yl)-methanone,
(91) ethyl [2,6-dichloro-4-(2,3-dihydrobenzo[1,4]oxazine-4-carbonyl)phenoxy]acetate,
(92) [2,6-dichloro-4-(2,3-dihydrobenzo[1,4]oxazine-4-carbonyl)phenoxy]acetic acid
(93) (3,5-dichloro-4-hydroxyphenyl)-(3-methyl-2,3-dihydrobenzo[1,4]oxazin-4-yl)-methanone,
(94) N-[2,6-dichloro-4-(2,3-dihydrobenzo[1,4]oxazine-4-carbonyl)phenyl]methanesulfonamide, and
(95) (3,5-dichloro-4-hydroxyphenyl)-(7,8-dihydro-6H-5-oxa-9-azabenzocyclohepten-9-yl)-methanone.

Of these, (3,5-dichloro-4-hydroxyphenyl)-(2,3-dihydrobenzo[1,4]oxazin-4-yl)-methanone is most preferable.

Compound [1] can also be obtained as a crystal. For example,

(1) (3-chloro-4-hydroxyphenyl)-(2,3-dihydrobenzo[1,4]oxazin-4-yl)-methanone having characteristic diffraction peaks at 12.94°, 17.36°, 23.50°, 26.10° and 26.94° of diffraction angle (2θ) as measured by powder X-ray crystal diffraction (Reference Example 1),
(2) (3-bromo-4-hydroxyphenyl)-(2,3-dihydrobenzo[1,4]oxazin-4-yl)-methanone having characteristic diffraction peaks at 16.96°, 17.54°, 21.66°, 25.68° and 26.62° of diffraction angle (2θ) as measured by powder X-ray crystal diffraction (Reference Example 2),
(3) (3,5-dichloro-4-hydroxyphenyl)-(2,3-dihydrobenzo[1,4]oxazin-4-yl)-methanone having characteristic diffraction peaks at 7.40°, 14.90°, 22.68°, 22.92° and 26.46° of diffraction angle (2θ) as measured by powder X-ray crystal diffraction (Reference Example 3),
(4) (3,5-dibromo-4-hydroxyphenyl)-(2,3-dihydrobenzo[1,4]oxazin-4-yl)-methanone having characteristic diffraction peaks at 7.40°, 14.92°, 16.64°, 22.68° and 26.12° of diffraction angle (2θ) as measured by powder X-ray crystal diffraction (Reference Example 4),
(5) (2,3-dihydrobenzo[1,4]oxazin-4-yl)-(4-hydroxy-3,5-diiodophenyl)-methanone having characteristic diffraction peaks at 7.28°, 15.84°, 23.10°, 29.54° and 37.16° of diffraction angle (2θ) as measured by powder X-ray crystal diffraction (Reference Example 5),
(6) (3,5-difluoro-4-hydroxyphenyl)-(2,3-dihydrobenzo[1,4]oxazin-4-yl)-methanone having characteristic diffraction peaks at 15.58°, 17.92°, 18.48°, 19.86° and 25.90° of diffraction angle (2θ) as measured by powder X-ray crystal diffraction (Reference Example 6),
(7) (2,3-dihydrobenzo[1,4]oxazin-4-yl)-(4-hydroxy-3,5-dimethylphenyl)-methanone having characteristic diffraction peaks at 12.46°, 23.38°, 23.98°, 24.32° and 25.24° of diffraction angle (2θ) as measured by powder X-ray crystal diffraction (Reference Example 7),
(8) (3,5-dichloro-4-hydroxyphenyl)-(1,1-dioxo-2,3-dihydro-1H-1λ⁶-benzo[1,4]thiazin-4-yl)-methanone having characteristic diffraction peaks at 16.62°, 18.64°, 19.20°, 21.60° and 23.14° of diffraction angle (2θ) as measured by powder X-ray crystal diffraction (Reference Example 10),
(9) (3,5-dichloro-4-hydroxyphenyl)-(6-methyl-2,3-dihydrobenzo[1,4]oxazin-4-yl)-methanone having characteristic diffraction peaks at 11.58°, 14.36°, 22.18°, 22.48° and 23.36° of diffraction angle (2θ) as measured by powder X-ray crystal diffraction (Reference Example 12),
(10) (3,5-dichloro-4-hydroxyphenyl)-(5-methyl-2,3-dihydrobenzo[1,4]oxazin-4-yl)-methanone having characteristic diffraction peaks at 11.82°, 14.88°, 22.62°, 25.56° and 26.62° of diffraction angle (2θ) as measured by powder X-ray crystal diffraction (Reference Example 14),
(11) (3,5-dichloro-4-hydroxyphenyl)-(8-methyl-2,3-dihydrobenzo[1,4]oxazin-4-yl)-methanone having characteristic diffraction peaks at 7.02°, 14.14°, 21.30°, 21.80° and 26.56° of diffraction angle (2θ) as measured by powder X-ray crystal diffraction (Reference Example 15),
(12) (3,5-dichloro-4-hydroxyphenyl)-(2,3-dihydronaphtho[2,1-b][1,4]oxazin-1-yl)-methanone having characteristic diffraction peaks at 12.40°, 18.36°, 21.34°, 23.66° and 24.24° of diffraction angle (2θ) as measured by powder X-ray crystal diffraction (Reference Example 16),
(13) (3,5-dichloro-4-hydroxyphenyl)-(6-methoxy-2,3-dihydrobenzo[1,4]oxazin-4-yl)-methanone having characteristic diffraction peaks at 10.54°, 11.24°, 21.24°, 21.60° and 24.38° of diffraction angle (2θ) as measured by powder X-ray crystal diffraction (Reference Example 17),
(14) (3,5-dichloro-4-hydroxyphenyl)-(7-methoxy-2,3-dihydrobenzo[1,4]oxazin-4-yl)-methanone having characteristic diffraction peaks at 12.32°, 13.52°, 22.70°, 24.88° and 26.10° of diffraction angle (2θ) as measured by powder X-ray crystal diffraction (Reference Example 18),
(15) (3,5-dichloro-4-hydroxyphenyl)-(6-hydroxy-2,3-dihydrobenzo[1,4]oxazin-4-yl)-methanone having characteristic diffraction peaks at 11.16°, 14.58°, 21.38°, 22.54° and 22.76° of diffraction angle (2θ) as measured by powder X-ray crystal diffraction (Reference Example 19),
(16) (3,5-dichloro-4-hydroxyphenyl)-(7-hydroxy-2,3-dihydrobenzo[1,4]oxazin-4-yl)-methanone having characteristic diffraction peaks at 15.46°, 20.90°, 22.92°, 24.68° and 25.24° of diffraction angle (2θ) as measured by powder X-ray crystal diffraction (Reference Example 20),
(17) 4-(3,5-dichloro-4-hydroxybenzoyl)-3,4-dihydro-2H-benzo[1,4]oxazine-6-sulfonic acid diethylamide having characteristic diffraction peaks at 13.48°, 15.52°, 19.18°, 20.58° and 21.80° of diffraction angle (2θ) as measured by powder X-ray crystal diffraction (Reference Example 21),
(18) (3,5-dichloro-4-hydroxyphenyl)-(3,4-dihydro-2H-quinolin-1-yl)-methanone having characteristic diffraction peaks at 11.66°, 22.20°, 22.48°, 24.68° and 25.52° of diffraction angle (2θ) as measured by powder X-ray crystal diffraction (Reference Example 26),
(19) (3,5-dichloro-4-hydroxyphenyl)-(2,3,4,5-tetrahydrobenzo[b]azepin-1-yl)-methanone having characteristic diffraction peaks at 17.60°, 21.78°, 22.78°, 24.96° and 32.98° of diffraction angle (2θ) as measured by powder X-ray crystal diffraction (Reference Example 27),
(20) (3-chloro-4-hydroxy-5-nitrophenyl)-(2,3-dihydrobenzo[1,4]oxazin-4-yl)-methanone having characteristic diffraction peaks at 12.16°, 14.90°, 22.16°, 23.46° and 24.52° of diffraction angle (2θ) as measured by powder X-ray crystal diffraction (Reference Example 31),
(21) (3,5-dichloro-4-hydroxyphenyl)-(2,3-dihydropyrido[3,2-b][1,4]oxazin-4-yl)-methanone having characteristic diffraction peaks at 11.58°, 14.78°, 18.80°, 23.66° and 25.52° of diffraction angle (2θ) as measured by powder X-ray crystal diffraction (Reference Example 36),
(22) (3,5-dichloro-2-hydroxyphenyl)-(2,3-dihydrobenzo[1,4]oxazin-4-yl)-methanone having characteristic diffraction peaks at 19.94°, 21.86°, 22.52°, 23.84° and 26.14° of diffraction angle (2θ) as measured by powder X-ray crystal diffraction (Reference Example 38),
(23) (2,3-dihydrobenzo[1,4]oxazin-4-yl)-(4-hydroxy-3-trifluoromethylphenyl)-methanone having characteristic diffraction peaks at 7.58°, 12.10°, 15.24°, 22.30° and 24.40° of diffraction angle (2θ) as measured by powder X-ray crystal diffraction (Reference Example 39),
(24) (3-chloro-4-hydroxy-5-methoxyphenyl)-(2,3-dihydrobenzo[1,4]oxazin-4-yl)-methanone having characteristic diffraction peaks at 10.06°, 17.10°, 17.48°, 21.78° and 22.26° of diffraction angle (2θ) as measured by powder X-ray crystal diffraction (Reference Example 40),
(25) (4-chloro-3-hydroxyphenyl)-(2,3-dihydrobenzo[1,4]oxazin-4-yl)-methanone having characteristic diffraction peaks at 19.06°, 20.76°, 22.30°, 26.58° and 27.60° of diffraction angle (2θ) as measured by powder X-ray crystal diffraction (Reference Example 41),
(26) (3,5-dichloro-4-hydroxyphenyl)-(6-fluoro-2,3-dihydrobenzo[1,4]oxazin-4-yl)-methanone having characteristic diffraction peaks at 11.70°, 22.44°, 22.74°, 23.58° and 23.90° of diffraction angle (2θ) as measured by powder X-ray crystal diffraction (Reference Example 44),
(27) (3,5-dichloro-2,4-dihydroxyphenyl)-(2,3-dihydrobenzo[1,4]oxazin-4-yl)-methanone having characteristic diffraction peaks at 7.26°, 22.62°, 25.12°, 25.70° and 27.92° of diffraction angle (2θ) as measured by powder X-ray crystal diffraction (Reference Example 50),
(28) (6-chloro-2,3-dihydrobenzo[1,4]oxazin-4-yl)-(3,5-dichloro-4-hydroxyphenyl)-methanone having characteristic diffraction peaks at 11.72°, 21.78°, 22.54°, 22.82° and 23.68° of diffraction angle (2θ) as measured by powder X-ray crystal diffraction (Reference Example 51),
(29) (3,5-dichloro-4-hydroxyphenyl)-(6-hydroxymethyl-2,3-dihydrobenzo[1,4]oxazin-4-yl)-methanone having characteristic diffraction peaks at 21.04°, 22.22°, 23.74°, 24.720 and 27.38° of diffraction angle (2θ) as measured by powder X-ray crystal diffraction (Reference Example 56),
(30) (3,5-dichloro-4-hydroxyphenyl)-(6,8-dimethyl-2,3-dihydrobenzo[1,4]oxazin-4-yl)-methanone having characteristic diffraction peaks at 14.80°, 16.44°, 22.28°, 22.76° and 24.14° of diffraction angle (2θ) as measured by powder X-ray crystal diffraction (Reference Example 66),
(31) (3,5-dichloro-4-hydroxyphenyl)-(6-nitro-2,3-dihydrobenzo[1,4]oxazin-4-yl)-methanone having characteristic diffraction peaks at 14.52°, 21.52°, 21.88°, 27.84° and 30.92° of diffraction angle (2θ) as measured by powder X-ray crystal diffraction (Reference Example 67),
(32) (3,5-dibromo-4-hydroxyphenyl)-(6-nitro-2,3-dihydrobenzo[1,4]oxazin-4-yl)-methanone having characteristic diffraction peaks at 7.16°, 14.40°, 20.96°, 27.58° and 34.62° of diffraction angle (2θ) as measured by powder X-ray crystal diffraction (Reference Example 69),
(33) 1-[4-(3,5-dichloro-4-hydroxybenzoyl)-3,4-dihydro-2H-quinoxalin-1-yl]-ethanone having characteristic diffraction peaks at 15.48°, 19.24°, 22.48°, 25.54° and 30.30° of diffraction angle (2θ) as measured by powder X-ray crystal diffraction (Reference Example 73),
(34) (3,5-dichloro-4-hydroxyphenyl)-(2-methyl-2,3-dihydroindol-1-yl)-methanone having characteristic diffraction peaks at 10.48°, 11.32°, 20.18°, 22.84° and 25.76° of diffraction angle (2θ) as measured by powder X-ray crystal diffraction (Reference Example 76),
(35) (3,5-dichloro-4-hydroxyphenyl)-(2,3-dihydroindol-1-yl)-methanone having characteristic diffraction peaks at 12.10°, 22.48°, 23.40°, 24.42° and 25.98° of diffraction angle (2θ) as measured by powder X-ray crystal diffraction (Reference Example 77),
(36) (3,5-dibromo-4-hydroxyphenyl)-(6-fluoro-2,3-dihydrobenzo[1,4]oxazin-4-yl)-methanone having characteristic diffraction peaks at 9.30°, 14.52°, 18.64°, 23.96° and 27.12° of diffraction angle (2θ) as measured by powder X-ray crystal diffraction (Reference Example 79),
(37) (3,5-dibromo-4-hydroxyphenyl)-(2,3-dihydronaphtho[2,1-b][1,4]oxazin-1-yl)-methanone having characteristic diffraction peaks at 8.92°, 17.98°, 18.26°, 21.10° and 23.40° of diffraction angle (2θ) as measured by powder X-ray crystal diffraction (Reference Example 80),
(38) (3,5-dibromo-4-hydroxyphenyl)-(6-methyl-2,3-dihydrobenzo[1,4]oxazin-4-yl)-methanone having characteristic diffraction peaks at 7.12°, 14.32°, 21.62°, 21.92° and 22.80° of diffraction angle (2θ) as measured by powder X-ray crystal diffraction (Reference Example 81),
(39) (6-chloro-2,3-dihydrobenzo[1,4]oxazin-4-yl)-(3,5-dibromo-4-hydroxyphenyl)-methanone having characteristic diffraction peaks at 7.22°, 14.54°, 22.34°, 29.08° and 33.22° of diffraction angle (2θ) as measured by powder X-ray crystal diffraction (Reference Example 82),
(40) (3,5-dichloro-4-hydroxyphenyl)-(6-trifluoromethyl-2,3-dihydrobenzo[1,4]oxazin-4-yl)-methanone having characteristic diffraction peaks at 17.58°, 19.00°, 21.04°, 21.52° and 23.24° of diffraction angle (2θ) as measured by powder X-ray crystal diffraction (Reference Example 85),
(41) 2,6-dichloro-4-(2,3-dihydrobenzo[1,4]oxazine-4-carbonyl)phenyl acetate having characteristic diffraction peaks at 15.76°, 16.38°, 24.12°, 25.88° and 27.62° of diffraction angle (2θ) as measured by powder X-ray crystal diffraction (Reference Example 87), and the like can be mentioned.

The “pharmaceutically acceptable salt thereof” may be any as long as it forms nontoxic salts with compound [1] (hereinafter including compound [2]) and, for example, salts with inorganic acids such as hydrochloric acid, sulfuric acid, phosphoric acid, hydrobromic acid and the like; salts with organic acids such as oxalic acid, malonic acid, citric acid, fumaric acid, lactic acid, malic acid, succinic acid, tartaric acid, acetic acid, trifluoroacetic acid, gluconic acid, ascorbic acid, methanesulfonic acid, benzenesulfonic acid and the like; salts with inorganic bases such as sodium hydroxide, potassium hydroxide, calcium hydroxide, magnesium hydroxide, ammonium hydroxide and the like; salts with organic bases such as methylamine, diethylamine, triethylamine, triethanolamine, ethylenediamine, tris(hydroxymethyl)methylamine, guanidine, choline, cinchonine and the like; salts with amino acids such as lysine, arginine, alanine and the like, and the like can be mentioned.

Compound [1] also encompasses water-containing products, hydrates and solvates thereof.

In addition, compound [1] has various isomers. For example, E form and Z form can be present as geometric isomers, when an asymmetric carbon atom is present, enantiomer and diastereomer are present as stereoisomers based thereon, and tautomers can also be present. Therefore, compound [1] encompasses all of these isomers and mixtures thereof. Moreover, prodrug compounds of such compounds as equivalent compounds of compound [1] can also be useful pharmaceutical agents.

As used herein, the “prodrug” is a derivative having a chemically or metabolically decomposable group, which shows a pharmaceutical activity upon decomposition by hydrolysis or solvolysis, or under physiological conditions. A prodrug is used for, for example, improving absorption by oral administration or targeting the object site. Inasmuch as what the chemically or metabolically decomposable group is, and how to introduced the group into a compound have been sufficiently established in the field of pharmaceutical agents, such known techniques can be employed in compound [1]. As the moiety to be modified for producing a prodrug, for example, highly reactive functional groups such as a hydroxyl group, a carboxyl group, an amino group, a thiol group and the like in the compound of the present invention can be mentioned. For example, compound [1] wherein —OR¹³ is a hydroxyl group, compound [2] wherein —OR¹³ or —OR¹³′ is a hydroxyl group, and the like can be mentioned.

For example, a derivative wherein a substituent such as —CO—C₁₋₆ alkyl group, —CO₂—C₁₋₆ alkyl group, —CONH—C₁₋₆ alkyl group, —CO—C₂₋₆ alkenyl group, —CO₂—C₂₋₆ alkenyl group, —CONH—C₂₋₆ alkenyl group, —CO-aryl group, —CO₂-aryl group, —CONH-aryl group, —CO-heterocyclic group, —CO₂-heterocyclic group, —CONH-heterocyclic group (the C₁₋₆ alkyl group, C₂₋₆ alkenyl group, aryl group and heterocyclic group are each optionally substituted by halogen atom, C₁₋₆ alkyl group, hydroxyl group, C₁₋₆ alkoxy group, carboxy group, amino group, amino acid residue, —PO₃H₂, —SO₃H, —CO-polyethylene glycol residue, —CO₂-polyethylene glycol residue, —CO-polyethylene glycol monoalkyl ether residue, —CO₂-polyethylene glycol monoalkyl ether residue and the like) and the like has been introduced into a hydroxyl group can be mentioned.

In addition, a derivative wherein a substituent such as —CO—C₁₋₆ alkyl group, —CO₂—C₁₋₆ alkyl group, —CO—C₂₋₆ alkenyl group, —CO₂—C₂₋₆ alkenyl group, —CO-aryl group, —CO₂-aryl group, —CO-heterocyclic group, —CO₂-heterocyclic group (the C₁₋₆ alkyl group, C₂₋₆ alkenyl group, aryl group and heterocyclic group are each optionally substituted by halogen atom, C₁₋₆ alkyl group, hydroxyl group, C₁₋₆ alkoxy group, carboxy group, amino group, amino acid residue, —PO₃H₂, —SO₃H, —CO-polyethylene glycol residue, —CO₂-polyethylene glycol residue, —CO-polyethylene glycol monoalkyl ether residue, —CO₂-polyethylene glycol monoalkyl ether residue, —PO₃H₂ and the like) and the like has been introduced into an amino group can be mentioned.

Furthermore, a derivative wherein a substituent such as C₁₋₆ alkoxy group, aryloxy group (the C₁₋₆ alkoxy group and aryloxy group are each optionally substituted by halogen atom, C₁₋₆ alkyl group, hydroxyl group, C₁₋₆ alkoxy group, carboxy group, amino group, amino acid residue, —PO₃H₂, —SO₃H, polyethylene glycol residue, polyethylene glycol monoalkyl ether residue and the like) and the like has been introduced into a carboxyl group can be mentioned.

The pharmaceutical composition of the present invention is effective for the prophylaxis or treatment of pathology showing involvement of uric acid since

(1) it does not substantially inhibit CYP, and
(2) it inhibits a URAT1 activity and decreases the blood uric acid level.

To not “substantially inhibit CYP” means that the function of the drug metabolizing enzyme, cytochrome P450 (CYP), preferably CYP2D6, CYP2C8, CYP2C9, CYP2C19 or CYP3A4, more preferably CYP2C9, is not substantially inhibited and that, for example, the concentration of a substance that dose not substantially inhibit CYP necessary for inhibiting CYP by 50% is not less than 1 μM, preferably not less than 3 μM, more preferably not less than 10 μM, still more preferably not less than 25 μM, particularly preferably not less than 50 μM, based on the conditions of the below-mentioned Reference Experimental Example 2, preferably under the conditions of Reference Experimental Example 2.

To “inhibit URAT1 activity” means to specifically inhibit the function of URAT1 as a uric acid transporter to eliminate or attenuate the activity and means, for example, to specifically inhibit the function of URAT1 based on the conditions of the below-mentioned Reference Experimental Example 1 and preferably means that the concentration necessary for inhibiting URAT1 by 50% is less than 3 μM, more preferably less than 1 μM, more preferably less than 0.3 μM, still more preferably less than 0.1 μM, yet more preferably less than 0.03 μM, under the conditions of the below-mentioned Reference Experimental Example 1. The URAT1 activity inhibitor does not include biological substrates of URAT1, such as uric acid and the like.

To “decrease the blood uric acid level” means to decrease uric acid (including uric acid salt) in blood (including in serum and plasma), preferably to decrease high blood uric acid level, more preferably serum uric acid level, to less than 8 mg/dL (preferably less than 7 mg/dl, more preferably less than 6 mg/dL, as serum uric acid level).

The “pathology showing involvement of uric acid” means pathology in which uric acid (including uric acid salt) in the body such as blood (including in serum and plasma) or urine is involved. Specifically, pathology caused by high blood (including in serum and plasma) uric acid level or urine uric acid level such as hyperuricemia, gouty tophus, acute gouty arthritis, chronic gouty arthritis, gouty kidney, urolithiasis, renal function disorder, coronary artery disease, ischemic heart disease and the like can be mentioned.

For example, the “high blood uric acid level” means a serum uric acid level of not less than 6 mg/dL, preferably not less than 7 mg/dL, more preferably not less than 8 mg/dL. The “pathology caused by high blood uric acid level” is pathology caused by high blood uric acid level, or to which high blood uric acid level contributes. For example, according to “Guideline for the management of hyperuricemia and gout (1st Edit.)” (Gout and Nucleic Acid Metabolism, vol. 26, suppl. 1 (2002), Japanese Society of Gout and Nucleic Acid Metabolism), 7 mg/dL, which is a dissolution concentration of uric acid in plasma, is the normal upper limit, irrespective of sex•age, and any value exceeding this level is defined to be hyperuricemia, and concludes that the serum uric acid level should be desirably controlled to not more than 6 mg/dL for the treatment of hyperuricemia or gout and for the prevention of the onset of gout arthritis.

The “pharmaceutical composition” includes, besides what is called a “composition” comprising an active ingredient as a pharmaceutical agent and a pharmaceutically acceptable additive and the like, a combination agent with other pharmaceutical agents, and the like. It is needless to say that the pharmaceutical composition of the present invention can be concurrently used with any other pharmaceutical agent within the acceptable range in clinical situations. Therefore, the present pharmaceutical composition can also be considered a pharmaceutical composition to be combined with other pharmaceutical agents.

Compound [1] can be contained as an active ingredient of a pharmaceutical composition, a URAT1 activity inhibitor, an agent for decreasing a blood uric acid value, or an agent for the prophylaxis or treatment of pathology showing involvement of uric acid, along with one or more pharmaceutically acceptable additives.

The present inventors have found that, as shown in the following Experimental Examples, compound [1] is decomposed when compound [1] comes into contact with a basic additive. Accordingly, the pharmaceutical composition of the present invention is characterized in that compound [1] does not (substantially) contact with a basic additive so as to improve time-course stability of compound [1] during the manufacturing process or storage of the pharmaceutical composition.

By the “compound [1] is not (substantially) in contact with a basic additive” are meant, for example, the following embodiments.

(1) Embodiment wherein a layer containing compound [1] is not in contact with a layer containing a basic additive

This embodiment specifically includes

(a) containing compound [1] and the basic additive separately in the layers free of contact with each other in a multi-layer tablet (for example, first and third layers etc.),
(b) containing compound [1] and the basic additive separately in the layers free of contact with each other in a dry-coated tablet (for example, coated inner core tablet and outer layer etc.)
and the like.
(2) Embodiment wherein the probability of contact between compound [1] and a basic additive is decreased by setting the content of the basic additive to a level far lower than the amount generally used for pharmaceutical preparations, for example, the amount as an excipient (for example, about 10-30 parts by weight per 1 part by weight compound of the active ingredient)

This embodiment specifically includes

(a) excluding a basic additive (all additives are not basic additives, for example, all additives are selected from the group consisting of acidic additives and neutral additives),
(b) containing a basic additive at an acceptable content of less than 1 part by weight (preferably, less than 0.5 part by weight, more preferably less than 0.1 part by weight, more preferably less than 0.025 part by weight) per 1 part by weight of compound [1],
and the like.

While the amount of the basic additive to be contained in the pharmaceutical composition of the present invention is not particularly limited, it is preferably 0-15 w/w %, more preferably 0-5 w/w %, relative to the total amount of the pharmaceutical composition.

In the pharmaceutical composition of the present invention, an embodiment wherein the probability of contact between compound [1] and a basic additive is decreased by setting the content of the basic additive to a level far lower than the amount generally used for pharmaceutical preparations is preferable. Particularly, it is more preferable to not add a basic additive to the pharmaceutical composition of the present invention. In this way, the time-course stability of compound [1] during the manufacturing process or storage of the pharmaceutical composition can be improved.

The “basic additive” means an additive that shows a pH exceeding 8 at ambient temperature (15-25° C.), when it is prepared into an aqueous solution or suspension by adding water (100 mL) to the additive (5.0 g). While the measured value varies depending on the measurement device, measurement conditions and the like, it is considered to fall within the above-mentioned pH range as long as it is within regular error range.

In addition, it also means an additive having a standard pH value exceeding 8, which is described in The Japanese Pharmacopoeia, 14th Edition or Japanese Pharmaceutical Excipients 2003.

Specific examples of the basic additive include alkali metal hydroxides (e.g., sodium hydroxide, potassium hydroxide etc.), alkaline earth metal hydroxides (e.g., magnesium hydroxide, barium hydroxide etc.), alkali metal carbonates (e.g., sodium carbonate, potassium carbonate etc.), alkaline earth metal carbonates (e.g., magnesium carbonate, calcium carbonate etc.), alkali metal hydrogencarbonates (e.g., sodium hydrogencarbonate etc.), alkaline earth metal hydrogencarbonates (e.g., magnesium hydrogencarbonate, barium hydrogencarbonate etc.), alkali metal silicates (e.g., sodium silicate etc.), alkaline earth metal silicates (e.g., magnesium silicate, calcium silicate etc.) and the like.

Particularly specific examples of the basic additive include alkali metal silicate, alkaline earth metal silicate and the like, and concrete examples thereof include calcium silicate.

The “acidic additive” and “neutral additive” mean additives other than the “basic additive” defined above.

Specific examples of the “acidic additive” and “neutral additive” include D-mannitol, crystalline cellulose, low-substituted hydroxypropylcellulose, light anhydrous silicic acid, crospovidone, magnesium stearate, lactose, cornstarch, croscarmellose sodium, carmellose, carmellose sodium, carmellose calcium, carboxymethyl starch sodium, hydroxypropylcellulose, hydroxypropylmethylcellulose, povidone, titanium oxide, macrogol and the like.

As pH of these acidic additives and neutral additives, for example, The Japanese Pharmacopoeia, 14th Edition or Japanese Pharmaceutical Excipients 2003 indicates the following values.

D-mannitol (D-mannitol injection): pH 4.5-7.0

crystalline cellulose (supernatant after adding 40 mL of boiled and cooled water to 5.0 g thereof and stirring the mixture by shaking): pH 5.0-7.0

low-substituted hydroxypropylcellulose (solution after adding 100 mL of boiled and cooled water to 1.0 g thereof and stirring the mixture by shaking): pH 5.0-7.5

crospovidone (suspension of 1 g thereof in 100 mL of water): pH 5.0-8.0

cornstarch (suspension obtained by adding 50 mL of water to 1.0 g thereof and boiling and cooling the mixture): neutral

carmellose (suspension obtained by adding 100 mL of water to 1.0 g thereof and stirring the mixture by shaking): pH 3.5-5.0

carmellose sodium (solution obtained by adding 1.0 g thereof to 100 mL of warm water, dissolving same by stirring, and cooling the mixture): pH 6.0-8.0

carmellose calcium (suspension obtained by adding 100 mL of water to 1.0 g thereof and stirring the mixture by shaking): pH 4.5-6.0

carboxymethyl starch sodium (solution obtained by adding 100 mL of water to 1.0 g thereof and stirring the mixture with heating): pH 5.5-8.0

hydroxypropylcellulose (solution obtained by dissolving 1.0 g thereof in 50 mL of boiled and cooled water): pH 5.0-7.5

hydroxypropylmethylcellulose (solution obtained by adding 100 mL of hot water to 1.0 g thereof, stirring the mixture by shaking to give a suspension, followed by cooling): pH 5.0-8.0

povidone (solution obtained by dissolving 1.0 g thereof in 20 mL of water): pH 3.0-7.0

titanium oxide (solution obtained by adding 10 mL of water to 1.0 g thereof and stirring the mixture by shaking): neutral

macrogol (solution obtained by dissolving 1.0 g thereof in 20 mL of water): pH 4.0-7.5

According to The Japanese Pharmacopoeia, 14th Edition or Japanese Pharmaceutical Excipients 2003, these acidic additives and neutral additives show a pH of, for example, not more than 8 when they are prepared into an aqueous solution or suspension by adding water (100 mL) to 1.0-5.0 g of the additives.

By adding any one (preferably both) of low-substituted hydroxypropylcellulose or crospovidone to the pharmaceutical composition of the present invention, compound [1] can be rapidly dissolved out as shown in the Experimental Examples.

Particularly, a pharmaceutical composition containing compound [1], D-mannitol, crystalline cellulose, low-substituted hydroxypropylcellulose, light anhydrous silicic acid, hydroxypropylmethylcellulose, crospovidone and magnesium stearate is preferable.

While the amount of compound [1] (including solvate such as hydrate and the like) contained in the pharmaceutical composition of the present invention is not particularly limited, it is preferably 10 to 60 w/w %, more preferably 20 to 50 w/w %, of the total amount of the pharmaceutical composition. Furthermore, when the pharmaceutical composition is a tablet, it is preferably 10 to 200 mg/tablet, more preferably 10, 25, 50, 100 or 200 mg/tablet.

While the amount of D-mannitol contained as an additive in the pharmaceutical composition of the present invention is not particularly limited, it is preferably 0.05 to 100 parts by weight, more preferably 0.5 to 50 parts by weight, per 1 part by weight of compound [1] (including solvate such as hydrate and the like). The amount is preferably 5 to 60 w/w %, more preferably 10 to 40 w/w %, of the total amount of the pharmaceutical composition. Furthermore, when the pharmaceutical composition is a tablet, the amount is preferably 10 to 90 mg/tablet, more preferably about 10 to 50 mg/tablet. When crystalline cellulose, low-substituted hydroxypropylcellulose, light anhydrous silicic acid, hydroxypropylmethylcellulose, crospovidone, magnesium stearate and the like are used as additives such as excipient, binder, disintegrant and the like, such additives can be appropriately blended according to the amount of the above-mentioned D-mannitol.

While the amount of crystalline cellulose contained as an additive in the pharmaceutical composition of the present invention, which is blended during the granulation step, is not particularly limited, it is preferably 0 to 100 parts by weight, more preferably 0 to 50 parts by weight, per 1 part by weight of compound [1] (including solvate such as hydrate and the like). The amount is preferably 0 to 60 w/w %, more preferably 0 to 40 w/w %, of the total amount of the pharmaceutical composition. Furthermore, when the dosage form of the pharmaceutical composition is a tablet, the amount is preferably 0 to 90 mg/tablet, more preferably 0 to 50 mg/tablet. When D-mannitol, low-substituted hydroxypropylcellulose, light anhydrous silicic acid, hydroxypropylmethylcellulose, crospovidone, magnesium stearate and the like are used as additives such as excipient, lubricant, glidant, binder, disintegrant and the like, such additives can be appropriately blended according to the amount of the above-mentioned crystalline cellulose.

While the amount of crystalline cellulose contained as an additive in the pharmaceutical composition of the present invention, which is blended during the mixing step for obtaining a tableting powder, is not particularly limited, it is preferably 0.01 to 20 parts by weight, more preferably 0.1 to 10 parts by weight, per 1 part by weight of compound [1] (including solvate such as hydrate and the like). The amount is preferably 1 to 20 w/w %, more preferably 2.5 to 10 w/w %, of the total amount of the pharmaceutical composition. Furthermore, when the dosage form of the pharmaceutical composition is a tablet, the amount is preferably 1 to 15 mg/tablet, more preferably 6.5 mg/tablet. When D-mannitol, low-substituted hydroxypropylcellulose, light anhydrous silicic acid, hydroxypropylmethylcellulose, crospovidone, magnesium stearate and the like are used as additives such as excipient, lubricant, glidant, binder, disintegrant and the like, such additives can be appropriately blended according to the amount of the above-mentioned crystalline cellulose.

While the amount of low-substituted hydroxypropylcellulose contained as an additive in the pharmaceutical composition of the present invention is not particularly limited, it is preferably 0.05 to 100 parts by weight, more preferably 0.1 to 50 parts by weight, relative to compound [1] (including solvate such as hydrate and the like). The amount is preferably 1 to 50 w/w %, more preferably 10 to 32 w/w %, still more preferably 20 to 32 w/w %, particularly preferably 27 to 32 w/w %, most preferably 28.6 w/w %, of the total amount of the pharmaceutical composition. When the dosage form of the pharmaceutical composition is a tablet, the amount is preferably 10 to 100 mg/tablet, more preferably 40 mg/tablet. When the dosage form of the pharmaceutical composition is a coated tablet, the amount is preferably 20 to 32 w/w %, more preferably 27 to 32 w/w %, particularly preferably 29.6 w/w %, of the total amount of a core tablet before coating. When D-mannitol, crystalline cellulose, light anhydrous silicic acid, hydroxypropylmethylcellulose, crospovidone, magnesium stearate and the like are used as additives such as excipient, binder, disintegrant and the like, such additives can be appropriately blended according to the amount of the above-mentioned low-substituted hydroxypropylcellulose.

While the amount of light anhydrous silicic acid contained as an additive in the pharmaceutical composition of the present invention is not particularly limited, it is preferably added in an amount of preferably 0.01 to 10 parts by weight, more preferably about 0.01 to 5 parts by weight, per 1 part by weight of compound [1] (including solvate such as hydrate and the like). The amount is preferably about 1 to 10 w/w %, more preferably 1 to 5 w/w %, of the total amount of the pharmaceutical composition. Furthermore, when the dosage form of the pharmaceutical composition is a tablet, the amount is preferably 1 to 10 mg/tablet, more preferably 2.5 mg/tablet. When D-mannitol, crystalline cellulose, low-substituted hydroxypropylcellulose, hydroxypropylmethylcellulose, crospovidone, magnesium stearate and the like are used as additives such as excipient, lubricant, glidant, binder, disintegrant and the like, such additives can be appropriately blended according to the amount of the above-mentioned light anhydrous silicic acid.

While the amount of hydroxypropylmethylcellulose contained as an additive in the pharmaceutical composition of the present invention is not particularly limited, it is preferably 0.01 to 10 parts by weight, more preferably 0.05 to 5 parts by weight, per 1 part by weight of compound [1] (including solvate such as hydrate and the like). The amount is preferably 1 to 10 w/w %, more preferably 1.5 to 6 w/w %, of the total amount of the pharmaceutical composition. Furthermore, when the dosage form of the pharmaceutical composition is a tablet, the amount is preferably 2 to 10 mg/tablet, more preferably 4 mg/tablet. When D-mannitol, crystalline cellulose, light anhydrous silicic acid, low-substituted hydroxypropylcellulose, crospovidone, magnesium stearate and the like are used as additives such as excipient, binder, disintegrant and the like, such additives can be appropriately blended according to the amount of the above-mentioned hydroxypropylmethylcellulose.

While the amount of crospovidone contained as an additive in the pharmaceutical composition of the present invention is not particularly limited, it is preferably 0.1 to 50 parts by weight, more preferably 0.1 to 30 parts by weight, relative to compound [1] (including solvate thereof such as hydrate and the like). The amount is preferably 1 to 30 w/w %, more preferably 1 to 20 w/w %, still more preferably 5 to 20 w/w %, yet more preferably 5 to 17 w/w %, particularly preferably 10 to 17 w/w %, most preferably 10 w/w %, of the total amount of the pharmaceutical composition. Furthermore, when the dosage form of the pharmaceutical composition is a tablet, the amount of crospovidone is preferably 1 to 30 mg/tablet, more preferably 14 mg/tablet. When the dosage form of the pharmaceutical composition is a coated tablet, the amount is preferably 5 to 20 w/w %, more preferably 5 to 17 w/w %, still more preferably 10 to 17 w/w %, particularly preferably 10.4 w/w %, of the total amount of a core tablet before coating. When D-mannitol, crystalline cellulose, light anhydrous silicic acid, low-substituted hydroxypropylcellulose, hydroxypropylmethylcellulose, magnesium stearate and the like are used as additives such as excipient, disintegrant and the like, such additives can be appropriately blended according to the amount of the above-mentioned crospovidone.

While the amount of magnesium stearate contained as an additive in the pharmaceutical composition of the present invention is not particularly limited, it is preferably 0.005 to 5 parts by weight, more preferably 0.01 to 3 parts by weight, per 1 part by weight of compound [1] (including solvate thereof such as hydrate and the like). The amount is preferably 0.2 to 2 w/w %, more preferably 0.5 to 1.5 w/w %, of the total amount of the pharmaceutical composition. Furthermore, when the dosage form of the pharmaceutical composition is a tablet, the amount is preferably 0.5 to 3 mg/tablet, more preferably 1.5 mg/tablet. When D-mannitol, crystalline cellulose, light anhydrous silicic acid, low-substituted hydroxypropylcellulose, hydroxypropylmethylcellulose, crospovidone and the like are used as additives such as excipient, glidant and the like, such additives can be appropriately blended according to the amount of the above-mentioned magnesium stearate.

Unless associated with particular difficulty, various organic or inorganic carrier substances conventionally used as preparation materials are used as the additive in addition to the above-mentioned components, and blended as excipient, lubricant, glidant, binder, disintegrant, solvent, coating agent and the like. Where necessary, other preparation additives such as stabilizer, preservative, antioxidant, sweetening agent, colorant, light shielding agent, corrigent, adsorbent, wetting agent, antistatic agent and the like can also be used.

Examples of the excipient include D-mannitol, crystalline cellulose, low-substituted hydroxypropylcellulose, light anhydrous silicic acid, hydroxypropylmethylcellulose, crospovidone, magnesium stearate, as well as lactose, sucrose, starch and the like.

Examples of the lubricant include crystalline cellulose, light anhydrous silicic acid, magnesium stearate, as well as hydroxypropylcellulose, calcium stearate, talc and the like.

Examples of the glidant include crystalline cellulose, light anhydrous silicic acid, magnesium stearate, as well as talc and the like.

Examples of the binder include D-mannitol, crystalline cellulose, low-substituted hydroxypropylcellulose, light anhydrous silicic acid, hydroxypropylmethylcellulose, as well as sucrose, dextrin, hydroxypropylcellulose, polyvinylpyrrolidone and the like.

Examples of the disintegrant include D-mannitol, crystalline cellulose, low-substituted hydroxypropylcellulose, light anhydrous silicic acid, hydroxypropylmethylcellulose, crospovidone, as well as starch, carboxymethylcellulose, calcium carboxymethylcellulose, croscarmellose sodium, carboxymethyl starch sodium and the like.

Examples of the solvent include water, alcohol, propylene glycol, macrogol, sesame oil, corn oil, propylene glycol fatty acid ester and the like.

Examples of the coating agent include opadry (R)OY-7300 (mixture of hydroxypropylmethylcellulose2910.titanium oxide.macrogol 400) and the like.

The pharmaceutical composition of the present invention encompasses, for example, preparations in the dosage form of tablet, pill, powder, fine granules, granule, suppository, capsule, troche and the like.

The pharmaceutical composition of the present invention can be obtained by mixing compound [1] with one or more kinds of pharmaceutically acceptable additives. When the dosage form of the pharmaceutical composition of the present invention is a tablet, a preferable, specific example of the method is as follows. Firstly, compound [1] is micronized by a jet mill. The micronized compound [1], D-mannitol, low-substituted hydroxypropylcellulose, light anhydrous silicic acid and, where necessary, crystalline cellulose, are mixed to give a mixed powder. A binder solution is added to the obtained mixed powder and the mixture is granulated to give a granulated powder. The obtained granulated powder is sized and then dried to give a sized powder. Crospovidone and crystalline cellulose are added to and mixed with the obtained sized powder. Magnesium stearate is added thereto and they are further mixed. The resulting mixture is subjected to tableting to give core tablets. The core tablets are spray-coated with a coating solution to give film-coated tablets containing compound [1].

The pharmaceutical composition of the present invention can be systemically or topical, and orally or parenterally administered. While the dose varies depending on the age, body weight, condition, treatment effect and the like, for example, it can be generally administered within the range of 0.1 mg to 1 g per administration to an adult once to several times a day. The composition of the present invention can also be used as a drug for the treatment or prophylaxis of the aforementioned diseases in, not to mention human, animals other than human, particularly mammals. The same applies when the pharmaceutical composition of the present invention is used as a drug for the prophylaxis or treatment of hyperlipidemia, diabetes, obesity or cardiovascular disease, such as hypertension, coronary artery disease, vascular endothelial disorder, ischemic heart disease and the like.

Moreover, the pharmaceutical composition of the present invention can be administered to not only human but also other mammals (mouse, rat, hamster, rabbit, cat, dog, bovine, horse, sheep, monkey etc.). Therefore, the pharmaceutical composition of the present invention is also useful as a pharmaceutical product for animals, not to mention human.

The pharmaceutical composition, URAT1 activity inhibitor, agent for decreasing a blood uric acid value, or agent for the prophylaxis or treatment of pathology showing involvement of uric acid of the present invention can be used in combination with other pharmaceutical composition or pharmaceutical agent (hereinafter to be also referred to as a concomitant drug).

The “use in combination” means use of multiple pharmaceutical agents in combination as active ingredients, and use as a combination drug, use as a kit, use in a combination characterized by independent administration of each by the same or different administration routes and the like can be mentioned.

The administration time of the pharmaceutical composition, URAT1 activity inhibitor, agent for decreasing a blood uric acid value, or agent for the prophylaxis or treatment of pathology showing involvement of uric acid of the present invention and a concomitant drug is not limited, and these may be administered simultaneously to the subject of administration or administered in a staggered manner. The dose of the concomitant drug can be determined according to the dose employed clinically, and appropriately determined depending on the subject of administration, the age and body weight of the subject of administration, condition, administration time, dosage form, administration method, combination and the like. The administration mode of the concomitant drug is not particularly limited, and it is only necessary to combine the pharmaceutical composition, URAT1 activity inhibitor, agent for decreasing a blood uric acid value, or agent for the prophylaxis or treatment of pathology showing involvement of uric acid of the present invention and a concomitant drug at the time of administration.

As the concomitant drug,

(1) an agent for the prophylaxis and/or treatment of hyperuricemia,
(2) an agent for the prophylaxis and/or treatment of gout arthritis,
(3) an agent for the prophylaxis and/or treatment of gouty kidney,
(4) an agent for the prophylaxis and/or treatment of urolithiasis,
(5) an agent for the prophylaxis and/or treatment of hypertension or hypertensive complications,
(6) an agent for the prophylaxis and/or treatment of hyperlipidemia or hyperlipidemic complications,
(7) an agent for the prophylaxis and/or treatment of diabetes or diabetic complications,
(8) an agent for the prophylaxis and/or treatment of obesity or obesity complications,
(9) an agent for the prophylaxis and/or treatment of primary disease causing decreased uric acid excretion secondary hyperuricemia,
(10) an agent for the prophylaxis and/or treatment of kidney failure, cardiovascular disorder, cerebrovascular disorder caused by hyperuricemia, and
(11) a nucleic acid metabolic antagonist can be mentioned. One to three of these agents and the compound of the present invention can be used in combination.

As the “agent for the prophylaxis and/or treatment of hyperuricemia”, for example, uric acid production suppressants such as xanthine oxidase inhibitor etc., uricosuric agents and the like can be mentioned, and allopurinol, probenecid, bucolome, febuxostat, benzbromarone, oxipurinol and the like can be specifically mentioned.

As the “agent for the prophylaxis and/or treatment of gout arthritis”, for example, NSAIDs such as indomethacin, naproxen, fenbufen, pranoprofen, oxaprozin and the like, colchicine, corticosteroid and the like can be mentioned.

As the “agent for the prophylaxis and/or treatment of gouty kidney”, for example, uric acid production suppressants such as xanthine oxidase inhibitor etc., uricosuric agents, alkalinizing urine agents such as citric acid preparation, sodium bicarbonate etc., and the like can be mentioned, and allopurinol, probenecid, bucolome, febuxostat, benzbromarone and oxipurinol can be specifically mentioned.

As the “agent for the prophylaxis and/or treatment of urolithiasis”, for example, alkalinizing urine agents such as citric acid preparation, sodium bicarbonate etc., and the like can be mentioned.

As the “agent for the prophylaxis and/or treatment of hypertension or hypertensive complications”, for example, loop diuretics, angiotensin-convertase inhibitors, angiotensin II receptor antagonists, Cα antagonists, β blockers, α,β blockers, α blockers and the like can be mentioned. More specifically, for example, sustained-release furosemide preparation, captopril, sustained-release captopril preparation, enalapril maleate, alacepril, delapril hydrochloride, cilazapril, lisinopril, benazepril hydrochloride, imidapril hydrochloride, temocapril hydrochloride, quinapril hydrochloride, trandolapril, perindopril erbumine, losartan potassium, candesartan cilexetil, nicardipine hydrochloride, sustained-release nicardipine hydrochloride preparation, nilvadipine, nifedipine, sustained-release nifedipine preparation, benidipine hydrochloride, diltiazem hydrochloride, sustained-release diltiazem hydrochloride preparation, nisoldipine, nitrendipine, manidipine hydrochloride, barnidipine hydrochloride, efonidipine hydrochloride, amlodipine besylate, felodipine, cilnidipine, aranidipine, propranolol hydrochloride, sustained-release propranolol hydrochloride preparation, pindolol, sustained-release pindolol preparation, indenolol hydrochloride, carteolol hydrochloride, sustained-release carteolol hydrochloride preparation, bunitrolol hydrochloride, sustained-release bunitrolol hydrochloride preparation, atenolol, acebutolol hydrochloride, metoprolol tartrate, sustained-release metoprolol tartrate preparation, nipradilol, penbutolol sulfate, tilisolol hydrochloride, carvedilol, bisoprolol fumarate, betaxolol hydrochloride, celiprolol hydrochloride, bopindolol malonate, bevantolol hydrochloride, labetalol hydrochloride, arotinolol hydrochloride, amosulalol hydrochloride, prazosin hydrochloride, terazosin hydrochloride, doxazosin mesilate, bunazosin hydrochloride, sustained-release bunazosin hydrochloride preparation, urapidil, phentolamine mesylate and the like can be mentioned.

As the “agent for the prophylaxis and/or treatment of hyperlipidemia or hyperlipidemic complications”, for example, statin pharmaceutical agents, anion exchange resins, probucol, nicotinic acid preparations, fibrate pharmaceutical agents, eicosapentaenoic acid preparations and the like can be mentioned. More specifically, for example, lovastatin, simvastatin, pravastatin, fluvastatin, atorvastatin, cerivastatin, colestimide, colestyramine, niceritrol, nicomol, fenofibrate, bezafibrate, clinofibrate, clofibrate, ethyl icosapentate and the like can be mentioned.

As the “agent for the prophylaxis and/or treatment of diabetes or diabetic complications”, for example, insulin preparations, sulfonylureas, insulin secretagogues, sulfonamides, biguanides, α glucosidase inhibitors, insulin sensitizers, angiotensin-convertase inhibitors, aldose reductase inhibitors, antiarrhythmic drugs and the like can be mentioned. More specifically, for example, insulin, chlorpropamide, glibenclamide, glipizide, tolbutamide, glyclopyramide, acetohexamide, glimepiride, tolazamide, gliclazide, nateglinide, glybuzole, metformin hydrochloride, buformin hydrochloride, voglibose, acarbose, pioglitazone hydrochloride, mexiletine and the like can be mentioned.

As the “agent for the prophylaxis and/or treatment of obesity or obesity complications”, for example, mazindol, acarbose, voglibose, orlistat and the like can be mentioned.

As the “agent for the prophylaxis and/or treatment of primary disease causing decreased uric acid excretion secondary hyperuricemia”, for example, agents for the prophylaxis or treatment of chronic renal disease, polycyctic kidney, toxemia of pregnancy, lead nephropathy, hyperlactacidemia, Down's syndrome, sarcoidosis, glycogenosis I type (via hyperlactacidemia), dehydrating etc., and the like can be mentioned.

As the “agent for the prophylaxis and/or treatment of kidney failure, cardiovascular disorder, cerebrovascular disorder caused by hyperuricemia”, for example, loop diuretics (e.g., furosemide), citric acid preparations, sodium bicarbonate, cation exchange resins, aluminum hydroxide, alfacalcidol, β-blockers (e.g., propranolol hydrochloride), ACE inhibitors (e.g., captopril), cardiac stimulants (e.g., digoxin), angina pectoris therapeutic agents (e.g., isosorbide nitrate), Ca antagonists (e.g., diltiazem hydrochloride), uric acid production suppressants (e.g., allopurinol), amino acid preparations, hyperammonemia improvers, therapeutic agents for antiarrhythmic (e.g., mexiletine), therapeutic agents for anemia (e.g., mepitiostane, erythropoietin), other “agents for the prophylaxis and/or treatment of hypertension or hypertensive complications”, “agents for the prophylaxis and/or treatment of hyperlipidemia or hyperlipidemic complications”, “agents for the prophylaxis and/or treatment of diabetes or diabetic complications”, “agents for the prophylaxis and/or treatment of obesity or obesity complications” and the like can be mentioned.

As the “nucleic acid metabolic antagonist”, for example, azathiopurine, mizoribine, mycophenolic acid and the like can be mentioned.

Furthermore, the pharmaceutical composition, URAT1 activity inhibitor, agent for decreasing a blood uric acid value, and agent for the prophylaxis or treatment of pathology showing involvement of uric acid of the present invention can be used in combination with a pharmaceutical agent that increases the blood uric acid level, thereby to suppress increase in the blood uric acid level.

As the “pharmaceutical agent that increases the blood uric acid level”, nucleic acid metabolic antagonists, hypotensive diuretics, anti-tuberculosis, anti-inflammatory analgesic drugs, hyperlipidemic drugs, therapeutic drugs for asthma, immunosuppressants, salicylic acid, pyrazinamide, ethambutol, nicotinic acid, ethanol, cyclosporine and the like can be mentioned.

Compound [1] is preferably in the form of a crystal. The crystal of compound [1] only needs to be contained in a pharmaceutical composition, and it preferably contains substantially most of the crystal of the present invention.

By “contains substantially most of the crystal of compound [1]” is meant that the crystal of compound [1] is contained in a proportion of not less than 80%, preferably not less than 90%, more preferably not less than 95%.

The production methods of the compound of compound [1] are specifically explained below. It is needless to say that the present invention is not limited to the production methods below. For production of compound [1], the order of the reaction can be appropriately changed. The reaction only needs to be carried out from a step or a position that seems to be reasonable.

In addition, a step for appropriately changing substituents (change or further modification of substituents) may be inserted between respective steps. When a reactive functional group is involved, appropriate protection or deprotection may be conducted. To promote progress of the reaction, moreover, reagents other than those exemplified can be appropriately used. The starting material compounds, for which a production method is not described, are commercially available or can be easily produced by combining known synthetic reactions.

The compound obtained in each step can be isolated and purified by conventional methods such as crystallization, recrystallization, column chromatography, preparative HPLC and the like. In some cases, it is possible to proceed to the next step without isolation and purification.

In the following production methods, the “room temperature” means 1-40° C.

### Production Method 1

A compound [1] wherein X² is an oxygen atom and Y is C═O can be produced by the following Steps:

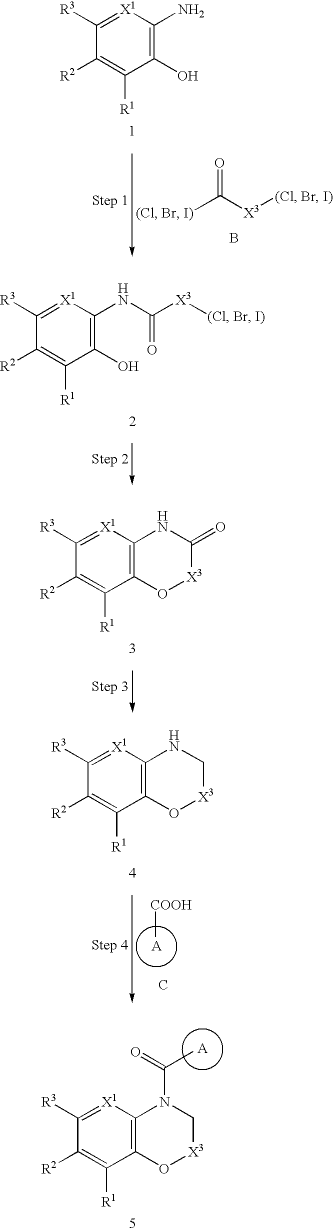

wherein each symbol is as defined above.

### Step 1

Compound 2 can be obtained by amidating compound 1 with acid halide B in a solvent in the presence of a base.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethyl sulfoxide, water and the like; and the like can be mentioned. These can be used alone or in a mixture of two or more kinds thereof. The preferable solvents for this reaction include methylene chloride, chloroform, toluene, ethyl acetate, water and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned, with preference given to triethylamine, pyridine, sodium hydroxide and sodium hydrogencarbonate.

The reaction temperature is about 0° C. to 80° C., preferably about 0° C. to room temperature.

The reaction time is about 10 min to 48 hr, preferably about 30 min to 24 hr.

### Step 2

Compound 3 can be obtained by subjecting compound 2 to cyclization in a solvent in the presence of a base, as necessary in the presence of a catalytic amount of sodium iodide, potassium iodide and the like.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include acetone and N,N-dimethylformamide.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal carbonates such as sodium carbonate, potassium carbonate and the like; alkali metal hydrogencarbonates such as sodium hydrogencarbonate, potassium hydrogencarbonate and the like; alkali metal carboxylates such as sodium acetate, potassium acetate and the like; alkali metal hydrides such as sodium hydride, potassium hydride and the like; alkali metal alkoxides such as sodium ethoxide, sodium methoxide, potassium tert-butoxide and the like; alkyllithiums such as n-butyllithium, s-butyllithium and the like; alkali metal amides such as lithium diisopropylamide, sodium amide, lithium bistrimethylsilylamide and the like; and the like can be mentioned, with preference given to potassium carbonate.

The reaction temperature is about 0° C. to 150° C., preferably room temperature to 100° C.

The reaction time is about 10 min to 48 hr, preferably about 1 hr to 24 hr.

### Step 3

Compound 4 can be obtained by reducing compound 3 with a reducing agent in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; alcohols such as methanol, ethanol, isopropyl alcohol, tert-butanol and the like; and the like can be mentioned, with preference given to toluene, diethyl ether and tetrahydrofuran.

As the reducing agent to be used for the reaction, for example, lithium aluminum hydride, sodium borohydride, diborane, diisobutylaluminum hydride, borane-tetrahydrofuran complex, sodium bis(2-methoxyethoxy)aluminum hydride and the like can be mentioned, with preference given to sodium bis(2-methoxyethoxy)aluminum hydride, lithium aluminum hydride and borane-tetrahydrofuran complex.

The reaction temperature is about 0° C. to 150° C., preferably room temperature to 130° C.

The reaction time is about 10 min to 48 hr, preferably about 30 min to 24 hr.

### Step 4

Compound 5 can be obtained by converting carboxylic acid compound C to the acid chloride with oxalyl chloride, thionyl chloride and the like in a solvent, and amidating compound 4 with the acid chloride in a solvent, as necessary in the presence of a base.

As the solvent to be used for the reaction to obtain the acid chloride, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; and the like can be mentioned. These can be used alone or in a mixture of two or more kinds thereof. The preferable solvents for this reaction include 1,2-dimethoxyethane, ethyl acetate, and methylene chloride, chloroform, toluene, 1,2-dimethoxyethane and ethyl acetate each containing a catalytic amount of N,N-dimethylformamide.

The reaction temperature is about −20° C. to 120° C., preferably about 0° C. to 80° C.

The reaction time is about 10 min to 48 hr, preferably about 30 min to 24 hr.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate, N,N-dimethylformamide and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. This reaction is preferably carried out without a base, or in the presence of triethylamine, pyridine, sodium hydroxide, sodium hydride or sodium hydrogencarbonate.

The reaction temperature is about 0° C. to 120° C., preferably about 0° C. to 95° C.

The reaction time is about 10 min to 48 hr, preferably about 30 min to 24 hr.

Compound 5 can also be obtained by subjecting compound 4 and carboxylic acid compound C to condensation with, for example, water-soluble carbodiimide (WSC HCl: 1-ethyl-3-(3′-dimethylaminopropyl)carbodiimide hydrochloride), dicyclohexylcarbodiimide (DCC), diphenylphosphoryl azide (DPPA), carbonyldiimidazole (CDI), 1-hydroxy-1H-benzotriazole (HOBT), 4-dimethylaminopyridine (DMAP) and the like; or by converting carboxylic acid compound C to a mixed acid anhydride with a chloroformate such as ethyl chloroformate, isopropyl chloroformate, isobutyl chloroformate and the like in the presence of a base such as triethylamine and the like, and reacting compound 4 with the mixed acid anhydride in the presence of a base.

As regards a compound wherein X⁴ is other than —CH₂—, the corresponding compound can be obtained by a method similar to Step 4 and using known compounds.

Step 1 and Step 2 can be conducted in a single step according to Synthesis, 10, 851-852 (1984) to give compound 3.

### Production Method 2

A compound [1] wherein X² is an oxygen atom and Y is C═O can be produced by the following Steps:

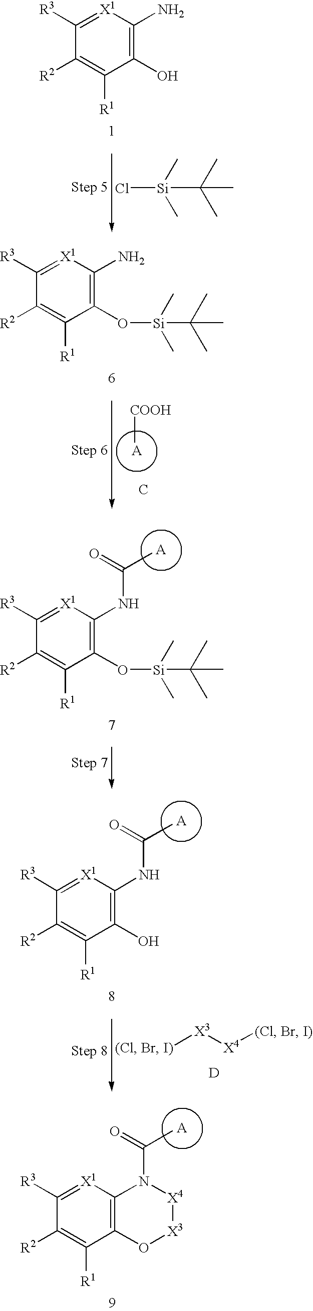

wherein each symbol is as defined above.

### Step 5

Compound 6 can be obtained by subjecting compound 1 to silylation with tert-butylchlorodimethylsilane in a solvent in the presence of a base.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include N,N-dimethylformamide.

As the base to be used for the reaction, for example, organic bases such as triethylamine, pyridine, imidazole, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. The preferable base for this reaction is imidazole.

The reaction temperature is about 0° C. to 150° C., preferably about 0° C. to room temperature.

The reaction time is about 10 min to 24 hr, preferably about 30 min to 12 hr.

### Step 6

Compound 7 can be obtained by converting carboxylic acid compound C to the acid chloride with oxalyl chloride, thionyl chloride and the like in a solvent, and amidating compound 6 with the acid chloride in a solvent, as necessary in the presence of a base.

As the solvent to be used for the reaction to obtain the acid chloride, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; and the like can be mentioned. These can be used alone or in a mixture of two or more kinds thereof. The preferable solvents for this reaction include 1,2-dimethoxyethane, ethyl acetate, and methylene chloride, chloroform, toluene, 1,2-dimethoxyethane and ethyl acetate each containing a catalytic amount of N,N-dimethylformamide.

The reaction temperature is about −20° C. to 120° C., preferably about 0° C. to 80° C.

The reaction time is about 10 min to 48 hr, preferably about 30 min to 24 hr.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. This reaction is preferably carried out without a base, or in the presence of triethylamine, pyridine, sodium hydroxide or sodium hydrogencarbonate.

The reaction temperature is about 0° C. to 120° C., preferably about 0° C. to 95° C.

The reaction time is about 10 min to 48 hr, preferably about 30 min to 24 hr.

Compound 7 can also be obtained by subjecting compound 6 and carboxylic acid compound C to condensation with, for example, water-soluble carbodiimide (WSC HCl: 1-ethyl-3-(3′-dimethylaminopropyl)carbodiimide hydrochloride), dicyclohexylcarbodiimide (DCC), diphenylphosphoryl azide (DPPA), carbonyldiimidazole (CDI), 1-hydroxy-1H-benzotriazole (HOBT), 4-dimethylaminopyridine (DMAP) and the like; or by converting carboxylic acid compound C to a mixed acid anhydride with a chloroformate such as ethyl chloroformate, isopropyl chloroformate, isobutyl chloroformate and the like in the presence of a base such as triethylamine and the like, and reacting compound 6 with the mixed acid anhydride in the presence of a base.

### Step 7

Compound 8 can be obtained by subjecting compound 7 to desilylation in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; alcohols such as methanol, ethanol, isopropyl alcohol, tert-butanol and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include N,N-dimethylformamide.

As the reagent to be used for the reaction, for example, potassium carbonate, tetrabutylammonium fluoride and the like can be mentioned, with preference given to potassium carbonate.

The reaction temperature is about 0° C. to 150° C., preferably room temperature to 80° C.

The reaction time is about 10 min to 48 hr, preferably about 30 min to 24 hr.

### Step 8

Compound 9 can be obtained by subjecting compound 8 to cyclization with halide D in a solvent in the presence of a base.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include N,N-dimethylformamide.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. The preferable base for this reaction is potassium carbonate.

The reaction temperature is about 0° C. to 150° C., preferably room temperature to 100° C.

The reaction time is about 10 min to 48 hr, preferably about 3 hr to 24 hr.

### Production Method 3

A compound [1] wherein X² is a carbon atom or a sulfur atom and Y is C═O can be produced by the following Steps:

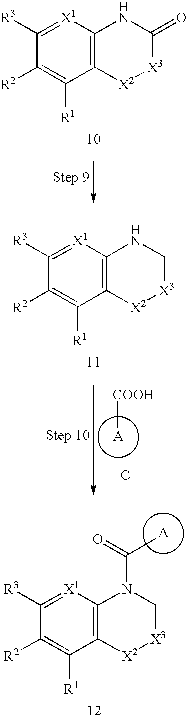

wherein each symbol is as defined above.

### Step 9

Compound 11 can be obtained by reducing known compound 10 with a reducing agent in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; alcohols such as methanol, ethanol, isopropyl alcohol, tert-butanol and the like; and the like can be mentioned, with preference given to toluene, diethyl ether and tetrahydrofuran.

As the reducing agent to be used for the reaction, for example, lithium aluminum hydride, sodium borohydride, diborane, diisobutylaluminum hydride, borane-tetrahydrofuran complex, sodium bis(2-methoxyethoxy)aluminum hydride and the like can be mentioned, with preference given to sodium bis(2-methoxyethoxy)aluminum hydride, lithium aluminum hydride and borane-tetrahydrofuran complex.

The reaction temperature is about 0° C. to 150° C., preferably room temperature to 130° C.

The reaction time is about 10 min to 48 hr, preferably about 30 min to 24 hr.

### Step 10

Compound 12 can be obtained by converting carboxylic acid compound C to the acid chloride with oxalyl chloride, thionyl chloride and the like in a solvent, and amidating compound 11 with the acid chloride in a solvent, as necessary in the presence of a base.

As the solvent to be used for the reaction to obtain the acid chloride, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; and the like can be mentioned. These can be used alone or in a mixture of two or more kinds thereof. The preferable solvents for this reaction include 1,2-dimethoxyethane, ethyl acetate, and methylene chloride, chloroform, toluene, 1,2-dimethoxyethane and ethyl acetate each containing a catalytic amount of N,N-dimethylformamide.

The reaction temperature is about −20° C. to 120° C., preferably about 0° C. to 80° C.

The reaction time is about 10 min to 48 hr, preferably about 30 min to 24 hr.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate, N,N-dimethylformamide and tetrahydrofuran.

As the base to be used for the reaction, for example, organic bases such as triethylamine, pyridine, dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. This reaction is preferably carried out without a base, or in the presence of triethylamine, pyridine, sodium hydroxide, sodium hydride or sodium hydrogencarbonate.

The reaction temperature is about 0° C. to 120° C., preferably about 0° C. to 95° C.

The reaction time is about 10 min to 48 hr, preferably about 30 min to 24 hr.

The compound 12 can also be obtained by subjecting compound 11 and carboxylic acid compound C to condensation with, for example, water-soluble carbodiimide (WSC HCl: 1-ethyl-3-(3′-dimethylaminopropyl)carbodiimide hydrochloride), dicyclohexylcarbodiimide (DCC), diphenylphosphoryl azide (DPPA), carbonyldiimidazole (CDI), 1-hydroxy-1H-benzotriazole (HOBT), 4-dimethylaminopyridine (DMAP) and the like; or by converting carboxylic acid compound C to a mixed acid anhydride with a chloroformate such as ethyl chloroformate, isopropyl chloroformate, isobutyl chloroformate and the like in the presence of a base such as triethylamine and the like, and reacting compound 11 with the mixed acid anhydride in the presence of a base.

As regards a compound wherein X⁴ is other than —CH₂—, the corresponding compound can be obtained by a method similar to Step 10 and using known compounds.

### Production Method 4

A compound [1] wherein X² is S═O or S(═O)₂ and Y is C═O can be produced by the following Step:

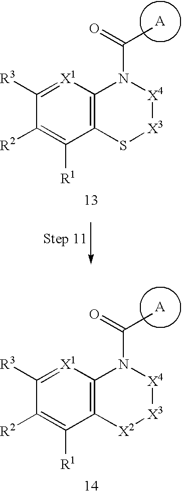

wherein each symbol is as defined above.

### Step 11

Compound 14 can be obtained by oxidizing compound 13 with an oxidant in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; alcohols such as methanol, ethanol, isopropyl alcohol, tert-butanol and the like; esters such as ethyl acetate, methyl acetate, butyl acetate and the like; polar solvents such as acetone, N,N-dimethylformamide, dimethyl sulfoxide, water, acetic acid and the like; and the like can be mentioned. These can be used alone or in a mixture of two or more kinds thereof. The preferable solvents for this reaction include methylene chloride and chloroform.

As the oxidant to be used for the reaction, for example, 3-chloroperbenzoic acid, hydrogen peroxide, sodium periodate, tert-butylhydroperoxide and the like can be mentioned, with preference given to 3-chloroperbenzoic acid.

The reaction temperature is about 0° C. to 80° C., preferably about 0° C. to room temperature.

The reaction time is about 10 min to 48 hr, preferably about 2 hr to 24 hr.

### Production Method 5

A compound [1] wherein X² is NR⁵, N(COR⁶), N(S(═O)₂R⁶ or N(CONR⁷R⁸) and Y is C═O can be produced by the following Step:

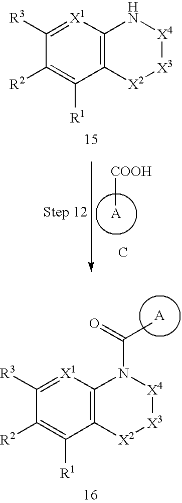

wherein each symbol is as defined above.

### Step 12

Compound 16 can be obtained by converting carboxylic acid compound C to the acid chloride with oxalyl chloride, thionyl chloride and the like in a solvent, and amidating known compound 15 with the acid chloride in a solvent, as necessary in the presence of a base.

As the solvent to be used for the reaction to obtain the acid chloride, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; and the like can be mentioned. These can be used alone or in a mixture of two or more kinds thereof. The preferable solvents for this reaction include 1,2-dimethoxyethane, ethyl acetate, and methylene chloride, chloroform, toluene, 1,2-dimethoxyethane and ethyl acetate each containing a catalytic amount of N,N-dimethylformamide.

The reaction temperature is about −20° C. to 120° C., preferably about 0° C. to 80° C.

The reaction time is about 10 min to 48 hr, preferably about 30 min to 24 hr.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate, N,N-dimethylformamide and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. This reaction is preferably carried out without a base, or in the presence of triethylamine, pyridine, sodium hydroxide, sodium hydride or sodium hydrogencarbonate.

The reaction temperature is about 0° C. to 120° C., preferably about 0° C. to 95° C.

The reaction time is about 10 min to 48 hr, preferably about 30 min to 24 hr.

Compound 16 can also be obtained by subjecting known compound 15 and carboxylic acid compound C to condensation with, for example, water-soluble carbodiimide (WSC HCl: 1-ethyl-3-(3′-dimethylaminopropyl)carbodiimide hydrochloride), dicyclohexylcarbodiimide (DCC), diphenylphosphoryl azide (DPPA), carbonyldiimidazole (CDI), 1-hydroxy-1H-benzotriazole (HOBT), 4-dimethylaminopyridine (DMAP) and the like; or by converting carboxylic acid compound C to a mixed acid anhydride with a chloroformate such as ethyl chloroformate, isopropyl chloroformate, isobutyl chloroformate and the like in the presence of a base such as triethylamine and the like, and reacting known compound 15 with the mixed acid anhydride in the presence of a base.

Note that R⁵, COR⁶, S(═O)₂R⁶ and CONR⁷R⁸ can be introduced into the nitrogen atom of X² before and after Step 12, and the reactions can be carried out in a reasonable order.

### Production Method 6

A compound [1] wherein Y is C═S can be produced by the following Step:

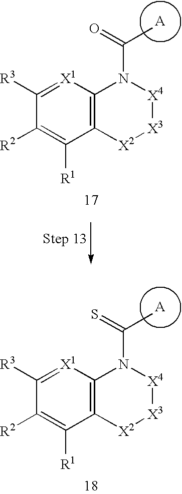

wherein each symbol is as defined above.

### Step 13

Compound 18 can be obtained by reacting compound 17 with Lawesson reagent, diphosphorus pentasulfide and the like in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; alcohols such as methanol, ethanol, isopropyl alcohol, tert-butanol and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include tetrahydrofuran and toluene.

The reaction temperature is about 0° C. to 150° C., preferably room temperature to 120° C.

The reaction time is about 10 min to 24 hr, preferably about 30 min to 12 hr.

### Production Method 7

A compound [1] wherein Y is S(═O)₂ can be produced by the following Step:

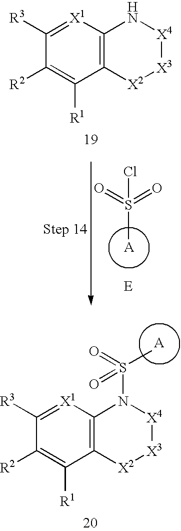

wherein each symbol is as defined above.

### Step 14

Compound 20 can be obtained by amidating compound 19 with known acid chloride E in a solvent, as necessary in the presence of a base.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate, N,N-dimethylformamide and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. Preferably, this reaction is carried out without a base, or in the presence of triethylamine, pyridine, sodium hydroxide, sodium hydride or sodium hydrogencarbonate.

The reaction temperature is about 0° C. to 120° C., preferably about 0° C. to 95° C.

The reaction time is about 10 min to 48 hr, preferably about 30 min to 24 hr.

### Production Method 8

A compound [1] wherein X² is an oxygen atom and Y is C═O can be produced by the following Steps:

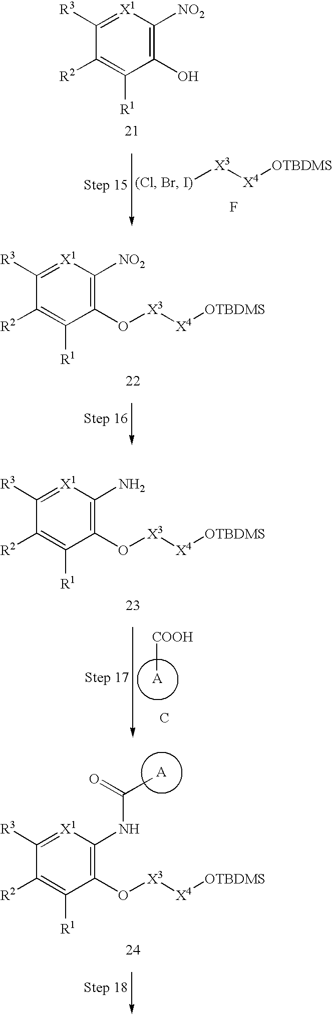

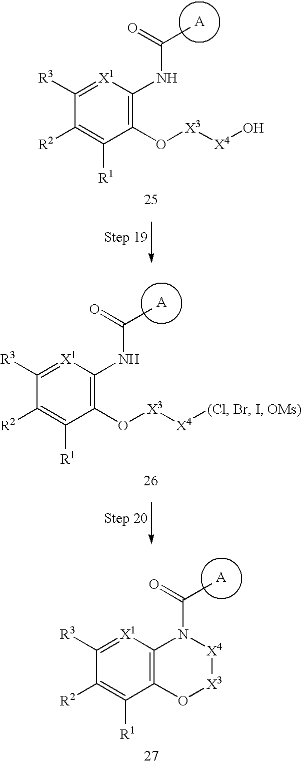

wherein TBDMS is a tert-butyldimethylsilyl group, —OMs is —OS(═O)₂—CH₃, and the other symbols are as defined above.

### Step 15

Compound 22 can be obtained by reacting compound 21 with halide F in a solvent in the presence of a base, as necessary in the presence of sodium iodide, potassium iodide and the like.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include acetone and N,N-dimethylformamide.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal carbonates such as sodium carbonate, potassium carbonate and the like; alkali metal hydrogencarbonates such as sodium hydrogencarbonate, potassium hydrogencarbonate and the like; alkali metal carboxylates such as sodium acetate, potassium acetate and the like; alkali metal hydrides such as sodium hydride, potassium hydride and the like; alkali metal alkoxides such as sodium ethoxide, sodium methoxide, potassium tert-butoxide and the like; alkyllithiums such as n-butyllithium, s-butyllithium and the like; alkali metal amides such as lithium diisopropylamide, sodium amide, lithium bistrimethylsilylamide and the like; and the like can be mentioned, with preference given to sodium hydride and potassium carbonate.

The reaction temperature is about 0° C. to 150° C., preferably room temperature to 130° C.

The reaction time is about 10 min to 48 hr, preferably about 1 hr to 24 hr.

### Step 16

Compound 23 can be obtained by reducing compound 22 in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; alcohols such as methanol, ethanol, isopropyl alcohol, tert-butanol and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include tetrahydrofuran, ethyl acetate and ethanol.

As the reduction reaction, for example, hydrogenation using a noble metal catalyst (e.g., palladium carbon, palladium-barium sulfate, palladium black, platinum carbon, platinum oxide, rhodium carbon, Raney-nickel etc.) and the like, or reduction reaction using tin dichloride, iron, sodium hydrosulfite and the like, and the like can be mentioned, with preference given to hydrogenation using a noble metal catalyst (palladium carbon).

The reaction temperature is about 0° C. to 150° C., preferably room temperature to 100° C.

The reaction time is about 10 min to 48 hr, preferably about 30 min to 24 hr.

### Step 17

Compound 24 can be obtained by converting carboxylic acid compound C to the acid chloride with oxalyl chloride, thionyl chloride and the like in a solvent, and amidating compound 23 with the acid chloride in a solvent, as necessary in the presence of a base.

As the solvent to be used for the reaction to obtain the acid chloride, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; and the like can be mentioned. These can be used alone or in a mixture of two or more kinds thereof. The preferable solvents for this reaction include 1,2-dimethoxyethane, ethyl acetate, and methylene chloride, chloroform, toluene, 1,2-dimethoxyethane and ethyl acetate each containing a catalytic amount of N,N-dimethylformamide.

The reaction temperature is about −20° C. to 120° C., preferably about 0° C. to 80° C.

The reaction time is about 10 min to 48 hr, preferably about 30 min to 24 hr.

As the solvent to be used for the amidation reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include methylene chloride, chloroform, toluene, ethyl acetate and tetrahydrofuran.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal hydrides such as sodium hydride, potassium hydride and the like; alkali metal carbonates such as sodium carbonate, potassium carbonate and the like; alkali metal hydrogencarbonates such as sodium hydrogencarbonate, potassium hydrogencarbonate and the like; and the like can be mentioned. This reaction is preferably carried out without a base, or in the presence of triethylamine, pyridine, sodium hydroxide or sodium hydrogencarbonate.

The reaction temperature is about 0° C. to 120° C., preferably about 0° C. to 95° C.

The reaction time is about 10 min to 48 hr, preferably about 30 min to 24 hr.

The compound 24 can also be obtained by subjecting compound 23 and carboxylic acid compound C to condensation with, for example, water-soluble carbodiimide (WSC HCl: 1-ethyl-3-(3′-dimethylaminopropyl)carbodiimide hydrochloride), dicyclohexylcarbodiimide (DCC), diphenylphosphoryl azide (DPPA), carbonyldiimidazole (CDI), 1-hydroxy-1H-benzotriazole (HOBT), 4-dimethylaminopyridine (DMAP) and the like; or by converting carboxylic acid compound C to a mixed acid anhydride with a chloroformate such as ethyl chloroformate, isopropyl chloroformate, isobutyl chloroformate and the like in the presence of a base such as triethylamine and the like, and reacting compound 23 with the mixed acid anhydride in the presence of a base.

### Step 18

Compound 25 can be obtained by subjecting compound 24 to desilylation in a solvent.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; alcohols such as methanol, ethanol, isopropyl alcohol, tert-butanol and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include tetrahydrofuran and N,N-dimethylformamide.

As the reagent to be used for the reaction, for example, potassium carbonate, tetrabutylammonium fluoride and the like can be mentioned, with preference given to tetrabutylammonium fluoride.

The reaction temperature is about 0° C. to 150° C., preferably room temperature to 80° C.

The reaction time is about 10 min to 48 hr, preferably about 30 min to 24 hr.

### Step 19

Compound 25 can be converted to compound 26.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. Preferably, this reaction is carried out without a solvent or in the presence of methylene chloride or chloroform.

As the reagent to be used for the reaction, for example, carbon tetrachloride in the presence of triphenylphosphine; N-chlorosuccinimide (NCS) in the presence of triphenylphosphine; thionyl chloride; carbon tetrabromide in the presence of triphenylphosphine; N-bromosuccinimide (NBS) in the presence of triphenylphosphine; phosphorus tribromide; phosphorus bromide; iodine in the presence of triphenylphosphine and imidazole; methanesulfonyl chloride in the presence of a base (pyridine, triethylamine etc.); and the like can be mentioned, with preference given to methanesulfonyl chloride in the presence of a base (pyridine, triethylamine etc.)

The reaction temperature is about 0° C. to 150° C., preferably room temperature to 100° C.

The reaction time is about 10 min to 48 hr, preferably about 30 min to 24 hr.

### Step 20

Compound 27 can be obtained by subjecting compound 26 to cyclization in a solvent in the presence of a base, as necessary in the presence of sodium iodide, potassium iodide and the like.

As the solvent to be used for the reaction, for example, ethers such as diethyl ether, tetrahydrofuran, dioxane, 1,2-dimethoxyethane, diglyme and the like; hydrocarbons such as benzene, toluene, hexane, xylene and the like; halogenated hydrocarbons such as methylene chloride, chloroform, carbon tetrachloride, 1,2-dichloroethane and the like; esters such as ethyl acetate, methyl acetate, butyl acetate and the like; polar solvents such as acetone, N,N-dimethylformamide, dimethylsulfoxide and the like; and the like can be mentioned. These can be used alone or in a mixture of two or more kinds thereof. The preferable solvents for this reaction include tetrahydrofuran and N,N-dimethylformamide.

As the base to be used for the reaction, for example, organic bases such as triethylamine, pyridine, 4-dimethylaminopyridine, N-methylmorpholine and the like; alkali metal hydroxides such as lithium hydroxide, sodium hydroxide, potassium hydroxide and the like; alkali metal carbonates such as sodium carbonate, potassium carbonate and the like; alkali metal hydrogencarbonates such as sodium hydrogencarbonate, potassium hydrogencarbonate and the like; alkali metal carboxylates such as sodium acetate, potassium acetate and the like; alkali metal hydrides such as sodium hydride, potassium hydride and the like; alkali metal alkoxides such as sodium ethoxide, sodium methoxide, potassium tert-butoxide and the like; alkyllithiums such as n-butyllithium, s-butyllithium and the like; alkali metal amides such as lithium diisopropylamide, sodium amide, lithium bistrimethylsilylamide and the like; and the like can be mentioned, with preference given to potassium carbonate and sodium hydride.

The reaction temperature is about 0° C. to 150° C., preferably room temperature to 100° C.

The reaction time is about 10 min to 48 hr, preferably about 30 min to 24 hr.

While compound [1] obtained by the production method of the present invention may be in the form of an amorphous form or a crystal by a crystallization method known per se, with preference given to a crystal.

Here, as the crystallization method, for example, crystallization from a solution, crystallization from vapor, crystallization from a molten form and the like can be mentioned.

For the “crystallization from a solution”, a method comprising shifting from a non-saturation state to a supersaturation state by changing the factors (solvent composition, pH, temperature, ionic strength, oxidation-reduction state etc.) relating to the solubility of the compound or the amount of solvent is generally employed. Specifically, for example, concentration method, slow cooling method, reaction methods (diffusion method, electrolysis method), hydrothermal growth method, fusing agent method and the like can be mentioned. As the solvent to be used, for example, aromatic hydrocarbon solvents (e.g., benzene, toluene, xylene etc.), halogenated hydrocarbon solvents (e.g., dichloromethane, chloroform etc.), saturated hydrocarbon solvents (e.g., hexane, heptane, cyclohexane etc.), ether solvents (e.g., diethyl ether, diisopropyl ether, tetrahydrofuran, dioxane, anisole etc.), nitrile solvents (e.g., acetonitrile etc.), ketone solvents (e.g., acetone, methyl ethyl ketone, methyl isobutyl ketone etc.), sulfoxide solvents (e.g., dimethyl sulfoxide etc.), acid amide solvents (e.g., N,N-dimethylformamide etc.), ester solvents (e.g., methyl acetate, ethyl acetate, n-butyl acetate etc.), alcohol solvents (e.g., methanol, ethanol, 1-propanol, 2-propanol, 1-butanol, 2-butanol, 1-pentanol, 2-methyl-1-propanol), organic acid solvent solvents (e.g., acetic acid, formic acid etc.), organic base solvents (e.g., pyridine etc.), water and the like can be mentioned. These solvents are used alone or a mixture of two or more thereof at a suitable ratio (e.g., 1:1 to 1:100 (volume ratio)).

For the “crystallization from vapor”, for example, gasification methods (sealed tube method, gas stream method), gas phase reaction method, chemical transportation method and the like can be mentioned.

For the “crystallization from a molten form”, for example, normal freezing methods (pulling-up method, temperature gradient method, Bridgman method), zone melting methods (zone leveling method, float zone method), special growth methods (VLS method, liquid phase epitaxis method) and the like can be mentioned.

Moreover, compound [1] in an amorphous or crystal form can be further purified by the above-mentioned crystallization method. For this object, “crystallization from a solution” is preferable.

As the solvent to be used, from the aspect of the solubility of compound [1] (particularly, (3,5-dichloro-4-hydroxyphenyl)-(2,3-dihydrobenzo[1,4]oxazin-4-yl)-methanone), aromatic hydrocarbon solvents, halogenated hydrocarbon solvents, ether solvents, nitrile solvents, ketone solvents, sulfoxide solvents, acid amide solvents, ester solvents, alcohol solvents, organic acid solvent solvents and the like are preferable, and ester solvents, ether solvents, alcohol solvents and ketone solvents are more preferable. Of these, alcohol solvents and ketone solvents are particularly preferable. As the alcohol solvent, 1-propanol and 1-butanol are preferable, and as the ketones solvent, methyl ethyl ketone, and methyl isobutyl ketone are preferable.

These solvents may be used alone or in a mixture of two or more kinds thereof at a suitable ratio (e.g., 1:1 to 1:100 (volume ratio)). Alternatively, a mixed solvent of these solvents and water or a saturated hydrocarbon solvent may be used.

The amount of the solvent to be used from the viewpoint of industrial practice is 1-100 mL, preferably 1-50 mL, more preferably 1-30 mL, particularly preferably 20-30 mL, per 1 g of compound [1].

When the difference between the dissolution temperature of compound [1] and the boiling point of the solvent is small, stable dissolution of the compound becomes difficult. In addition, the production operability may be degraded because crystals may precipitate during dust removal filtration to be performed before crystallization. In consideration of this aspect, the difference between the boiling point of the solvent to be used and the dissolution temperature of compound [1] (particularly, (3,5-dichloro-4-hydroxyphenyl)-(2,3-dihydrobenzo[1,4]oxazin-4-yl)-methanone) is preferably not less than 5° C., more preferably not less than 10° C., particularly preferably not less than 20° C. Moreover, the solvent to be used preferably has a boiling point of not more than 150° C. because the residual solvent in the obtained crystal may degrade the quality.

Specific examples of the purification method of compound [1] by crystallization include a method comprising dissolving compound [1] (e.g., (3,5-dichloro-4-hydroxyphenyl)-(2,3-dihydrobenzo[1,4]oxazin-4-yl)-methanone) in a suitable solvent (e.g., aromatic hydrocarbon solvent, halogenated hydrocarbon solvent, ether solvent, nitrile solvent, ketone solvent, sulfoxide solvent, acid amide solvent, ester solvent, alcohol solvent, organic acid solvent and the like, preferably ester solvent, ether solvent, ketone solvent, alcohol solvent and the like, more preferably ketone solvent, alcohol solvent and the like, more preferably, methyl isobutyl ketone, 1-butanol, particularly preferably 1-butanol) at a temperature of 90-100° C., and cooling the obtained solution to a temperature not more than the dissolution temperature (e.g., 0-90° C., preferably 20-30° C.), a method using a poor solvent (e.g., saturated hydrocarbon solvent, water etc.) and the like, and a combination of these.

The crystal of compound [1] thus purified can be isolated, for example, by filtration and the like.

Compound [1] obtained by the production method of the present invention may develop color due to impurities. Moreover, a crystal obtained by the above-mentioned purification may still retain color due to the impurities remaining therein. In such a case, however, the impurities causing the color can be removed by treating compound [1] with an adsorbent (e.g., activated carbon, alumina, activated earth, silica gel, celite etc., preferably activated carbon). The treatment with an adsorbent can be performed together with the above-mentioned purification step, or separately before or after the above-mentioned purification step.

As a treatment method with an adsorbent, compound [1] (e.g., (3,5-dichloro-4-hydroxyphenyl)-(2,3-dihydrobenzo[1,4]oxazin-4-yl)-methanone) is dissolved in a suitable solvent (e.g., aromatic hydrocarbon solvent, halogenated hydrocarbon solvent, ether solvent, nitrile solvent, ketone solvent, sulfoxide solvent, acid amide solvent, ester solvent, alcohol solvent, organic acid solvent and the like, preferably ester solvent, ether solvent, ketone solvent, alcohol solvent and the like, more preferably ketone solvent, alcohol solvent and the like, more preferably methyl isobutyl ketone and 1-butanol, particularly preferably 1-butanol), an adsorbent is added, and the mixture is treated at room temperature to under heating (preferably, 90-100° C.) for 0.5-24 hr, preferably 1-10 hr.

While the amount of the adsorbent to be used is not particularly limited, it is 1-20 wt %, preferably 5-15 wt %, relative to compound [1] from the aspects of removal of impurities and efficient separation of adsorbent.

### EXAMPLES

The compounds used in the present invention are concretely explained by referring to Reference Examples and Reference Experimental Examples, which are not to be construed as limitative. In the Reference Examples, the room temperature means 1-40° C.

### Reference Example 1

### Production of (3-chloro-4-hydroxyphenyl)-(2,3-dihydrobenzo[1,4]oxazin-4-yl)-methanone

### Step 1

### Production of 3-chloro-4-methoxybenzoyl chloride

Chloroform (20 mL) was added to 3-chloro-4-methoxybenzoic acid (2.0 g), and oxalyl chloride (1.84 mL) and N,N-dimethylformamide (1 drop) were added under ice-cooling. The mixture was stirred at room temperature for 3 hrs, concentrated and azeotroped with toluene to give the title compound (2.063 g).

### Step 2

### Production of 3,4-dihydro-2H-benzo[1,4]oxazine

Synthesis was performed in reference to Australian journal of chemistry, 9, 397-405 (1956). To be specific, lithium aluminum hydride (3 g) was suspended in tetrahydrofuran (120 mL), and 2H-1,4-benzoxazin-3(4H)-one (6 g) was added by small portions under ice-cooling. After heating under reflux for 10 hrs, water (3 mL), 15% aqueous sodium hydroxide (3 mL) and water (9 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=4:1) to give the title compound (4.9239 g) as an orange oil.

### Step 3

### Production of (3-chloro-4-methoxyphenyl)-(2,3-dihydrobenzo[1,4]oxazin-4-yl)-methanone

3,4-Dihydro-2H-benzo[1,4]oxazine (525 mg) was dissolved in chloroform (10 mL), and triethylamine (0.65 mL) and 3-chloro-4-methoxybenzoyl chloride (836 mg) were added under ice-cooling. The mixture was stirred at room temperature for 12 hrs, and the reaction mixture was purified by silica gel chromatography (n-hexane-ethyl acetate=4:1) to give the title compound (1.180 g) as a white solid.

### Step 4

### Production of (3-chloro-4-hydroxyphenyl)-(2,3-dihydrobenzo[1,4]oxazin-4-yl)-methanone

(3-Chloro-4-methoxyphenyl)-(2,3-dihydrobenzo[1,4]oxazin-4-yl)-methanone (1.175 g) was dissolved in chloroform (10 mL). Boron tribromide (1.0 M methylene chloride solution, 7.74 mL) was added dropwise at −45° C., and the mixture was stirred at room temperature for 3 hrs. Water and ethyl acetate were added to the reaction mixture, and the mixture was extracted with chloroform. The obtained chloroform layer was washed with water and saturated aqueous sodium hydrogencarbonate, and dried over anhydrous sodium sulfate. The solvent was evaporated, and the residue was purified by silica gel chromatography (n-hexane-ethyl acetate=65:35) to give the title compound (124 mg) as white crystals.

### Reference Example 2

### Production of (3-bromo-4-hydroxyphenyl)-(2,3-dihydrobenzo[1,4]oxazin-4-yl)-methanone

### Step 1

### Production of 3-bromo-4-hydroxybenzoyl chloride

1,2-Dimethoxyethane (30 mL) was added to 3-bromo-4-hydroxybenzoic acid (3.25 g) to dissolve same by heating the mixture to 80° C. Thionyl chloride (1.6 mL) was added, and the mixture was stirred overnight at 80° C. The reaction mixture was concentrated under reduced pressure, azeotroped with toluene, and dried to give the title compound (3.6181 g) as a white solid.

### Step 2

### Production of (3-bromo-4-hydroxyphenyl)-(2,3-dihydrobenzo[1,4]oxazin-4-yl)-methanone

3,4-Dihydro-2H-benzo[1,4]oxazine (203 mg) obtained in Step 2 of Reference Example 1 and 3-bromo-4-hydroxybenzoyl chloride (353 mg) were dissolved in ethyl acetate (4 mL), and the mixture was stirred overnight at 95° C. The reaction mixture was allowed to cool to room temperature, and the precipitated solid was collected by filtration to give the title compound (236.7 mg) as beige crystals.

### Reference Example 3

### Production of (3,5-dichloro-4-hydroxyphenyl)-(2,3-dihydrobenzo[1,4]oxazin-4-yl)-methanone

### Step 1

### Production of 3,5-dichloro-4-hydroxybenzoyl chloride

1,2-Dimethoxyethane (30 mL) was added to 3,5-dichloro-4-hydroxybenzoic acid (1.242 g) to dissolve the same by heating the mixture to 80° C. Thionyl chloride (0.57 mL) was added, and the mixture was stirred overnight at 80° C. The reaction mixture was concentrated under reduced pressure, azeotroped with toluene, and dried to give the title compound (1.358 g) as a white solid.

### Step 2

### Production of (3,5-dichloro-4-hydroxyphenyl)-(2,3-dihydrobenzo[1,4]oxazin-4-yl)-methanone

3,4-Dihydro-2H-benzo[1,4]oxazine (135 mg) obtained in Step 2 of Reference Example 1 and 3,5-dichloro-4-hydroxybenzoyl chloride (225 mg) were dissolved in ethyl acetate (3.2 mL), and the mixture was stirred overnight at 95° C. The reaction mixture was allowed to cool to room temperature, and the precipitated solid was collected by filtration to give the title compound (282 mg) as white crystals.

### Reference Example 4

### Production of (3,5-dibromo-4-hydroxyphenyl)-(2,3-dihydrobenzo[1,4]oxazin-4-yl)-methanone

### Step 1

### Production of 3,5-dibromo-4-hydroxybenzoyl chloride

1,2-Dimethoxyethane (20 mL) was added to 3,5-dibromo-4-hydroxybenzoic acid (2.96 g) to dissolve same by heating the mixture to 80° C. Thionyl chloride (1.1 mL) was added, and the mixture was stirred overnight at 80° C. The reaction mixture was concentrated under reduced pressure, azeotroped with toluene, and dried to give the title compound (3.1562 g) as a white solid.

### Step 2

### Production of (3,5-dibromo-4-hydroxyphenyl)-(2,3-dihydrobenzo[1,4]oxazin-4-yl)-methanone

3,4-Dihydro-2H-benzo[1,4]oxazine (270 mg) obtained in Step 2 of Reference Example 1 and 3,5-dibromo-4-hydroxybenzoyl chloride (629 mg) were dissolved in ethyl acetate (10 mL), and the mixture was stirred overnight at 95° C. The reaction mixture was allowed to cool to room temperature, and the precipitated solid was collected by filtration to give the title compound (700.9 mg) as pale-orange crystals.

### Reference Example 5

### Production of (2,3-dihydrobenzo[1,4]oxazin-4-yl)-(4-hydroxy-3,5-diiodophenyl)-methanone

### Step 1

### Production of 4-hydroxy-3,5-diiodobenzoyl chloride

1,2-Dimethoxyethane (12 mL) was added to 4-hydroxy-3,5-diiodobenzoic acid (2.34 g) to dissolve same by heating the mixture to 80° C. Thionyl chloride (0.66 mL) was added, and the mixture was stirred overnight at 80° C. The reaction mixture was concentrated under reduced pressure, azeotroped with toluene, and dried to give the title compound (2.4922 g) as a cream color solid.

### Step 2

### Production of (2,3-dihydrobenzo[1,4]oxazin-4-yl)-(4-hydroxy-3,5-diiodophenyl)-methanone

3,4-Dihydro-2H-benzo[1,4]oxazine (406 mg) obtained in Step 2 of Reference Example 1 and 4-hydroxy-3,5-diiodobenzoyl chloride (1.26 g) were dissolved in ethyl acetate (15 mL), and the mixture was stirred overnight at 95° C. The reaction mixture was allowed to cool to room temperature, and the precipitated solid was collected by filtration to give the title compound (1.2887 g) as pale-orange crystals.

### Reference Example 6

### Production of (3,5-difluoro-4-hydroxyphenyl)-(2,3-dihydrobenzo[1,4]oxazin-4-yl)-methanone

### Step 1

### Production of 3,5-difluoro-4-methoxybenzoyl chloride

Chloroform (20 mL) was added to 3,5-difluoro-4-methoxybenzoic acid (2 g), and oxalyl chloride (1.87 mL) and N,N-dimethylformamide (1 drop) were added under ice-cooling. After stirring at room temperature for 3 hrs, the reaction mixture was concentrated under reduced pressure, and azeotroped with toluene to give the title compound as an oil.

### Step 2

### Production of (3,5-difluoro-4-methoxyphenyl)-(2,3-dihydrobenzo[1,4]oxazin-4-yl)-methanone

3,4-Dihydro-2H-benzo[1,4]oxazine (300 mg) obtained in Step 2 of Reference Example 1 was dissolved in chloroform (6 mL) and triethylamine (0.371 mL) and 3,5-difluoro-4-methoxybenzoyl chloride (459 mg) were added under ice-cooling. The mixture was stirred at room temperature for 12 hrs, and the reaction mixture was purified by silica gel chromatography (n-hexane-ethyl acetate=4:1) to give the title compound (709 mg) as an oil.

### Step 3

### Production of (3,5-difluoro-4-hydroxyphenyl)-(2,3-dihydrobenzo[1,4]oxazin-4-yl)-methanone

(3,5-Difluoro-4-methoxyphenyl)-(2,3-dihydrobenzo[1,4]oxazin-4-yl)-methanone (672 mg) was dissolved in methylene chloride (7 mL). After cooling to −78° C., boron tribromide (1.0 M methylene chloride solution, 3.3 mL) was added dropwise, and the mixture was stirred at room temperature for 20 hrs. The reaction mixture was poured into water, and the mixture was extracted with chloroform. The extract was washed successively with water and saturated brine, dried over anhydrous sodium sulfate, and concentrated. The obtained residue was purified by silica gel chromatography (n-hexane-ethyl acetate=7:3) to give the title compound (258 mg) as white crystals.

### Reference Example 7

### Production of (2,3-dihydrobenzo[1,4]oxazin-4-yl)-(4-hydroxy-3,5-dimethylphenyl)-methanone

### Step 1

### Production of 4-benzyloxy-3,5-dimethylbenzoyl chloride

Methylene chloride (8 mL) was added to 4-benzyloxy-3,5-dimethylbenzoic acid (256 mg), and oxalyl chloride (0.1 mL) and N,N-dimethylformamide (1 drop) were added under ice-cooling. After stirring overnight at room temperature, the reaction mixture was concentrated under reduced pressure, and azeotroped with toluene to give the title compound (276.7 mg) as a pale-yellow solid.

### Step 2

### Production of (4-benzyloxy-3,5-dimethylphenyl)-(2,3-dihydrobenzo[1,4]oxazin-4-yl)-methanone

3,4-Dihydro-2H-benzo[1,4]oxazine (135 mg) obtained in Step 2 of Reference Example 1 was dissolved in methylene chloride (7 mL), and triethylamine (0.17 mL) and 4-benzyloxy-3,5-dimethylbenzoyl chloride (275 mg) were added under ice-cooling. After stirring overnight at room temperature, the reaction mixture was purified by silica gel chromatography (n-hexane-ethyl acetate=4:1) to give the title compound (378.3 mg) as an oil.

### Step 3

### Production of (2,3-dihydrobenzo[1,4]oxazin-4-yl)-(4-hydroxy-3,5-dimethylphenyl)-methanone

(4-Benzyloxy-3,5-dimethylphenyl)-(2,3-dihydrobenzo[1,4]oxazin-4-yl)-methanone (370.1 mg) was dissolved in tetrahydrofuran (10 mL). 7.5% Palladium-carbon (37 mg) was added to this solution and, under a hydrogen atmosphere, the mixture was stirred at room temperature for 1 hr. The reaction mixture was filtered through celite, and the residue was washed with tetrahydrofuran. The filtrate and washing solution were combined and the mixture was concentrated under reduced pressure. The obtained solid was crystallized from ethyl acetate to give the title compound (220.1 mg) as white crystals.

### Reference Example 8

### Production of (3,5-dichloro-4-hydroxyphenyl)-(2,3-dihydrobenzo[1,4]thiazin-4-yl)-methanone

### Step 1

### Production of ethyl 4-benzyloxy-3,5-dichlorobenzoate

Ethyl 3,5-dichloro-4-hydroxybenzoate (55.64 g) was dissolved in N,N-dimethylformamide (280 mL) and potassium carbonate (42.56 g) was added. Under ice-cooling, benzyl bromide (36 mL) was added dropwise, and the mixture was stirred overnight at 70° C. The solvent was evaporated, and the mixture was partitioned between water and ethyl acetate. The ethyl acetate layer was washed with saturated brine, dried over anhydrous sodium sulfate, and concentrated. The obtained residue was recrystallized from n-hexane to give the title compound (32.45 g). In addition, the filtrate was concentrated to give the title compound (42.19 g).

### Step 2

### Production of 4-benzyloxy-3,5-dichlorobenzoic acid

Ethyl 4-benzyloxy-3,5-dichlorobenzoate (42.19 g) was dissolved in methanol (70 mL) and tetrahydrofuran (140 mL). Under ice-cooling, 2N aqueous lithium hydroxide (130 mL) was added dropwise, and the mixture was stirred overnight at room temperature. A small amount of an insoluble material was removed by filtration. The filtrate was concentrated. Water was added, and the mixture was acidified by dropwise addition of 1N hydrochloric acid under ice-cooling. The precipitated solid was collected by filtration to give the title compound (34.83 g).

### Step 3

### Production of 4-benzyloxy-3,5-dichlorobenzoyl chloride

Chloroform (175 mL) was added to 4-benzyloxy-3,5-dichlorobenzoic acid (34.83 g), and oxalyl chloride (15.3 mL) and N,N-dimethylformamide (2 drops) were added under ice-cooling. The mixture was stirred at room temperature for 4 hrs, an insoluble material was removed by filtration, the filtrate was concentrated and azeotroped with toluene to give the title compound (37.371 g) as a pale-yellow solid.

### Step 4

### Production of 3,4-dihydro-2H-benzo[1,4]thiazine

Lithium aluminum hydride (1 g) was suspended in tetrahydrofuran (40 mL), 4H-benzo[1,4]thiazin-3-one (2 g) was added under ice-cooling by small portions. The mixture was heated under reflux for 8 hrs, and water (1 mL), 15% aqueous sodium hydroxide (1 mL) and water (3 mL) were successively added under ice-cooling, and the mixture was stirred at room temperature. The mixture was dried over anhydrous sodium sulfate, and concentrated to give the title compound (1.9181 g) as a yellow oil.

### Step 5

### Production of (4-benzyloxy-3,5-dichlorophenyl)-(2,3-dihydrobenzo[1,4]thiazin-4-yl)-methanone

3,4-Dihydro-2H-benzo[1,4]thiazine (1 g) was dissolved in chloroform (19 mL), and triethylamine (1.1 mL) and 4-benzyloxy-3,5-dichlorobenzoyl chloride (2.08 g) were added under ice-cooling. After stirring overnight at room temperature, the reaction mixture was purified by silica gel chromatography (n-hexane-ethyl acetate=5:1) to give the title compound (2.3350 g) as an oil.

### Step 6

### Production of (3,5-dichloro-4-hydroxyphenyl)-(2,3-dihydrobenzo[1,4]thiazin-4-yl)-methanone

(4-Benzyloxy-3,5-dichlorophenyl)-(2,3-dihydrobenzo[1,4]thiazin-4-yl)-methanone (223.7 mg) was dissolved in toluene (2 mL), and trifluoroacetic acid (2 mL) was added at room temperature. After stirring with heating at 80° C. for 1.5 hrs, the mixture was concentrated. The obtained solid was crystallized from ethyl acetate to give the title compound (101.6 mg) as white crystals.

### Reference Example 9

### Production of (3,5-dichloro-4-hydroxyphenyl)-(1-oxo-2,3-dihydro-1H-1λ⁴-benzo[1,4]thiazin-4-yl)-methanone

### Step 1

### Production of (4-benzyloxy-3,5-dichlorophenyl)-(1-oxo-2,3-dihydro-1H-1λ⁴-benzo[1,4]thiazin-4-yl)-methanone

(4-Benzyloxy-3,5-dichlorophenyl)-(2,3-dihydrobenzo[1,4]thiazin-4-yl)-methanone (426 mg) obtained in Step 5 of Reference Example 8 was dissolved in chloroform (10 mL), 3-chloroperbenzoic acid (171 mg) was added under ice-cooling, and the mixture was stirred overnight at room temperature. Saturated aqueous sodium hydrogencarbonate was added to the reaction solution, and the mixture was extracted with chloroform. The extract was dried over anhydrous sodium sulfate, and concentrated. The obtained residue was purified by silica gel chromatography (n-hexane-ethyl acetate=1:3) to give the title compound (398.4 mg) as a white amorphous form.

### Step 2

### Production of (3,5-dichloro-4-hydroxyphenyl)-(1-oxo-2,3-dihydro-1H-1λ⁴-benzo[1,4]thiazin-4-yl)-methanone

(4-Benzyloxy-3,5-dichlorophenyl)-(1-oxo-2,3-dihydro-1H-1,4-benzo[1,4]thiazin-4-yl)-methanone (210.9 mg) was dissolved in toluene (2 mL), and trifluoroacetic acid (2 mL) was added at room temperature. After stirring with heating at 80° C. for 1.5 hrs, the mixture was concentrated. The obtained solid was crystallized from ethyl acetate to give the title compound (157.5 mg) as pale-blue crystals.

### Reference Example 10

### Production of (3,5-dichloro-4-hydroxyphenyl)-(1,1-dioxo-2,3-dihydro-1H-1λ⁶-benzo[1,4]thiazin-4-yl)-methanone

### Step 1

### Production of (4-benzyloxy-3,5-dichlorophenyl)-(1,1-dioxo-2,3-dihydro-1H-1λ⁶-benzo[1,4]thiazin-4-yl)-methanone

(4-Benzyloxy-3,5-dichlorophenyl)-(2,3-dihydrobenzo[1,4]thiazin-4-yl)-methanone (496.6 mg) obtained in Step 5 of Reference Example 8 was dissolved in chloroform (15 mL), 3-chloroperbenzoic acid (597 mg) was added under ice-cooling, and the mixture was stirred overnight at room temperature. The insoluble material was removed by filtration, saturated aqueous sodium hydrogencarbonate was added to the filtrate, and the mixture was extracted with chloroform. The extract was dried over anhydrous sodium sulfate, and concentrated. The obtained residue was purified by silica gel chromatography (n-hexane-ethyl acetate=2:1) to give the title compound (363.9 mg) as a white solid.

### Step 2

### Production of (3,5-dichloro-4-hydroxyphenyl)-(1,1-dioxo-2,3-dihydro-1H-1λ⁶-benzo[1,4]thiazin-4-yl)-methanone

(4-Benzyloxy-3,5-dichlorophenyl)-(1,1-dioxo-2,3-dihydro-1H-1λ⁶-benzo[1,4]thiazin-4-yl)-methanone (245 mg) was dissolved in toluene (2.5 mL), and trifluoroacetic acid (2.5 mL) was added at room temperature. After stirring with heating at 80° C. for 2 hrs, and the mixture was concentrated. The obtained solid was crystallized from ethyl acetate to give the title compound (112 mg) as white crystals.

### Reference Example 11

### Production of (3,5-dichloro-4-hydroxyphenyl)-(2,3-dihydrobenzo[1,4]oxazin-4-yl)-methanethione

### Step 1

### Production of (4-benzyloxy-3,5-dichlorophenyl)-(2,3-dihydrobenzo[1,4]oxazin-4-yl)-methanone

3,4-Dihydro-2H-benzo[1,4]oxazine (0.4 g) obtained in Step 2 of Reference Example 1 was dissolved in chloroform (20 mL), triethylamine (0.5 mL) and 4-benzyloxy-3,5-dichlorobenzoyl chloride (0.947 g) obtained in Step 3 of Reference Example 8 were added under ice-cooling. After stirring overnight at room temperature, the reaction mixture was purified by silica gel chromatography (n-hexane-ethyl acetate=6:1) to give the title compound (1.0635 g) as a white solid.

### Step 2

### Production of (4-benzyloxy-3,5-dichlorophenyl)-(2,3-dihydrobenzo[1,4]oxazin-4-yl)-methanethione

(4-Benzyloxy-3,5-dichlorophenyl)-(2,3-dihydrobenzo[1,4]oxazin-4-yl)-methanone (400 mg) and Lawesson reagent (235 mg) were suspended in tetrahydrofuran (3 mL), and the mixture was stirred with heating at 95° C. for 2 hrs. The reaction mixture was purified by silica gel chromatography (n-hexane-ethyl acetate=6:1) to give the title compound (372.4 mg) as an orange amorphous form.

### Step 3

### Production of (3,5-dichloro-4-hydroxyphenyl)-(2,3-dihydrobenzo[1,4]oxazin-4-yl)-methanethione

(4-Benzyloxy-3,5-dichlorophenyl)-(2,3-dihydrobenzo[1,4]oxazin-4-yl)-methanethione (366.8 mg) was dissolved in toluene (4 mL), and trifluoroacetic acid (4 mL) was added at room temperature. After stirring with heating at 80° C. for 2 hrs, and the mixture was concentrated. The obtained solid was crystallized from ethyl acetate to give the title compound (84.6 mg) as orange crystals.

### Reference Example 12

### Production of (3,5-dichloro-4-hydroxyphenyl)-(6-methyl-2,3-dihydrobenzo[1,4]oxazin-4-yl)-methanone

### Step 1

### Production of 6-methyl-3,4-dihydro-2H-benzo[1,4]oxazine

Lithium aluminum hydride (0.8 g) was suspended in tetrahydrofuran (50 mL), and 6-methyl-4H-benzo[1,4]oxazin-3-one (1.76 g) was added under ice-cooling by small portions. After heating under reflux for 6 hrs, water (0.8 mL), 15% aqueous sodium hydroxide (0.8 mL) and water (2.4 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (1.5719 g) as an orange oil.

### Step 2

### Production of (3,5-dichloro-4-hydroxyphenyl)-(6-methyl-2,3-dihydrobenzo[1,4]oxazin-4-yl)-methanone

6-Methyl-3,4-dihydro-2H-benzo[1,4]oxazine (298 mg) and 3,5-dichloro-4-hydroxybenzoyl chloride (451 mg) obtained in Step 1 of Reference Example 3 were dissolved in ethyl acetate (7 mL), and the mixture was stirred overnight at 95° C. The reaction mixture was allowed to cool to room temperature, and the precipitated solid was collected by filtration to give the title compound (606.4 mg) as pale-orange crystals.

### Reference Example 13

### Production of (3,5-dichloro-4-hydroxyphenyl)-(7-methyl-2,3-dihydrobenzo[1,4]oxazin-4-yl)-methanone

### Step 1

### Production of 7-methyl-4H-benzo[1,4]oxazin-3-one

2-Amino-5-methylphenol (2.463 g) and benzyltriethylammonium chloride (4.56 g) were suspended in chloroform (50 mL), sodium hydrogencarbonate (13.44 g) and chloroacetyl chloride (1.9 mL) were added under ice-cooling and the mixture was stirred under ice-cooling for 1 hr. Thereafter, and the mixture was stirred overnight with heating at 55° C. The reaction mixture was concentrated, water was added and the mixture was extracted with ethyl acetate. The obtained ethyl acetate layer was washed successively with 1N hydrochloric acid, water, saturated aqueous sodium hydrogencarbonate, water and saturated brine, dried over anhydrous sodium sulfate, and concentrated. The obtained solid was crystallized from ethyl acetate to give the title compound (1.7636 g) as a khaki solid.

### Step 2

### Production of 7-methyl-3,4-dihydro-2H-benzo[1,4]oxazine

Lithium aluminum hydride (0.8 g) was suspended in tetrahydrofuran (50 mL), and 7-methyl-4H-benzo[1,4]oxazin-3-one (1.76 g) was added under ice-cooling by small portions. After heating under reflux for 6 hrs, water (0.8 mL), 15% aqueous sodium hydroxide (0.8 mL) and water (2.4 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (1.5038 g) as an orange-tan oil.

### Step 3

### Production of (3,5-dichloro-4-hydroxyphenyl)-(7-methyl-2,3-dihydrobenzo[1,4]oxazin-4-yl)-methanone

7-Methyl-3,4-dihydro-2H-benzo[1,4]oxazine (298 mg) and 3,5-dichloro-4-hydroxybenzoyl chloride (451 mg) obtained in Step 1 of Reference Example 3 were dissolved in ethyl acetate (7 mL), and the mixture was stirred overnight at 95° C. The reaction mixture was purified by silica gel chromatography (n-hexane-ethyl acetate=4:1) to give the title compound (609.3 mg) as a white amorphous solid.

### Reference Example 14

### Production of (3,5-dichloro-4-hydroxyphenyl)-(5-methyl-2,3-dihydrobenzo[1,4]oxazin-4-yl)-methanone

### Step 1

### Production of 2-chloro-N-(2-hydroxy-6-methylphenyl)-acetamide

2-Amino-3-methylphenol (737 mg) was dissolved in tetrahydrofuran (20 mL), triethylamine (1 mL) and chloroacetyl chloride (0.5 mL) were added under ice-cooling, and the mixture was stirred overnight at room temperature. The reaction mixture was poured into water, and the mixture was extracted with ethyl acetate. The obtained ethyl acetate layer was washed successively with 1N hydrochloric acid, water and saturated brine, and dried over anhydrous sodium sulfate. The solvent was evaporated to give the title compound (1.2217 g) as a khaki solid.

### Step 2

### Production of 5-methyl-4H-benzo[1,4]oxazin-3-one

2-Chloro-N-(2-hydroxy-6-methylphenyl)-acetamide (1.2217 g) was dissolved in N,N-dimethylformamide (7 mL), and potassium carbonate (0.99 g) and sodium iodide (catalytic amount) were added at room temperature. After stirring overnight at 80° C., the mixture was poured into water. The mixture was extracted with ethyl acetate. The obtained ethyl acetate layer was washed successively with 1N hydrochloric acid and water, then successively washed with saturated aqueous sodium hydrogencarbonate, water and saturated brine, and dried over anhydrous sodium sulfate. The solvent was evaporated to give the title compound (813.5 mg) as a pale-yellow solid.

### Step 3

### Production of 5-methyl-3,4-dihydro-2H-benzo[1,4]oxazine

Lithium aluminum hydride (0.4 g) was suspended in tetrahydrofuran (40 mL) and, under ice-cooling, 5-methyl-4H-benzo[1,4]oxazin-3-one (810 mg) was added by small portions. After heating under reflux for 7 hrs, water (0.4 mL), 15% aqueous sodium hydroxide (0.4 mL) and water (1.2 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (471.7 mg) as a red brown oil.

### Step 4

### Production of (3,5-dichloro-4-hydroxyphenyl)-(5-methyl-2,3-dihydrobenzo[1,4]oxazin-4-yl)-methanone

5-Methyl-3,4-dihydro-2H-benzo[1,4]oxazine (224 mg) and 3,5-dichloro-4-hydroxybenzoyl chloride (338 mg) obtained in Step 1 of Reference Example 3 were dissolved in ethyl acetate (3.5 mL), and the mixture was stirred overnight at 95° C. The reaction mixture was allowed to cool to room temperature, and the precipitated solid was collected by filtration to give the title compound (331.9 mg) as gray crystals.

### Reference Example 15

### Production of (3,5-dichloro-4-hydroxyphenyl)-(8-methyl-2,3-dihydrobenzo[1,4]oxazin-4-yl)-methanone

### Step 1

### Production of 4-bromo-2-methylphenol

o-Cresol (5 g) was dissolved in acetic acid (50 mL) and 48% aqueous hydrogen bromide (25 mL), dimethyl sulfoxide (25 mL) was added dropwise at room temperature. After stirring at room temperature for 1 hr, the reaction mixture was neutralized with sodium carbonate. Water was added, and the mixture was extracted with ethyl ether. The obtained ethyl ether layer was washed successively with water and saturated brine, and dried over magnesium sulfate. The solvent was evaporated, and the residue was used for the next Step without purification.

### Step 2

### Production of 4-bromo-2-methyl-6-nitrophenol

To a mixture of 4-bromo-2-methylphenol produced in Step 1 and sodium nitrite (10.4 g) were added n-hexane (75 mL), isopropyl ether (35 mL) and water (50 mL), and 4.5N sulfuric acid (110 mL) was added dropwise at room temperature. After stirring at room temperature for 15 hrs, the reaction mixture was washed successively with saturated aqueous sodium hydrogencarbonate, water and saturated brine, and dried over anhydrous sodium sulfate. The solvent was evaporated, and the obtained residue was purified by silica gel chromatography (n-hexane-ethyl acetate=5:1) to give the title compound (7.14 g) as a yellow solid.

### Step 3

### Production of 2-amino-6-methylphenol hydrobromide

4-Bromo-2-methyl-6-nitrophenol (7.1 g) was dissolved in methanol (50 mL). 7.5% Palladium-carbon (1.5 g) was added to this solution, and under a hydrogen atmosphere (2 kgf/cm²), and the mixture was stirred at room temperature for 18 hrs. The reaction mixture was filtered through celite, and the filtrate was concentrated under reduced pressure. The obtained solid was crystallized from ethyl acetate to give the title compound (5.40 g) as a brown solid.

### Step 4

### Production of 2-chloro-N-(2-hydroxy-3-methylphenyl)-acetamide

2-Amino-6-methylphenol hydrobromide (408 mg) was suspended in tetrahydrofuran (10 mL), triethylamine (0.7 mL) and chloroacetyl chloride (0.17 mL) were added under ice-cooling, and the mixture was stirred overnight at room temperature. The reaction mixture was poured into water, and the mixture was extracted with ethyl acetate. The obtained ethyl acetate layer was washed successively with 1N hydrochloric acid, water and saturated brine, and dried over anhydrous sodium sulfate. The solvent was evaporated to give the title compound (384.6 mg) as a dark brown solid.

### Step 5

### Production of 8-methyl-4H-benzo[1,4]oxazin-3-one

2-Chloro-N-(2-hydroxy-3-methylphenyl)-acetamide (380 mg) was dissolved in N,N-dimethylformamide (4 mL), potassium carbonate (315 mg) and sodium iodide (catalytic amount) were added at room temperature. After stirring overnight at 80° C., the mixture was poured into water and the mixture was extracted with ethyl acetate. The obtained ethyl acetate layer washed successively with 1N hydrochloric acid and water, and successively with saturated aqueous sodium hydrogencarbonate, water and saturated brine, and dried over anhydrous sodium sulfate. The solvent was evaporated to give the title compound (284.3 mg) as a red brown solid.

### Step 6

### Production of 8-methyl-3,4-dihydro-2H-benzo[1,4]oxazine

Lithium aluminum hydride (150 mg) was suspended in tetrahydrofuran (25 mL), 8-methyl-4H-benzo[1,4]oxazin-3-one (280 mg) was added under ice-cooling by small portions. After heating under reflux for 7 hrs, water (0.15 mL), 15% aqueous sodium hydroxide (0.15 mL) and water (0.45 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (145.2 mg) as a red brown oil.

### Step 7

### Production of (3,5-dichloro-4-hydroxyphenyl)-(8-methyl-2,3-dihydrobenzo[1,4]oxazin-4-yl)-methanone

8-Methyl-3,4-dihydro-2H-benzo[1,4]oxazine (116.3 mg) and 3,5-dichloro-4-hydroxybenzoyl chloride (176 mg) obtained in Step 1 of Reference Example 3 were dissolved in ethyl acetate (2 mL), and the mixture was stirred overnight at 95° C. The reaction mixture was allowed to cool to room temperature, and the precipitated solid was collected by filtration to give the title compound (121.3 mg) as pale-brown crystals.

### Reference Example 16

### Production of (3,5-dichloro-4-hydroxyphenyl)-(2,3-dihydronaphtho[2,1-b][1,4]oxazin-1-yl)-methanone

### Step 1

### Production of 2-chloro-N-(2-hydroxynaphthalen-1-yl)-acetamide

1-Aminonaphthalen-2-ol hydrochloride (1.1739 g) was suspended in tetrahydrofuran (20 mL), triethylamine (2 mL) and chloroacetyl chloride (0.5 mL) were added under ice-cooling, and the mixture was stirred overnight at room temperature. The reaction mixture was poured into water, and the mixture was extracted with ethyl acetate. The obtained ethyl acetate layer was washed successively with 1N hydrochloric acid, water and saturated brine, and dried over anhydrous sodium sulfate. The solvent was evaporated to give the title compound (1.227 g) as a dark brown solid.

### Step 2

### Production of 1H-naphtho[2,1-b][1,4]oxazin-2-one

2-Chloro-N-(2-hydroxynaphthalen-1-yl)-acetamide (1.22 g) was dissolved in N,N-dimethylformamide (10 mL), and potassium carbonate (860 mg) and sodium iodide (catalytic amount) were added at room temperature. After stirring overnight at 80° C., and the mixture was poured into water, and the mixture was extracted with ethyl acetate. The obtained ethyl acetate layer was washed successively with 1N hydrochloric acid and water, subsequently with saturated aqueous sodium hydrogencarbonate, water and saturated brine, and dried over anhydrous sodium sulfate. The solvent was evaporated to give the title compound (1.0553 g) as a dark brown solid.

### Step 3

### Production of 2,3-dihydro-1H-naphtho[2,1-b][1,4]oxazine

Lithium aluminum hydride (400 mg) was suspended in tetrahydrofuran (40 mL), 1H-naphtho[2,1-b][1,4]oxazin-2-one (1.055 g) was added under ice-cooling by small portions. After heating under reflux for 7 hrs, water (0.4 mL), 15% aqueous sodium hydroxide (0.4 mL) and water (1.2 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (361.4 mg) as a dark brown oil.

### Step 4

### Production of (3,5-dichloro-4-hydroxyphenyl)-(2,3-dihydronaphtho[2,1-b][1,4]oxazin-1-yl)-methanone

2,3-Dihydro-1H-naphtho[2,1-b][1,4]oxazine (185 mg) and 3,5-dichloro-4-hydroxybenzoyl chloride (225 mg) obtained in Step 1 of Reference Example 3 were dissolved in ethyl acetate (3 mL), and the mixture was stirred overnight at 95° C. The reaction mixture was allowed to cool to room temperature, and the precipitated solid was collected by filtration to give the title compound (334.5 mg) as gray crystals.

### Reference Example 17

### Production of (3,5-dichloro-4-hydroxyphenyl)-(6-methoxy-2,3-dihydrobenzo[1,4]oxazin-4-yl)-methanone

### Step 1

### Production of 2-amino-4-methoxyphenol

4-Methoxy-2-nitrophenol (3.38 g) was dissolved in tetrahydrofuran (100 mL). 7.5% Palladium-carbon (0.34 g) was added to this solution and, under a hydrogen atmosphere, and the mixture was stirred at room temperature for 3.5 hrs. The reaction mixture was filtered through celite, and the residue was washed with tetrahydrofuran. The filtrate and washing solution were combined and the mixture was concentration under reduced pressure to give the title compound (2.8528 g) as a beige solid.

### Step 2

### Production of 6-methoxy-4H-benzo[1,4]oxazin-3-one

2-Amino-4-methoxyphenol (2.85 g) and benzyltriethylammonium chloride (4.56 g) were suspended in chloroform (50 mL), sodium hydrogencarbonate (6.72 g) and chloroacetyl chloride (1.9 mL) were added under ice-cooling, and the mixture was stirred under ice-cooling for 1 hr. Thereafter, the mixture was stirred overnight with heating at 55° C. The reaction mixture was concentrated, water was added and the mixture was extracted with ethyl acetate. The obtained ethyl acetate layer was washed successively with 1N hydrochloric acid, water, saturated aqueous sodium hydrogencarbonate, water and saturated brine, dried over anhydrous sodium sulfate, and concentrated. The obtained solid was crystallized from ethyl acetate to give the title compound (1.3884 g) as a pale-orange solid. The mother liquor was purified by silica gel chromatography (n-hexane-ethyl acetate=3:1) to give the title compound (151 mg) as a white solid.

### Step 3

### Production of 6-methoxy-3,4-dihydro-2H-benzo[1,4]oxazine

Lithium aluminum hydride (700 mg) was suspended in tetrahydrofuran (50 mL), 6-methoxy-4H-benzo[1,4]oxazin-3-one (1.53 g) was added under ice-cooling by small portions. After heating under reflux for 6 hrs, water (0.7 mL), 15% aqueous sodium hydroxide (0.7 mL) and water (2.1 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=3:1) to give the title compound (1.3294 g) as a pale-yellow solid.

### Step 4

### Production of (3,5-dichloro-4-hydroxyphenyl)-(6-methoxy-2,3-dihydrobenzo[1,4]oxazin-4-yl)-methanone

6-Methoxy-3,4-dihydro-2H-benzo[1,4]oxazine (600 mg) and 3,5-dichloro-4-hydroxybenzoyl chloride (818 mg) obtained in Step 1 of Reference Example 3 were dissolved in ethyl acetate (14 mL), and the mixture was stirred overnight at 95° C. The reaction mixture was allowed to cool to room temperature, and the precipitated solid was collected by filtration to give the title compound (1.059 g) as white crystals.

### Reference Example 18

### Production of (3,5-dichloro-4-hydroxyphenyl)-(7-methoxy-2,3-dihydrobenzo[1,4]oxazin-4-yl)-methanone

### Step 1

### Production of 2-amino-5-methoxyphenol

5-Methoxy-2-nitrophenol (1.059 g) was dissolved in tetrahydrofuran (30 mL). 7.5% Palladium-carbon (0.1 g) was added to this solution and, under a hydrogen atmosphere, the mixture was stirred at room temperature for 3 hrs. The reaction mixture was filtered through celite, and the residue was washed with tetrahydrofuran. The filtrate and washing solution were combined and the mixture was concentrated under reduced pressure to give the title compound (0.9615 g) as a pale-orange solid.

### Step 2

### Production of 2-chloro-N-(2-hydroxy-4-methoxyphenyl)-acetamide

2-Amino-5-methoxyphenol (0.96 g) was dissolved in tetrahydrofuran (35 mL), triethylamine (1.15 mL) and chloroacetyl chloride (0.58 mL) were added under ice-cooling, and the mixture was stirred overnight at room temperature. The reaction mixture was poured into water, and the mixture was extracted with ethyl acetate. The obtained ethyl acetate layer was washed successively with 1N hydrochloric acid, water and saturated brine, and dried over anhydrous sodium sulfate. The solvent was evaporated to give the title compound (1.5106 g) as a bright yellow solid.

### Step 3

### Production of 7-methoxy-4H-benzo[1,4]oxazin-3-one

2-Chloro-N-(2-hydroxy-4-methoxyphenyl)-acetamide (1.51 g) was dissolved in N,N-dimethylformamide (7 mL), and potassium carbonate (1.04 g) and sodium iodide (catalytic amount) were added at room temperature. After stirring overnight at 80° C., and the mixture was poured into water, and the mixture was extracted with ethyl acetate. The obtained ethyl acetate layer was washed successively with 1N hydrochloric acid and water, and sequentially washed with saturated aqueous sodium hydrogencarbonate, water and saturated brine, and dried over anhydrous sodium sulfate. The solvent was evaporated, and the obtained solid was crystallized from ethyl acetate to give the title compound (682.6 mg) as a bright yellow solid.

### Step 4

### Production of 7-methoxy-3,4-dihydro-2H-benzo[1,4]oxazine

Lithium aluminum hydride (300 mg) was suspended in tetrahydrofuran (20 mL), 7-methoxy-4H-benzo[1,4]oxazin-3-one (680 mg) was added under ice-cooling by small portions. After heating under reflux for 6 hrs, water (0.3 mL), 15% aqueous sodium hydroxide (0.3 mL) and water (0.9 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=2:1) to give the title compound (588.1 mg) as a red brown oil.

### Step 5

### Production of (3,5-dichloro-4-hydroxyphenyl)-(7-methoxy-2,3-dihydrobenzo[1,4]oxazin-4-yl)-methanone

7-Methoxy-3,4-dihydro-2H-benzo[1,4]oxazine (367.6 mg) and 3,5-dichloro-4-hydroxybenzoyl chloride (496 mg) obtained in Step 1 of Reference Example 3 were dissolved in ethyl acetate (4 mL), and the mixture was stirred overnight at 95° C. The reaction mixture was allowed to cool to room temperature, and the precipitated solid was collected by filtration to give the title compound (518.2 mg) as light purple crystals.

### Reference Example 19

### Production of (3,5-dichloro-4-hydroxyphenyl)-(6-hydroxy-2,3-dihydrobenzo[1,4]oxazin-4-yl)-methanone

(3,5-Dichloro-4-hydroxyphenyl)-(6-methoxy-2,3-dihydrobenzo[1,4]oxazin-4-yl)-methanone (500 mg) obtained in Step 4 of Reference Example 17 was dissolved in methylene chloride (10 mL). After cooling to −78° C., boron tribromide (1.0 M methylene chloride solution, 2.1 mL) was added dropwise, and the mixture was stirred overnight at room temperature. The reaction mixture was poured into water, and the mixture was extracted with chloroform, and dried over anhydrous sodium sulfate. The solvent was evaporated, and the obtained residue was purified by silica gel chromatography (chloroform-methanol=10:1) to give the title compound (157.6 mg) as pale-blue crystals.

### Reference Example 20

### Production of (3,5-dichloro-4-hydroxyphenyl)-(7-hydroxy-2,3-dihydrobenzo[1,4]oxazin-4-yl)-methanone

(3,5-Dichloro-4-hydroxyphenyl)-(7-methoxy-2,3-dihydrobenzo[1,4]oxazin-4-yl)-methanone (504 mg) obtained in Step 5 of Reference Example 18 was dissolved in methylene chloride (10 mL). After cooling to −78° C., boron tribromide (1.0 M methylene chloride solution, 4.2 mL) was added dropwise, and the mixture was stirred overnight at room temperature. The reaction mixture was poured into water, and the precipitated solid was collected by filtration. The collected solid was purified by silica gel chromatography (chloroform-methanol=30:1) to give the title compound (355.7 mg) as white crystals.

### Reference Example 21

### Production of 4-(3,5-dichloro-4-hydroxybenzoyl)-3,4-dihydro-2H-benzo[1,4]oxazine-6-sulfonic acid diethylamide

### Step 1

### Production of 3-amino-N,N-diethyl-4-hydroxybenzenesulfonamide

3-Amino-N,N-diethyl-4-methoxybenzenesulfonamide (5 g) was dissolved in methylene chloride (150 mL). Under ice-cooling, boron tribromide (1.0 M methylene chloride solution, 38.7 mL) was added dropwise, and the mixture was stirred overnight at room temperature. Water (150 mL) was added dropwise to the reaction mixture under ice-cooling, and the aqueous layer was washed with chloroform. The obtained aqueous layer was weak acidified with 4N aqueous sodium hydroxide under ice-cooling. The precipitated solid was collected by filtration to give the title compound (4.0344 g) as a pale-beige solid.

### Step 2

### Production of 3-amino-4-(tert-butyldimethylsilyloxy)-N,N-diethylbenzenesulfonamide

3-Amino-N,N-diethyl-4-hydroxybenzenesulfonamide (1.5 g) was dissolved in N,N-dimethylformamide (8 mL), and imidazole (0.61 g) and tert-butylchlorodimethylsilane (1.18 g) were added under ice-cooling. After stirring at room temperature for 1.5 hrs, the mixture was poured into water, and the mixture was extracted with ethyl ether. The obtained ethyl ether layer was washed with water and saturated brine, dried over anhydrous sodium sulfate and concentrated. The obtained residue was purified by silica gel chromatography (n-hexane-ethyl acetate=7:1) to give the title compound (2.0654 g) as a white solid.

### Step 3

### Production of 4-benzyloxy-3,5-dichloro-N-(5-diethylsulfamoyl-2-hydroxyphenyl)-benzamide

3-Amino-4-(tert-butyldimethylsilyloxy)-N,N-diethylbenzenesulfonamide (1.5 g) was dissolved in methylene chloride (40 mL), pyridine (0.41 mL) and 4-benzyloxy-3,5-dichlorobenzoyl chloride (1.32 g) obtained in Step 3 of Reference Example 8 were added under ice-cooling, and the mixture was stirred overnight at room temperature. The solvent was evaporated and the residue was dissolved in N,N-dimethylformamide (5 mL), and potassium carbonate (2.89 g) was added at room temperature. After stirring with heating at 60° C. for 1.5 hrs, the mixture was acidified with 10% aqueous citric acid solution and water under ice-cooling. The precipitated solid was collected by filtration to give the title compound (2.1455 g) as a cream color solid.

### Step 4

### Production of 4-(4-benzyloxy-3,5-dichlorobenzoyl)-3,4-dihydro-2H-benzo[1,4]oxazine-6-sulfonic acid diethylamide

4-Benzyloxy-3,5-dichloro-N-(5-diethylsulfamoyl-2-hydroxyphenyl)-benzamide (2.136 g) was dissolved in N,N-dimethylformamide (40 mL), and potassium carbonate (1.41 g) and 1,2-dibromoethane (0.42 mL) were added at room temperature. After stirring overnight at 70° C., the mixture was poured into water, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate=5:2) to give the title compound (1.6181 g) as a white amorphous form.

### Step 5

### Production of 4-(3,5-dichloro-4-hydroxybenzoyl)-3,4-dihydro-2H-benzo[1,4]oxazine-6-sulfonic acid diethylamide

4-(4-Benzyloxy-3,5-dichlorobenzoyl)-3,4-dihydro-2H-benzo[1,4]oxazine-6-sulfonic acid diethylamide (1.6151 g) was dissolved in tetrahydrofuran (70 mL). 7.5% Palladium-carbon (0.16 g) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from ethyl ether to give the title compound (1.2863 g) as white crystals.

### Reference Example 22

### Production of 2,6-dichloro-4-(2,3-dihydrobenzo[1,4]oxazine-4-sulfonyl)phenol

3,4-Dihydro-2H-benzo[1,4]oxazine (246 mg) obtained in Step 2 of Reference Example 1 and 3,5-dichloro-4-hydroxybenzenesulfonyl chloride (475 mg) were dissolved in chloroform (8 mL), and the mixture was stirred overnight at room temperature. The reaction mixture was purified by silica gel chromatography (n-hexane-ethyl acetate=2:1) to give the title compound (146.8 mg) as beige crystals.

### Reference Example 23

### Production of (6-tert-butyl-2,3-dihydrobenzo[1,4]oxazin-4-yl)-(3,5-dichloro-4-hydroxyphenyl)-methanone

The title compound (539.6 mg) was obtained as white crystals by a method similar to Reference Example 14 and using 2-amino-4-tert-butylphenol instead of 2-amino-3-methylphenol.

### Reference Example 24

### Production of 4-(3,5-dichloro-4-hydroxybenzoyl)-4H-benzo[1,4]oxazin-3-one

### Step 1

### Production of 4-(4-benzyloxy-3,5-dichlorobenzoyl)-4H-benzo[1,4]oxazin-3-one

4H-Benzo[1,4]oxazin-3-one (224 mg) was dissolved in tetrahydrofuran (10 mL), 60% sodium hydride (78 mg) and 4-benzyloxy-3,5-dichlorobenzoyl chloride (473 mg) obtained in Step 3 of Reference Example 8 were successively added with stirring under ice-cooling, and the mixture was stirred overnight at room temperature. Under ice-cooling, water was added to the reaction mixture, and the mixture was extracted with ethyl acetate. The obtained ethyl acetate layer was washed successively with saturated aqueous sodium hydrogencarbonate, water and saturated brine, dried over anhydrous sodium sulfate, and the mixture was concentrated. The obtained residue was purified by silica gel chromatography (n-hexane-ethyl acetate=9:1) to give the title compound (294.3 mg) as a yellow solid.

### Step 2

### Production of 4-(3,5-dichloro-4-hydroxybenzoyl)-4H-benzo[1,4]oxazin-3-one

4-(4-Benzyloxy-3,5-dichlorobenzoyl)-4H-benzo[1,4]oxazin-3-one (290 mg) was dissolved in tetrahydrofuran (12 mL). 7.5% Palladium-carbon (29 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from ethyl acetate to give the title compound (176.8 mg) as yellow crystals.

### Reference Example 25

### Production of 4-(3,5-dichloro-4-hydroxybenzoyl)-3,4-dihydro-2H-benzo[1,4]oxazine-6-sulfonamide

The title compound (160.6 mg) was obtained as white crystals by a method similar to Step 2 to 5 of Reference Example 21 and using 3-amino-4-hydroxybenzenesulfonamide instead of 3-amino-N,N-diethyl-4-hydroxybenzenesulfonamide.

### Reference Example 26

### Production of (3,5-dichloro-4-hydroxyphenyl)-(3,4-dihydro-2H-quinolin-1-yl)-methanone

### Step 1

### Production of (4-benzyloxy-3,5-dichlorophenyl)-(3,4-dihydro-2H-quinolin-1-yl)-methanone

1,2,3,4-Tetrahydroquinoline (147 mg) was dissolved in chloroform (6 mL), and triethylamine (0.18 mL) and 4-benzyloxy-3,5-dichlorobenzoyl chloride (347 mg) obtained in Step 3 of Reference Example 8 were added under ice-cooling. After stirring overnight at room temperature, the reaction mixture was purified by silica gel chromatography (n-hexane-ethyl acetate=6:1) to give the title compound (407.9 mg) as a white amorphous form.

### Step 2

### Production of (3,5-dichloro-4-hydroxyphenyl)-(3,4-dihydro-2H-quinolin-1-yl)-methanone

(4-Benzyloxy-3,5-dichlorophenyl)-(3,4-dihydro-2H-quinolin-1-yl)-methanone (398.7 mg) was dissolved in tetrahydrofuran (12 mL). 7.5% Palladium-carbon (40 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from ethyl acetate to give the title compound (267.4 mg) as white crystals.

### Reference Example 27

### Production of (3,5-dichloro-4-hydroxyphenyl)-(2,3,4,5-tetrahydrobenzo[b]azepin-1-yl)-methanone

### Step 1

### Production of 2,3,4,5-tetrahydro-1H-benzo[b]azepine

Lithium aluminum hydride (100 mg) was suspended in tetrahydrofuran (10 mL), 1,3,4,5-tetrahydrobenzo[b]azepin-2-one (245 mg) was added under ice-cooling by small portions. After heating under reflux for 5.5 hrs, water (0.1 mL), 15% aqueous sodium hydroxide (0.1 mL) and water (0.3 mL) were successively added under ice-cooling, and the mixture was stirred at room temperature. The mixture was dried over anhydrous sodium sulfate, and concentrated to give the title compound (301.6 mg) as a yellow oil.

### Step 2

### Production of (4-benzyloxy-3,5-dichlorophenyl)-(2,3,4,5-tetrahydrobenzo[b]azepin-1-yl)-methanone

2,3,4,5-Tetrahydro-1H-benzo[b]azepine (301 mg) was dissolved in methylene chloride (10 mL), triethylamine (0.25 mL) and 4-benzyloxy-3,5-dichlorobenzoyl chloride (480 mg) obtained in Step 3 of Reference Example 8 were added under ice-cooling. After stirring overnight at room temperature, the reaction mixture was purified by silica gel chromatography (n-hexane-ethyl acetate=6:1) to give the title compound (531.9 mg) as a yellow green oil.

### Step 3

### Production of (3,5-dichloro-4-hydroxyphenyl)-(2,3,4,5-tetrahydrobenzo[b]azepin-1-yl)-methanone

(4-Benzyloxy-3,5-dichlorophenyl)-(2,3,4,5-tetrahydrobenzo[b]azepin-1-yl)-methanone (530 mg) was dissolved in tetrahydrofuran (15 mL). 7.5% Palladium-carbon (53 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from ethyl acetate to give the title compound (259.6 mg) as white crystals.

### Reference Example 28

### Production of (4-amino-3,5-dichlorophenyl)-(2,3-dihydrobenzo[1,4]oxazin-4-yl)-methanone

3,4-Dihydro-2H-benzo[1,4]oxazine (270 mg) obtained in Step 2 of Reference Example 1, 4-amino-3,5-dichlorobenzoic acid (412 mg) and N,N-dimethylaminopyridine (269 mg) were dissolved in chloroform (8 mL), and WSC.HCl (422 mg) was added under ice-cooling. After stirring overnight at room temperature, the reaction mixture was purified by silica gel chromatography (n-hexane-ethyl acetate=5:1) to give the title compound (412.5 mg) as bright yellow crystals.

### Reference Example 29

### Production of (5-chloro-6-hydroxypyridin-3-yl)-(2,3-dihydrobenzo[1,4]oxazin-4-yl)-methanone

3,4-Dihydro-2H-benzo[1,4]oxazine (270 mg) obtained in Step 2 of Reference Example 1, 5-chloro-6-hydroxynicotinic acid (347 mg) and 4-dimethylaminopyridine (269 mg) were dissolved in chloroform (12 mL), and WSC.HCl (422 mg) was added under ice-cooling. After stirring overnight at room temperature, the reaction mixture was purified by silica gel chromatography (chloroform-methanol=20:1) to give the title compound (295.7 mg) as cream color crystals.

### Reference Example 30

### Production of (2,3-dihydrobenzo[1,4]oxazin-4-yl)-(4-hydroxy-3,5-dinitrophenyl)-methanone

### Step 1

### Production of 4-hydroxy-3,5-dinitrobenzoyl chloride

1,2-Dimethoxyethane (5 mL) was added to 4-hydroxy-3,5-dinitrobenzoic acid (1 g) to dissolve same by heating the mixture to 70° C. Thionyl chloride (0.415 mL) was added, and the mixture was stirred overnight at 70° C. The reaction mixture was concentrated under reduced pressure, azeotroped with toluene, and dried to give the title compound as a yellow solid.

### Step 2

### Production of (2,3-dihydrobenzo[1,4]oxazin-4-yl)-(4-hydroxy-3,5-dinitrophenyl)-methanone

3,4-Dihydro-2H-benzo[1,4]oxazine (135 mg) obtained in Step 2 of Reference Example 1 and 4-hydroxy-3,5-dinitrobenzoyl chloride (271 mg) ware dissolved in ethyl acetate (4 mL), and the mixture was heated under reflux for 3 hrs. The solvent was evaporated, and the obtained solid was crystallized from methanol to give the title compound (211 mg) as pale-yellow crystals.

### Reference Example 31

### Production of (3-chloro-4-hydroxy-5-nitrophenyl)-(2,3-dihydrobenzo[1,4]oxazin-4-yl)-methanone

### Step 1

### Production of methyl 3-chloro-4-methoxy-5-nitrobenzoate

Concentrated sulfuric acid (200 mL) was added to methyl 3-chloro-4-methoxybenzoate (24.6 g) under ice-cooling and a mixture of fuming nitric acid (10.3 mL) and concentrated sulfuric acid (20 mL) was successively added dropwise under ice-cooling. After stirring under ice-cooling, the reaction mixture was poured into ice water (1 L). The precipitated solid was collected by filtration to give the title compound (28.8 g) as a cream color solid.

### Step 2

### Production of 3-chloro-4-hydroxy-5-nitrobenzoic acid

Methyl 3-chloro-4-methoxy-5-nitrobenzoate (28.8 g) was suspended in dimethyl sulfoxide (130 mL), and 50% aqueous potassium hydroxide (130 mL) was added under ice-cooling. After stirring with heating at 80° C. for 1.5 hrs, the mixture was ice-cooled, and 6N hydrochloric acid (200 mL) and water were added. The mixture was extracted with ethyl acetate, and the extract was washed successively with water and saturated brine, and dried over anhydrous sodium sulfate. The solvent was evaporated, and the obtained solid was crystallized from n-hexane to give the title compound (21.3 g) as a lemon solid.

### Step 3

### Production of 3-chloro-4-hydroxy-5-nitrobenzoyl chloride

1,2-Dimethoxyethane (5 mL) was added to 3-chloro-4-hydroxy-5-nitrobenzoic acid (1 g) to dissolve same by heating the mixture to 70° C. Thionyl chloride (0.436 mL) was added, and the mixture was stirred overnight at 70° C. The reaction mixture was concentrated under reduced pressure, azeotroped with toluene, and dried to give the title compound (1.10 g) as a yellow solid.

### Step 4

### Production of (3-chloro-4-hydroxy-5-nitrophenyl)-(2,3-dihydrobenzo[1,4]oxazin-4-yl)-methanone

3,4-Dihydro-2H-benzo[1,4]oxazine (270 mg) obtained in Step 2 of Reference Example 1 and 3-chloro-4-hydroxy-5-nitrobenzoyl chloride (519 mg) were dissolved in ethyl acetate (6 mL), and the mixture was stirred overnight at 95° C. The reaction mixture was allowed to cool to room temperature, and the precipitated solid was collected by filtration to give the title compound (537 mg) as yellow crystals.

### Reference Example 32

### Production of (3,5-dichloro-4-hydroxyphenyl)-(2,8-diisopropyl-2,3-dihydrobenzo[1,4]oxazin-4-yl)-methanone

### Step 1

### Production of 4-bromo-2-isopropylphenol

2-Isopropylphenol (25 g) was dissolved in acetic acid (250 mL) and 48% aqueous hydrogen bromide (125 mL), and dimethyl sulfoxide (125 mL) was added dropwise at room temperature. After stirring at room temperature for 2 hrs, the reaction mixture was neutralized with sodium carbonate (257 g). Water was added, and the mixture was extracted with ethyl ether. The obtained ethyl ether layer was washed successively with water and saturated brine, and dried over anhydrous sodium sulfate. The solvent was evaporated to give the title compound (40.2 g) as a pale-yellow solid.

### Step 2

### Production of 4-bromo-2-isopropyl-6-nitrophenol

To a mixture of 4-bromo-2-isopropylphenol (40.2 g) produced in Step 1 and sodium nitrite (41.5 g) were added n-hexane (300 mL), isopropyl ether (130 mL) and water (200 mL), and 4.5N sulfuric acid (430 mL) was added dropwise at room temperature. After stirring at room temperature for 1 hr, the reaction mixture was washed successively with saturated aqueous sodium hydrogencarbonate, water and saturated brine, and dried over anhydrous sodium sulfate. The solvent was evaporated, and the obtained residue was purified by silica gel chromatography (n-hexane-ethyl acetate=20:1) to give the title compound (41.1 g) as a yellow oil.

### Step 3

### Production of 2-amino-6-isopropylphenol hydrobromide

4-Bromo-2-isopropyl-6-nitrophenol (41.1 g) was dissolved in methanol (300 mL). 7.5% Palladium-carbon (8 g) was added to this solution and, under a hydrogen atmosphere (2 kgf/cm²), the mixture was stirred overnight at room temperature. The reaction mixture was filtered through celite, and the filtrate was concentrated under reduced pressure. The obtained solid was crystallized from ethyl acetate to give the title compound (27.4 g) as a beige solid.

### Step 4

### Production of 2-bromo-3-methylbutyryl chloride

Chloroform (200 mL) was added to 2-bromo-3-methylbutyric acid (25 g), and oxalyl chloride (14.5 mL) and N,N-dimethylformamide (3 drops) were added under ice-cooling. After stirring overnight at room temperature, the reaction mixture was concentrated under reduced pressure, and azeotroped with toluene to give the title compound as a yellow oil.

### Step 5

### Production of 2-bromo-N-(2-hydroxy-3-isopropylphenyl)-3-methylbutyramide

Ethyl acetate (70 mL) and water (80 mL) were added to 2-amino-6-isopropylphenol hydrobromide (6.14 g) obtained in Step 3 and dissolved therein. Sodium hydrogencarbonate (6.68 g) and 2-bromo-3-methylbutyryl chloride (5.80 g) obtained in Step 4 were added at room temperature, and the mixture was stirred at room temperature for 2 hrs. The reaction mixture was partitioned. The obtained ethyl acetate layer was washed successively with saturated aqueous sodium hydrogencarbonate, water and saturated brine, and dried over anhydrous sodium sulfate. The solvent was evaporated to give the title compound (9.0 g) as a pale-pink solid.

### Step 6

### Production of 2,8-diisopropyl-4H-benzo[1,4]oxazin-3-one

2-Bromo-N-(2-hydroxy-3-isopropylphenyl)-3-methylbutyramide (9.0 g) was dissolved in N,N-dimethylformamide (45 mL), potassium carbonate (4.94 g) was added, and the mixture was stirred overnight at room temperature. The reaction mixture was neutralized with 1N hydrochloric acid, and water was added. The precipitated solid was collected by filtration. The obtained solid was crystallized from n-hexane-isopropyl ether to give the title compound (4.5 g) as a white solid.

### Step 7

### Production of 2,8-diisopropyl-3,4-dihydro-2H-benzo[1,4]oxazine

2,8-Diisopropyl-4H-benzo[1,4]oxazin-3-one (1.0 g) was dissolved in tetrahydrofuran (10 mL), borane-tetrahydrofuran complex (1M tetrahydrofuran solution, 5.14 mL) was added, and the mixture was heated under reflux for 14.5 hrs. 6N Hydrochloric acid (5 mL) was added, and the mixture was stirred with heating again. The mixture was allowed to cool to room temperature, neutralized with sodium hydrogencarbonate, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate=6:1) to give the title compound (768 mg) as a colorless oil.

### Step 8

### Production of (4-benzyloxy-3,5-dichlorophenyl)-(2,8-diisopropyl-2,3-dihydrobenzo[1,4]oxazin-4-yl)-methanone

2,8-Diisopropyl-3,4-dihydro-2H-benzo[1,4]oxazine (150 mg) was dissolved in chloroform (5 mL), and pyridine (0.066 mL) and 4-benzyloxy-3,5-dichlorobenzoyl chloride (227 mg) obtained in Step 3 of Reference Example 8 were added under ice-cooling. After stirring overnight at room temperature, water was added to the reaction mixture, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate-9:1) to give the title compound (375 mg) as an oil.

### Step 9

### Production of (3,5-dichloro-4-hydroxyphenyl)-(2,8-diisopropyl-2,3-dihydrobenzo[1,4]oxazin-4-yl)-methanone

(4-Benzyloxy-3,5-dichlorophenyl)-(2,8-diisopropyl-2,3-dihydrobenzo[1,4]oxazin-4-yl)-methanone (370 mg) was dissolved in tetrahydrofuran (5 mL). 7.5% Palladium-carbon (70 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from n-hexane to give the title compound (212 mg) as white crystals.

### Reference Example 33

### Production of (3,5-dichloro-4-hydroxyphenyl)-[6-(pyrrolidine-1-sulfonyl)-2,3-dihydrobenzo[1,4]oxazin-4-yl]-methanone

### Step 1

### Production of 4-methoxy-3-nitrobenzenesulfonyl chloride

1-Methoxy-2-nitrobenzene (11 mL) was added dropwise to chlorosulfonic acid (25 mL) under ice-cooling, and the mixture was stirred at room temperature for 1.5 hrs. The reaction mixture was poured into ice water and the mixture was extracted with ethyl ether. The obtained ethyl ether layer was washed with water, and dried over anhydrous sodium sulfate. The solvent was evaporated to give the title compound (15.1527 g) as a red brown oil.

### Step 2

### Production of 1-(4-methoxy-3-nitrobenzenesulfonyl)pyrrolidine

4-Methoxy-3-nitrobenzenesulfonyl chloride (5.1531 g) was dissolved in pyridine (17 mL), pyrrolidine (2.05 g) was added under ice-cooling, and the mixture was stirred overnight at room temperature. The reaction mixture was concentrated under reduced pressure, the residue was poured into water. The mixture was extracted with chloroform, and dried over anhydrous sodium sulfate. The solvent was evaporated, and the obtained residue was purified by silica gel chromatography (n-hexane-ethyl acetate=1:1) to give the title compound (4.158 g) as a yellow solid.

### Step 3

### Production of 2-nitro-4-(pyrrolidine-1-sulfonyl)phenol

1-(4-Methoxy-3-nitrobenzenesulfonyl)pyrrolidine (2.054 g) was dissolved in dimethyl sulfoxide (40 mL), and 50% aqueous potassium hydroxide (40 mL) was added under ice-cooling. After stirring with heating at 80° C. for 6 hrs, the mixture was ice-cooled, and 6N hydrochloric acid (80 mL) was added. The mixture was extracted with chloroform. The extract was washed successively with water and saturated brine, and dried over anhydrous sodium sulfate. The solvent was evaporated to give the title compound (2.198 g) as a yellow solid.

### Step 4

### Production of 2-amino-4-(pyrrolidine-1-sulfonyl)phenol

2-Nitro-4-(pyrrolidine-1-sulfonyl)phenol (2.19 g) was dissolved in tetrahydrofuran (50 mL). 7.5% Palladium-carbon (0.2 g) was added to this solution and, under a hydrogen atmosphere, the mixture was stirred at room temperature for 4 hrs. The reaction mixture was filtered through celite, and the residue was washed with tetrahydrofuran. The filtrate and washing solution were combined and the mixture was concentrated under reduced pressure. The obtained residue was purified by silica gel chromatography (n-hexane-ethyl acetate=1:l) to give the title compound (1.5494 g) as a cream color solid.

### Step 5

### Production of 2-(tert-butyldimethylsilyloxy)-5-(pyrrolidine-1-sulfonyl)phenylamine

2-Amino-4-(pyrrolidine-1-sulfonyl)phenol (1.54 g) was dissolved in N,N-dimethylformamide (9 mL), and imidazole (0.649 g) and tert-butylchlorodimethylsilane (1.254 g) were added under ice-cooling. After stirring at room temperature of 1.5 hrs, the mixture was poured into water, and the mixture was extracted with ethyl ether. The obtained ethyl ether layer was washed with water and saturated brine, dried over anhydrous sodium sulfate, and concentrated. The obtained residue was purified by silica gel chromatography (n-hexane-ethyl acetate=7:1) to give the title compound (2.1549 g) as a white solid.

### Step 6

### Production of 4-benzyloxy-3,5-dichloro-N-[2-hydroxy-5-(pyrrolidine-1-sulfonyl)phenyl]benzamide

2-(tert-Butyldimethylsilyloxy)-5-(pyrrolidine-1-sulfonyl)phenylamine (727.8 mg) was dissolved in methylene chloride (17 mL), pyridine (0.2 mL) and 4-benzyloxy-3,5-dichlorobenzoyl chloride (644 mg) obtained in Step 3 of Reference Example 8 were added under ice-cooling, and the mixture was stirred overnight at room temperature. The reaction mixture was concentrated under reduced pressure, and the residue was dissolved in N,N-dimethylformamide (4 mL). Potassium carbonate (1.41 g) was added at room temperature. After stirring with heating at 60° C. for 1.5 hrs, the mixture was acidified with 10% aqueous citric acid solution and water under ice-cooling. The precipitated solid was collected by filtration to give the title compound (1.0619 g) as a cream color solid.

### Step 7

### Production of (4-benzyloxy-3,5-dichlorophenyl)-[6-(pyrrolidine-1-sulfonyl)-2,3-dihydrobenzo[1,4]oxazin-4-yl]-methanone

4-Benzyloxy-3,5-dichloro-N-[2-hydroxy-5-(pyrrolidine-1-sulfonyl)phenyl]benzamide (500 mg) was dissolved in N,N-dimethylformamide (5 mL), and potassium carbonate (332 mg) and 1,2-dibromoethane (0.099 mL) were added at room temperature. After stirring overnight at 70° C., the mixture was poured into water. The mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate=2:1) to give the title compound (459.5 mg) as a white amorphous form.

### Step 8

### Production of (3,5-dichloro-4-hydroxyphenyl)-[6-(pyrrolidine-1-sulfonyl)-2,3-dihydrobenzo[1,4]oxazin-4-yl]-methanone

(4-Benzyloxy-3,5-dichlorophenyl)-[6-(pyrrolidine-1-sulfonyl)-2,3-dihydrobenzo[1,4]oxazin-4-yl]-methanone (442.5 mg) was dissolved in tetrahydrofuran (20 mL). 7.5% Palladium-carbon (45 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained residue was purified by silica gel chromatography (n-hexane-ethyl acetate=1:1) to give the title compound (384.5 mg) as a white amorphous solid.

### Reference Example 34

### Production of 4-(3,5-dichloro-4-hydroxybenzoyl)-3,4-dihydro-2H-benzo[1,4]oxazine-6-sulfonic acid ethylamide

The title compound (341.8 mg) was obtained as a white amorphous solid by a method similar to Steps 2 to 8 of Reference Example 33 and using ethylamine instead of pyrrolidine.

### Reference Example 35

### Production of 4-(3,5-dichloro-4-hydroxybenzoyl)-3,4-dihydro-2H-benzo[1,4]oxazine-6-sulfonic acid dimethylamide

The title compound (379.2 mg) was obtained as a white amorphous solid by a method similar to Steps 2 to 8 of Reference Example 33 and using dimethylamine instead of pyrrolidine.

### Reference Example 36

### Production of (3,5-dichloro-4-hydroxyphenyl)-(2,3-dihydropyrido[3,2-b][1,4]oxazin-4-yl)-methanone

### Step 1

### Production of 3,4-dihydro-2H-pyrido[3,2-b][1,4]oxazine

Lithium aluminum hydride (2 g) was suspended in tetrahydrofuran (80 mL), and 4H-pyrido[3,2-b][1,4]oxazin-3-one (3.956 g) was added under ice-cooling by small portions. After heating under reflux for 2 hrs, water (2 mL), 15% aqueous sodium hydroxide (2 mL) and water (6 mL) were successively added under ice-cooling, and the mixture was stirred at room temperature. The mixture was dried over anhydrous sodium sulfate. The solvent was evaporated, and the residue was purified by silica gel chromatography (n-hexane-ethyl acetate=1:9) to give the title compound (3.407 g) as a white solid.

### Step 2

### Production of (4-benzyloxy-3,5-dichlorophenyl)-(2,3-dihydropyrido[3,2-b][1,4]oxazin-4-yl)-methanone

3,4-Dihydro-2H-pyrido[3,2-b][1,4]oxazine (272 mg) was dissolved in chloroform (15 mL), and triethylamine (0.335 mL) and 4-benzyloxy-3,5-dichlorobenzoyl chloride (662 mg) obtained in Step 3 of Reference Example 8 were added under ice-cooling. The mixture was stirred at room temperature for 12 hrs, and the reaction mixture was purified by silica gel chromatography (n-hexane-ethyl acetate=4:1) to give the title compound (646 mg) as a white solid.

### Step 3

### Production of (3,5-dichloro-4-hydroxyphenyl)-(2,3-dihydropyrido[3,2-b][1,4]oxazin-4-yl)-methanone

(4-Benzyloxy-3,5-dichlorophenyl)-(2,3-dihydropyrido[3,2-b][1,4]oxazin-4-yl)-methanone (415 mg) was dissolved in tetrahydrofuran (10 mL). 7.5% Palladium-carbon (40 mg) was added to this solution and, under a hydrogen atmosphere, the mixture was stirred at room temperature for 0.5 hr. The reaction mixture was filtered through celite, and the residue was washed with tetrahydrofuran. The filtrate and washing solution were combined and the mixture was concentrated under reduced pressure. The obtained solid was crystallized from ethyl acetate-tetrahydrofuran to give the title compound (266 mg) as white crystals.

### Reference Example 37

### Production of 5-(3,5-dichloro-4-hydroxybenzoyl)-1,3,4,5-tetrahydrobenzo[b][1,4]diazepin-2-one

### Step 1

### Production of 1,3,4,5-tetrahydrobenzo[b][1,4]diazepin-2-one

Acrylic acid (8.6 g) and benzene-1,2-diamine (5.4 g) were added to polyphosphoric acid (1.5 g), and the mixture was heated under reflux for 3 hrs. Water (100 mL), chloroform (200 mL) and N,N-dimethylformamide (50 mL) were added to the reaction mixture, and the mixture was extracted with chloroform. The obtained chloroform layer was washed successively with water, saturated aqueous sodium hydrogencarbonate and water, and dried over anhydrous sodium sulfate. The solvent was evaporated and the obtained residue was purified by silica gel chromatography (n-hexane-ethyl acetate=1:4) to give the title compound (490 mg).

### Step 2

### Production of 5-(3,5-dichloro-4-hydroxybenzoyl)-1,3,4,5-tetrahydrobenzo[b][1,4]diazepin-2-one

1,3,4,5-Tetrahydrobenzo[b][1,4]diazepin-2-one (194.4 mg) and 3,5-dichloro-4-hydroxybenzoyl chloride (177.99 mg) obtained in Step 1 of Reference Example 3 were dissolved in ethyl acetate (3 mL), and the mixture was stirred with heating at 95° C. for 12 hrs. The reaction mixture was purified by silica gel chromatography to give the title compound (118 mg) as white crystals.

### Reference Example 38

### Production of (3,5-dichloro-2-hydroxyphenyl)-(2,3-dihydrobenzo[1,4]oxazin-4-yl)-methanone

### Step 1

### Production of 3,5-dichloro-2-hydroxybenzoyl chloride

3,5-Dichloro-2-hydroxybenzoic acid (600 mg) was suspended in toluene (6 mL), and thionyl chloride (0.275 mL) and N,N-dimethylformamide (1 drop) were added. After stirring with heating at 70° C. for 2 hrs, and the mixture was concentrated and azeotroped with toluene to give the title compound as a pale-yellow solid.

### Step 2

### Production of (3,5-dichloro-2-hydroxyphenyl)-(2,3-dihydrobenzo[1,4]oxazin-4-yl)-methanone

3,4-Dihydro-2H-benzo[1,4]oxazine (391.7 mg) obtained in Step 2 of Reference Example 1 and 3,5-dichloro-2-hydroxybenzoyl chloride obtained in the previous Step were dissolved in ethyl acetate (6 mL), and the mixture was stirred with heating at 90° C. for 1 hr. Ethyl acetate (6 mL) was added to the reaction mixture, and the mixture was washed successively with water, 1N hydrochloric acid, water, saturated aqueous sodium hydrogencarbonate, water and saturated brine, and dried over anhydrous sodium sulfate. The solvent was evaporated, and the obtained residue was purified by silica gel chromatography (n-hexane-ethyl acetate=85:15) to give the title compound (457 mg) as pale-yellow crystals.

### Reference Example 39

### Production of (2,3-dihydrobenzo[1,4]oxazin-4-yl)-(4-hydroxy-3-trifluoromethylphenyl)-methanone

### Step 1

### Production of 4-hydroxy-3-trifluoromethylbenzoyl chloride

4-Hydroxy-3-trifluoromethylbenzoic acid (610 mg) was suspended in toluene (6 mL), and thionyl chloride (0.28 mL) and N,N-dimethylformamide (1 drop) were added. After stirring with heating at 70° C. for 2 hrs, the mixture was concentrated and azeotroped with toluene to give the title compound.

### Step 2

### Production of (2,3-dihydrobenzo[1,4]oxazin-4-yl)-(4-hydroxy-3-trifluoromethylphenyl)-methanone

3,4-Dihydro-2H-benzo[1,4]oxazine (400 mg) obtained in Step 2 of Reference Example 1 and 4-hydroxy-3-trifluoromethylbenzoyl chloride obtained in the previous Step were dissolved in ethyl acetate (6 mL), and the mixture was stirred with heating at 90° C. for 1 hr. Ethyl acetate was added to the reaction mixture, and the mixture was washed successively with water, 1N hydrochloric acid, water, saturated aqueous sodium hydrogencarbonate and saturated brine, and dried over anhydrous sodium sulfate. The solvent was evaporated, and the obtained solid was crystallized from ethyl acetate to give the title compound (359 mg) as crystals.

### Reference Example 40

### Production of (3-chloro-4-hydroxy-5-methoxyphenyl)-(2,3-dihydrobenzo[1,4]oxazin-4-yl)-methanone

### Step 1

### Production of 3-chloro-4-hydroxy-5-methoxybenzoyl chloride

3-Chloro-4-hydroxy-5-methoxybenzoic acid (598 mg) was suspended in toluene (6 mL), and thionyl chloride (0.28 mL) and N,N-dimethylformamide (1 drop) were added. After stirring with heating at 70° C. for 2 hrs, the mixture was concentrated and azeotroped with toluene to give the title compound.

### Step 2

### Production of (3-chloro-4-hydroxy-5-methoxyphenyl)-(2,3-dihydrobenzo[1,4]oxazin-4-yl)-methanone

3,4-Dihydro-2H-benzo[1,4]oxazine (400 mg) obtained in Step 2 of Reference Example 1 and 3-chloro-4-hydroxy-5-methoxybenzoyl chloride obtained in the previous Step were dissolved in ethyl acetate (6 mL), and the mixture was stirred with heating at 90° C. for 1 hr. Ethyl acetate was added to the reaction mixture, and the mixture was washed successively with water, 1N hydrochloric acid, water, saturated aqueous sodium hydrogencarbonate, water and saturated brine, and dried over anhydrous sodium sulfate. The solvent was evaporated, and the obtained solid was crystallized from ethyl acetate to give the title compound (428 mg) as crystals.

### Reference Example 41

### Production of (4-chloro-3-hydroxyphenyl)-(2,3-dihydrobenzo[1,4]oxazin-4-yl)-methanone

### Step 1

### Production of 4-chloro-3-hydroxybenzoyl chloride

4-Chloro-3-hydroxybenzoic acid (510 mg) was suspended in toluene (6 mL), and thionyl chloride (0.28 mL) and N,N-dimethylformamide (1 drop) were added. After stirring with heating at 70° C. for 2 hrs, and the mixture was concentrated and azeotroped with toluene to give the title compound.

### Step 2

### Production of (4-chloro-3-hydroxyphenyl)-(2,3-dihydrobenzo[1,4]oxazin-4-yl)-methanone

3,4-Dihydro-2H-benzo[1,4]oxazine (400 mg) obtained in Step 2 of Reference Example 1 and 4-chloro-3-hydroxybenzoyl chloride obtained in the previous Step were dissolved in ethyl acetate (6 mL), and the mixture was stirred with heating at 90° C. for 1 hr. Ethyl acetate was added to the reaction mixture, and the mixture was washed successively with water, 1N hydrochloric acid, water, saturated aqueous sodium hydrogencarbonate, water and saturated brine, and dried over anhydrous sodium sulfate. The solvent was evaporated, and the obtained solid was crystallized from ethyl acetate to give the title compound (583 mg) as crystals.

### Reference Example 42

### Production of (2,6-dichloropyridin-4-yl)-(2,3-dihydrobenzo[1,4]oxazin-4-yl)-methanone

3,4-Dihydro-2H-benzo[1,4]oxazine (270 mg) obtained in Step 2 of Reference Example 1, 2,6-dichloroisonicotinoic acid (384 mg) and 4-dimethylaminopyridine (269 mg) were dissolved in chloroform (12 mL), and WSC.HCl (422 mg) was added under ice-cooling. After stirring overnight at room temperature, the reaction mixture was purified by silica gel chromatography (n-hexane-ethyl acetate=4:1) to give the title compound (419.8 mg) as pale-yellow crystals.

### Reference Example 43

### Production of (2,3-dihydrobenzo[1,4]oxazin-4-yl)-(4-nitrophenyl)-methanone

3,4-Dihydro-2H-benzo[1,4]oxazine (135 mg) obtained in Step 2 of Reference Example 1 and 4-nitrobenzoyl chloride (185 mg) were dissolved in ethyl acetate (3 mL), and the mixture was stirred overnight with heating at 95° C. The reaction mixture was purified by silica gel chromatography (n-hexane-ethyl acetate=5:1) to give the title compound (288.1 mg) as a yellow solid.

### Reference Example 44

### Production of (3,5-dichloro-4-hydroxyphenyl)-(6-fluoro-2,3-dihydrobenzo[1,4]oxazin-4-yl)-methanone

### Step 1

### Production of 6-fluoro-3,4-dihydro-2H-benzo[1,4]oxazine

6-Fluoro-4H-benzo[1,4]oxazin-3-one (1.5 g) was dissolved in tetrahydrofuran (20 mL), borane-tetrahydrofuran complex (1M tetrahydrofuran solution, 11 mL) was added under ice-cooling, and the mixture was stirred overnight at room temperature. 6N Hydrochloric acid (5 mL) was added, and the mixture was stirred with heating at 70° C. The mixture was allowed to cool to room temperature, weak-alkalized with 4N aqueous sodium hydroxide and saturated aqueous sodium hydrogencarbonate, and the mixture was extracted with ethyl acetate. The obtained ethyl acetate layer was washed successively with water, saturated aqueous sodium hydrogencarbonate, water and saturated brine, and dried over anhydrous sodium sulfate. The solvent was evaporated, and the obtained residue was purified by silica gel chromatography (n-hexane-ethyl acetate=4:1) to give the title compound (954 g) as an oil.

### Step 2

### Production of (3,5-dichloro-4-hydroxyphenyl)-(6-fluoro-2,3-dihydrobenzo[1,4]oxazin-4-yl)-methanone

6-Fluoro-3,4-dihydro-2H-benzo[1,4]oxazine (230 mg) and 3,5-dichloro-4-hydroxybenzoyl chloride (338 mg) obtained in Step 1 of Reference Example 3 were dissolved in ethyl acetate (3.5 mL), and the mixture was stirred overnight at 95° C. The reaction mixture was allowed to cool to room temperature, and the precipitated solid was collected by filtration to give the title compound (374.9 mg) as pale-beige crystals.

### Reference Example 45

### Production of 2-(2,3-dihydrobenzo[1,4]oxazine-4-carbonyl)benzoic acid

3,4-Dihydro-2H-benzo[1,4]oxazine (300 mg) obtained in Step 2 of Reference Example 1 and phthalic anhydride (329 mg) were dissolved in toluene (3 mL), and the mixture was heated under reflux for 4 hrs. The reaction mixture was allowed to cool, and the precipitated solid was collected by filtration to give the title compound (519 mg) as crystals.

### Reference Example 46

### Production of methyl 4-(2,3-dihydrobenzo[1,4]oxazine-4-carbonyl)benzoate

3,4-Dihydro-2H-benzo[1,4]oxazine (154 mg) obtained in Step 2 of Reference Example 1 was dissolved in chloroform (6 mL), triethylamine (0.18 mL) and methyl 4-chlorocarbonylbenzoate (226 mg) was added under ice-cooling. After stirring overnight at room temperature, the reaction mixture was purified by silica gel chromatography (n-hexane-ethyl acetate=7:2) to give the title compound (287.4 mg) as a pale-orange solid.

### Reference Example 47

### Production of 4-(2,3-dihydrobenzo[1,4]oxazine-4-carbonyl)benzoic acid

Methyl 4-(2,3-dihydrobenzo[1,4]oxazine-4-carbonyl)benzoate (283 mg) obtained in Reference Example 46 was dissolved in methanol (10 mL) and tetrahydrofuran (5 mL), and 4N aqueous lithium hydroxide (1.5 mL) was added. After stirring overnight at room temperature, the reaction mixture was concentrated under reduced pressure, and the residue was acidified with 1N hydrochloric acid under ice-cooling. The precipitated solid was collected by filtration to give the title compound (207.6 mg) as a white solid.

### Reference Example 48

### Production of methyl 3-(2,3-dihydrobenzo[1,4]oxazine-4-carbonyl)benzoate

### Step 1

### Production of methyl 3-chlorocarbonylbenzoate

Chloroform (5 mL) was added to isophthalic acid monomethyl ester (198 mg), and oxalyl chloride (0.12 mL) and N,N-dimethylformamide (1 drop) were added under ice-cooling. After stirring at room temperature for 7 hrs, the mixture was concentrated and azeotroped with toluene to give the title compound.

### Step 2

### Production of methyl 3-(2,3-dihydrobenzo[1,4]oxazine-4-carbonyl)benzoate

3,4-Dihydro-2H-benzo[1,4]oxazine (154 mg) obtained in Step 2 of Reference Example 1 was dissolved in chloroform (6 mL), and triethylamine (0.18 mL) and methyl 3-chlorocarbonylbenzoate obtained in the previous Step were added under ice-cooling. After stirring overnight at room temperature, the reaction mixture was purified by silica gel chromatography (n-hexane-ethyl acetate=3:1) to give the title compound (323.7 mg) as a white solid.

### Reference Example 49

### Production of 3-(2,3-dihydrobenzo[1,4]oxazine-4-carbonyl)benzoic acid

Methyl 3-(2,3-dihydrobenzo[1,4]oxazine-4-carbonyl)benzoate (293 mg) obtained in Step 2 of Reference Example 48 was dissolved in methanol (10 mL) and tetrahydrofuran (5 mL), and 4N aqueous lithium hydroxide (1.5 mL) was added. After stirring overnight at room temperature, the reaction mixture was concentrated under reduced pressure, and the residue was acidified with 1N hydrochloric acid under ice-cooling. The precipitated solid was collected by filtration, and the obtained solid was purified by silica gel chromatography (chloroform-methanol=10:1) to give the title compound (71.8 mg) as a white amorphous solid.

### Reference Example 50

### Production of (3,5-dichloro-2,4-dihydroxyphenyl)-(2,3-dihydrobenzo[1,4]oxazin-4-yl)-methanone

### Step 1

### Production of 3,5-dichloro-2,4-dihydroxybenzoic acid

2,4-Dihydroxybenzoic acid (25.0 g) was dissolved in ethyl acetate (400 mL), tert-butyl hypochlorite (61.9 g) was added dropwise under ice-cooling, and the mixture was stirred for 2 hrs. The reaction mixture was washed with saturated brine, dried over anhydrous sodium sulfate, and concentrated. The obtained residue was crystallized from ethyl ether-n-hexane to give the title compound (11.88 g) as a solid.

### Step 2

### Production of 3,5-dichloro-2,4-dihydroxybenzyl chloride

3,5-Dichloro-2,4-dihydroxybenzoic acid (605 mg) was suspended in toluene (6 mL), and thionyl chloride (0.25 mL) and N,N-dimethylformamide (1 drop) were added. After heating under reflux for 1 hr, the mixture was concentrated and azeotroped with toluene to give the title compound.

### Step 3

### Production of (3,5-dichloro-2,4-dihydroxyphenyl)-(2,3-dihydrobenzo[1,4]oxazin-4-yl)-methanone

3,4-Dihydro-2H-benzo[1,4]oxazine (366 mg) obtained in Step 2 of Reference Example 1 and 3,5-dichloro-2,4-dihydroxybenzyl chloride obtained in the previous Step were dissolved in ethyl acetate (6 mL), and the mixture was heated under reflux for 1 hr. The reaction mixture was washed successively with water, 1N hydrochloric acid, water, saturated aqueous sodium hydrogencarbonate, water and saturated brine, and dried over anhydrous sodium sulfate. The solvent was evaporated, and the obtained residue was crystallized from ethyl acetate to give the title compound (171 mg) as crystals.

### Reference Example 51

### Production of (6-chloro-2,3-dihydrobenzo[1,4]oxazin-4-yl)-(3,5-dichloro-4-hydroxyphenyl)-methanone

### Step 1

### Production of 2-chloro-N-(5-chloro-2-hydroxyphenyl)-acetamide

2-Amino-4-chlorophenol (1.0061 g) was dissolved in ethyl acetate (10 mL), water (10 mL) and sodium hydrogencarbonate (1.185 g) were added under ice-cooling, and chloroacetyl chloride (0.67 mL) was added dropwise. After stirring at room temperature for 2.5 hrs, water (20 mL) was added, and the mixture was extracted with ethyl acetate. The obtained ethyl acetate layer was washed with saturated brine, and dried over anhydrous sodium sulfate. The solvent was evaporated to give the title compound (1.5455 g) as a yellow solid.

### Step 2

### Production of 6-chloro-4H-benzo[1,4]oxazin-3-one

2-Chloro-N-(5-chloro-2-hydroxyphenyl)-acetamide (1.54 g) was dissolved in N,N-dimethylformamide (15 mL), and potassium carbonate (1.26 g) was added at room temperature. After stirring overnight at room temperature, 1N hydrochloric acid (12 mL) was added under ice-cooling, and the mixture was extracted with ethyl acetate. The obtained ethyl acetate layer was washed with water and saturated brine, and dried over anhydrous sodium sulfate. The solvent was evaporated to give the title compound (1.281 g) as a pale-yellow solid.

### Step 3

### Production of 6-chloro-3,4-dihydro-2H-benzo[1,4]oxazine

6-Chloro-4H-benzo[1,4]oxazin-3-one (764 mg) was dissolved in tetrahydrofuran (8 mL), borane-tetrahydrofuran complex (1M tetrahydrofuran solution, 6.2 mL) was added under ice-cooling, and the mixture was stirred overnight at room temperature. After stirring with heating at 70° C. for 1 hr, methanol (3 mL) was added dropwise at the same temperature, and the mixture was further stirred with heating for 1 hr. Then, while maintaining at 70° C., 1N hydrochloric acid (6.2 mL) was added dropwise and, after stirring with heating for 0.5 hr, the mixture was allowed to cool to room temperature. The mixture was extracted with ethyl acetate, and the obtained ethyl acetate layer was washed with water and saturated brine, and dried over anhydrous sodium sulfate. The solvent was evaporated, and the obtained residue was purified by silica gel chromatography (chloroform-methanol=99:1) to give the title compound (640.1 mg) as a solid.

### Step 4

### Production of (6-chloro-2,3-dihydrobenzo[1,4]oxazin-4-yl)-(3,5-dichloro-4-hydroxyphenyl)-methanone

6-Chloro-3,4-dihydro-2H-benzo[1,4]oxazine (168.4 mg) and 3,5-dichloro-4-hydroxybenzoyl chloride (242.7 mg) obtained in Step 1 of Reference Example 3 were dissolved in ethyl acetate (3 mL), and the mixture was heated under reflux overnight. The solvent was evaporated, and the obtained solid was crystallized from methanol to give the title compound (318.4 mg) as white crystals.

### Reference Example 52

### Production of (7-chloro-2,3-dihydrobenzo[1,4]oxazin-4-yl)-(3,5-dichloro-4-hydroxyphenyl)-methanone

The title compound (749.3 mg) was obtained as white crystals by a method similar to Steps 1 to 4 of Reference Example 51 and using 2-amino-5-chlorophenol instead of 2-amino-4-chlorophenol.

### Reference Example 53

### Production of [4-(3,5-dichloro-4-hydroxybenzoyl)-3,4-dihydro-2H-quinoxalin-1-yl]-(3,5-dichloro-4-hydroxyphenyl)-methanone

1,2,3,4-Tetrahydroquinoxaline (68.1 mg) and 3,5-dichloro-4-hydroxybenzoyl chloride (250.5 mg) obtained in Step 1 of Reference Example 3 were dissolved in ethyl acetate (5 mL), and the mixture was heated under reflux overnight. The solvent was evaporated, and the obtained solid was crystallized from methanol to give the title compound (122.0 mg) as pale-gray crystals.

### Reference Example 54

### Production of (3,5-dichloro-4-hydroxyphenyl)-(3,4-dihydro-2H-quinoxalin-1-yl)-methanone

### Step 1

### Production of (4-benzyloxy-3,5-dichlorophenyl)-(3,4-dihydro-2H-quinoxalin-1-yl)-methanone

1,2,3,4-Tetrahydroquinoxaline (805.3 mg) was dissolved in ethyl acetate (30 mL), triethylamine (1.0 mL) and 4-benzyloxy-3,5-dichlorobenzoyl chloride (1.8988 g) obtained in Step 3 of Reference Example 8 were added under ice-cooling, and the mixture was stirred overnight at room temperature. Methanol was added to the reaction mixture, and the mixture was concentrated. The obtained solid was crystallized from ethyl acetate-water to give the title compound (2.042 g) as a pale-yellow solid.

### Step 2

### Production of (3,5-dichloro-4-hydroxyphenyl)-(3,4-dihydro-2H-quinoxalin-1-yl)-methanone

(4-Benzyloxy-3,5-dichlorophenyl)-(3,4-dihydro-2H-quinoxalin-1-yl)-methanone (287.7 mg) was dissolved in tetrahydrofuran (6 mL). 7.5% Palladium-carbon (26 mg) was added to this solution and, under a hydrogen atmosphere, the mixture was stirred at room temperature for 1 hr. The reaction mixture was filtered through celite, and the residue was washed with tetrahydrofuran. The filtrate and washing solution were combined and the mixture was concentrated under reduced pressure. The obtained solid was crystallized from methanol to give the title compound (194.1 mg) as pale-yellow crystals.

### Reference Example 55

### Production of methyl 4-(3,5-dichloro-4-hydroxybenzoyl)-3,4-dihydro-2H-benzo[1,4]oxazine-6-carboxylate

### Step 1

### Production of methyl 4-hydroxy-3-nitrobenzoate

Methanol (50 mL) and concentrated sulfuric acid (0.5 mL) were added to 4-hydroxy-3-nitrobenzoic acid (5.0022 g), and the mixture was stirred overnight at 80° C. The solvent was evaporated, and the residue was partitioned between ethyl acetate and saturated aqueous sodium hydrogencarbonate. The aqueous layer was acidified with 1N hydrochloric acid, and the mixture was extracted with ethyl acetate. The obtained ethyl acetate layer was washed with water and saturated brine, and dried over anhydrous sodium sulfate. The solvent was evaporated to give the title compound (5.3436 g) as a yellow solid.

### Step 2

### Production of methyl 3-amino-4-hydroxybenzoate

Methyl 4-hydroxy-3-nitrobenzoate (5.3436 g) was dissolved in tetrahydrofuran (27 mL) and methanol (27 mL). 7.5% Palladium-carbon (276.6 mg) was added to this solution and, under a hydrogen atmosphere, the mixture was stirred overnight at room temperature. The reaction mixture was filtered through celite, and the residue was washed with tetrahydrofuran. The filtrate and washing solution were combined and the mixture was concentrated under reduced pressure to give the title compound (4.6803 g) as a solid.

### Step 3

### Production of methyl 3-oxo-3,4-dihydro-2H-benzo[1,4]oxazine-6-carboxylate

Methyl 3-amino-4-hydroxybenzoate (4.6803 g) and benzyltriethylammonium chloride (6.1787 g) were suspended in chloroform (50 mL), sodium hydrogencarbonate (9.10 g) and chloroacetyl chloride (2.6 mL) were added under ice-cooling, and the mixture was stirred under ice-cooling for 1 hr. Thereafter, the mixture was stirred with heating at 70° C. The reaction mixture was concentrated, water and ethyl acetate were added, and the precipitated solid was collected by filtration. The mother liquor was extracted with ethyl acetate, and the obtained ethyl acetate layer was washed with water and saturated brine, and dried over anhydrous sodium sulfate. The solvent was evaporated, and the obtained residue and the solid collected earlier by filtration were combined and the mixture was crystallized from methanol to give the title compound (4.8085 g) as a solid.

### Step 4

### Production of methyl 3,4-dihydro-2H-benzo[1,4]oxazine-6-carboxylate

Methyl 3-oxo-3,4-dihydro-2H-benzo[1,4]oxazine-6-carboxylate (1.009 g) was added to borane-tetrahydrofuran complex (1M tetrahydrofuran solution, 11 mL) under ice-cooling, and the mixture was stirred with heating at 70° C. for 3.5 hrs. Methanol (3 mL) was added dropwise and the mixture was further stirred with heating for 3 hrs. The mixture was extracted with ethyl acetate, and the obtained ethyl acetate layer was washed with saturated brine, and dried over anhydrous sodium sulfate. The solvent was evaporated, and the obtained residue was purified by silica gel chromatography (chloroform-methanol=98:2) to give the title compound (281.3 mg) as a pink solid and (3,4-dihydro-2H-benzo[1,4]oxazin-6-yl)methanol (413.8 mg) as an oil.

### Step 5

### Production of methyl 4-(3,5-dichloro-4-hydroxybenzoyl)-3,4-dihydro-2H-benzo[1,4]oxazine-6-carboxylate

Methyl 3,4-dihydro-2H-benzo[1,4]oxazine-6-carboxylate (81.2 mg) was dissolved in ethyl acetate (3 mL), 3,5-dichloro-4-hydroxybenzoyl chloride (108.3 mg) obtained in Step 1 of Reference Example 3 was added under ice-cooling, and the mixture was stirred overnight at 80° C. The solvent was evaporated, and the obtained residue was purified by silica gel chromatography (chloroform-methanol=95:5) to give the title compound (167.8 mg) as a white amorphous solid.

### Reference Example 56

### Production of (3,5-dichloro-4-hydroxyphenyl)-(6-hydroxymethyl-2,3-dihydrobenzo[1,4]oxazin-4-yl)-methanone

### Step 1

### Production of 6-(tert-butyldimethylsilyloxymethyl)-3,4-dihydro-2H-benzo[1,4]oxazine

(3,4-Dihydro-2H-benzo[1,4]oxazin-6-yl)methanol (406.1 mg) obtained in Step 4 of Reference Example 55 was dissolved in N,N-dimethylformamide (4 mL), and imidazole (203.8 mg) and tert-butylchlorodimethylsilane (443.4 mg) were added under ice-cooling. After stirring at room temperature for 1 hr, the mixture was poured into water, and the mixture was extracted with ethyl acetate. The obtained ethyl acetate layer was washed with water and saturated brine, dried over anhydrous sodium sulfate, and concentrated. The obtained residue was purified by silica gel chromatography (n-hexane-ethyl acetate=4:1) to give the title compound (659.0 mg) as a pale-yellow oil.

### Step 2

### Production of [6-(tert-butyldimethylsilyloxymethyl)-2,3-dihydrobenzo[1,4]oxazin-4-yl]-(3,5-dichloro-4-hydroxyphenyl)-methanone

6-(tert-Butyldimethylsilyloxymethyl)-3,4-dihydro-2H-benzo[1,4]oxazine (279.1 mg) was dissolved in ethyl acetate (3 mL), triethylamine (0.167 mL) and 3,5-dichloro-4-hydroxybenzoyl chloride (249.4 mg) obtained in Step 1 of Reference Example 3 were added under ice-cooling, and the mixture was stirred overnight at 80° C. The solvent was evaporated, and water and ethyl acetate were added and the mixture was extracted with ethyl acetate. The obtained ethyl acetate layer was washed with water and saturated brine, dried over anhydrous sodium sulfate, and concentrated. The obtained residue was purified by silica gel chromatography (n-hexane-ethyl acetate=4:1) to give the title compound (131.7 mg) as a pale-yellow solid.

### Step 3

### Production of (3,5-dichloro-4-hydroxyphenyl)-(6-hydroxymethyl-2,3-dihydrobenzo[1,4]oxazin-4-yl)-methanone

[6-(tert-Butyldimethylsilyloxymethyl)-2,3-dihydrobenzo[1,4]oxazin-4-yl]-(3,5-dichloro-4-hydroxyphenyl)-methanone (131.7 mg) was dissolved in tetrahydrofuran (1.5 mL), 1M tetrabutylammonium fluoride/tetrahydrofuran solution (0.34 mL) was added under ice-cooling, and the mixture was stirred overnight at room temperature. The solvent was evaporated, and the obtained residue was purified by silica gel chromatography (chloroform-methanol=97:3) to give the title compound (50.0 mg) as crystals.

### Reference Example 57

### Production of 4-(3,5-dichloro-4-hydroxybenzoyl)-3,4-dihydro-2H-benzo[1,4]oxazine-6-carboxylic acid

Methyl 4-(3,5-dichloro-4-hydroxybenzoyl)-3,4-dihydro-2H-benzo[1,4]oxazine-6-carboxylate (140.8 mg) obtained in Step 5 of Reference Example 55 was dissolved in methanol (1.5 mL) and tetrahydrofuran (1.5 mL), and 2N aqueous sodium hydroxide (0.55 mL) was added. After stirring overnight at room temperature, the solvent was evaporated, and the residue was acidified with 10% aqueous citric acid solution under ice-cooling. The precipitated solid was collected by filtration, and the obtained solid was crystallized from methanol to give the title compound (81.3 mg) as crystals.

### Reference Example 58

### Production of methyl 4-(3,5-dichloro-4-hydroxybenzoyl)-3,4-dihydro-2H-benzo[1,4]oxazine-5-carboxylate

The title compound (246.0 mg) was obtained as a white amorphous solid by a method similar to Steps 1 to 5 of Reference Example 55 and using 3-hydroxy-2-nitrobenzoic acid instead of 4-hydroxy-3-nitrobenzoic acid.

### Reference Example 59

### Production of methyl 4-(3,5-dichloro-4-hydroxybenzoyl)-3,4-dihydro-2H-benzo[1,4]oxazine-7-carboxylate

The title compound (317.9 mg) was obtained as white crystals by a method similar to Steps 1 to 5 of Reference Example 55 and using 3-hydroxy-4-nitrobenzoic acid instead of 4-hydroxy-3-nitrobenzoic acid.

### Reference Example 60

### Production of 4-(3,5-dichloro-4-hydroxybenzoyl)-3,4-dihydro-2H-benzo[1,4]oxazine-7-carboxylic acid

Methyl 4-(3,5-dichloro-4-hydroxybenzoyl)-3,4-dihydro-2H-benzo[1,4]oxazine-7-carboxylate (257.4 mg) obtained in Reference Example 59 was dissolved in methanol (2.5 mL) and tetrahydrofuran (2.5 mL), and 2N aqueous sodium hydroxide (1.0 mL) was added. After stirring with heating at 60° C. for 2.5 hrs, the solvent was evaporated, and the residue was acidified with 10% aqueous citric acid solution under ice-cooling. The precipitated solid was collected by filtration, and the obtained solid was crystallized from methanol to give the title compound (76.6 mg) as crystals.

### Reference Example 61

### Production of methyl 4-(3,5-dichloro-4-hydroxybenzoyl)-3,4-dihydro-2H-benzo[1,4]oxazine-8-carboxylate

The title compound (313.8 mg) was obtained as white crystals by a method similar to Steps 1 to 5 of Reference Example 55 and using 2-hydroxy-3-nitrobenzoic acid instead of 4-hydroxy-3-nitrobenzoic acid.

### Reference Example 62

### Production of 4-(3,5-dichloro-4-hydroxybenzoyl)-3,4-dihydro-2H-benzo[1,4]oxazine-8-carboxylic acid

Methyl 4-(3,5-dichloro-4-hydroxybenzoyl)-3,4-dihydro-2H-benzo[1,4]oxazine-8-carboxylate (244.2 mg) obtained in Reference Example 61 was dissolved in methanol (2.5 mL) and tetrahydrofuran (2.5 mL), and 2N aqueous sodium hydroxide (0.96 mL) was added. After stirring overnight at room temperature, the solvent was evaporated, and the residue was acidified with 10% aqueous citric acid solution under ice-cooling. The precipitated solid was collected by filtration, and the obtained solid was crystallized from ethyl acetate to give the title compound (186.6 mg) as crystals.

### Reference Example 63

### Production of (3,5-dichlorophenyl)-(2,3-dihydrobenzo[1,4]oxazin-4-yl)-methanone

3,4-Dihydro-2H-benzo[1,4]oxazine (250.1 mg) obtained in Step 2 of Reference Example 1 and 3,5-dichlorobenzoyl chloride (426.9 mg) were dissolved in ethyl acetate (5 mL), and the mixture was stirred overnight with heating at 80° C. The solvent was evaporated, and the obtained residue was crystallized from methanol to give the title compound (469.6 mg) as pale-beige crystals.

### Reference Example 64

### Production of (3,5-dichloro-4-hydroxyphenyl)-phenoxazin-10-ylmethanone

Phenoxazine (276.1 mg) and 3,5-dichloro-4-hydroxybenzoyl chloride (370.8 mg) obtained in Step 1 of Reference Example 3 were dissolved in ethyl acetate (3 mL), and the mixture was stirred overnight with heating at 80° C. The solvent was evaporated, and the obtained residue was purified by silica gel chromatography (n-hexane-ethyl acetate=4:1) to give the title compound (397.6 mg) as a yellow green solid.

### Reference Example 65

### Production of (3,5-dichloro-4-hydroxyphenyl)-(6-phenyl-2,3-dihydrobenzo[1,4]oxazin-4-yl)-methanone

The title compound (160.0 mg) was obtained as white crystals by a method similar to Steps 2 to 5 of Reference Example 21 and using 2-amino-4-phenylphenol instead of 3-amino-N,N-diethyl-4-hydroxybenzenesulfonamide.

### Reference Example 66

### Production of (3,5-dichloro-4-hydroxyphenyl)-(6,8-dimethyl-2,3-dihydrobenzo[1,4]oxazin-4-yl)-methanone

The title compound (152.7 mg) was obtained as crystals by a method similar to Steps 1 to 4 of Reference Example 51 and using 2-amino-4,6-dimethylphenol instead of 2-amino-4-chlorophenol.

### Reference Example 67

### Production of (3,5-dichloro-4-hydroxyphenyl)-(6-nitro-2,3-dihydrobenzo[1,4]oxazin-4-yl)-methanone

### Step 1

### Production of 6-nitro-4H-benzo[1,4]oxazin-3-one

2-Amino-4-nitrophenol (4.6283 g) and benzyltriethylammonium chloride (6.8772 g) were suspended in chloroform (46 mL), sodium hydrogencarbonate (10.10 g) and chloroacetyl chloride (4.0707 g) were added under ice-cooling, and the mixture was stirred for 1 hr under ice-cooling. Thereafter, the mixture was stirred with heating at 70° C. The reaction mixture was concentrated, water and ethyl acetate were added, and the precipitated solid was collected by filtration. The mother liquor was extracted with ethyl acetate, and the obtained ethyl acetate layer was washed with water and saturated brine, and dried over anhydrous sodium sulfate. The solvent was evaporated, the obtained residue and the solid collected earlier by filtration were combined and the mixture was crystallized from ethanol to give the title compound (5.4344 g) as a solid.

### Step 2

### Production of 6-nitro-3,4-dihydro-2H-benzo[1,4]oxazine

To borane-tetrahydrofuran complex (1M tetrahydrofuran solution, 33.5 mL) was added 6-nitro-4H-benzo[1,4]oxazin-3-one (3.0084 g) under ice-cooling, and the mixture was stirred with heating at 70° C. for 5 hrs. Methanol (5 mL) was added dropwise, and the mixture was further stirred with heating for 2.5 hrs. Concentrated hydrochloric acid (5 mL) was added dropwise, and the mixture was stirred with heating for 1.5 hrs. The mixture was extracted with ethyl acetate, and the obtained ethyl acetate layer was washed with water and saturated brine, and dried over anhydrous sodium sulfate. The solvent was evaporated, and the obtained residue was crystallized from n-hexane-ethyl acetate to give the title compound (2.2092 g) as a solid.

### Step 3

### Production of (3,5-dichloro-4-hydroxyphenyl)-(6-nitro-2,3-dihydrobenzo[1,4]oxazin-4-yl)-methanone

6-Nitro-3,4-dihydro-2H-benzo[1,4]oxazine (376.1 mg) was dissolved in ethyl acetate (3 mL), 3,5-dichloro-4-hydroxybenzoyl chloride (270.7 mg) obtained in Step 1 of Reference Example 3 was added under ice-cooling, and the mixture was stirred overnight at 80° C. The reaction mixture was allowed to cool to room temperature, and the precipitated solid was collected by filtration to give the title compound (538.0 mg) as pale-yellow crystals.

### Reference Example 68

### Production of (6-amino-2,3-dihydrobenzo[1,4]oxazin-4-yl)-(3,5-dichloro-4-hydroxyphenyl)-methanone

(3,5-Dichloro-4-hydroxyphenyl)-(6-nitro-2,3-dihydrobenzo[1,4]oxazin-4-yl)-methanone (200.0 mg) obtained in Step 3 of Reference Example 67 was dissolved in tetrahydrofuran (12 mL). 7.5% Palladium-carbon (19.4 mg) was added to this solution and, under a hydrogen atmosphere, the mixture was stirred overnight at room temperature. The reaction mixture was filtered through celite, and the residue was washed with tetrahydrofuran. The filtrate and washing solution were combined and the mixture was concentrated under reduced pressure. The obtained residue was purified by silica gel chromatography (chloroform-methanol=95:5) to give the title compound (6.4 mg) as crystals.

### Reference Example 69

### Production of (3,5-dibromo-4-hydroxyphenyl)-(6-nitro-2,3-dihydrobenzo[1,4]oxazin-4-yl)-methanone

6-Nitro-3,4-dihydro-2H-benzo[1,4]oxazine (179.8 mg) obtained in Step 2 of Reference Example 67 was dissolved in ethyl acetate (5 mL), 3,5-dibromo-4-hydroxybenzoyl chloride (346.6 mg) obtained in Step 1 of Reference Example 4 was added under ice-cooling, and the mixture was stirred overnight at 80° C. The reaction mixture was allowed to cool to room temperature, and the precipitated solid was collected by filtration to give the title compound (437.0 mg) as yellow crystals.

### Reference Example 70

### Production of (3,5-dichloro-4-hydroxyphenyl)-(7-nitro-2,3-dihydrobenzo[1,4]oxazin-4-yl)-methanone

The title compound (485.2 mg) was obtained as yellow crystals by a method similar to Steps 1 to 3 of Reference Example 67 and using 2-amino-5-nitrophenol instead of 2-amino-4-nitrophenol.

### Reference Example 71

### Production of (7-amino-2,3-dihydrobenzo[1,4]oxazin-4-yl)-(3,5-dichloro-4-hydroxyphenyl)-methanone

(3,5-Dichloro-4-hydroxyphenyl)-(7-nitro-2,3-dihydrobenzo[1,4]oxazin-4-yl)-methanone (303.1 mg) obtained in Reference Example 70 was dissolved in tetrahydrofuran (6 mL) and methanol (3 mL). 7.5% Palladium-carbon (30 mg) was added to this solution and, under a hydrogen atmosphere, and the mixture was stirred at room temperature for 1.5 hrs. The reaction mixture was filtered through celite, and the residue was washed with tetrahydrofuran. The filtrate and washing solution were combined and the mixture was concentrated under reduced pressure. The obtained residue was crystallized from ethanol to give the title compound (257.9 mg) as yellow crystals.

### Reference Example 72

### Production of N-[4-(3,5-dichloro-4-hydroxybenzoyl)-3,4-dihydro-2H-benzo[1,4]oxazin-7-yl]-methanesulfonamide

(7-Amino-2,3-dihydrobenzo[1,4]oxazin-4-yl)-(3,5-dichloro-4-hydroxyphenyl)-methanone (251.7 mg) obtained in Reference Example 71 was suspended in methylene chloride (5 mL), pyridine (0.0776 mL) and methanesulfonyl chloride (0.0689 mL) were added under ice-cooling, and the mixture was stirred overnight at room temperature. Methanol was added to the reaction mixture, and the mixture was concentrated under reduced pressure. The obtained residue was purified by silica gel chromatography (chloroform-methanol=95:5) to give the title compound (173.6 mg) as a pale-orange amorphous solid.

### Reference Example 73

### Production of 1-[4-(3,5-dichloro-4-hydroxybenzoyl)-3,4-dihydro-2H-quinoxalin-1-yl]-ethanone

### Step 1

### Production of 1-[4-(4-benzyloxy-3,5-dichlorobenzoyl)-3,4-dihydro-2H-quinoxalin-1-yl]-ethanone

(4-Benzyloxy-3,5-dichlorophenyl)-(3,4-dihydro-2H-quinoxalin-1-yl)-methanone (400 mg) obtained in Step 1 of Reference Example 54 was suspended in methylene chloride (8 mL), triethylamine (0.162 mL) and acetyl chloride (0.082 mL) were added under ice-cooling, and the mixture was stirred overnight at room temperature. Methanol was added to the reaction mixture, and the mixture was concentrated under reduced pressure. Water was added and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (chloroform-methanol=99:1) to give the title compound (295.9 mg) as a pale-yellow amorphous form.

### Step 2

### Production of 1-[4-(3,5-dichloro-4-hydroxybenzoyl)-3,4-dihydro-2H-quinoxalin-1-yl]-ethanone

1-[4-(4-Benzyloxy-3,5-dichlorobenzoyl)-3,4-dihydro-2H-quinoxalin-1-yl]-ethanone (288.6 mg) was dissolved in tetrahydrofuran (6 mL). 7.5% Palladium-carbon (27.7 mg) was added to this solution and, under a hydrogen atmosphere, the mixture was stirred at room temperature for 1 hr. The reaction mixture was filtered through celite, and the residue was washed with tetrahydrofuran. The filtrate and washing solution were combined and the mixture was concentrated under reduced pressure. The obtained residue was purified by silica gel chromatography (chloroform-methanol=97:3) and crystallized from n-hexane-ethyl acetate to give the title compound (108.1 mg) as white crystals.

### Reference Example 74

### Production of (3,5-dichloro-4-hydroxyphenyl)-(4-methyl-3,4-dihydro-2H-quinoxalin-1-yl)-methanone

### Step 1

### Production of (4-benzyloxy-3,5-dichlorophenyl)-(4-methyl-3,4-dihydro-2H-quinoxalin-1-yl)-methanone

(4-Benzyloxy-3,5-dichlorophenyl)-(3,4-dihydro-2H-quinoxalin-1-yl)-methanone (401.6 mg) obtained in Step 1 of Reference Example 54 was dissolved in N,N-dimethylformamide (8 mL), potassium carbonate (335.7 mg) and iodomethane (0.076 mL) were added, and the mixture was stirred with heating at 50° C. The mixture was poured into water, and the mixture was extracted with ethyl acetate. The obtained ethyl acetate layer was washed with water and saturated brine, dried over anhydrous sodium sulfate, and concentrated under reduced pressure. The obtained residue was purified by silica gel chromatography (chloroform-methanol=99:1) to give the title compound (114.7 mg) as an amorphous form.

### Step 2

### Production of (3,5-dichloro-4-hydroxyphenyl)-(4-methyl-3,4-dihydro-2H-quinoxalin-1-yl)-methanone

(4-Benzyloxy-3,5-dichlorophenyl)-(4-methyl-3,4-dihydro-2H-quinoxalin-1-yl)-methanone (114.7 mg) was dissolved in tetrahydrofuran (5 mL). 7.5% Palladium-carbon (10.0 mg) was added to this solution and, under a hydrogen atmosphere, the mixture was stirred at room temperature for 1 hr. The reaction mixture was filtered through celite, and the residue was washed with tetrahydrofuran. The filtrate and washing solution were combined and the mixture was concentrated under reduced pressure. The obtained residue was crystallized from ethyl acetate to give the title compound (56.3 mg) as pale-yellow crystals.

### Reference Example 75

### Production of (2,3-dihydrobenzo[1,4]oxazin-4-yl)-(4-hydroxy-3-nitrophenyl)-methanone

### Step 1

### Production of 4-hydroxy-3-nitrobenzoyl chloride

1,2-Dimethoxyethane (20 mL) was added to 4-hydroxy-3-nitrobenzoic acid (1.83 g) to dissolve same by heating the mixture to 80° C. Thionyl chloride (1.1 mL) was added, and the mixture was stirred overnight at 80° C. The reaction mixture was concentrated under reduced pressure, and azeotroped with toluene to give the title compound (2.0551 g) as a yellow oil.

### Step 2

### Production of (2,3-dihydrobenzo[1,4]oxazin-4-yl)-(4-hydroxy-3-nitrophenyl)-methanone

3,4-Dihydro-2H-benzo[1,4]oxazine (203 mg) obtained in Step 2 of Reference Example 1 and 4-hydroxy-3-nitrobenzoyl chloride (302 mg) were dissolved in ethyl acetate (2 mL), and the mixture was heated under reflux overnight. The reaction mixture was purified by silica gel chromatography (n-hexane-ethyl acetate=4:1) to give the title compound (460.7 mg) as a yellow amorphous solid.

### Reference Example 76

### Production of (3,5-dichloro-4-hydroxyphenyl)-(2-methyl-2,3-dihydroindol-1-yl)-methanone

### Step 1

### Production of (4-benzyloxy-3,5-dichlorophenyl)-(2-methyl-2,3-dihydroindol-1-yl)-methanone

2-Methyl-2,3-dihydro-1H-indole (139.3 mg) was dissolved in methylene chloride (2.5 mL), pyridine (0.1 mL) and 4-benzyloxy-3,5-dichlorobenzoyl chloride (396.6 mg) obtained in Step 3 of Reference Example 8 were added, and the mixture was stirred overnight at room temperature. The reaction mixture was concentrated, water was added, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate=6:1) to give the title compound (449.4 mg) as a yellow oil.

### Step 2

### Production of (3,5-dichloro-4-hydroxyphenyl)-(2-methyl-2,3-dihydroindol-1-yl)-methanone

(4-Benzyloxy-3,5-dichlorophenyl)-(2-methyl-2,3-dihydroindol-1-yl)-methanone (437.1 mg) was dissolved in toluene (4 mL), and trifluoroacetic acid (2.5 mL) was added at room temperature. After stirring with heating at 80° C., the mixture was concentrated. The obtained solid was crystallized from n-hexane-ethyl acetate to give the title compound (300.3 mg) as white crystals.

### Reference Example 77

### Production of (3,5-dichloro-4-hydroxyphenyl)-(2,3-dihydroindol-1-yl)-methanone

The title compound (230.6 mg) was obtained as white crystals by a method similar to Steps 1 and 2 of Reference Example 76 and using 2,3-dihydro-1H-indole instead of 2-methyl-2,3-dihydro-1H-indole.

### Reference Example 78

### Production of (5-amino-2,3-dihydroindol-1-yl)-(3,5-dichloro-4-hydroxyphenyl)-methanone

### Step 1

### Production of (4-benzyloxy-3,5-dichlorophenyl)-(5-nitro-2,3-dihydroindol-1-yl)-methanone

5-Nitro-2,3-dihydro-1H-indole (329.5 mg) was dissolved in methylene chloride (5 mL), pyridine (0.194 mL) and 4-benzyloxy-3,5-dichlorobenzoyl chloride (762.1 mg) obtained in Step 3 of Reference Example 8 were added, and the mixture was stirred overnight at room temperature. The reaction mixture was concentrated, water was added, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (n-hexane-ethyl acetate=4:1) to give the title compound (539.7 mg) as a yellow solid.

### Step 2

### Production of (5-amino-2,3-dihydroindol-1-yl)-(4-benzyloxy-3,5-dichlorophenyl)-methanone

(4-Benzyloxy-3,5-dichlorophenyl)-(5-nitro-2,3-dihydroindol-1-yl)-methanone (539.7 mg) was dissolved in tetrahydrofuran (10 mL). Platinum oxide (IV) (14 mg) was added to this solution and, under a hydrogen atmosphere, the mixture was stirred overnight at room temperature. The reaction mixture was filtered through celite, and the residue was washed with tetrahydrofuran. The filtrate and washing solution were combined and the mixture was concentrated under reduced pressure. Water was added, and the mixture was extracted with chloroform. The obtained chloroform layer was washed with saturated brine, and dried over anhydrous sodium sulfate. The solvent was evaporated to give a mixture (489.5 mg) of the title compound and (5-amino-2,3-dihydroindol-1-yl)-(3,5-dichloro-4-hydroxyphenyl)-methanone as a yellow amorphous form.

### Step 3

### Production of (5-amino-2,3-dihydroindol-1-yl)-(3,5-dichloro-4-hydroxyphenyl)-methanone

A mixture (489.5 mg) of (5-amino-2,3-dihydroindol-1-yl)-(4-benzyloxy-3,5-dichlorophenyl)-methanone and (5-amino-2,3-dihydroindol-1-yl)-(3,5-dichloro-4-hydroxyphenyl)-methanone was dissolved in toluene (5 mL), and trifluoroacetic acid (2.75 mL) was added at room temperature. After stirring with heating at 80° C. for 2.5 hrs, the mixture was concentrated. The obtained solid was crystallized from ethyl acetate to give the title compound (282.4 mg) as pale-gray crystals.

### Reference Example 79

### Production of (3,5-dibromo-4-hydroxyphenyl)-(6-fluoro-2,3-dihydrobenzo[1,4]oxazin-4-yl)-methanone

6-Fluoro-3,4-dihydro-2H-benzo[1,4]oxazine (230 mg) obtained in Step 1 of Reference Example 44 and 3,5-dibromo-4-hydroxybenzoyl chloride (472 mg) obtained in Step 1 of Reference Example 4 were dissolved in ethyl acetate (3.5 mL), and the mixture was stirred overnight at 95° C. The reaction mixture was allowed to cool to room temperature, and the precipitated solid was collected by filtration to give the title compound (533.7 mg) as white crystals.

### Reference Example 80

### Production of (3,5-dibromo-4-hydroxyphenyl)-(2,3-dihydronaphtho[2,1-b][1,4]oxazin-1-yl)-methanone

2,3-Dihydro-1H-naphtho[2,1-b][1,4]oxazine (140.9 mg) obtained in Step 3 of Reference Example 16 and 3,5-dibromo-4-hydroxybenzoyl chloride (239 mg) obtained in Step 1 of Reference Example 4 were dissolved in ethyl acetate (2.3 mL), and the mixture was stirred overnight at 95° C. The reaction mixture was allowed to cool to room temperature, and the precipitated solid was collected by filtration to give the title compound (298.8 mg) as black blue crystals.

### Reference Example 81

### Production of (3,5-dibromo-4-hydroxyphenyl)-(6-methyl-2,3-dihydrobenzo[1,4]oxazin-4-yl)-methanone

6-Methyl-3,4-dihydro-2H-benzo[1,4]oxazine (149 mg) obtained in Step 1 of Reference Example 12 and 3,5-dibromo-4-hydroxybenzoyl chloride (314 mg) obtained in Step 1 of Reference Example 4 were dissolved in ethyl acetate (3.5 mL), and the mixture was stirred overnight at 95° C. The reaction mixture was allowed to cool to room temperature, and the precipitated solid was collected by filtration to give the title compound (368.2 mg) as beige crystals.

### Reference Example 82

### Production of (6-chloro-2,3-dihydrobenzo[1,4]oxazin-4-yl)-(3,5-dibromo-4-hydroxyphenyl)-methanone

6-Chloro-3,4-dihydro-2H-benzo[1,4]oxazine (170.6 mg) obtained in Step 3 of Reference Example 51 and 3,5-dibromo-4-hydroxybenzoyl chloride (349.3 mg) obtained in Step 1 of Reference Example 4 were dissolved in ethyl acetate (5 mL), and the mixture was heated under reflux overnight. The solvent was evaporated, and the obtained solid was crystallized from methanol to give the title compound (441.0 mg) as crystals.

### Reference Example 83

### Production of (3,5-dichloro-4-hydroxyphenyl)-(4-methanesulfonyl-3,4-dihydro-2H-quinoxalin-1-yl)-methanone

### Step 1

### Production of (4-benzyloxy-3,5-dichlorophenyl)-(4-methanesulfonyl-3,4-dihydro-2H-quinoxalin-1-yl)-methanone

(4-Benzyloxy-3,5-dichlorophenyl)-(3,4-dihydro-2H-quinoxalin-1-yl)-methanone (399.7 mg) obtained in Step 1 of Reference Example 54 was dissolved in methylene chloride (8 mL), triethylamine (0.162 mL) and methanesulfonyl chloride (0.094 mL) were added under ice-cooling, and the mixture was stirred overnight at room temperature. The reaction mixture was concentrated, water was added, and the mixture was extracted with ethyl acetate. The obtained ethyl acetate layer was washed with water and saturated brine, and dried over anhydrous sodium sulfate. The solvent was evaporated, and the obtained residue was purified by silica gel chromatography (chloroform-methanol=98:2) to give the title compound (451.5 mg) as a pale-yellow amorphous form.

### Step 2

### Production of (3,5-dichloro-4-hydroxyphenyl)-(4-methanesulfonyl-3,4-dihydro-2H-quinoxalin-1-yl)-methanone

(4-Benzyloxy-3,5-dichlorophenyl)-(4-methanesulfonyl-3,4-dihydro-2H-quinoxalin-1-yl)-methanone (451.5 mg) was dissolved in tetrahydrofuran (10 mL). 7.5% Palladium-carbon (43.1 mg) was added to this solution and, under a hydrogen atmosphere, the mixture was stirred at room temperature for 1 hr. The reaction mixture was filtered through celite, and the residue was washed with tetrahydrofuran. The filtrate and washing solution were combined and the mixture was concentrated under reduced pressure. The obtained residue was purified by silica gel chromatography (chloroform-methanol=95:5) to give the title compound (335.9 mg) as a pale-yellow amorphous solid.

### Reference Example 84

### Production of (3,5-dichloro-4-hydroxyphenyl)-(6-ethanesulfonyl-2,3-dihydrobenzo[1,4]oxazin-4-yl)-methanone

### Step 1

### Production of 2-chloro-N-(5-ethanesulfonyl-2-hydroxyphenyl)acetamide

2-Amino-4-ethanesulfonylphenol (1.5063 g) was dissolved in ethyl acetate (15 mL), water (15 mL) and sodium hydrogencarbonate (1.2588 g) were added under ice-cooling, and chloroacetyl chloride (0.715 mL) was added dropwise. After stirring at room temperature for 5 hrs, water (20 mL) was added, and the mixture was extracted with ethyl acetate. The obtained ethyl acetate layer was washed with saturated brine, and dried over anhydrous sodium sulfate. The solvent was evaporated to give the title compound (1.9375 g) as a brown solid.

### Step 2

### Production of 6-ethanesulfonyl-4H-benzo[1,4]oxazin-3-one

2-Chloro-N-(5-ethanesulfonyl-2-hydroxyphenyl)acetamide (1.9375 g) was dissolved in N,N-dimethylformamide (20 mL), and potassium carbonate (1.2520 g) was added at room temperature. After stirring overnight at room temperature, water (20 mL) was added, and the mixture was extracted with ethyl acetate. The obtained ethyl acetate layer was washed with water and saturated brine, and dried over anhydrous sodium sulfate. The solvent was evaporated, and the obtained solid was crystallized from methanol to give the title compound (1.3358 g) as a solid.

### Step 3

### Production of 6-ethanesulfonyl-3,4-dihydro-2H-benzo[1,4]oxazine

6-Ethanesulfonyl-4H-benzo[1,4]oxazin-3-one (802.3 mg) was dissolved in tetrahydrofuran (4 mL), and borane-tetrahydrofuran complex (1M tetrahydrofuran solution, 7 mL) was added under ice-cooling. After stirring with heating at 70° C. for 9 hrs, methanol (5 mL) was added dropwise at the same temperature, and the mixture was further stirred with heating for 1 hr. 1N Hydrochloric acid (5 mL) was added dropwise while heating at 70° C. and, after stirring with heating for 1 hr, the mixture was allowed to cool to room temperature. The mixture was extracted with ethyl acetate, and the obtained ethyl acetate layer was washed with water and saturated brine, and dried over anhydrous sodium sulfate. The solvent was evaporated, and the obtained residue was purified by silica gel chromatography (chloroform-methanol=95:5) to give the title compound (748.7 mg) as an orange oil.

### Step 4

### Production of (3,5-dichloro-4-hydroxyphenyl)-(6-ethanesulfonyl-2,3-dihydrobenzo[1,4]oxazin-4-yl)-methanone

6-Ethanesulfonyl-3,4-dihydro-2H-benzo[1,4]oxazine (233.5 mg) and 3,5-dichloro-4-hydroxybenzoyl chloride (255.4 mg) obtained Step 1 of Reference Example 3 were dissolved in ethyl acetate (5 mL), and the mixture was heated under reflux overnight. The solvent was evaporated, and the obtained residue was purified by silica gel chromatography (chloroform-methanol=95:5) to give the title compound (446.9 mg) as a white amorphous solid.

### Reference Example 85

### Production of (3,5-dichloro-4-hydroxyphenyl)-(6-trifluoromethyl-2,3-dihydrobenzo[1,4]oxazin-4-yl)-methanone

### Step 1

### Production of 2-amino-4-trifluoromethylphenol

2-Nitro-4-trifluoromethylphenol (3.0951 g) was dissolved in tetrahydrofuran (15 mL). 7.5% Palladium-carbon (299.8 mg) was added to this solution and, under a hydrogen atmosphere, the mixture was stirred overnight at room temperature. The reaction mixture was filtered through celite, and the residue was washed with tetrahydrofuran. The filtrate and washing solution were combined and the mixture was concentrated under reduced pressure to give the title compound (2.6346 g) as a gray solid.

### Step 2

### Production of 2-chloro-N-(2-hydroxy-5-trifluoromethylphenyl)acetamide

2-Amino-4-trifluoromethylphenol (1.0075 g) was dissolved in ethyl acetate (10 mL), water (10 mL) and sodium hydrogencarbonate (0.9531 g) were added under ice-cooling, and chloroacetyl chloride (0.55 mL) was added dropwise. The mixture was stirred overnight at room temperature, and extracted with ethyl acetate. The obtained ethyl acetate layer was washed with saturated brine, and dried over anhydrous sodium sulfate. The solvent was evaporated to give the title compound (1.4341 g) as a pale-brown solid.

### Step 3

### Production of 6-trifluoromethyl-4H-benzo[1,4]oxazin-3-one

2-Chloro-N-(2-hydroxy-5-trifluoromethylphenyl)acetamide (1.4244 g) was dissolved in N,N-dimethylformamide (14 mL), and potassium carbonate (1.0072 g) was added at room temperature. After stirring at room temperature for 2 hrs, water (20 mL) was added, and the mixture was extracted with ethyl acetate. The obtained ethyl acetate layer was washed with water and saturated brine, and dried over anhydrous sodium sulfate. The solvent was evaporated to give the title compound (1.2084 g) as a pale-gray solid.

### Step 4

### Production of 6-trifluoromethyl-3,4-dihydro-2H-benzo[1,4]oxazine

6-Trifluoromethyl-4H-benzo[1,4]oxazin-3-one (810.8 mg) was dissolved in tetrahydrofuran (4 mL), borane-tetrahydrofuran complex (1M tetrahydrofuran solution, 7.4 mL) was added under ice-cooling. After stirring with heating at 70° C. for 6 hrs, methanol (5 mL) was added dropwise at the same temperature, and the mixture was further stirred with heating for 1 hr. 1N Hydrochloric acid (5 mL) was added dropwise while heating at 70° C., and the mixture was stirred with heating for 30 min. The mixture was allowed to cool to room temperature and extracted with ethyl acetate. The obtained ethyl acetate layer was washed with water and saturated brine, and dried over anhydrous sodium sulfate. The solvent was evaporated, and the obtained residue was purified by silica gel chromatography (chloroform-methanol=99:1) to give the title compound (718.3 mg) as a white solid.

### Step 5

### Production of (3,5-dichloro-4-hydroxyphenyl)-(6-trifluoromethyl-2,3-dihydrobenzo[1,4]oxazin-4-yl)-methanone

6-Trifluoromethyl-3,4-dihydro-2H-benzo[1,4]oxazine (205.3 mg) and 3,5-dichloro-4-hydroxybenzoyl chloride (252.5 mg) obtained in Step 1 of Reference. Example 3 were dissolved in ethyl acetate (5 mL), and the mixture was heated under reflux overnight. The solvent was evaporated, and the obtained residue was purified by silica gel chromatography (chloroform-methanol=95:5) to give the title compound (286.5 mg) as white crystals.

### Reference Example 86

### Production of (3,5-dichloro-4-methoxyphenyl)-(2,3-dihydrobenzo[1,4]oxazin-4-yl)-methanone

(3,5-Dichloro-4-hydroxyphenyl)-(2,3-dihydrobenzo[1,4]oxazin-4-yl)-methanone (100 mg) obtained in Step 2 of Reference Example 3 was dissolved in acetone (5 mL), potassium carbonate (47 mg) and iodomethane (0.096 mL) were added, and the mixture was stirred with heating at 45° C. overnight. The reaction mixture was purified by silica gel chromatography (n-hexane-ethyl acetate=5:1) to give the title compound (109.2 mg) as a white solid.

### Reference Example 87

### Production of 2,6-dichloro-4-(2,3-dihydrobenzo[1,4]oxazine-4-carbonyl)-phenyl acetate

(3,5-Dichloro-4-hydroxyphenyl)-(2,3-dihydrobenzo[1,4]oxazin-4-yl)-methanone (100 mg) obtained in Step 2 of Reference Example 3 was dissolved in chloroform (5 mL), triethylamine (0.064 mL) and acetyl chloride (0.026 mL) were added under ice-cooling, and the mixture was stirred overnight at room temperature. The reaction mixture was purified by silica gel chromatography (n-hexane-ethyl acetate=5:1) to give the title compound (123.1 mg) as white crystals.

### Reference Example 88

### Production of (2,3-dihydrobenzo[1,4]oxazin-4-yl)-(4-hydroxyphenyl)-methanone

### Step 1

### Production of (4-benzyloxyphenyl)-(2,3-dihydrobenzo[1,4]oxazin-4-yl)-methanone

3,4-Dihydro-2H-benzo[1,4]oxazine (270 mg) obtained in Step 2 of Reference Example 1, 4-benzyloxybenzoic acid (457 mg) and 4-dimethylaminopyridine (269 mg) were dissolved in chloroform (7 mL), and WSC.HCl (422 mg) was added under ice-cooling. After stirring overnight at room temperature, the reaction mixture was purified by silica gel chromatography (n-hexane-ethyl acetate=3:1) to give the title compound (636.2 mg) as an orange oil.

### Step 2

### Production of (2,3-dihydrobenzo[1,4]oxazin-4-yl)-(4-hydroxyphenyl)-methanone

(4-Benzyloxyphenyl)-(2,3-dihydrobenzo[1,4]oxazin-4-yl)-methanone (630 mg) was dissolved in tetrahydrofuran (20 mL). 7.5% Palladium-carbon (70 mg) was added to this solution and, under a hydrogen atmosphere, the mixture was stirred at room temperature for 6 hrs. The reaction mixture was filtered through celite, and the residue was washed with tetrahydrofuran. The filtrate and washing solution were combined and the mixture was concentrated under reduced pressure. The obtained residue was crystallized from ethyl acetate to give the title compound (149.1 mg) as white crystals.

### Reference Example 89

### Production of (3,5-dichloro-4-hydroxyphenyl)-(5-hydroxy-2,3-dihydrobenzo[1,4]oxazin-4-yl)-methanone

### Step 1

### Production of 2-chloro-N-(2,6-dihydroxyphenyl)-acetamide

2-Aminobenzene-1,3-diol hydrochloride (2.00 g) was dissolved in ethyl acetate (20 mL), water (20 mL) and sodium hydrogencarbonate (2.50 g) were added, chloroacetyl chloride (1.02 mL) was added dropwise under ice-cooling. After stirring at room temperature for 12 hrs, and the mixture was extracted with ethyl acetate. The obtained ethyl acetate layer was washed successively with water and saturated brine, and dried over anhydrous sodium sulfate. The solvent was evaporated and the obtained residue was purified by silica gel chromatography (n-hexane-ethyl acetate=65:35) to give the title compound (1.508 g) as a pale-orange solid.

### Step 2

### Production of 5-hydroxy-4H-benzo[1,4]oxazin-3-one

2-Chloro-N-(2,6-dihydroxyphenyl)-acetamide (1.274 g) was dissolved in N,N-dimethylformamide (10 mL), potassium carbonate (873 mg) was added at room temperature. After stirring at room temperature for 2.5 hrs, water (30 mL) was added. The mixture was stirred under ice-cooling for 0.5 hr, and the precipitated solid was collected by filtration to give the title compound (915 mg) as a pale-orange solid.

### Step 3

### Production of 5-benzyloxy-4H-benzo[1,4]oxazin-3-one

5-Hydroxy-4H-benzo[1,4]oxazin-3-one (200 mg) was dissolved in N,N-dimethylformamide (2 mL), potassium carbonate (167 mg) and benzyl bromide (158 mL) were added at room temperature. The mixture was stirred at room temperature for 3 hrs and stirred with heating at 60° C. for 1 hr, and water was added. The precipitated solid was collected by filtration to give the title compound (283 mg) as a pale-yellow solid.

### Step 4

### Production of 5-benzyloxy-3,4-dihydro-2H-benzo[1,4]oxazine

Lithium aluminum hydride (50 mg) was suspended in tetrahydrofuran (2 mL), and 5-benzyloxy-4H-benzo[1,4]oxazin-3-one (276 mg) was added by small portions under ice-cooling. The mixture was stirred at room temperature for 1 hr and stirred with heating at 60° C. for 1 hr. Water (0.05 mL), 15% aqueous sodium hydroxide (0.05 mL) and water (0.15 mL) were successively added under ice-cooling, and the mixture was stirred at room temperature. The mixture was dried over anhydrous sodium sulfate and concentrated. The obtained residue was purified by silica gel chromatography (n-hexane-ethyl acetate=9:1) to give the title compound (54 mg) as an oil.

### Step 5

### Production of (4-benzyloxy-3,5-dichlorophenyl)-(5-benzyloxy-2,3-dihydrobenzo[1,4]oxazin-4-yl)-methanone

5-Benzyloxy-3,4-dihydro-2H-benzo[1,4]oxazine (47 mg) was dissolved in chloroform (2 mL), triethylamine (0.0716 mL) and 4-benzyloxy-3,5-dichlorobenzoyl chloride (689.3 mg) obtained in Step 3 of Reference Example 8 were added. The mixture was stirred at room temperature for 25 hrs, water was added to the reaction mixture, and the mixture was extracted with ethyl acetate. The mixture was washed successively with water, saturated aqueous sodium hydrogencarbonate and saturated brine, dried over anhydrous sodium sulfate, and concentrated. The obtained residue was purified by silica gel chromatography (n-hexane-ethyl acetate=85:15) to give the title compound (54.7 mg) as a white solid.

### Step 6

### Production of (3,5-dichloro-4-hydroxyphenyl)-(5-hydroxy-2,3-dihydrobenzo[1,4]oxazin-4-yl)-methanone

(4-Benzyloxy-3,5-dichlorophenyl)-(5-benzyloxy-2,3-dihydrobenzo[1,4]oxazin-4-yl)-methanone (52 mg) was dissolved in tetrahydrofuran (2 mL). 7.5% Palladium-carbon (10 mg) was added to this solution and, under a hydrogen atmosphere, the mixture was stirred at room temperature for 1 hr. The reaction mixture was filtered through celite, and the residue was washed with tetrahydrofuran. The solvent was evaporated and the obtained solid was crystallized from ethyl ether to give the title compound (14.8 mg) as a pale-yellow solid.

### Reference Example 90

### Production of (3,5-dichloro-4-hydroxyphenyl)-(8-hydroxy-2,3-dihydrobenzo[1,4]oxazin-4-yl)-methanone

### Step 1

### Production of 2-methoxy-6-nitrophenol

1,2-Dimethoxyethane (100 mL) was added to 2-methoxyphenol (6.21 g), and the mixture was cooled to −50° C. Nitronium tetrafluoroborate (6.77 g) was added, and the mixture was stirred at −50° C. After the completion of reaction, the reaction mixture was poured into ice water, and ethyl acetate and ethyl ether were added. The insoluble material was removed by filtration, and the filtrate was extracted with ethyl acetate. The extract was washed successively with water and saturated brine, dried over anhydrous sodium sulfate, and concentrated. The obtained residue was purified by silica gel chromatography (n-hexane-ethyl acetate=4:1-3:1) to give the title compound (2.46 g) as a yellow solid.

### Step 2

### Production of 2-amino-6-methoxyphenol hydrochloride

2-Methoxy-6-nitrophenol (2.46 g) was dissolved in tetrahydrofuran (20 mL). 7.5% Palladium-carbon (400 mg) was added to this solution and, under a hydrogen atmosphere, and the mixture was stirred at room temperature for 7 hrs. The reaction mixture was filtered through celite, and the residue was washed with ethyl acetate. Under ice-cooling, 4N hydrogen chloride-ethyl acetate (10 mL) was added dropwise and the mixture was stirred for 30 min. The precipitated solid was collected by filtration to give the title compound (2.41 g) as a white solid.

### Step 3

### Production of 2-chloro-N-(2-hydroxy-3-methoxyphenyl)-acetamide

2-Amino-6-methoxyphenol hydrochloride (2.40 g) was dissolved in ethyl acetate (25 mL), water (30 mL) and sodium hydrogencarbonate (2.76 g) were added under ice-cooling, and chloroacetyl chloride (1.2 mL) was added dropwise. The mixture was stirred at room temperature for 0.5 hr, and extracted with ethyl acetate. The obtained ethyl acetate layer was washed successively with water and saturated brine, and dried over anhydrous sodium sulfate. The solvent was evaporated to give the title compound (2.95 g) as a pale-orange solid.

### Step 4

### Production of 8-methoxy-4H-benzo[1,4]oxazin-3-one

2-Chloro-N-(2-hydroxy-3-methoxyphenyl)-acetamide (2.95 g) was dissolved in N,N-dimethylformamide (20 mL), and potassium carbonate (2.46 g) was added under ice-cooling. The mixture was stirred at room temperature for 4 hrs, and water was added. The mixture was stirred at room temperature for 0.5 hr, and the precipitated solid was collected by filtration to give the title compound (2.06 g) as a pink solid.

### Step 5

### Production of 8-hydroxy-4H-benzo[1,4]oxazin-3-one

8-Methoxy-4H-benzo[1,4]oxazin-3-one (950 mg) was dissolved in methylene chloride (90 mL). After cooling to −78° C., boron tribromide (1.0M methylene chloride solution, 13.3 mL) was added dropwise, and the mixture was stirred at room temperature for 2 hrs. Under ice-cooling, the reaction mixture was poured into water, and the mixture was extracted with ethyl acetate. The extract was washed with saturated brine, and dried over anhydrous sodium sulfate. The solvent was evaporated to give the title compound (864 mg) as a khaki solid.

### Step 6

### Production of 8-(tert-butyldimethylsilyloxy)-4H-benzo[1,4]oxazin-3-one

8-Hydroxy-4H-benzo[1,4]oxazin-3-one (900 mg) was dissolved in N,N-dimethylformamide (10 mL), and imidazole (482 mg) and tert-butylchlorodimethylsilane (986 mg) were added. The mixture was stirred at room temperature for 1 hr, and water and 10% aqueous citric acid solution were added. The mixture was extracted with ethyl acetate, washed successively with water and saturated brine, dried over anhydrous sodium sulfate, and concentrated. The obtained residue was purified by silica gel chromatography (n-hexane-ethyl acetate=7:3) to give the title compound (1.41 g) as a white solid.

### Step 7

### Production of 8-(tert-butyldimethylsilyloxy)-3,4-dihydro-2H-benzo[1,4]oxazine

8-(tert-Butyldimethylsilyloxy)-4H-benzo[1,4]oxazin-3-one (700 mg) was dissolved in tetrahydrofuran (5 mL), borane-tetrahydrofuran complex (1M tetrahydrofuran solution, 3.76 mL) was added, and the mixture was heated under reflux for 2.5 hrs. The mixture was allowed to cool to room temperature, weak-alkalified with saturated aqueous sodium hydrogencarbonate and water, and extracted with ethyl acetate. The obtained ethyl acetate layer was washed successively with water and saturated brine, and dried over anhydrous sodium sulfate. The solvent was evaporated to give the title compound (675 mg) as an oil.

### Step 8

### Production of (4-benzyloxy-3,5-dichlorophenyl)-[8-(tert-butyldimethylsilyloxy)-2,3-dihydrobenzo[1,4]oxazin-4-yl]-methanone

8-(tert-Butyldimethylsilyloxy)-3,4-dihydro-2H-benzo[1,4]oxazine (792 mg) was dissolved in chloroform (10 mL), and pyridine (0.242 mL) and 4-benzyloxy-3,5-dichlorobenzoyl chloride (792 mg) obtained in Step 3 of Reference Example 8 were added. The mixture was stirred at room temperature for 1 hr, 10% aqueous citric acid solution was added, and the mixture was extracted with chloroform. The extract was washed successively with water and saturated brine, dried over anhydrous sodium sulfate, and concentrated. The obtained residue was purified by silica gel chromatography (n-hexane-ethyl acetate=8:1) to give the title compound (1.28 g) as a white solid.

### Step 9

### Production of (3,5-dichloro-4-hydroxyphenyl)-(8-hydroxy-2,3-dihydrobenzo[1,4]oxazin-4-yl)-methanone

(4-Benzyloxy-3,5-dichlorophenyl)-[8-(tert-butyldimethylsilyloxy)-2,3-dihydrobenzo[1,4]oxazin-4-yl]-methanone (545 mg) was dissolved in toluene (2 mL), and trifluoroacetic acid (4 mL) was added at room temperature. After stirring with heating at 85° C. for 7.5 hrs, and the mixture was concentrated. The obtained residue was dissolved in tetrahydrofuran, and the solution was treated with activated carbon. The solvent was evaporated, and the residue was purified by silica gel chromatography (n-hexane-ethyl acetate=1:1) and crystallized from ethyl ether was to give the title compound (220 mg) as a yellow solid.

### Reference Example 91

### Production of ethyl [2,6-dichloro-4-(2,3-dihydrobenzo[1,4]oxazine-4-carbonyl)phenoxy]acetate

(3,5-Dichloro-4-hydroxyphenyl)-(2,3-dihydrobenzo[1,4]oxazin-4-yl)-methanone (1.0067 g) obtained in Reference Example 3 was dissolved in N,N-dimethylformamide (10 mL), and potassium carbonate (0.6553 g) and ethyl bromoacetate (0.52 mL) were added at room temperature. After stirring overnight with heating at 70° C., ethyl acetate (20 mL) and 10% aqueous citric acid solution (20 mL) were added under ice-cooling, and the mixture was extracted with ethyl acetate. The extract was washed successively with water and saturated brine, dried over anhydrous sodium sulfate, and concentrated. The obtained residue was purified by silica gel chromatography (n-hexane-ethyl acetate=3:1) to give the title compound (1.2891 g) as a pale-yellow oil.

### Reference Example 92

### Production of [2,6-dichloro-4-(2,3-dihydrobenzo[1,4]oxazine-4-carbonyl)phenoxy]acetic acid

Ethyl [2,6-dichloro-4-(2,3-dihydrobenzo[1,4]oxazine-4-carbonyl)phenoxy]acetate (1.2753 g) obtained in Reference Example 91 was dissolved in methanol (6.5 mL), 2N aqueous sodium hydroxide (3.1 mL) was added, and the mixture was stirred at room temperature for 0.5 hr. Water was added to the reaction mixture, the mixture was washed with ethyl ether, and acidified with 10% aqueous citric acid solution. The mixture was extracted with ethyl acetate. The extract was washed with saturated brine, dried over anhydrous sodium sulfate, and concentrated. The obtained residue was crystallized from methanol to give the title compound (156.4 mg) as white crystals.

### Reference Example 93

### Production of (3,5-dichloro-4-hydroxyphenyl)-(3-methyl-2,3-dihydrobenzo[1,4]oxazin-4-yl)-methanone

### Step 1

### Production of 1-(2-nitrophenoxy)-propan-2-one

2-Nitrophenol (2.78 g) was dissolved in N,N-dimethylformamide (20 mL), potassium carbonate (3.34 g) and bromoacetone (1.85 mL) were added at room temperature, and the mixture was stirred overnight at room temperature. Water was added to the reaction mixture, and the mixture was extracted with ethyl ether. The extract was washed successively with water and saturated brine, dried over anhydrous sodium sulfate, and concentrated. The obtained residue was purified by silica gel chromatography (n-hexane-ethyl acetate=3:1) to give the title compound (3.2101 g) as a bright yellow solid.

### Step 2

### Production of 3-methyl-3,4-dihydro-2H-benzo[1,4]oxazine

1-(2-Nitrophenoxy)propan-2-one (501.1 mg) was dissolved in tetrahydrofuran (10 mL). 7.5% Palladium-carbon (49.1 mg) was added to this solution and, under a hydrogen atmosphere, the mixture was stirred at room temperature for 3 hrs. The reaction mixture was filtered through celite, and the residue was washed with tetrahydrofuran. The solvent was evaporated, and the obtained residue was purified by silica gel chromatography (n-hexane-ethyl acetate=3:1) to give the title compound (248.1 mg) as a yellow oil.

### Step 3

### Production of (3,5-dichloro-4-hydroxyphenyl)-(3-methyl-2,3-dihydrobenzo[1,4]oxazin-4-yl)-methanone

3-Methyl-3,4-dihydro-2H-benzo[1,4]oxazine (244.9 mg) and 3,5-dichloro-4-hydroxybenzoyl chloride (370.1 mg) obtained in Step 1 of Reference Example 3 were dissolved in ethyl acetate (7 mL), and the mixture was stirred with heating at 80° C. for 3 hrs. Ethyl acetate (15 mL) and water (20 mL) were added to partition the reaction mixture into layers, and the aqueous layer extracted with ethyl acetate. The extract was washed successively with water and saturated brine, dried over anhydrous sodium sulfate, and concentrated. The obtained solid was crystallized from methanol to give the title compound (320.2 mg) as white crystals.

### Reference Example 94

### Production of N-[2,6-dichloro-4-(2,3-dihydrobenzo[1,4]oxazine-4-carbonyl)phenyl]methanesulfonamide

### Step 1

### Production of N-methanesulfonyl-N-[2,6-dichloro-4-(2,3-dihydrobenzo[1,4]oxazine-4-carbonyl)phenyl]methanesulfonamide

(4-Amino-3,5-dichlorophenyl)-(2,3-dihydrobenzo[1,4]oxazin-4-yl)-methanone (323 mg) obtained in Reference Example 28 was dissolved in N,N-dimethylformamide (5 mL), 60% sodium hydride (52 mg) was added, and the mixture was stirred at room temperature for 0.5 hr. Methanesulfonyl chloride (0.0851 mL) was added, and the mixture was stirred at room temperature. 10% Aqueous citric acid solution was added to the reaction mixture, and the mixture was extracted with ethyl acetate and tetrahydrofuran. The extract was washed successively with water and saturated brine, dried over anhydrous sodium sulfate, and concentrated. The obtained residue was purified by silica gel chromatography (n-hexane-ethyl acetate=4:1), and crystallized from isopropyl ether to give the title compound (78 mg) as a white solid.

### Step 2

### Production of N-[2,6-dichloro-4-(2,3-dihydrobenzo[1,4]oxazine-4-carbonyl)phenyl]methanesulfonamide

N-Methanesulfonyl-N-[2,6-dichloro-4-(2,3-dihydrobenzo[1,4]oxazine-4-carbonyl)phenyl]methanesulfonamide (73 mg) was dissolved in tetrahydrofuran (3 mL), tetrabutylammonium fluoride (1M tetrahydrofuran solution, 0.168 mL) was added, and the mixture was stirred overnight at room temperature. 10% Aqueous citric acid solution and water added to the reaction mixture, and the mixture was extracted with ethyl acetate. The extract was washed successively with water and saturated brine, dried over anhydrous sodium sulfate, and concentrated. The obtained residue was crystallized from ethyl acetate to give the title compound (48 mg) as a white solid.

### Reference Example 95

### Production of (3,5-dichloro-4-hydroxyphenyl)-(7,8-dihydro-6H-5-oxa-9-azabenzocyclohepten-9-yl)-methanone

### Step 1

### Production of tert-butyldimethyl[3-(2-nitrophenoxy)propoxy]silane

2-Nitrophenol (3 g) was dissolved in N,N-dimethylformamide (20 mL), and sodium hydride (1.04 g) was added under ice-cooling. The mixture was stirred at room temperature for 0.5 hr, (3-bromopropoxy)-tert-butyldimethylsilane (5.49 mL) was added under ice-cooling, and the mixture was stirred with heating at 90° C. overnight. Potassium carbonate (1 g) and (3-bromopropoxy)-tert-butyldimethylsilane (2.5 mL) were added, and the mixture was stirred with heating at 90° C. for 1.5 hrs. 10% Aqueous citric acid solution and water were added to the reaction mixture, and the mixture was extracted with ethyl acetate. The extract was washed successively with water and saturated brine, dried over anhydrous sodium sulfate, and concentrated. The obtained residue was purified by silica gel chromatography (n-hexane-ethyl acetate=9:1) to give the title compound (6.71 g) as a yellow oil.

### Step 2

### Production of 2-[3-(tert-butyldimethylsilyloxy)propoxy]phenylamine

tert-Butyldimethyl[3-(2-nitrophenoxy)propoxy]silane (6.71 g) was dissolved in tetrahydrofuran (50 mL). 7.5% Palladium-carbon (1 g) was added to this solution and, under a hydrogen atmosphere, the mixture was stirred overnight at room temperature. The reaction mixture was filtered through celite, and the residue was washed with tetrahydrofuran. The solvent was evaporated, and the obtained residue was purified by silica gel chromatography (n-hexane-ethyl acetate=9:1) to give the title compound (5.88 g) as a pale-orange oil.

### Step 3

### Production of 4-benzyloxy-N-{2-[3-(tert-butyldimethylsilyloxy)propoxy]phenyl}-3,5-dichlorobenzamide

2-[3-(tert-Butyldimethylsilyloxy)propoxy]phenylamine (844 mg) was dissolved in chloroform (10 mL), and pyridine (0.314 mL) and 4-benzyloxy-3,5-dichlorobenzoyl chloride (947 mg) obtained in Step 3 of Reference Example 8 were added. The mixture was stirred at room temperature for 1 hr. The reaction mixture was concentrated, and the obtained residue was purified by silica gel chromatography (n-hexane-ethyl acetate=9:1) to give the title compound (1.63 g) as a pale-yellow oil.

### Step 4

### Production of 4-benzyloxy-3,5-dichloro-N-[2-(3-hydroxypropoxy)phenyl]benzamide

4-Benzyloxy-N-{2-[3-(tert-butyldimethylsilyloxy)propoxy]phenyl}-3,5-dichlorobenzamide (1.62 g) was dissolved in tetrahydrofuran (10 mL), tetrabutylammonium fluoride (1M tetrahydrofuran solution, 4.33 mL) was added, and the mixture was stirred at room temperature for 1 hr. 10% Aqueous citric acid solution was added to the reaction mixture, and extracted with ethyl acetate and tetrahydrofuran. The extract was washed successively with water and saturated brine, dried over anhydrous sodium sulfate, and concentrated. The obtained residue was crystallized from isopropyl ether to give the title compound (1.19 g) as a white solid.

### Step 5

### Production of 4-benzyloxy-3,5-dichloro-N-[2-(3-chloropropoxy)phenyl]benzamide

4-Benzyloxy-3,5-dichloro-N-[2-(3-hydroxypropoxy)phenyl]benzamide (1.00 g) was dissolved in pyridine (10 mL), methanesulfonyl chloride (0.225 mL) was added, and the mixture was stirred with heating at 70° C. for 2 hrs. Water was added to the reaction mixture, and the mixture was extracted with ethyl acetate. The extract was washed successively with 10% aqueous citric acid solution, water and saturated brine, and dried over anhydrous sodium sulfate. The solvent was evaporated to give the title compound (713 mg) as a beige solid.

### Step 6

### Production of (4-benzyloxy-3,5-dichlorophenyl)-(7,8-dihydro-6H-5-oxa-9-azabenzocyclohepten-9-yl)-methanone

4-Benzyloxy-3,5-dichloro-N-[2-(3-chloropropoxy)phenyl]benzamide (200 mg) was dissolved in N,N-dimethylformamide (3 mL), 60% sodium hydride (21 mg) and sodium iodide (64 mg) were added, and the mixture was stirred with heating at 60° C. 10% Aqueous citric acid solution was added to the reaction mixture, and the mixture was extracted with ethyl acetate. The extract was washed successively with water and saturated brine, dried over anhydrous sodium sulfate, and concentrated. The obtained residue was purified by silica gel chromatography (n-hexane-ethyl acetate=9:1) to give the title compound (121 mg) as an oil.

### Step 7

### Production of (3,5-dichloro-4-hydroxyphenyl)-(7,8-dihydro-6H-5-oxa-9-azabenzocyclohepten-9-yl)-methanone

(4-Benzyloxy-3,5-dichlorophenyl)-(7,8-dihydro-6H-5-oxa-9-azabenzocyclohepten-9-yl)-methanone (115 mg) was dissolved in tetrahydrofuran (5 mL). 7.5% Palladium-carbon (15 mg) was added to this solution and, under a hydrogen atmosphere, the mixture was stirred at room temperature for 0.5 hr. The reaction mixture was filtered through celite, and the residue was washed with tetrahydrofuran. The solvent was evaporated and the obtained residue was crystallized from isopropyl ether to give the title compound (69 mg) as a white solid.

The ¹H-NMR spectrum data of the compounds of Reference Examples 1-95 are shown in Table 1-Table 18.

The ¹H-NMR spectrum was measured in CDCl₃ or DMSO-d₆, with tetramethylsilane as an inner standard, and the total δ_{.} value are shown in ppm.

The symbols in the tables mean the following.

s: singlet
d: doublet
t: triplet
dd: double doublet
ddd: double double doublet
brs: broad singlet
m: multiplet
J: coupling constant

TABLE 1

| Ref. Ex. No. | Structural Formula | NMR |
|---|---|---|
| 1 | 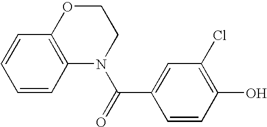 | (400 MHz, DMSO-d6) 3.87 (t, J = 4.50Hz, 2H), 4.30 (t, J = 4.50 Hz, 2H),6.74 (dd, J = 7.80, 7.80 Hz, 1H), 6.90(d, J = 8.30 Hz, 1H), 6.95-7.05 (m, 2H),7.10-7.20 (m, 1H), 7.34 (dd, J = 8.40,2.20 Hz, 1H), 7.54 (d, J = 2.0 Hz, 1H),10.82 (s, 1H) |
| 2 | 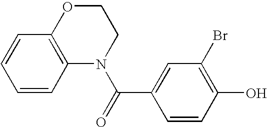 | (400 MHz, DMSO-d6) 3.87 (t, J = 4.63Hz, 2H), 4.30 (t, J = 4.63 Hz, 2H), 6.74(ddd, J = 8.34, 7.06, 1.39 Hz, 1H), 6.89(dd, J = 8.34, 1,62 Hz, 1H), 6.95-7.01(m, 2H), 7.15 (d, J = 7.87 Hz, 1H), 7.38(dd, J = 8.35, 2.07 Hz, 1H), 7.66 (d,J = 1.76 Hz, 1H), 10.89 (s, 1H) |
| 3 | 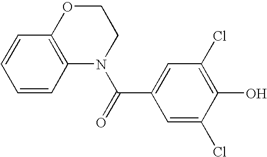 | (300 MHz, DMSO-d6) 3.87 (t, J = 4.58Hz, 2H), 4.31 (t, J = 4.58 Hz, 2H), 6.76(ddd, J = 7.20, 6.90, 1.50 Hz, 1H), 6.90(dd, J = 7.80, 1.50 Hz, 1H), 7.01 (ddd,J = 7.80, 6.90, 1.50 Hz, 1H), 7.18-7.25(m, 1H), 7.54 (s, 2H), 10.77 (s, 1H) |
| 4 | 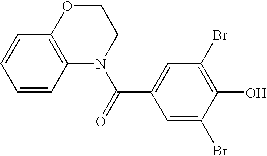 | (400 MHz, DMSO-d6) 3.87 (t, J = 4.55Hz, 2H), 4.31 (t, J = 4.55 Hz, 2H), 6.77(t, J = 8.33 Hz, 1H), 6.90 (d, J = 8.10 Hz,1H), 7.01 (t, J = 7.18 Hz, 1H), 7.21(brs, 1H), 7.70 (s, 2H), 10.54 (s, 1H) |
| 5 | 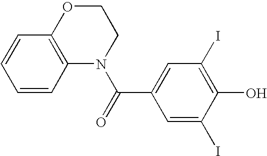 | (400 MHz, DMSO-d6) 3.85 (t, J = 4.40Hz, 2H), 4.30 (t, J = 4.40 Hz, 2H), 6.75(dd, J = 7.76, 7.76 Hz, 1H), 6.90 (dd,J = 8.10, 1.50 Hz, 1H), 7.01 (ddd, J =8.10, 7.76, 1.50 Hz, 1H), 7.19 (brs, 1H),7.86 (s, 2H), 10.05 (brs, 1H) |

TABLE 2

| Ref. Ex. No. | Structural Formula | NMR |
|---|---|---|
| 6 | 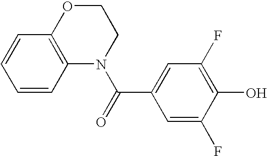 | (400 MHz, DMSO-d6) 3.87 (t, J = 4.65Hz, 2H), 4.30 (t, J = 4.65 Hz, 2H), 6.76(dd, J = 7.65, 7.65 Hz, 1H), 6.90 (d,J = 8.10 Hz, 1H), 7.01 (dd, J = 7.80,7.80 Hz, 1H), 7.10-7.35 (m, 3H), 10.85(s, 1H) |
| 7 | 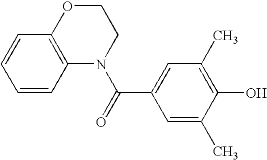 | (400 MHz, DMSO-d6) 2.12 (s, 6H), 3.82(t, J = 4.50 Hz, 2H), 4.20 (t, J = 4.50 Hz,2H), 6.70 (ddd, H = 7.00, 7.00, 1.60 Hz,1H), 6.84 (dd, J = 8.10, 1.40 Hz, 1H),6.93 (ddd, J = 7.00, 6.70, 1.60 Hz, 1H),7.10 (s, 2H), 7.19 (d, J = 8.10 Hz, 1H),8.85 (brs, 1H) |
| 8 | 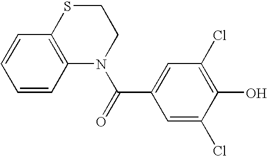 | (400 MHz, DMSO-d6) 3.30-3.35 (m, 2H),3.99-4.05 (m, 2H), 6.75 (d, J = 8.10 Hz,1H), 6.89 (dd, J = 8.10, 7.41 Hz, 1H),7.07 (dd, J = 7.87, 7.41 Hz, 1H), 7.20(s, 2H), 7.29 (d, J = 7.87 Hz, 1H),10.77 (s, 1H) |
| 9 | 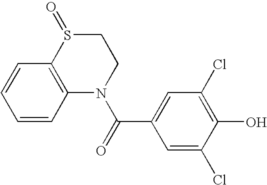 | (400 MHz, DMSO-d6) 3.28-3.34 (m, 1H),3.45-3.53 (m, 1H), 3.88-3.95 (m, 1H),4.24-4.31 (m, 1H), 6.98-7.00 (m, 1H),7.31-6.36 (m, 2H), 7.34 (s, 2H), 7.75-7.78(m, 1H), 10.76 (s, 1H) |
| 10 | 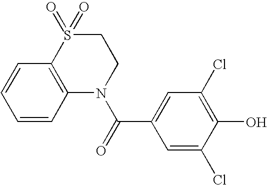 | (400 MHz, DMSO-d6) 3.92 (t, J = 6.25Hz, 2H), 4.34 (t, J = 6.25 Hz, 2H), 7.10(d, J = 7.87 Hz, 1H), 7.37-7.47 (m, 2H),7.38 (s, 2H), 7.85 (dd, J = 7.64, 1.62Hz, 1H), 10.90 (s, 1H) |
| 11 | 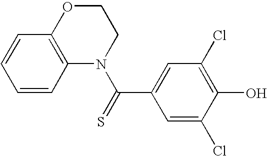 | (400 MHz, DMSO-d6) 4.51-4.58 (m, 4H),6.61-6.67 (m, 2H), 6.95 (d, J = 8.10 Hz,1H), 7.03-7.08 (m, 1H), 7.44 (s, 2H),10.74 (s, 1H) |

TABLE 3

| Ref. Ex. No. | Structural Formula | NMR |
|---|---|---|
| 12 | 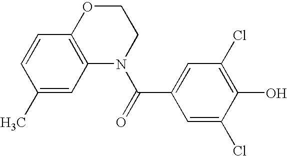 | (400 MHz, DMSO-d6) 2.12 (s, 3H), 3.86 (t,J = 3.60 Hz, 2H), 4.25 (t, J = 3.60 Hz, 2H),6.78-6.85 (m, 2H), 7.15 (brs, 1H), 7.54 (s,2H), 10.77 (brs, 1H) |
| 13 | 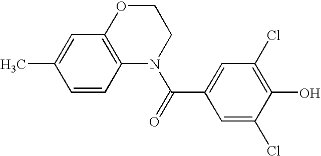 | (400 MHz, DMSO-d6) 2.20 (s, 3H), 3.84 (t,J = 4.50 Hz, 2H), 4.29 (t, J = 4.50 Hz, 2H),6.58 (d, J = 8.00 Hz, 1H), 6.72 (s, 1H), 7.09(brs, 1H), 7.52 (s, 2H), 10.76 (brs, 1H) |
| 14 | 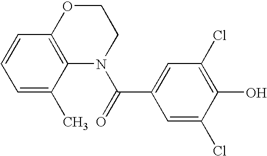 | (400 MHz, DMSO-d6) 1.99 (brs, 3H), 3.40-4.40 (m, 4H), 6.75 (d, J = 7.87 Hz, 2H),7.04 (t, J = 7.87 Hz, 1H), 7.64 (brs, 2H),10.89 (brs, 1H) |
| 15 | 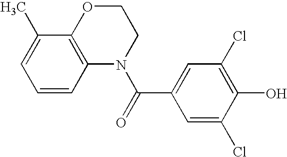 | (400 MHz, DMSO-d6) 2.16 (s, 3H), 3.86 (t,J = 4.63 Hz, 2H), 4.37 (t, J = 4.63 Hz, 2H),6.63 (t, J = 7.87 Hz, 1H), 6.89 (d, J = 7.18Hz, 1H), 6.94 (brs, 1H), 7.51 (s, 2H),10.76 (brs, 1H) |
| 16 | 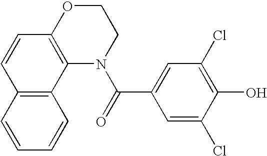 | (400 MHz, DMSO-d6) 3.40-4.45 (m, 4H),7.05-7.40 (m, 5H), 7.65-7.80 (m, 3H),10.88 (brs, 1H) |
| 17 | 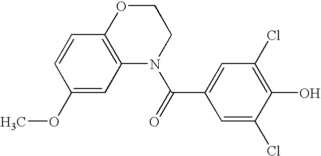 | (400 MHz, DMSO-d6) 3.53 (s, 3H), 3.84 (t,J = 4.40 Hz, 2H), 4.24 (t, J = 4.40 Hz, 2H),6.64 (dd, J = 8.80, 3.10 Hz, 1H), 6.83 (d,J = 8.80 Hz, 1H), 6.88 (brs, 1H), 7.56 (s,2H), 10.81 (brs, 1H) |
| 18 | 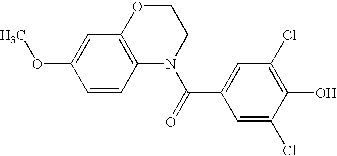 | (400 MHz, DMSO-d6) 3.69 (s, 3H), 3.84 (t,J = 4.40 Hz, 2H), 4.29 (t, J = 4.40 Hz, 2H),6.39 (d, J = 9.03 Hz, 1H), 6.47 (d, J = 2.78Hz, 1H), 7.14 (brs, 1H), 7.52 (s, 2H), 10.72(brs, 1H) |

TABLE 4

| Ref. | | |
|---|---|---|
| Ex. No. | Structural Formula | NMR |
| 19 | 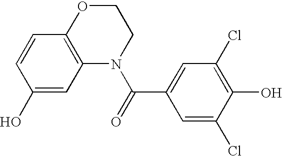 | (400 MHz, DMSO-d6) 3.80(t, J = 4.52 Hz, 2 H), 4.20(t, J = 4.52 Hz, 2 H), 6.44(dd, J = 8.70, 2.55 Hz, 1 H),6.70 (d, J = 8.70 Hz, 1 H),6.76 (brs, 1 H), 7.54 (s,2 H), 8.91 (s, 1 H), 10.77(brs, 1 H) |
| 20 | 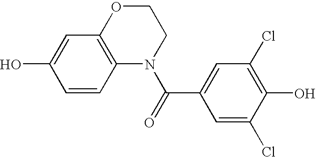 | (400 MHz, DMSO-d6) 3.81(t, J = 4.51 Hz, 2 H), 4.25(t, J = 4.51 Hz, 2 H), 6.17-6.22 (m, 1 H), 6.28 (d,J = 2.78 Hz, 1 H), 7.00 (brs,1 H), 7.49 (s, 2 H), 9.35(brs, 1 H), 10.71 (brs,1 H) |
| 21 | 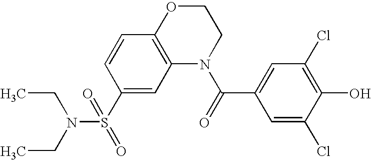 | (400 MHz, DMSO-d6) 1.00(t, J = 7.06 Hz, 6 H), 2.90(q, J = 7.06 Hz, 4 H), 3.91(t, J = 4.52 Hz, 2 H), 4.44(t, J = 4.52 Hz, 2 H), 7.09(d, J = 8.69 Hz, 1 H), 7.41(dd, J = 8.69, 2.32 Hz, 1 H),7.60 (s, 3 H), 10.89 (s, 1 H) |
| 22 | 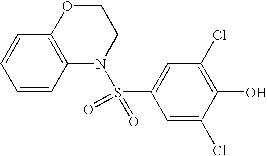 | (400 MHz, DMSO-d6) 3.78(t, J = 4.46 Hz, 2 H), 3.88(t, J = 4.46 Hz, 2 H), 6.81(d, J = 8.10 Hz, 1 H), 6.92(dd, J = 8.88, 7.41 Hz, 1 H),7.07 (dd, J = 8.10, 7.41 Hz,1 H), 7.55 (s, 2 H), 7.62(d, J = 8.33 Hz, 1 H), 11.65(brs, 1 H) |
| 23 | 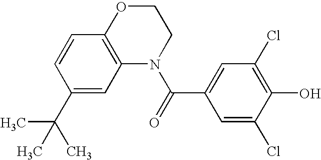 | (400 MHz, DMSO-d6) 1.02(s, 3 H), 3.90 (t,J = 4.17 Hz, 2 H), 4.35 (t,J = 4.17 Hz, 2 H), 6.81 (d,J = 8.56 Hz, 1 H), 6.62 (brs,1 H), 7.00 (dd, J = 8.56,2.32 Hz, 1 H), 7.47 (s,2 H), 10.73 (brs, 1 H) |
| 24 | 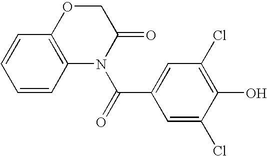 | (400 MHz, DMSO-d6) 4.83(s, 2 H), 6.90 (d,J = 8.33 Hz, 1 H), 7.00 (dd,J = 6.71, 6.71 Hz, 1 H), 7.09-7.16 (m, 2 H), 8.00 (s,2H) |

TABLE 5

| Ref. | | |
|---|---|---|
| Ex. No. | Structural Formula | NMR |
| 25 | 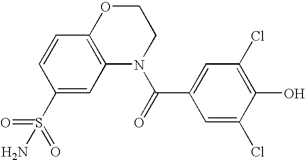 | (400 MHz, DMSO-d6) 3.88(t, J = 4.46 Hz, 2 H), 4.31(t, J = 4.46 Hz, 2 H), 7.08(d, J = 8.68 Hz, 1 H), 7.24(s, 2 H), 7.48 (dd,J = 8.68, 2.08 Hz, 1 H),7.62 (s, 2 H), 8.13 (s,1 H), 10.87 (s, 1 H) |
| 26 | 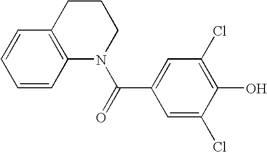 | (300 MHz, DMSO-d6) 1.94(quintet, J = 6.59 Hz, 2 H),2.81 (t, J = 6.59 Hz, 2 H),3.73 (t, J = 6.59 Hz, 2 H),6.78 (d, J = 7.87 Hz, 1 H),6.95 (dd, J = 7.87, 7.50 Hz,1 H), 7.03 (dd, J = 7.87,7.87 Hz, 1 H), 7.21 (d,J = 7.50, 1 H), 7.27 (s,2 H), 10.67 (s, 1 H) |
| 27 | 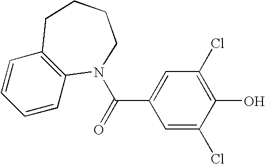 | (400 MHz, DMSO-d6) 1.30-1.45 (m, 1 H), 1.75-1.90(m, 2 H), 1.95-2.05 (m,1 H), 2.60-2.70 (m, 1 H),2.80-3.05 (m, 2 H), 4.70-4.85 (m, 1 H), 6.76-6.81(m, 1 H), 6.98-7.06(m, 1 H), 7.04 (s, 2 H),7.15 (t, J = 7.64 Hz, 1 H),7.32 (d, J = 7.64 Hz, 1 H),10.54 (brs, 1 H) |
| 28 | 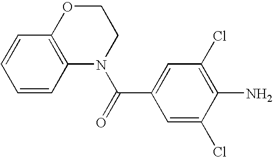 | (400 MHz, DMSO-d6) 3.88(t, J = 4.52 Hz, 2 H), 4.30(t, J = 4.52, 2 H), 6.08 (s,2 H), 6.75 (ddd, J = 8.34,8.34, 6.94 Hz, 1 H), 6.89(dd, J = 8.10, 1.39 Hz, 1 H),6.99 (ddd, J = 10.98, 7.03,1.39 Hz, 1 H), 7.15 (d,J = 7.30 Hz, 1 H), 7.42 (s,2 H) |

TABLE 6

| Ref. | | |
|---|---|---|
| Ex. No. | Structural Formula | NMR |
| 29 | 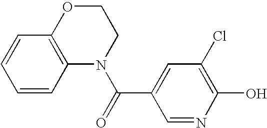 | (400 MHz, DMSO-d6) 3.90(t, J = 4.51 Hz, 2 H), 4.32(t, J = 4.51 Hz, 2 H), 6.79(ddd, J = 8.34, 7.06,1.39 Hz, 1 H), 6.90 (dd,J = 8.10, 1.39 Hz, 1 H), 7.01(ddd, J = 8.28, 7.12,1.62 Hz, 1 H), 7.23 (d,J = 8.10 Hz, 1 H), 7.72 (d,J = 2.32 Hz, 1 H), 7.80 (d,J = 2.32 Hz, 1 H), 12.51(brs, 1 H) |
| 30 | 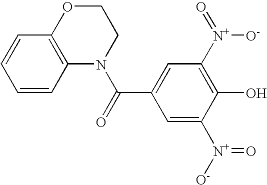 | (300 MHz, DMSO-d6) 3.90(t, J = 4.58 Hz, 2 H), 4.32(t, J = 4.58 Hz, 2 H), 5.00(brs, 1 H), 6.74-6.80(m, 1 H), 6.89-7.04 (m,2 H), 7.24 (dd, J = 8.80,1.50 Hz, 1 H), 8.20 (s, 2 H). |
| 31 | 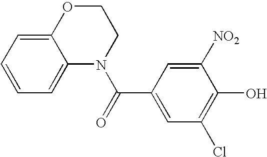 | (300 MHz, DMSO-d6) 3.87-3.90 (m, 2 H), 4.32-4.35(m, 2 H), 6.74-6.79 (m,1 H), 6.90-6.93 (m, 1 H),7.00-7.05 (m, 1 H), 7.24-7.27 (m, 1 H), 7.91 (d,J = 2.20 Hz, 1 H), 8.06 (d,J = 2.20 Hz, 1 H) |
| 32 | 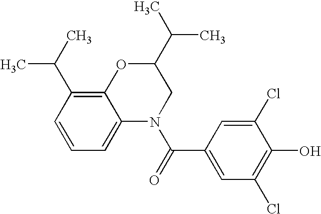 | (300 MHz, DMSO-d6) 0.95(d, J = 7.00 Hz, 3 H), 1.03(d, J = 6.60 Hz, 3 H), 1.18-1.22 (m, 6 H), 1.86-1.99(m, 1 H), 3.20-3.39 (m,2 H), 4.08-4.21 (m, 2 H),6.67 (dd, J = 8.10, 7.70 Hz,1 H), 6.86-6.89 (m, 1 H),6.95 (dd, J = 7.70, 1.30 Hz,1 H), 7.47 (s, 2 H), 10.76(brs, 1 H) |

TABLE 7

| Ref. | | |
|---|---|---|
| Ex. No. | Structural Formula | NMR |
| 33 | 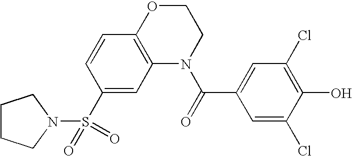 | (400 MHz, DMSO-d6) 1.62-1.66 (m, 4 H), 2.90-2.95(m, 4 H), 3.91 (t,J = 4.40 Hz, 2 H), 4.44 (t,J = 4.40 Hz, 2 H), 7.12 (d,J = 8.57 Hz, 1 H), 7.43 (dd,J = 8.57, 2.08 Hz, 1 H), 7.61(s, 2 H), 7.68 (brs, 1 H),10.86 (brs, 1 H) |
| 34 | 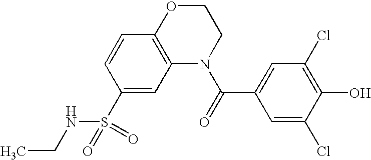 | (400 MHz, DMSO-d6) 0.95(t, J = 7.18 Hz, 3 H), 2.51-2.59 (m, 2 H), 3.91 (t,J = 4.40 Hz, 2 H), 4.40 (t,J = 4.40 Hz, 2 H), 7.11 (d,J = 8.80 Hz, 1 H), 7.36 (t,J = 5.79 Hz, 1 H), 7.41 (dd,J = 8.80, 2.31 Hz, 1 H), 7.60(s, 2 H), 7.73 (brs, 1 H),10.84 (s, 1 H) |
| 35 | 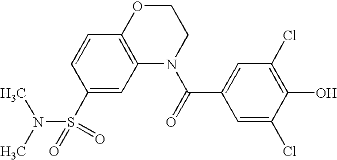 | (400 MHz, DMSO-d6) 2.47(s, 6 H), 3.92 (t,J = 3.94 Hz, 2 H), 4.42 (t,J = 3.94 Hz, 2 H), 7.14 (d,J = 8.57 Hz, 1 H), 7.38 (dd,J = 8.57, 2.08 Hz, 1 H), 7.62(s, 2 H), 7.68 (brs, 1 H),10.85 (brs, 1 H) |
| 36 | 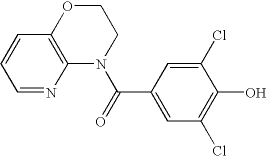 | (400 MHz, DMSO-d6) 3.96(t, J = 4.65 Hz, 2 H), 4.49(t, J = 4.65 Hz, 2 H), 7.03(dd, J = 7.90, 4.60 Hz, 1 H),7.33 (dd, J = 8.00, 1.50 Hz,1 H), 7.58 (dd, J = 4.60,1.40 Hz, 1 H), 10.72 (s, 1 H) |
| 37 | 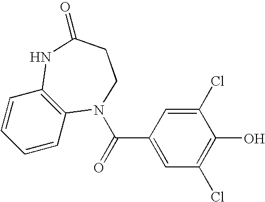 | (400 MHz, DMSO-d6) 2.20-2.80 (m, 2 H), 3.60-3.85(m, 1 H), 4.40-4.70 (m,1 H), 6.85-7.05 (m, 4 H),7.14 (d, J = 7.90 Hz, 1 H),7.28 (ddd, J = 7.40, 7.40,1.80 Hz, 1 H), 10.04 (s,1 H), 10.66 (s, 1 H) |

TABLE 8

| Ref. | | |
|---|---|---|
| Ex. No. | Structural Formula | NMR |
| 38 | 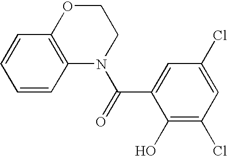 | (400 MHz, DMSO-d6) 3.50-3.90 (m, 2 H), 4.20-4.40(m, 2 H), 6.70-7.10 (m,3 H), 7.39 (s, 1 H), 7.62(s, 1 H), 10.31 (s, 1 H) |
| 39 | 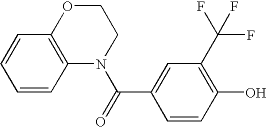 | (400 MHz, DMSO-d6) 3.88(t, J = 4.50 Hz, 2 H), 4.31(t, J = 4.50 Hz, 2 H), 6.74(dd, J = 7.65, 7.65 Hz, 1 H),6.90 (d, J = 8.30 Hz, 1 H),6.95-7.25 (m, 3 H), 7.64(d, J = 8.60 Hz, 1 H), 7.77(s, 1 H), 11.20 (s, 1 H) |
| 40 | 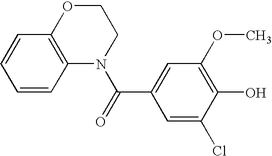 | (400 MHz, DMSO-d6) 3.79(s, 3 H), 3.88 (t,J = 4.60 Hz, 2 H), 4.30 (t,J = 4.60 Hz, 2 H), 6.76 (dd,J = 7.70, 7.70 Hz, 1 H), 6.90(d, J = 8.20 Hz, 1 H), 6.99(dd, J = 7.70, 7.70 Hz, 1 H),7.09 (s, 1 H), 7.14 (s,1 H), 7.15-7.30 (m, 1 H),10.01 (s, 1 H) |
| 41 | 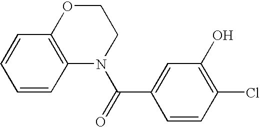 | (400 MHz, DMSO-d6) 3.85(t, J = 4.55 Hz, 2 H), 4.30(t, J = 4.55 Hz, 2 H), 6.75(dd, J = 7.80, 7.80 Hz, 1 H),6.90 (d, J = 8.20 Hz, 1 H),6.94 (d, J = 8.2 Hz, 1 H),7.00 (dd, J = 8.30, 8.30 Hz,1 H), 7.10 (s, 1 H), 7.15-7.30 (m, 1 H), 7.39 (d,J = 8.30 Hz, 1 H), 10.50 (s,1 H) |
| 42 | 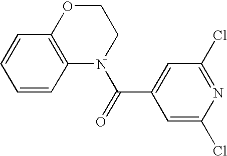 | (400 MHz, DMSO-d6) 3.81(brs, 2 H), 4.32 (brs,2 H), 6.84 (brs, 1 H), 6.93(dd, J = 8.10, 1.39 Hz, 1 H),7.08 (t, J = 8.10, 1 H), 7.77 (s, 2 H), 8.00 (brs, 1 H) |

TABLE 9

| Ref. | | |
|---|---|---|
| Ex. No. | Structural Formula | NMR |
| 43 | 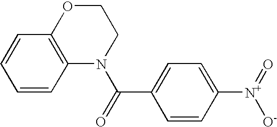 | (400 MHz, DMSO-d6) 3.83-3.89 (m, 2 H), 4.33-4.39(m, 2 H), 6.74 (brs, 1 H),6.92 (dd, J = 8.10, 1.39 Hz,1 H), 7.03 (ddd, J = 7.64,7.64, 1.39 Hz, 1 H), 7.20(brs, 1 H), 7.80 (d,J = 8.80 Hz, 2 H), 8.28 (d,J = 8.80 Hz, 2 H) |
| 44 | 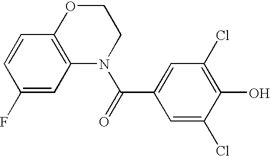 | (400 MHz, DMSO-d6) 3.84(t, J = 4.40 Hz, 2 H), 4.25(t, J = 4.40 Hz, 2 H), 6.87-6.95 (m, 2 H), 7.34 (d,J = 10.40 Hz, 1 H), 7.59 (s,2 H), 10.83 (brs, 1 H) |
| 45 | 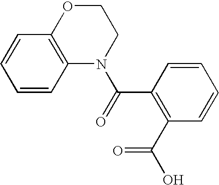 | (400 MHz, DMSO-d6) 3.20-3.60 (m, 2 H), 4.10-4.60(m, 2 H), 6.10-6.50 (m,1 H), 6.70-7.30 (m, 3 H),7.40-8.40 (m, 5 H),13.33 (s, 1 H) |
| 46 | 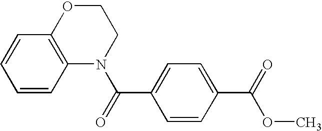 | (300 MHz, CDCl3) 3.93 (s,3 H), 4.02 (t, J = 4.80 Hz,2 H), 4.40 (t, J = 4.80 Hz,2 H), 6.63 (dd, J = 7.50,7.50 Hz, 1 H), 6.82 (brs,1 H), 6.91 (dd, J = 8.10,1.50 Hz, 1 H), 7.00 (ddd,J = 8.10, 7.50, 1.50 Hz,1 H), 7.56 (d, J = 8.10 Hz,2 H), 8.04 (d, J = 8.10 Hz, 2 H) |
| 47 | 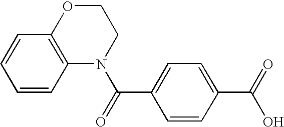 | (300 MHz, DMSO-d6) 3.86(t, J = 4.50 Hz, 2 H), 4.33(t, J = 4.50 Hz, 2 H), 6.73(dd, J = 7.20, 7.20 Hz, 1 H),6.91 (dd, J = 8.10, 1.50 Hz,1 H), 7.01 (ddd, J = 8.10,7.20, 1.50 Hz, 1 H), 7.22(brs, 1 H), 7.61 (d,J = 8.10 Hz, 2 H), 7.97 (d,J = 8.10 Hz, 2 H) |

TABLE 10

| Ref. | | |
|---|---|---|
| Ex. No. | Structural Formula | NMR |
| 48 | 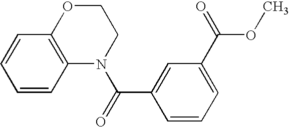 | (300 MHz, CDCl3) 3.92 (s,3 H), 4.02 (t, J = 4.80 Hz,2 H), 4.40 (t, J = 4.80 Hz,2 H), 6.65 (dd, J = 7.20,7.20 Hz, 1 H), 6.92 (dd,J = 8.40, 1.20 Hz, 1 H), 7.00(ddd, J = 8.40, 7.20,1.20 Hz, 1 H), 7.45 (dd,J = 7.80, 7.80 Hz, 1 H), 7.66(d, J = 7.80 Hz, 1 H), 8.11(d, J = 7.80 Hz, 1 H), 8.20(s, 1 H) |
| 49 | 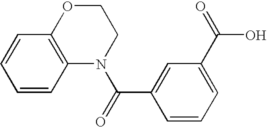 | (400 MHz, DMSO-d6) 3.87(t, J = 4.50 Hz, 2 H), 4.33(t, J = 4.50 Hz, 2 H), 6.72(dd, J = 7.80, 7.80 Hz, 1 H),6.91 (dd, J = 8.40, 1.20 Hz,1 H), 7.01 (ddd, J = 8.40,7.80, 1.20 Hz, 1 H), 7.20(brs, 1 H), 7.57 (dd,J = 7.80, 7.80 Hz, 1 H), 7.76(d, J = 7.80 Hz, 1 H), 8.03-8.05 (m, 2 H), 13.20 (brs,1 H) |
| 50 | 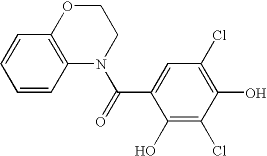 | (400 MHz, DMSO-d6) 3.78(t, J = 4.10 Hz, 2 H), 4.29(t, J = 4.10 Hz, 2 H), 6.79(dd, J = 7.70, 7.70 Hz, 1 H),6.89 (d, J = 8.4 Hz, 1 H),7.02 (dd, J = 7.70, 7.70 Hz,1 H), 7.30 (s, 1 H), 7.52(brs, 1 H), 10.14 (s,1 H), 10.41 (s, 1 H) |
| 51 | 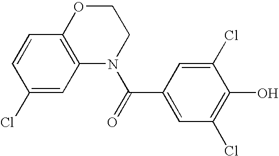 | (400 MHz, DMSO-d6) 3.84(t, J = 4.30 Hz, 2 H), 4.27(t, J = 4.30 Hz, 2 H), 6.95(d, J = 8.80 Hz, 1 H), 7.08(dd, J = 8.80, 2.60 Hz, 1 H),7.53-7.58 (m, 1 H), 7.59(s, 2 H), 10.84 (s, 1 H) |
| 52 | 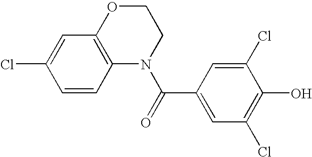 | (400 MHz, DMSO-d6) 3.86(t, J = 4.60 Hz, 2 H), 4.32(t, J = 4.60 Hz, 2 H), 6.86(dd, J = 8.90, 2.60 Hz, 1 H),7.02 (d, J = 2.60 Hz, 1 H),7.27-7.38 (m, 1 H), 7.57(s, 2 H), 10.81 (s, 1 H) |

TABLE 11

| Ref. | | |
|---|---|---|
| Ex. No. | Structural Formula | NMR |
| 53 | 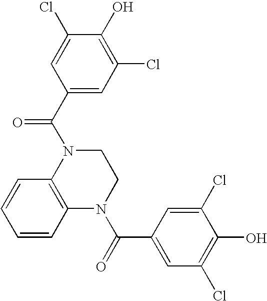 | (400 MHz, DMSO-d6) 3.99(s, 4 H), 6.84-6.91 (m,4 H), 7.47 (s, 4 H), 10.75(s, 2 H) |
| 54 | 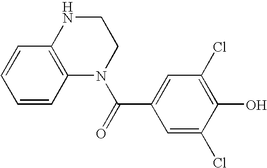 | (400 MHz, DMSO-d6) 3.38(t, J = 4.80 Hz, 2 H), 3.74(t, J = 4.80 Hz, 2 H), 6.20(s, 1 H), 6.29 (ddd,J = 8.10, 7.70, 1.50 Hz,1 H), 6.61 (dd, J = 8.10,1.50 Hz, 1H), 6.65 (s,1 H), 6.82 (ddd, J = 8.10,7.70, 1.50 Hz, 1 H), 7.35(s, 2 H), 10.67 (s, 1 H) |
| 55 | 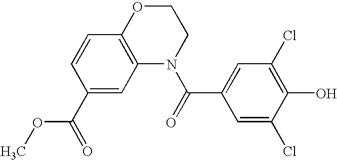 | (400 MHz, DMSO-d6) 3.78(s, 3 H), 3.88 (t,J = 4.50 Hz, 2 H), 4.35 (t,J = 4.50 Hz, 2 H), 7.02 (d,J = 8.60 Hz, 1 H), 7.61-7.64 (m, 3 H), 8.12 (s,1 H), 10.81 (s, 1 H) |
| 56 | 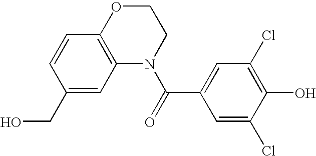 | (400 MHz, DMSO-d6) 3.84(t, J = 4.40 Hz, 2 H), 4.27(t, J = 4.40 Hz, 2 H), 4.27-4.35 (m, 2 H), 4.97-5.09(m, 1 H), 6.85 (d,J = 8.30 Hz, 1 H), 6.97 (dd,J = 8.30, 1.90 Hz, 1 H), 7.30-7.41 (m, 1 H), 7.55 (s,2 H), 10.78 (s, 1 H) |
| 57 | 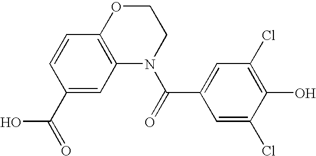 | (400 MHz, DMSO-d6) 3.88(t, J = 4.50 Hz, 2 H), 4.35(t, J = 4.50 Hz, 2 H), 7.00(d, J = 2.60 Hz, 1 H), 7.59-7.62 (m, 3 H), 8.06 (s,1 H), 10.80 (s, 1 H), 12.60(s, 1 H) |

TABLE 12

| Ref. | | |
|---|---|---|
| Ex. No. | Structural Formula | NMR |
| 58 | 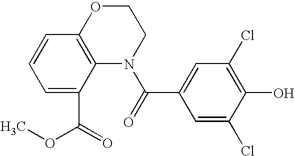 | (400 MHz, DMSO-d6) 3.56(s, 3 H), 3.79-4.02 (m,2 H), 4.13-4.35 (m,2 H), 7.15-7.54 (m, 5 H),10.83 (s, 1 H) |
| 59 | 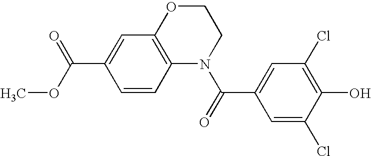 | (400 MHz, DMSO-d6) 3.82(s, 3 H), 3.90 (t,J = 4.30 Hz, 2 H), 4.35 (t,J = 4.30 Hz, 2 H), 7.37-7.47 (m, 3 H), 7.60 (s,2 H), 10.86 (s, 1 H) |
| 60 | 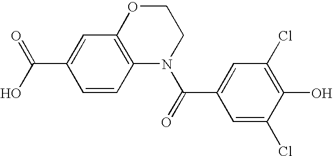 | (400 MHz, DMSO-d6) 3.89(t, J = 4.50 Hz, 2 H), 4.34(t, J = 4.50 Hz, 2 H), 7.34-7.41 (m, 3 H), 7.59 (s,2 H), 10.86 (s, 1 H), 12.85(s, 1 H) |
| 61 | 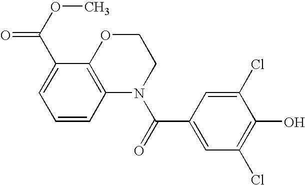 | (400 MHz, DMSO-d6) 3.79(s, 3 H), 3.90 (t,J = 4.50 Hz, 2 H), 4.39 (t,J = 4.50 Hz, 2 H), 6.84 (t,J = 8.00 Hz, 1 H), 7.34-7.41 (m, 1 H), 7.40 (dd,J = 7.70, 1.60 Hz, 1 H), 7.56(s, 2 H), 10.83 (s, 1 H) |
| 62 | 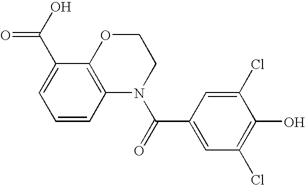 | (400 MHz, DMSO-d6) 3.89(t, J = 4.60 Hz, 2 H), 4.39(t, J = 4.60 Hz, 2 H), 6.77-6.84 (m, 1 H), 7.33 (s,1 H), 7.38 (dd, J = 7.90,1.60 Hz, 1 H), 7.55 (s,2 H), 10.83 (s, 1 H), 12.73(s, 1 H) |
| 63 | 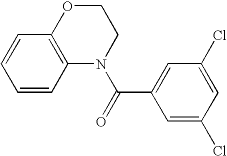 | (400 MHz, DMSO-d6) 3.84(t, J = 4.10 Hz, 2 H), 4.34(t, J = 4.10 Hz, 2 H), 6.78(t, J = 7.50 Hz, 1 H), 6.92(dd, J = 8.20, 1.40 Hz, 1 H),7.00-7.08 (m, 1 H), 7.60(d, J = 1.80 Hz, 2 H), 7.75(t, J = 1.80 Hz, 1 H) |

TABLE 13

| Ref. | | |
|---|---|---|
| Ex. No. | Structural Formula | NMR |
| 64 | 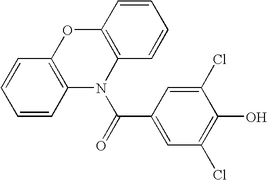 | (400 MHz, DMSO-d6) 7.02-7.11 (m, 2 H), 7.22-7.28(m, 4 H), 7.34 (s, 2 H),7.40-7.48 (m, 2 H),10.85 (s, 1 H) |
| 65 | 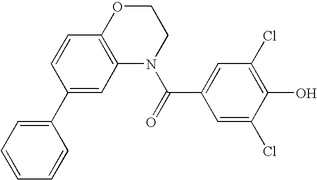 | (400 MHz, DMSO-d6) 3.92(t, J = 4.30 Hz, 2 H), 4.36(t, J = 4.30 Hz, 2 H), 6.99(d, J = 8.50 Hz, 1 H), 7.26-7.39 (m, 6 H), 7.48 (s,1 H), 7.60 (s, 2 H), 10.81(s, 1 H) |
| 66 | 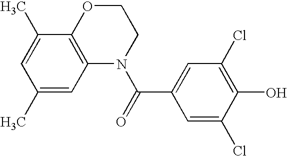 | (400 MHz, DMSO-d6) 2.06(s, 3 H), 2.12 (s, 3 H),3.82 (t, J = 4.50 Hz, 2 H),4.30 (t, J = 4.50 Hz, 2 H),6.72 (s, 1 H), 6.89 (s,1 H), 7.52 (s, 2 H), 10.76(s, 1 H) |
| 67 | 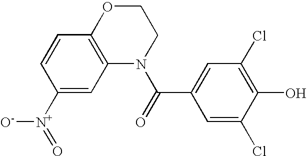 | (400 MHz, DMSO-d6) 3.91(t, J = 4.60 Hz, 2 H), 4.41(t, J = 4.60 Hz, 2 H), 7.15(d, J = 9.10 Hz, 1 H), 7.64(s, 2 H), 7.95 (dd,J = 9.10, 2.80 Hz, 1 H), 8.53(s, 1 H), 10.87 (s, 1 H) |
| 68 | 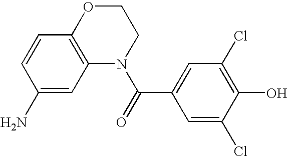 | (400 MHz, DMSO d6) 3.78(t, J = 4.40 Hz, 2 H), 4.16(t, J = 4.40 Hz, 2 H), 6.30(dd, J = 8.70, 2.70 Hz, 1 H),6.56 (s, 1 H), 6.60 (d,J = 8.70 Hz, 1 H), 7.51 (s, 2 H) |
| 69 | 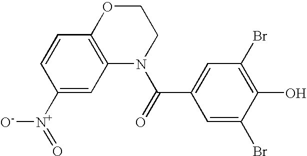 | (400 MHz, DMSO-d6) 3.91(t, J = 4.50 Hz, 2 H), 4.41(t, J = 4.50 Hz, 2 H), 7.15(d, J = 9.00 Hz, 1 H), 7.80(s, 2 H), 7.95 (dd,J = 9.00, 2.70 Hz, 1 H), 8.53(s, 1 H), 10.62 (s, 1 H) |
| 70 | 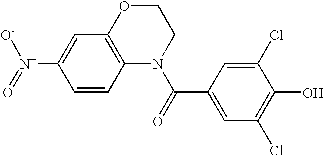 | (400 MHz, DMSO-d6) 3.93(t, J = 4.30 Hz, 2 H), 4.39(t, J = 4.30 Hz, 2 H), 7.61-7.74 (m, 3 H), 7.65 (s,2 H), 10.84 (s, 1 H) |

TABLE 14

| Ref. | | |
|---|---|---|
| Ex. No. | Structural Formula | NMR |
| 71 | 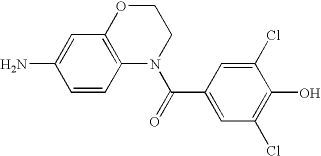 | (400 MHz, DMSO-d6) 3.79(t, J = 3.70 Hz, 2 H), 4.22(t, J = 3.70 Hz, 2H), 5.90-6.04 (m, 1 H), 6.04-6.16(m, 1 H), 6.86 (s, 1 H),7.47 (s, 1 H) |
| 72 | 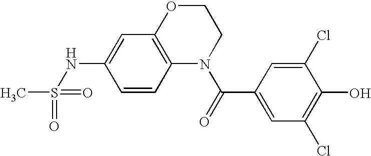 | (400 MHz, DMSO-d6) 2.95(s, 3 H), 3.85 (t,J = 4.20 Hz, 2 H), 4.29 (t,J = 4.20 Hz, 2 H), 6.63 (dd,J = 8.60, 2.30 Hz, 1 H), 6.77(d, J = 2.60 Hz, 1 H), 7.24(s, 1 H), 7.53 (s, 2 H),9.64 (s, 1 H), 10.75 (s,1 H) |
| 73 | 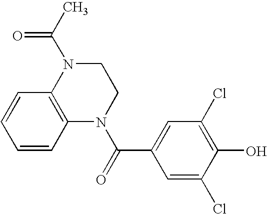 | (400 MHz, DMSO-d6) 2.25(s, 3 H), 3.92 (s, 4 H),6.87 (d, J = 7.90 Hz, 1 H),6.94-7.03 (m, 1 H), 7.09-7.21 (m, 1 H), 7.32 (s,2 H), 7.68 (s, 1 H), 10.77(s, 1 H) |
| 74 | 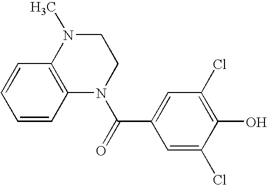 | (400 MHz, DMSO-d6) 2.94(s, 3 H), 3.40 (t,J = 5.40 Hz, 2 H), 3.84 (t,J = 5.40 Hz, 2 H), 6.35-6.43 (m, 1 H), 6.70 (s,1 H), 6.74 (dd, J = 8.40,1.20 Hz, 1 H), 6.92-6.99(m, 1 H), 7.33 (s, 2 H),10.70 (s, 1 H) |
| 75 | 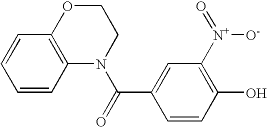 | (400 MHz, DMSO-d6) 3.89(t, J = 4.40 Hz, 2 H), 4.32(t, J = 4.40 Hz, 2 H), 6.75(dd, J = 7.64, 7.64 Hz, 1 H),6.91 (dd, J = 8.10, 1.62 Hz,1 H), 7.01 (ddd, J = 8.10,7.64, 1.62 Hz, 1 H), 7.14(d, J = 8.57 Hz, 1 H), 7.18(brs, 1 H), 7.68 (dd,J = 8.57, 2.20 Hz, 1 H), 8.05(d, J = 2.20 Hz, 1 H), 11.61(brs, 1 H) |

TABLE 15

| Ref. | | |
|---|---|---|
| Ex. No. | Structural Formula | NMR |
| 76 | 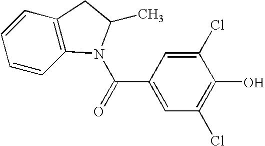 | (400 MHz, DMSO-d6) 1.07(s, 3 H), 2.56-2.70 (m,1 H), 3.38-3.51 (m, 1 H),4.54-4.69 (m, 1 H), 6.96-7.07 (m, 1 H), 7.07-7.20 (m, 1 H), 7.24-7.32(m, 1 H), 7.42 (s, 1 H),7.62 (s, 2 H), 10.74 (s, 1 H) |
| 77 | 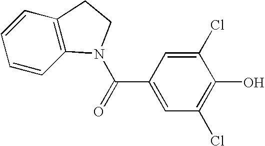 | (400 MHz, DMSO-d6) 3.08(t, J = 8.30 Hz, 2 H), 4.05(t, J = 8.30 Hz, 2 H), 6.98-7.09 (m, 1 H), 7.11-7.22(m, 1 H), 7.24-7.34 (m,1 H), 7.63 (s, 2 H), 7.88(s, 1 H), 10.73 (s, 1 H) |
| 78 | 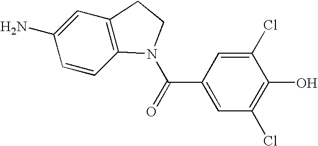 | (400 MHz, DMSO-d6) 3.06(t, J = 8.10 Hz, 2 H), 4.06(t, J = 8.10 Hz, 2 H), 6.90(d, J = 8.30 Hz, 1 H), 6.99(s, 1 H), 7.12-7.27 (m,1 H), 7.62 (s, 2 H) |
| 79 | 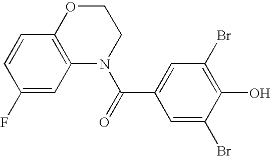 | (400 MHz, DMSO-d6) 3.84(t, J = 4.19 Hz, 2 H), 4.25(t, J = 4.19 Hz, 2 H), 6.87-6.95 (m, 2 H), 7.33 (d,J = 10.65 Hz, 1 H), 7.75 (s,2 H), 10.60 (brs, 1 H) |
| 80 | 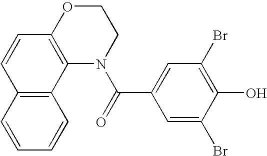 | (400 MHz, DMSO-d6) 3.70-4.50 (m, 4 H), 7.10-7.45(m, 5H), 7.74-8.05 (m,3 H), 10.73 (brs, 1 H) |
| 81 | 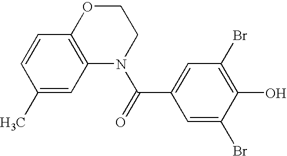 | (400 MHz, DMSO-d6) 2.11(s, 3 H), 3.86 (t,J = 4.17 Hz, 2 H), 4.25 (t,J = 4.17 Hz, 2 H), 6.78-6.85 (m, 2 H), 7.15 (brs,1 H), 7.70 (s, 2 H), 10.54(brs, 1 H) |
| 82 | 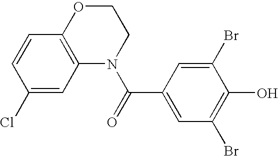 | (400 MHz, DMSO-d6) 3.84(t, J = 4.50 Hz, 2 H), 4.27(t, J = 4.50 Hz, 2 H), 6.95(d, J = 8.80 Hz, 1 H), 7.08(dd, J = 8.80, 2.50 Hz, 1 H),7.55 (s, 1 H), 7.75 (s,2 H), 10.60 (s, 1 H) |

TABLE 16

| Ref. | | |
|---|---|---|
| Ex. No. | Structural Formula | NMR |
| 83 | 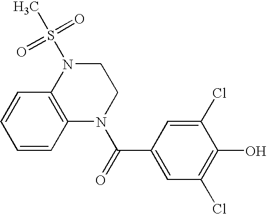 | (400 MHz, DMSO-d6) 3.20(s, 3 H), 3.87-4.00 (m,4 H), 6.94-7.03 (m, 1 H),7.09-7.18 (m, 2 H), 7.41(s, 2 H), 7.69 (d,J = 8.40 Hz, 1 H), 10.81 (s,1 H) |
| 84 | 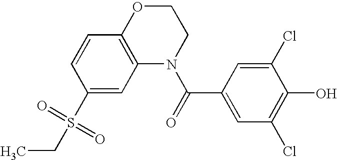 | (400 MHz, DMSO-d6) 0.99(t, J = 7.30 Hz, 3 H), 3.09(q, J = 7.30 Hz, 2 H), 3.92(t, J = 4.50 Hz, 2 H), 4.42(t, J = 4.50 Hz, 2 H), 7.16(d, J = 8.70 Hz, 1 H), 7.50(dd, J = 8.70, 2.20 Hz, 1 H),7.61 (s, 2 H), 7.80 (s,1 H), 10.86 (s, 1 H) |
| 85 | 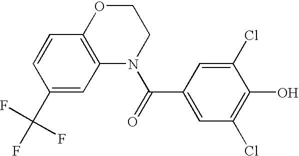 | (400 MHz, DMSO-d6) 3.90(t, J = 4.50 Hz, 2 H), 4.35(t, J = 4.50 Hz, 2 H), 7.11(d, J = 8.50 Hz, 1 H), 7.38(dd, J = 8.50, 2.10 Hz, 1 H),7.60 (s, 2 H), 7.83 (s,1 H), 10.85 (s, 1 H) |
| 86 | 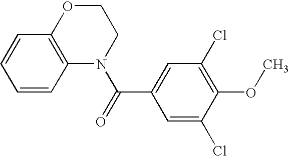 | (400 MHz, DMSO-d6) 3.85(t, J = 4.40 Hz, 2 H), 3.87(s, 3 H), 4.32 (t, J = 4.40,2 H), 6.79 (dd, J = 7.99,7.99 Hz, 1 H), 6.92 (dd,J = 8.10, 1.39 Hz, 1 H), 7.04(ddd, J = 8.10, 7.18,1.39 Hz, 1 H), 7.37 (brs,1 H), 7.68 (s, 2 H) |
| 87 | 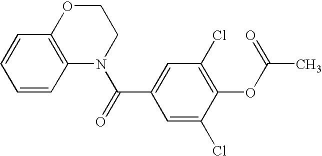 | (400 MHz, DMSO-d6) 2.44(s, 3 H), 3.85 (t,J = 4.18 Hz, 2 H), 4.33 (t,J = 4.18 Hz, 2 H), 6.80 (dd,J = 7.41, 7.41 Hz, 1 H), 6.92(dd, J = 8.34, 1.39 Hz, 1 H),7.05 (ddd, J = 8.10, 7.18,1.39 Hz, 1 H), 7.42 (brs,1 H), 7.80 (s, 2 H) |

TABLE 17

| Ref. | | |
|---|---|---|
| Ex. No. | Structural Formula | NMR |
| 88 | 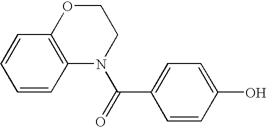 | (400 MHz, DMSO-d6) 3.87(t, J = 4.40 Hz, 2 H), 4.28(t, J = 4.40 Hz, 2 H), 6.72(ddd, J = 8.10, 7.18,1.62 Hz, 1 H), 6.78 (d,J = 8.57 Hz, 2 H), 6.88 (dd,J = 8.10, 1.62 Hz, 1 H), 6.97(ddd, J = 8.22, 7.18,1.62 Hz, 1 H), 7.13 (d,J = 8.10 Hz, 1 H), 7.39 (d,J = 8.57 Hz, 2 H), 10.06(brs, 1 H) |
| 89 | 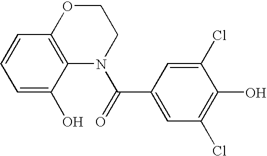 | (400 MHz, DMSO-d6) 3.85(brs, 2 H), 4.31 (t,J = 4.75 Hz, 2 H), 6.24 (dd,J = 8.11, 1.16 Hz, 1 H), 6.38(dd, J = 8.11, 1.16 Hz, 1 H),6.65 (s, 1 H), 6.87 (dd,J = 8.11, 8.11 Hz, 1 H), 7.39(s, 1 H), 9.39 (s, 1 H),10.69 (brs, 1 H) |
| 90 | 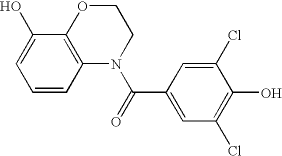 | (400 MHz, DMSO-d6) 3.86(t, J = 4.55 Hz, 2 H), 4.35(t, J = 4.55 Hz, 2 H), 6.42-6.59 (m, 3 H), 7.51 (s,2 H), 9.14 (s, 1 H), 10.77(brs, 1 H) |
| 91 | 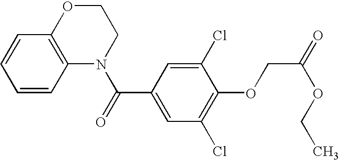 | (400 MHz, DMSO-d6) 1.24(t, J = 7.20 Hz, 3 H), 3.86(t, J = 4.20 Hz, 2 H), 4.22(q, J = 7.20 Hz, 2 H), 4.34(t, J = 4.20 Hz, 2 H), 4.76(s, 2 H), 6.78-6.82 (m,1 H), 6.92-6.94 (m, 1 H),7.03-7.07 (m, 1 H), 7.36(brs, 1 H), 7.69 (s, 2 H) |
| 92 | 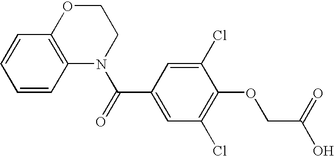 | (400 MHz, DMSO-d6) 3.85(t, J = 4.40 Hz, 2 H), 4.32(t, J = 4.40 Hz, 2 H), 4.64(s, 2 H), 6.80 (ddd,J = 8.00, 7.60, 1.60 Hz, 1 H),6.93 (dd, J = 7.60, 1.60 Hz,1 H), 7.05 (ddd, J = 8.00,7.60, 1.60 Hz, 1 H), 7.38(brs, 1 H), 7.68 (s, 2 H),13.14 (brs, 1 H) |

TABLE 18

| Ref. | | |
|---|---|---|
| Ex. No. | Structural Formula | NMR |
| 93 | 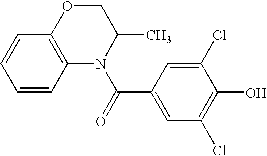 | (400 MHz, DMSO-d6) 1.16(d, J = 6.80 Hz, 3 H), 4.19-4.22 (m, 1 H), 4.34-4.37(m, 1 H), 4.54-4.56 (m,1 H), 6.71-6.75 (m, 1 H),6.91-7.04 (m, 3 H), 7.50(s, 2 H), 10.78 (s, 1 H) |
| 94 | 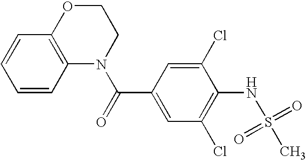 | (400 MHz, DMSO-d6) 3.20(s, 3 H), 3.80-3.89 (m,2 H), 4.28-4.38 (m, 2 H),6.73-6.85 (m, 1 H), 6.89-6.96 (m, 1 H), 7.00-7.08 (m, 1 H), 7.42 (brs,1 H), 7.73 (s, 2 H), 9.76(s, 1 H) |
| 95 | 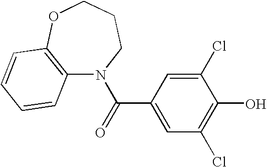 | (400 MHz, DMSO-d6) 1.72-2.12 (m, 2 H), 2.71-4.99(m, 4 H), 6.81-6.92 (m,2 H), 7.05 (s, 2 H), 7.11-7.22 (m, 2 H) |

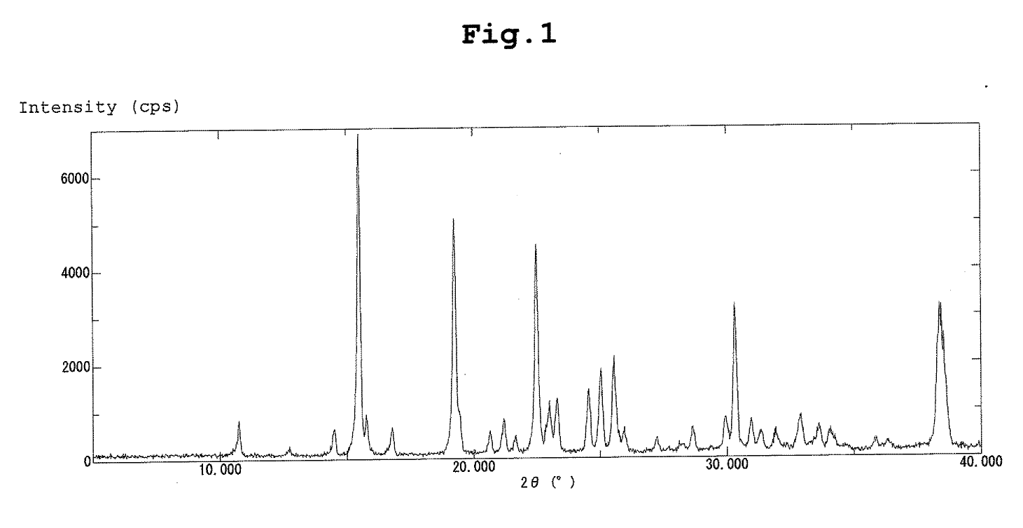

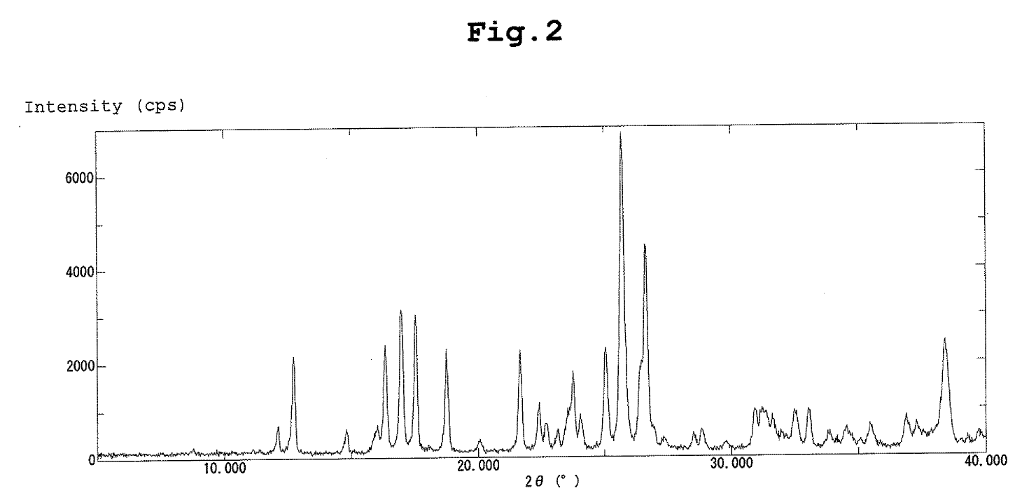

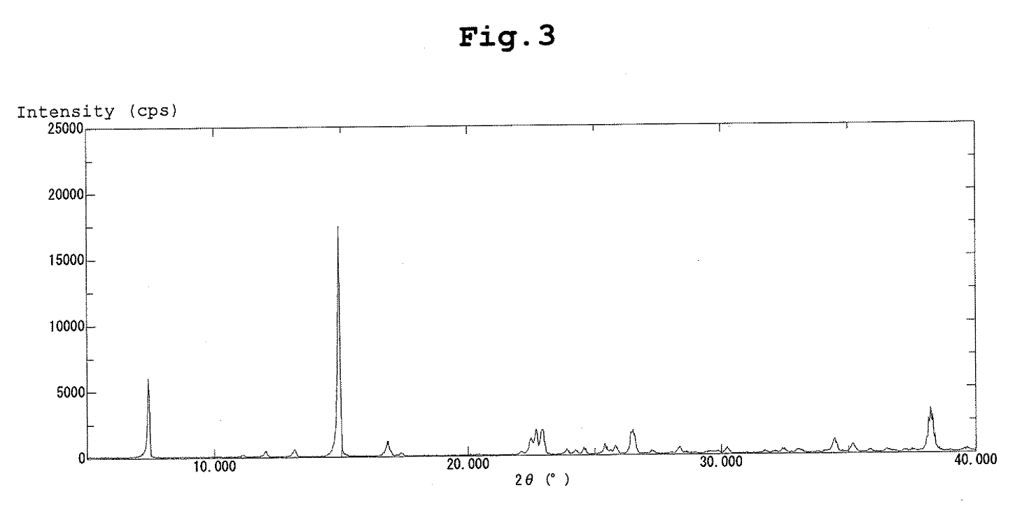

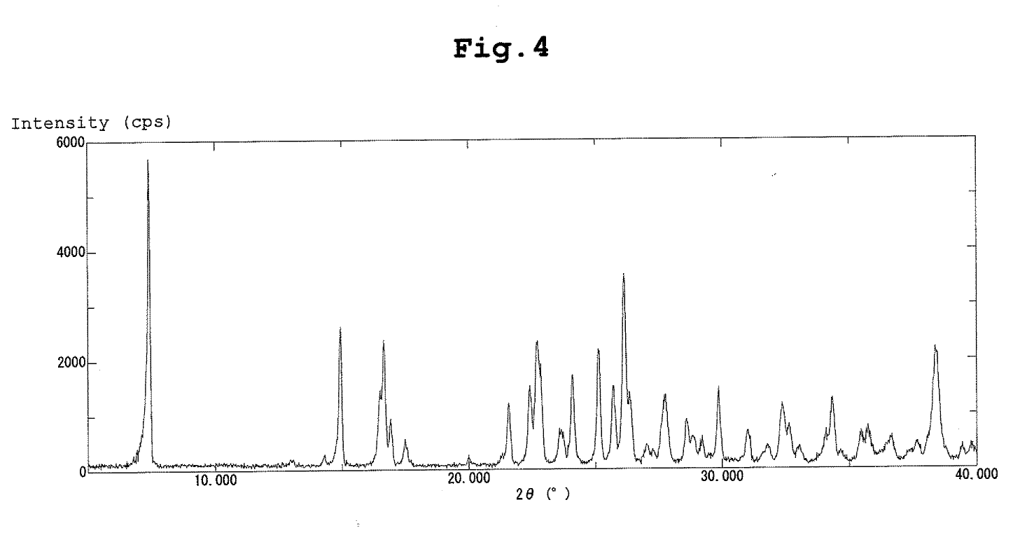

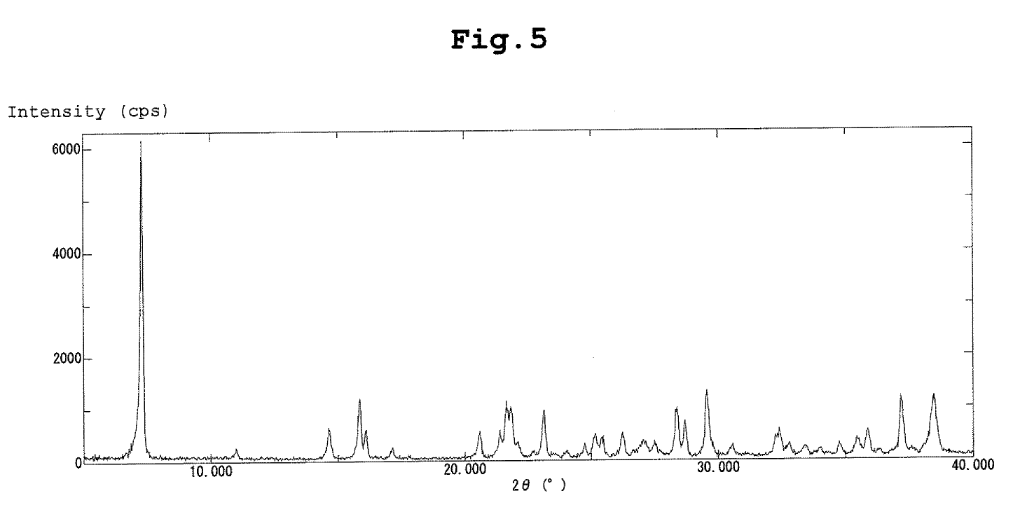

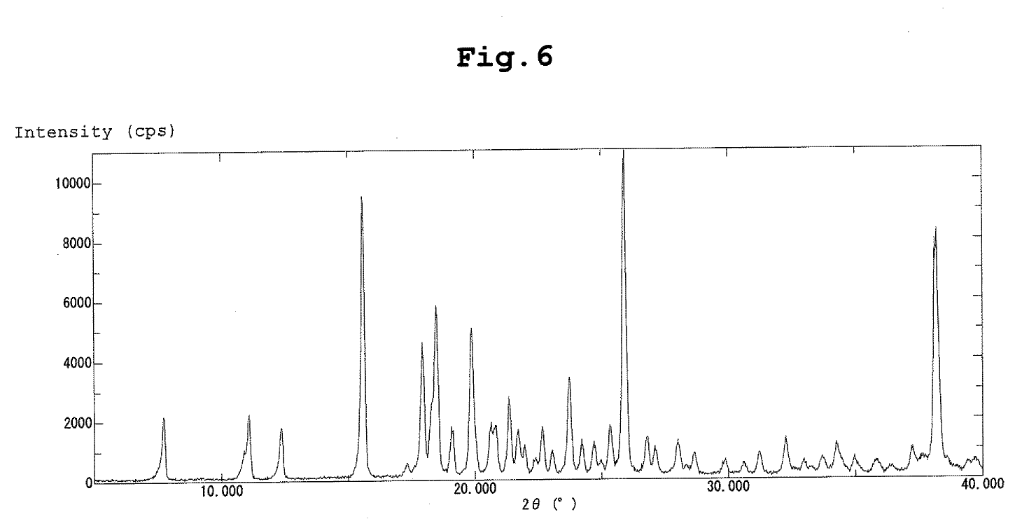

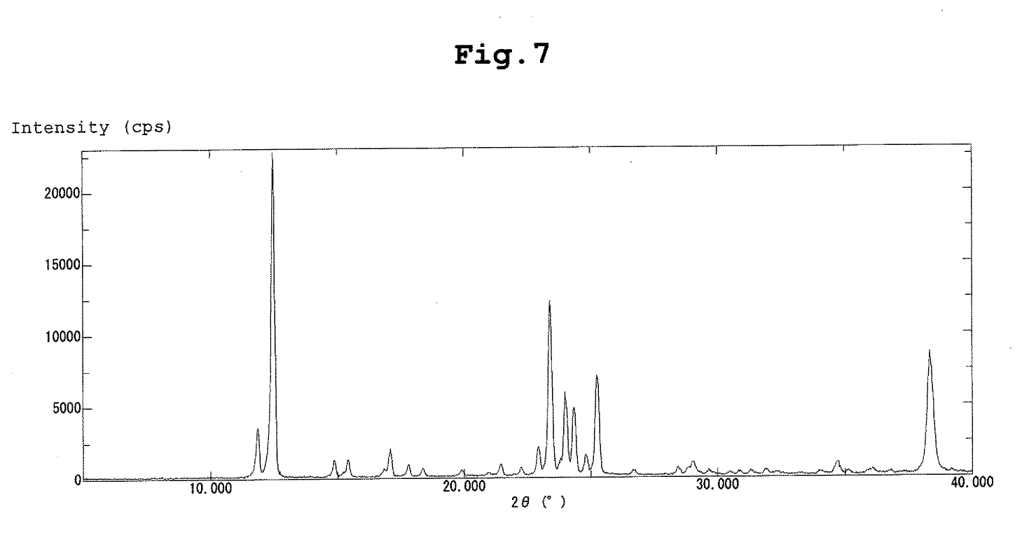

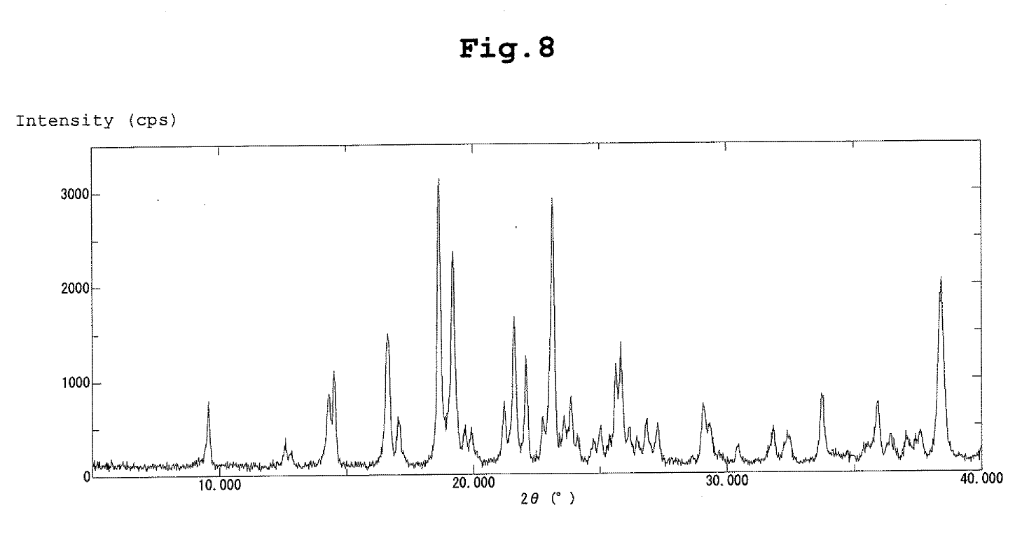

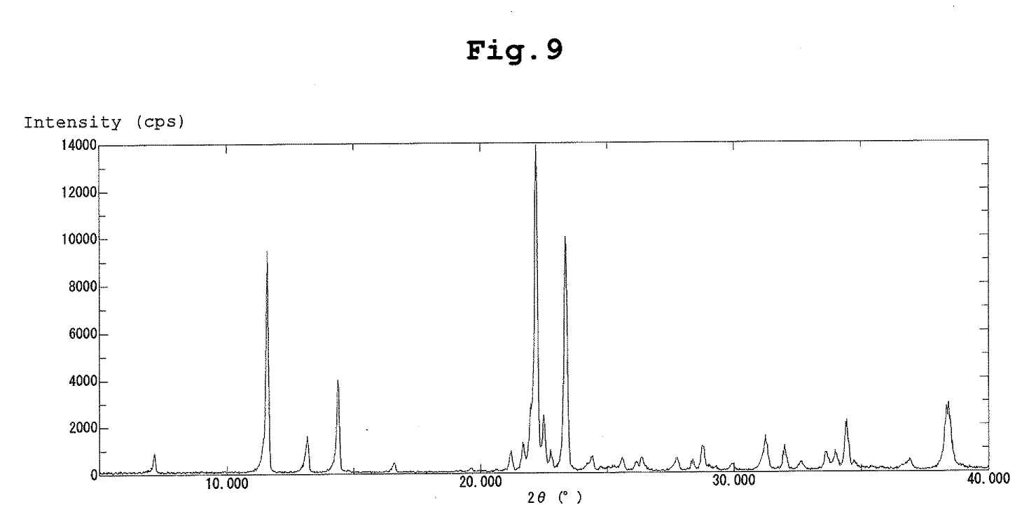

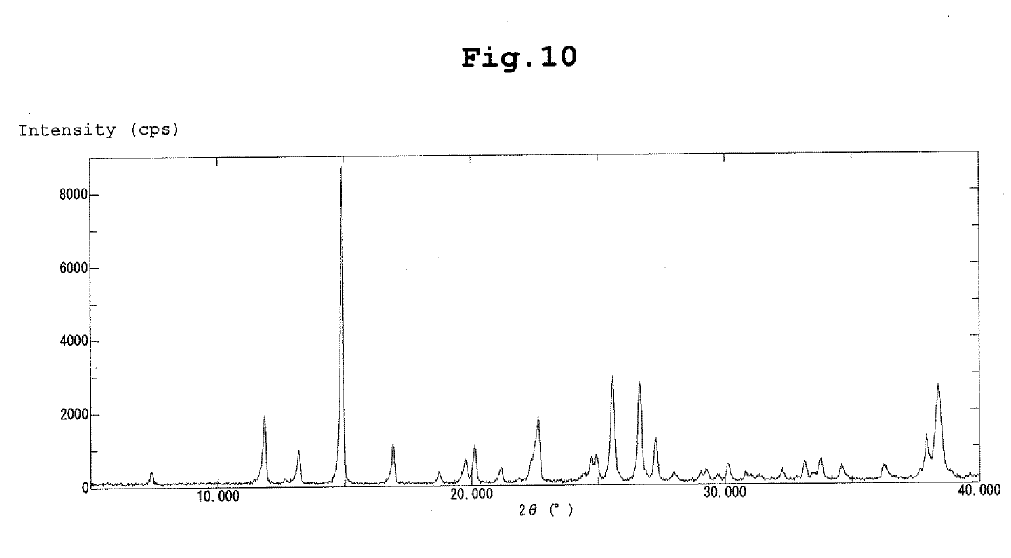

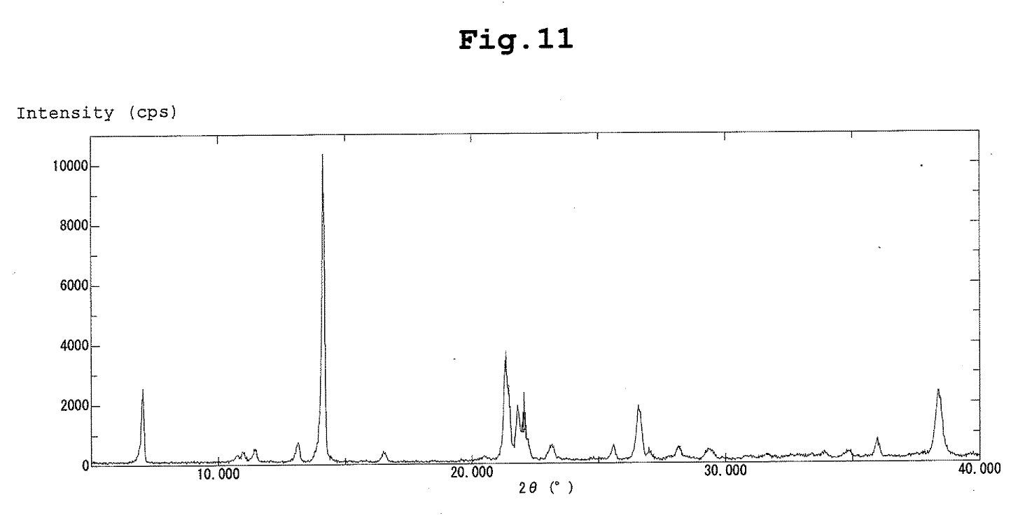

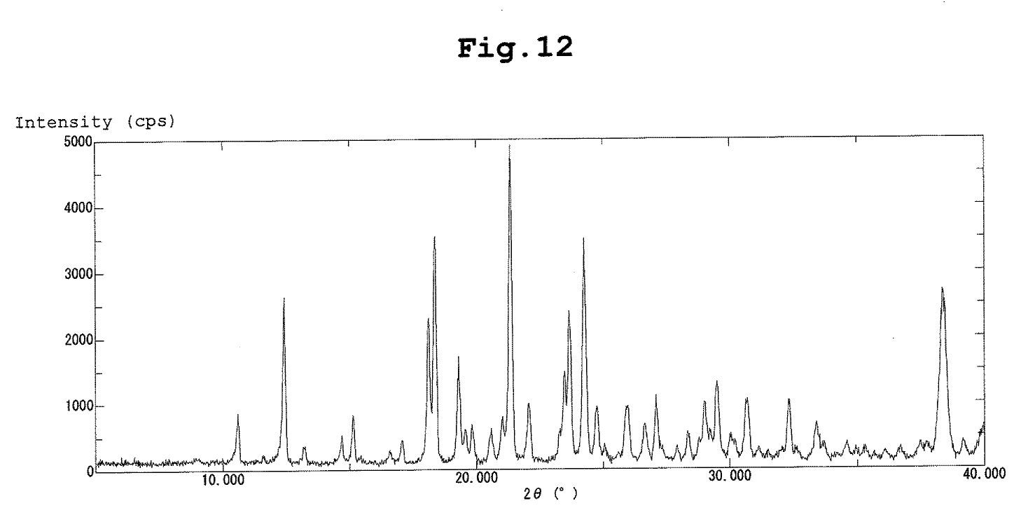

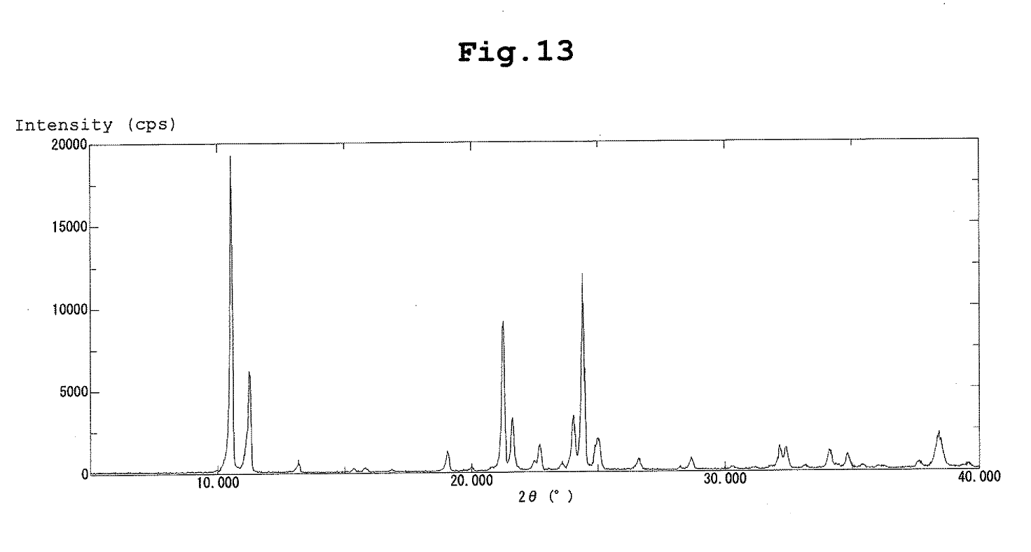

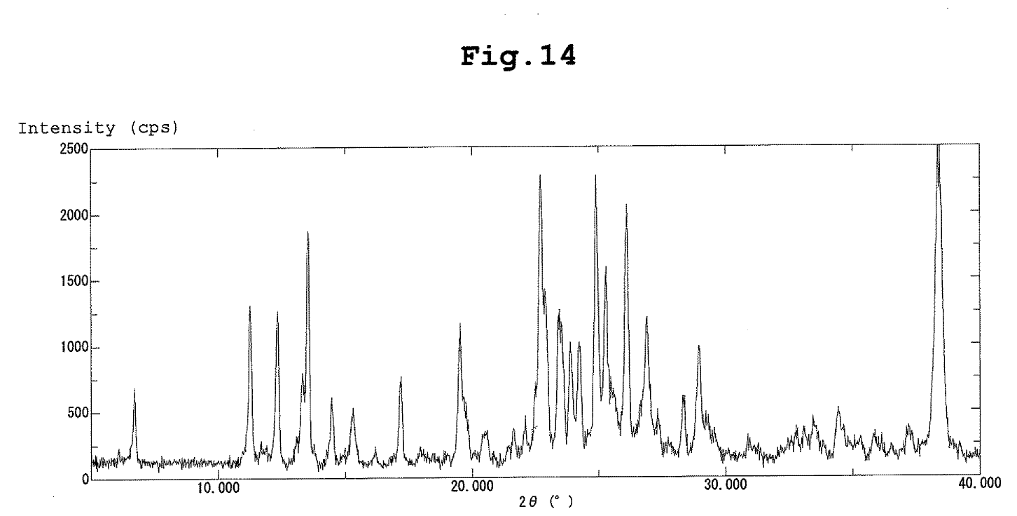

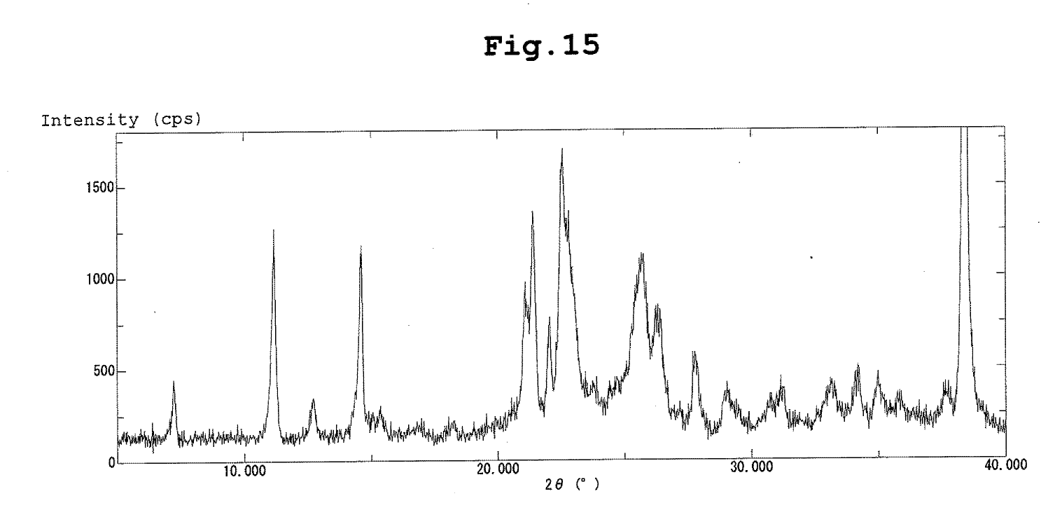

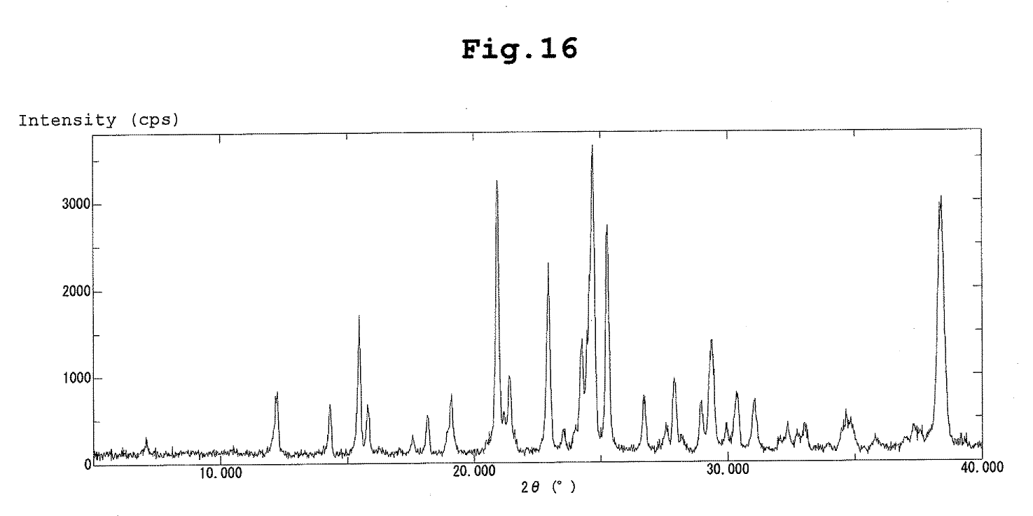

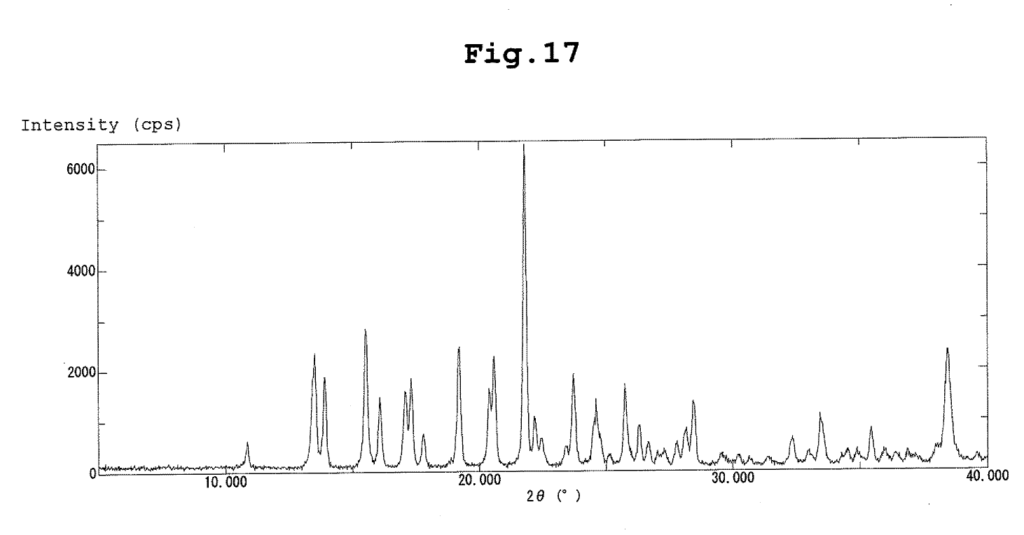

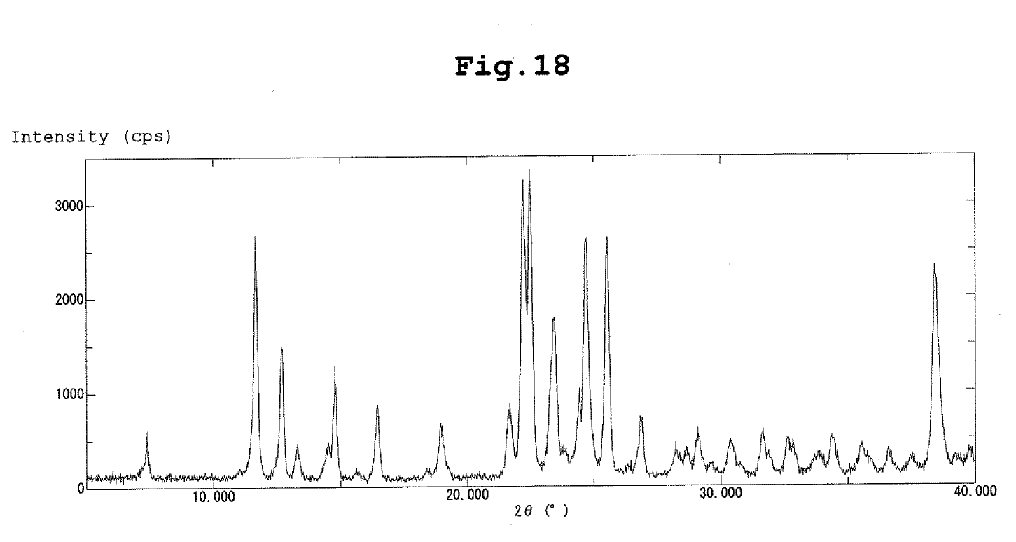

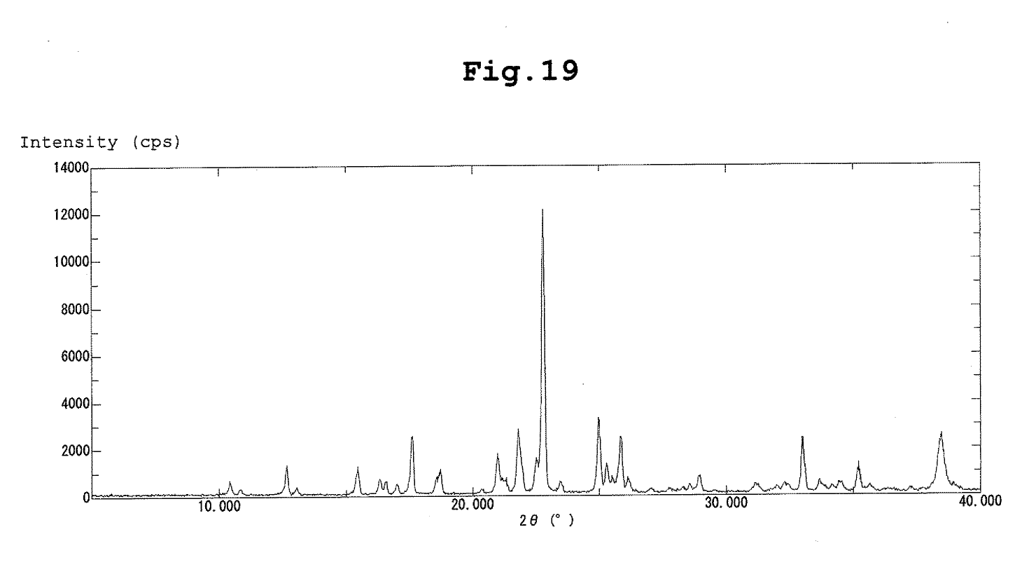

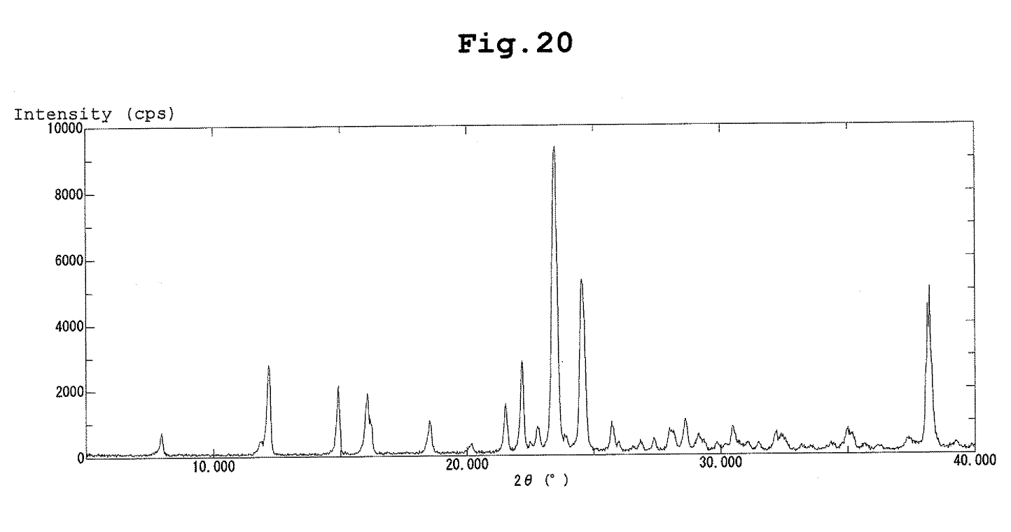

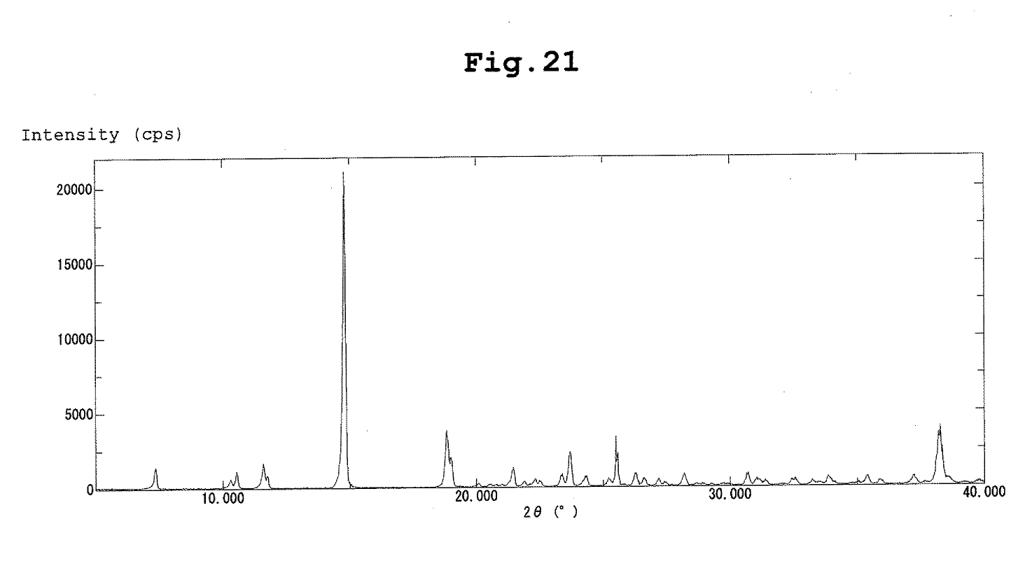

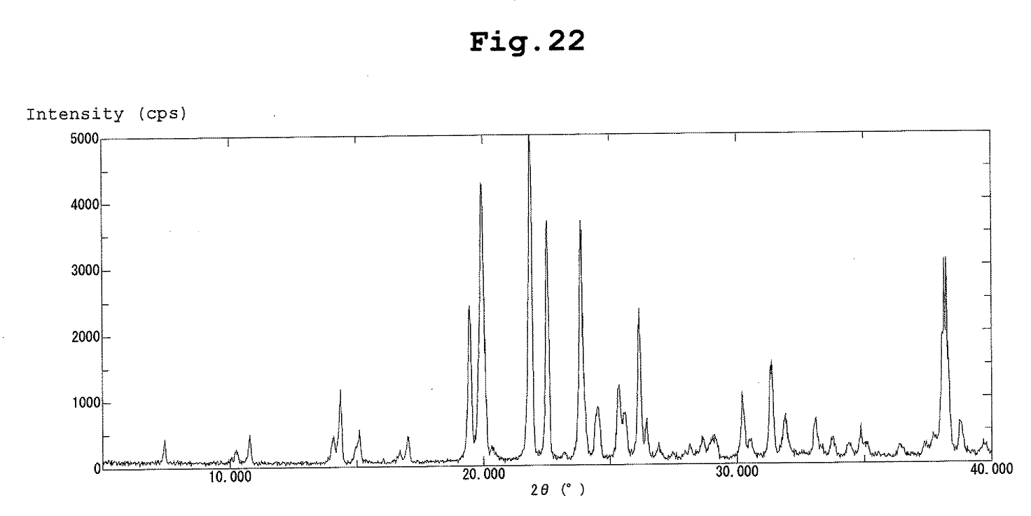

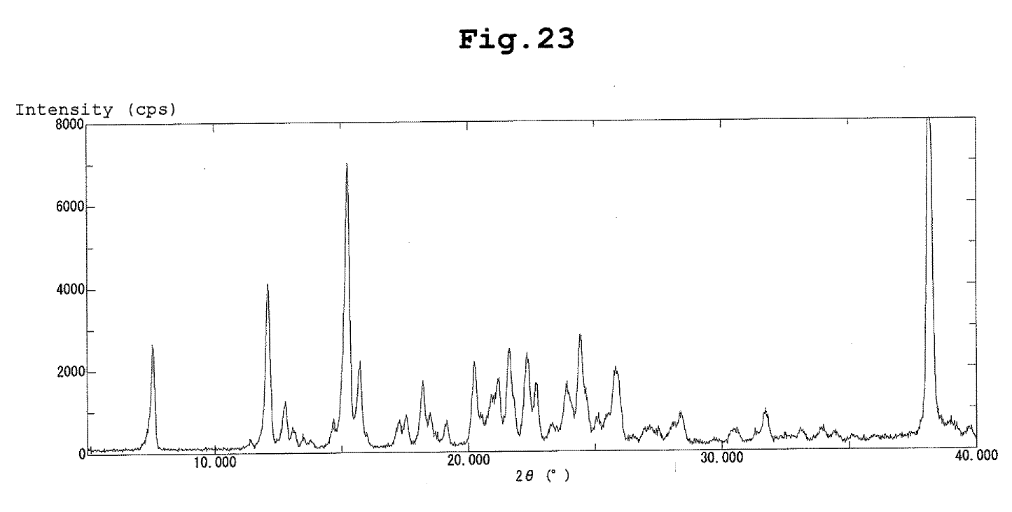

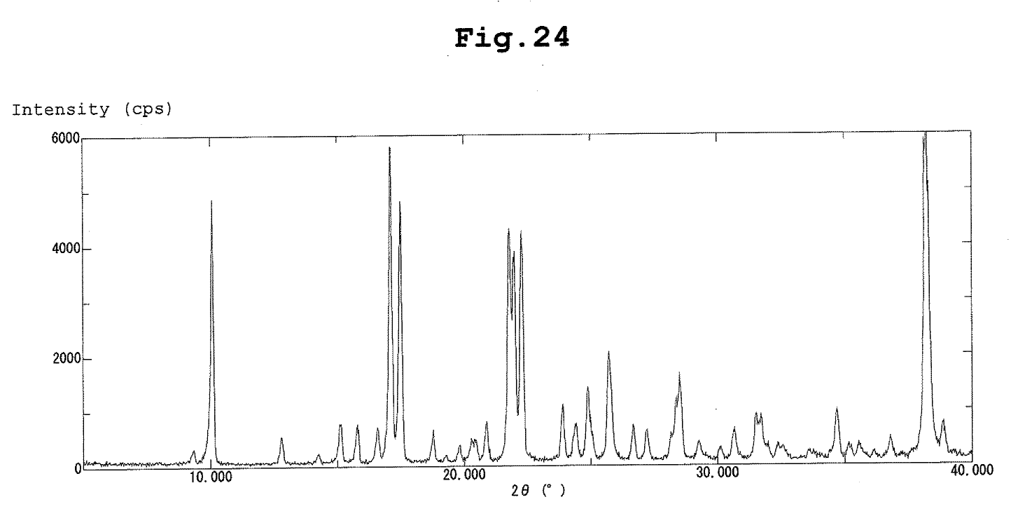

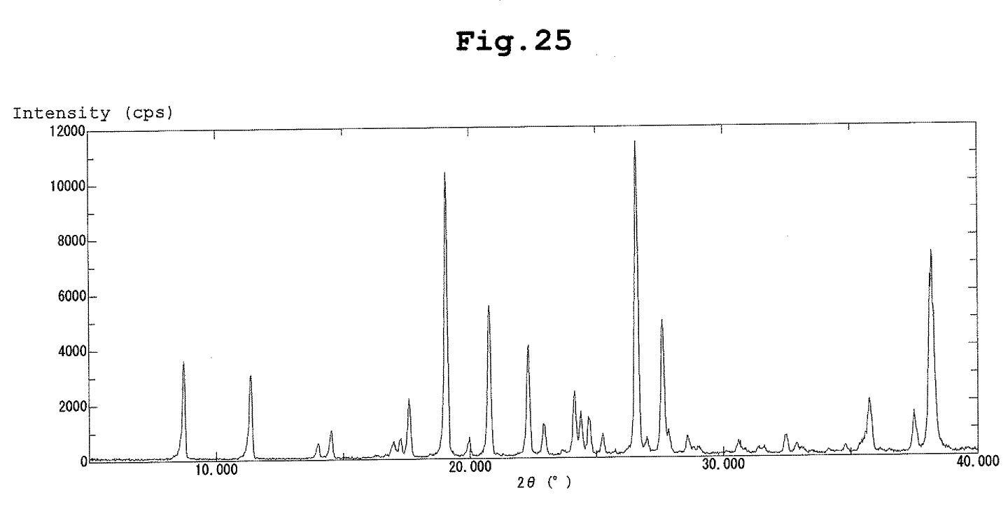

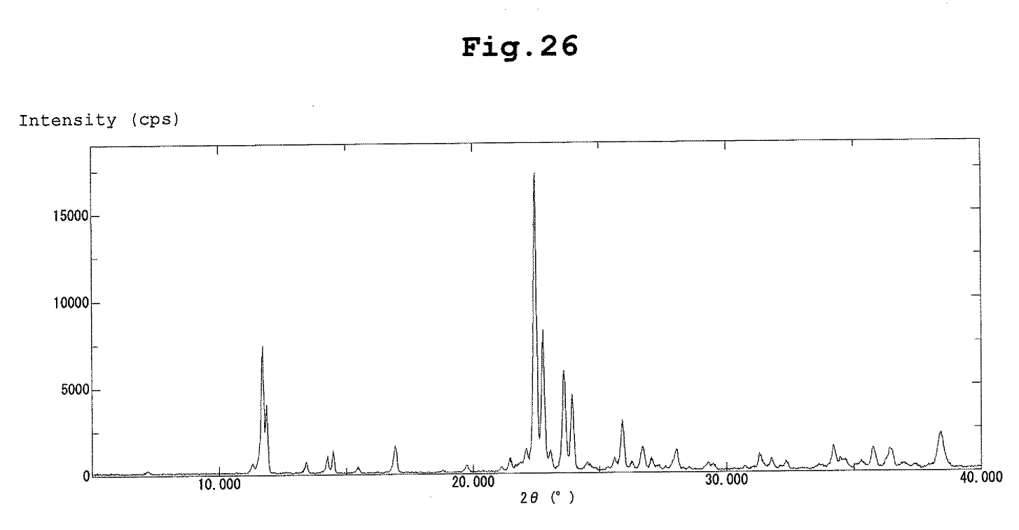

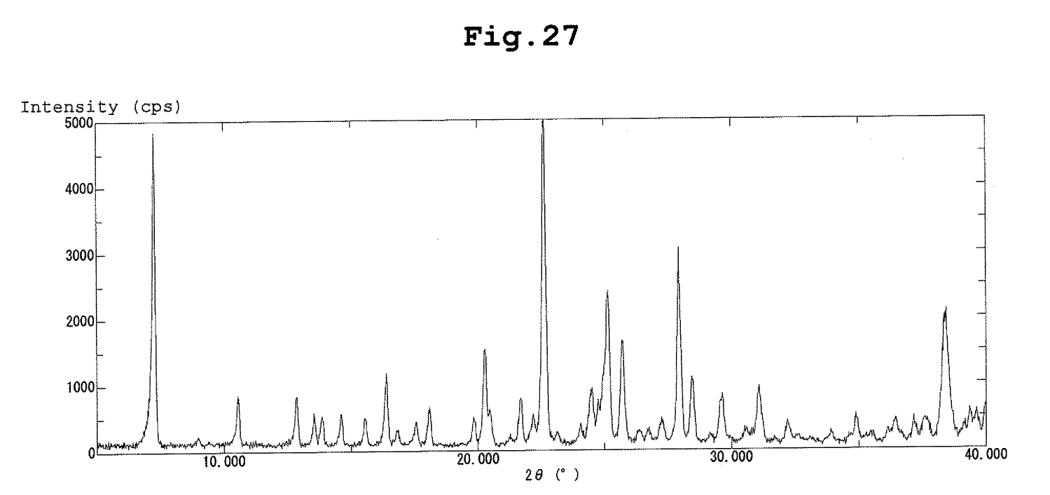

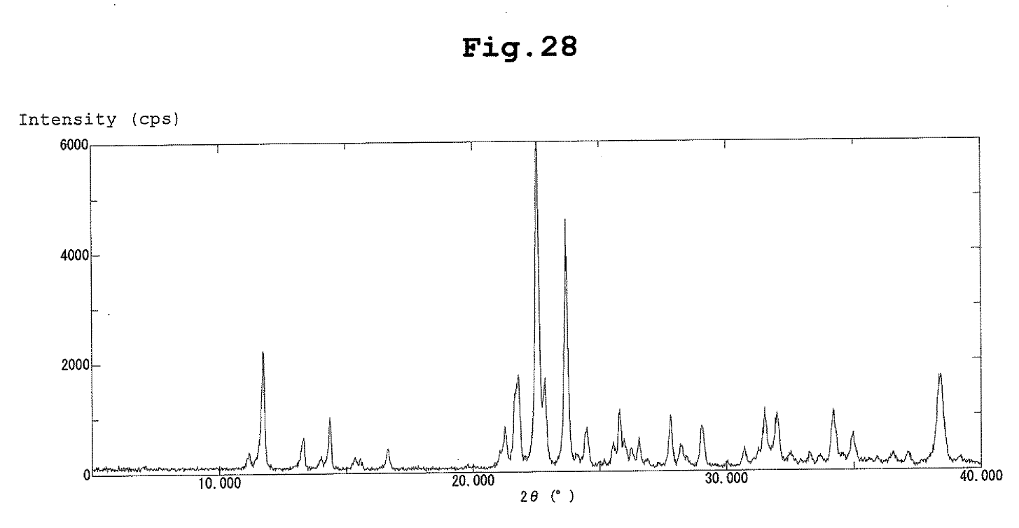

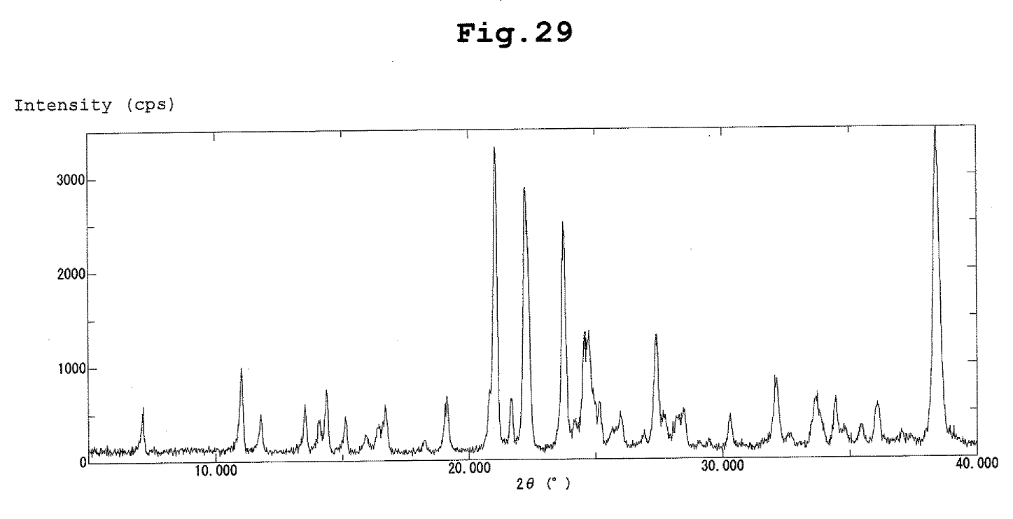

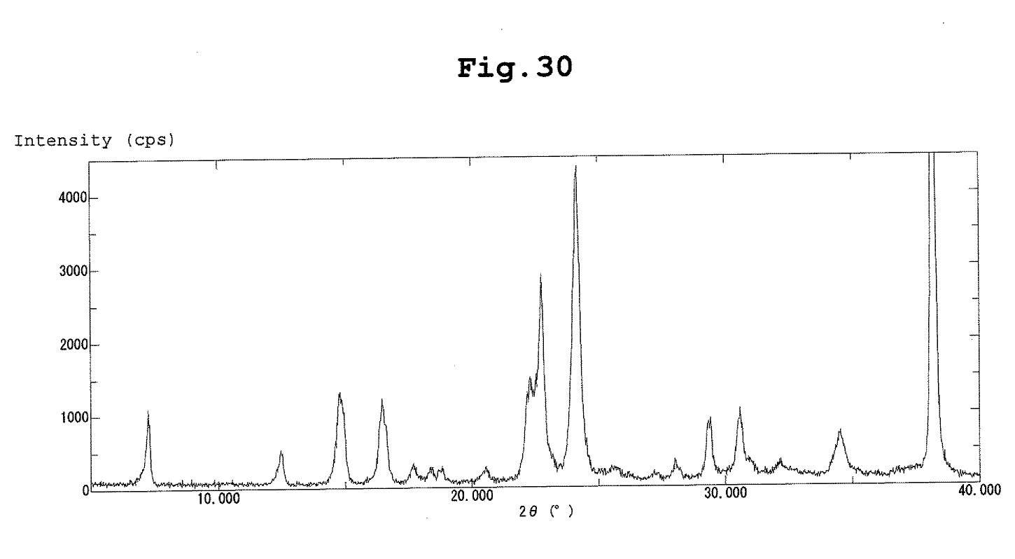

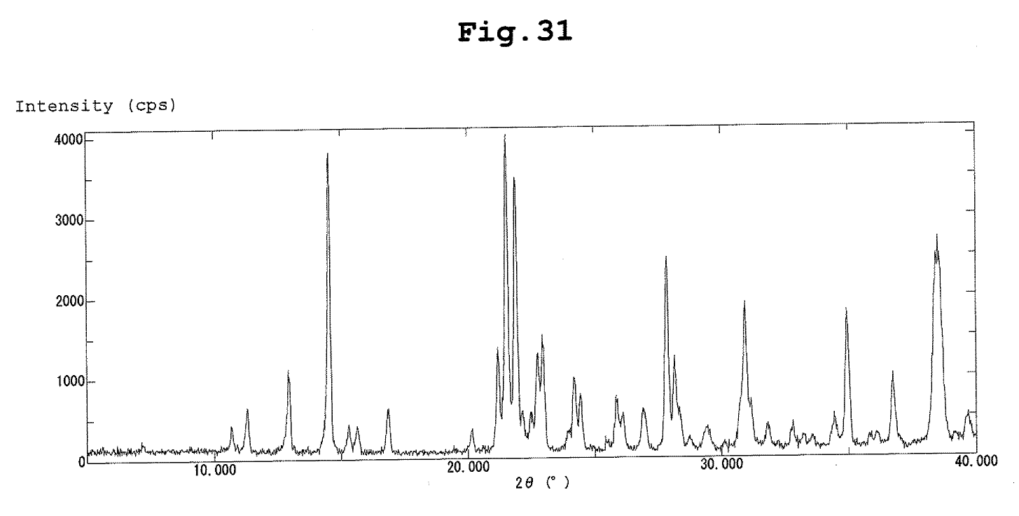

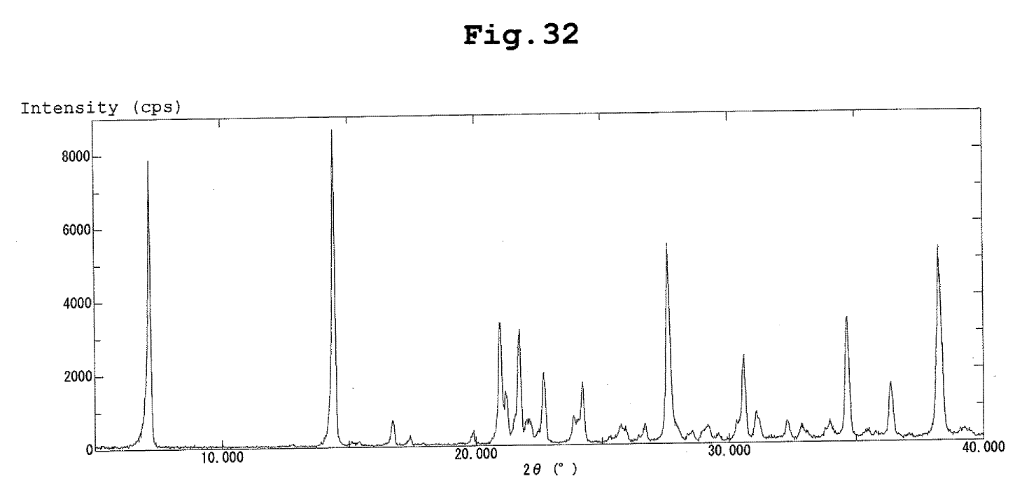

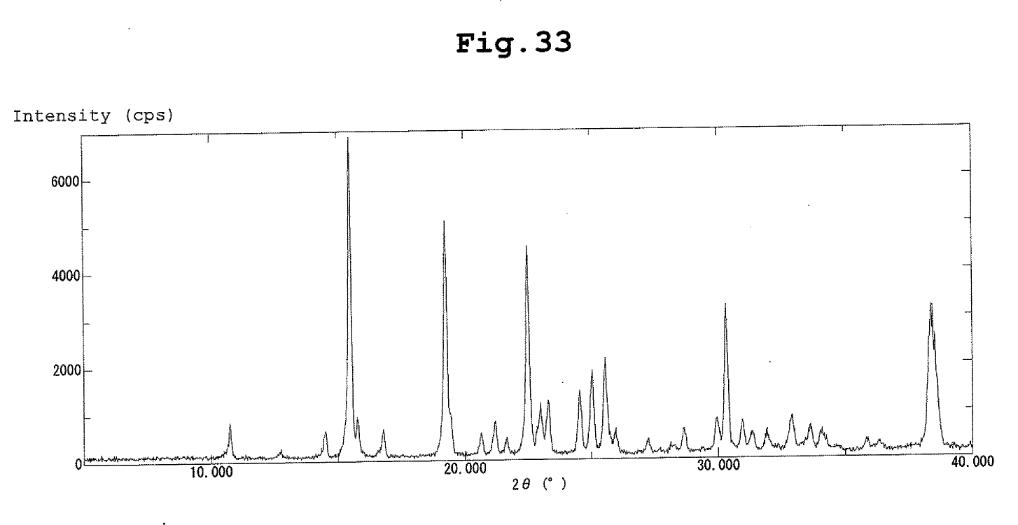

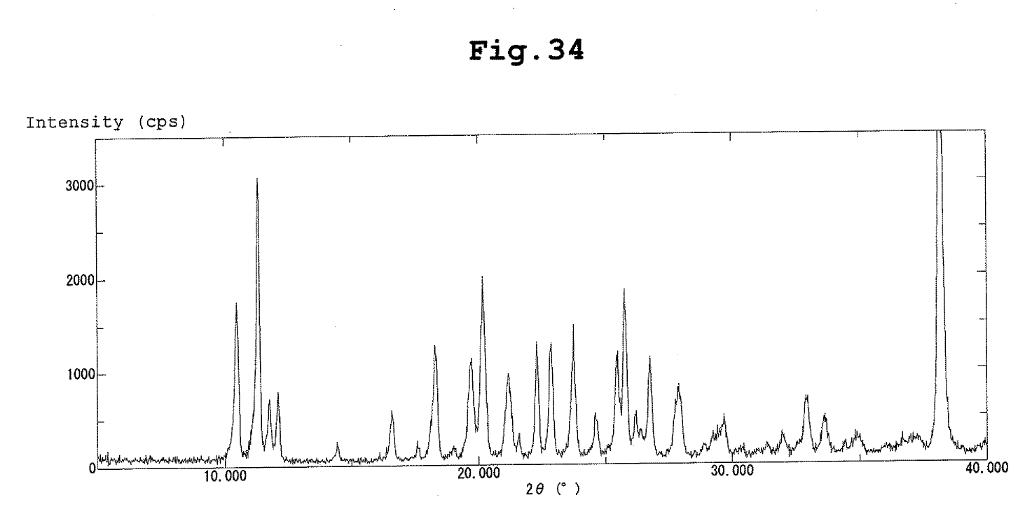

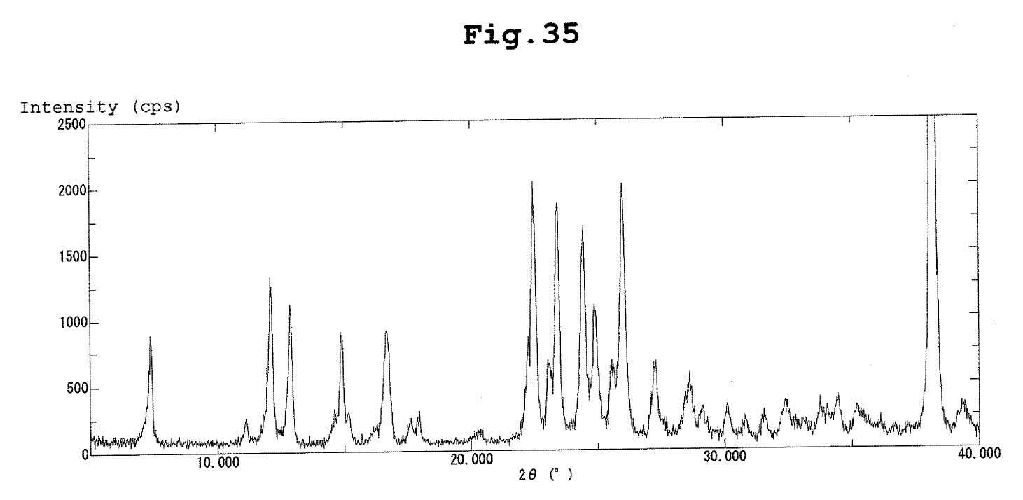

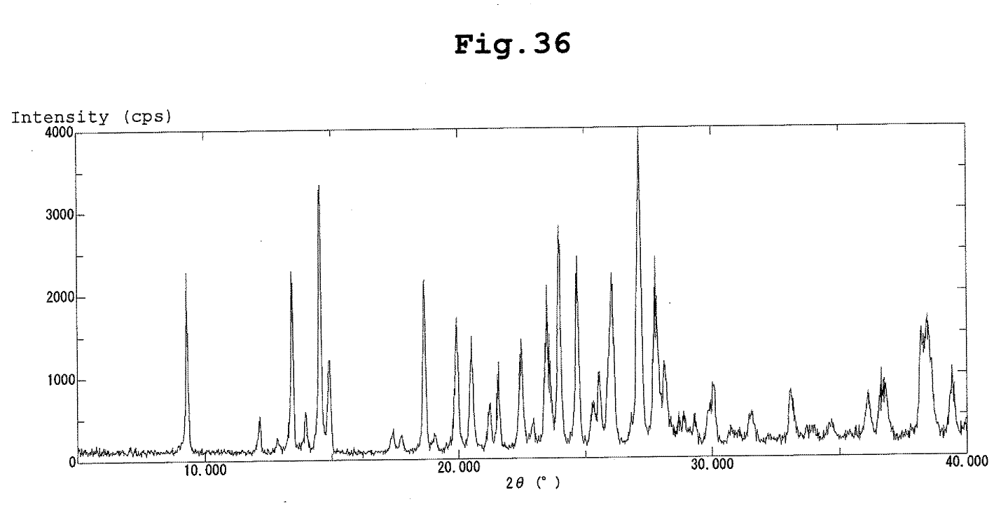

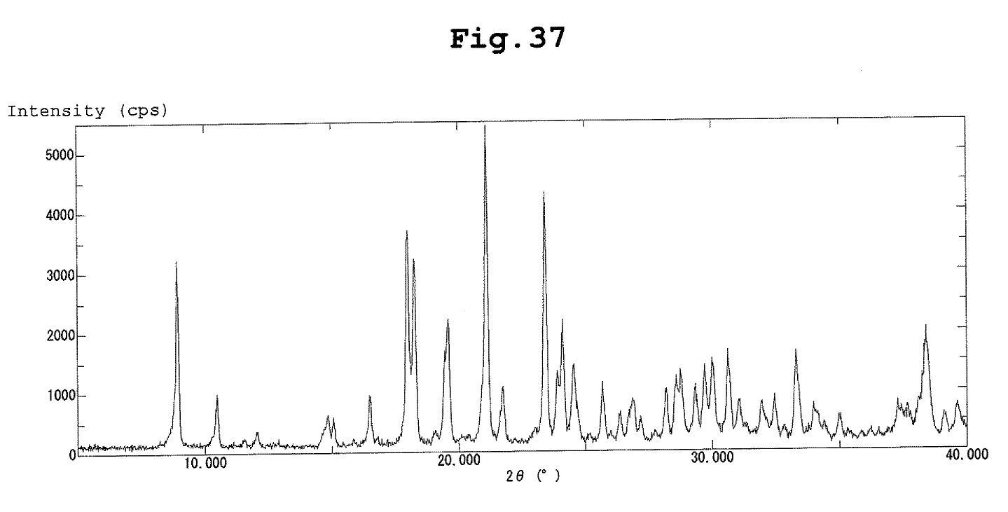

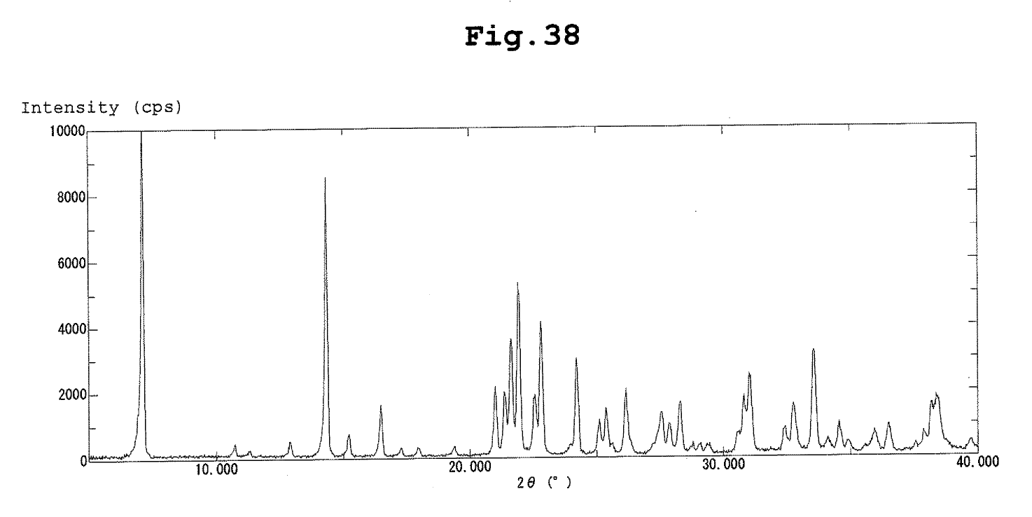

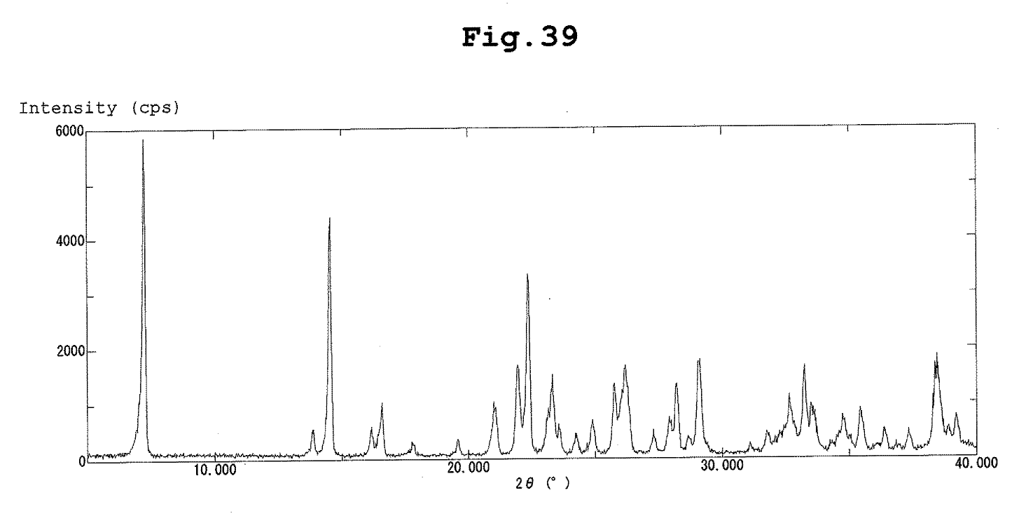

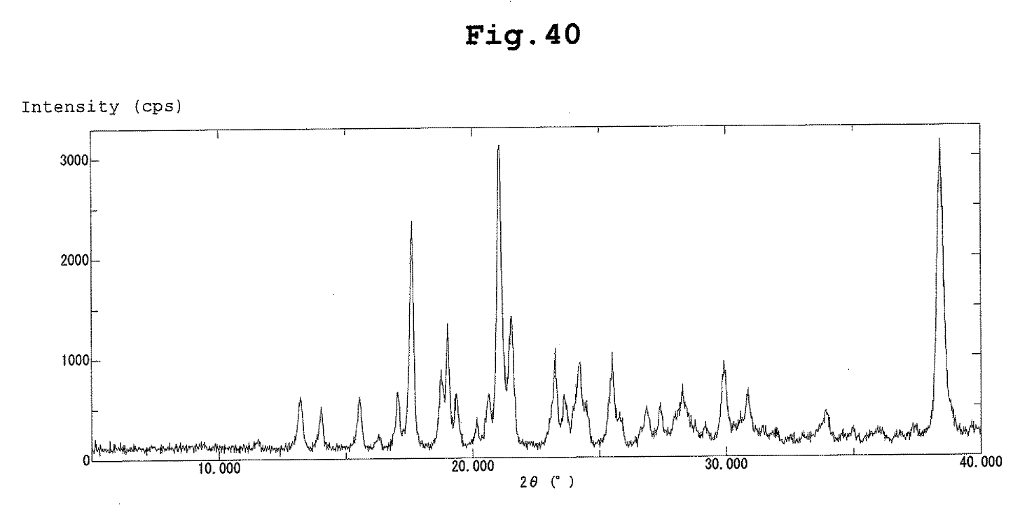

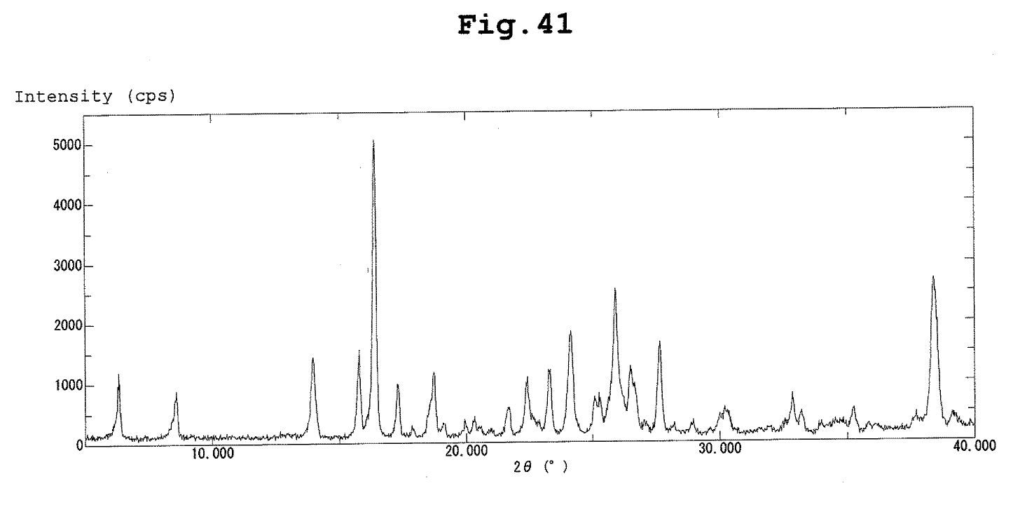

The powder X-ray diffraction patterns of the crystals of compound [1] are shown in FIG. 1-FIG. 41, wherein the horizontal axis shows the diffraction angle (2θ) and the vertical axis shows the peak intensity (cps).

Based on the powder X-ray diffraction patterns, the diffraction angles (2θ) of the characteristic diffraction peaks of each crystal are listed in Table 19-Table 22.

Each sample crystal of compound [1] was mounted on an aluminum cell, and the powder X-ray diffraction pattern was measured using a powder X-ray diffractometer (RINT2100 Ultima+, manufactured by Rigaku) at X-ray radiation: Cu-Kα1 ray, applied voltage: 40 kV, applied electric current: 40 mA, scanning rate: 5° per min, scanning step: 0.02°, scanning range: 5°-40°. Since a diffraction peak due to aluminum cell is observed at around 38.20°-38.40°, the diffraction peaks near this range are not listed as a characteristic diffraction peak of each crystal.

In general, diffraction angle (2θ) and peak intensity (cps) of powder X-ray diffraction pattern may vary depending on a measurement device, measurement conditions, and so on. The crystals in the present specification may show diffraction angle (2θ) and peak intensity of powder X-ray diffraction pattern different from those described in the present specification, as long as it is within a general error range.

Since compound [1] is superior in physical and chemical stability when it is in the form of a crystal, it can advantageously retain the quality for a long time, and permits easy preservation. In addition, the compound affords further advantages in that handling is easy during production of various pharmaceutical compositions and bulk drugs and the production cost can be reduced. Therefore, the crystals of compound [1] are extremely useful as a medicine.

TABLE 19

| Ref. | Diffraction angles 2θ (°) of main diffraction peaks | |
|---|---|---|
| Ex. No. | (characteristic diffraction peaks are underlined) | FIG. |
| 1 | 8.86, 12.94, 16.66, 17.36, 18.26, 18.94, 21.60, | 1 |
| | 22.24, 23.12, 23.50, 25.30, 26.10, 26.94, 28.88, 31.54, | |
| | 32.34, 33.28, 35.12 | |
| 2 | 12.08, 12.72, 14.80, 16.00, 16.32, 16.96, 17.54, | 2 |
| | 18.74, 21.66, 22.38, 22.68, 23.14, 23.56, 23.74, | |
| | 24.04, 25.04, 25.68, 26.40, 26.62, 26.92, 28.50, | |
| | 28.82, 30.90, 31.34, 31.62, 32.46, 33.02, 33.82, | |
| | 34.48, 35.46, 36.84, 37.26 | |
| 3 | 7.40, 12.00, 13.14, 14.90, 16.80, 22.46, 22.68, | 3 |
| | 22.92, 23.88, 24.58, 25.38, 25.80, 26.46, 27.24, | |
| | 28.32, 30.20, 34.42, 35.14, 36.48 | |
| 4 | 7.40, 14.92, 16.64, 16.90, 17.46, 21.56, 22.40, | 4 |
| | 22.68, 22.82, 23.62, 24.08, 25.12, 25.70, 26.12, | |
| | 26.36, 27.02, 27.74, 28.58, 28.82, 29.18, 29.84, | |
| | 30.98, 31.78, 32.32, 32.62, 34.04, 34.30, 35.42, | |
| | 35.70, 36.44, 36.62, 37.62, 39.40 | |
| 5 | 7.28, 14.64, 15.84, 16.08, 20.56, 21.62, 21.82, | 5 |
| | 23.10, 25.12, 25.42, 26.20, 27.48, 28.34, 28.66, | |
| | 29.54, 32.40, 35.44, 35.84, 37.16 | |
| 6 | 7.72, 11.08, 12.34, 15.58, 17.30, 17.92, 18.48, | 6 |
| | 19.08, 19.86, 20.60, 20.80, 21.34, 21.68, 21.96, | |
| | 22.36, 22.64, 23.04, 23.72, 24.20, 24.68, 25.32, | |
| | 25.90, 26.78, 27.10, 27.98, 28.64, 29.84, 30.58, | |
| | 31.20, 32.24, 32.94, 33.66, 34.24, 34.92, 35.78, | |
| | 37.20, 37.58, 39.38, 39.68 | |
| 7 | 11.84, 12.46, 14.86, 15.40, 17.06, 17.80, 18.36, | 7 |
| | 19.90, 21.44, 22.24, 22.92, 23.38, 23.98, 24.32, | |
| | 24.78, 25.24, 28.42, 28.80, 29.02, 29.64, 31.28, | |
| | 31.86, 34.68, 36.08 | |
| 10 | 9.56, 14.28, 14.50, 16.62, 17.04, 18.64, 19.20, | 8 |
| | 19.90, 21.20, 21.60, 22.06, 22.72, 23.14, 23.84, | |
| | 25.62, 25.82, 26.80, 27.24, 29.06, 29.32, 31.80, | |
| | 33.72, 35.90, 37.02, 37.58 | |
| 12 | 7.14, 11.58, 13.14, 14.36, 16.58, 21.16, 21.66, | 9 |
| | 22.18, 22.48, 22.74, 23.36, 24.38, 25.58, 26.12, | |
| | 27.72, 28.34, 28.74, 29.90, 31.22, 31.96, 32.60, | |
| | 33.58, 33.96, 34.38, 34.70, 36.88 | |
| 14 | 7.38, 11.82, 13.16, 14.88, 16.88, 18.70, 19.76, | 10 |
| | 20.10, 21.12, 22.62, 24.70, 24.92, 25.56, 26.62, | |
| | 27.24, 29.24, 30.10, 33.10, 33.72, 34.56, 36.20, | |
| | 37.90 | |

TABLE 20

| Ref. | Diffraction angles 2θ (°) of main diffraction peaks | |
|---|---|---|
| Ex. No. | (characteristic diffraction peaks are underlined) | FIG. |
| 15 | 7.02, 10.96, 11.44, 13.12, 14.14, 16.52, 21.30, | 11 |
| | 21.80, 22.04, 23.14, 25.56, 26.56, 28.12, 29.34, 35.92 | |
| 16 | 10.58, 12.40, 14.66, 15.12, 17.04, 18.10, 18.36, | 12 |
| | 19.28, 19.54, 19.82, 20.54, 21.00, 21.34, 22.06, | |
| | 23.46, 23.66, 24.24, 24.72, 25.92, 26.62, 27.08, | |
| | 28.34, 28.98, 29.46, 29.98, 30.64, 32.30, 33.38, | |
| | 39.16 | |
| 17 | 10.54, 11.24, 13.16, 19.04, 20.00, 21.24, 21.60, | 13 |
| | 22.68, 24.00, 24.38, 24.98, 26.58, 28.66, 32.14, | |
| | 32.38, 34.08, 34.78, 37.58 | |
| 18 | 6.70, 11.26, 12.32, 13.30, 13.52, 14.46, 15.28, | 14 |
| | 17.18, 19.50, 22.70, 23.42, 23.54, 23.88, 24.22, | |
| | 24.88, 25.26, 26.10, 26.88, 28.32, 28.94, 34.40 | |
| 19 | 7.22, 11.16, 14.58, 21.06, 21.38, 22.02, 22.54, | 15 |
| | 22.76, 22.98, 25.70, 26.24, 26.40, 27.76 | |
| 20 | 12.20, 14.30, 15.46, 15.80, 18.14, 19.08, 20.90, | 16 |
| | 21.38, 22.92, 24.22, 24.68, 25.24, 26.68, 27.88, | |
| | 28.92, 29.34, 30.34, 31.02, 32.34, 33.00 | |
| 21 | 10.84, 13.48, 13.90, 15.52, 16.06, 17.06, 17.30, | 17 |
| | 17.78, 19.18, 20.36, 20.58, 21.80, 22.18, 22.44, | |
| | 23.40, 23.70, 24.58, 25.74, 26.30, 26.64, 27.78, | |
| | 28.10, 28.44, 32.30, 33.44, 35.40, 36.84, 37.98 | |
| 26 | 7.38, 11.66, 12.68, 13.28, 14.48, 14.78, 16.44, | 18 |
| | 18.96, 21.66, 22.20, 22.48, 23.42, 24.68, 25.52, | |
| | 26.84, 29.06, 30.36, 31.62, 32.60, 32.82, 34.34, | |
| | 35.50, 36.56 | |
| 27 | 10.42, 12.64, 15.44, 16.32, 16.56, 17.00, 17.60, | 19 |
| | 18.70, 20.96, 21.28, 21.78, 22.78, 23.46, 24.96, | |
| | 25.28, 25.82, 26.12, 28.54, 28.92, 31.12, 32.24, | |
| | 32.98, 33.64, 34.42, 35.18, 35.62 | |
| 31 | 7.94, 11.84, 12.16, 14.90, 16.04, 18.52, 21.50, | 20 |
| | 22.16, 22.78, 23.46, 23.88, 24.52, 25.70, 26.82, | |
| | 27.36, 27.96, 28.60, 29.10, 30.46, 32.14, 32.38, | |
| | 34.96, 35.16, 37.34 | |
| 36 | 7.34, 10.30, 10.54, 11.58, 14.78, 18.80, 21.42, | 21 |
| | 21.88, 22.28, 22.52, 23.34, 23.66, 24.30, 25.22, | |
| | 25.52, 26.26, 26.60, 27.16, 28.18, 30.68, 31.04, | |
| | 31.40, 32.40, 33.22, 33.84, 35.38, 35.88, 37.20 | |

TABLE 21

| Ref. | Diffraction angles 2θ (°) of main diffraction peaks | |
|---|---|---|
| Ex. No. | (characteristic diffraction peaks are underlined) | FIG. |
| 38 | 7.42, 10.78, 14.04, 14.34, 15.08, 17.00, 19.46, | 22 |
| | 19.94, 21.86, 22.52, 23.84, 24.50, 25.32, 25.56, | |
| | 26.14, 26.42, 30.20, 31.34, 31.86, 33.04, 34.84, 37.70 | |
| 39 | 7.58, 12.10, 12.76, 14.64, 15.24, 15.70, 17.24, | 23 |
| | 17.54, 18.18, 18.48, 19.12, 20.22, 20.88, 21.16, | |
| | 21.60, 22.30, 22.66, 23.86, 24.40, 25.12, 25.46, | |
| | 25.80, 26.92, 27.48, 28.36, 30.58, 31.30, 31.66, | |
| | 33.88 | |
| 40 | 10.06, 12.80, 15.10, 15.76, 16.56, 17.10, 17.48, | 24 |
| | 18.76, 20.26, 20.44, 20.86, 21.78, 22.26, 23.86, | |
| | 24.38, 24.86, 25.70, 26.66, 27.18, 28.14, 28.52, | |
| | 29.24, 30.62, 31.68, 34.66, 35.52, 36.78 | |
| 41 | 8.72, 11.34, 13.98, 14.50, 16.96, 17.24, 17.58, | 25 |
| | 19.06, 19.94, 20.76, 22.30, 22.90, 24.12, 24.36, | |
| | 24.70, 25.22, 26.58, 26.98, 27.60, 28.58, 30.58, | |
| | 31.34, 32.44, 32.86, 35.72, 37.48 | |
| 44 | 11.30, 11.70, 13.40, 14.24, 14.48, 15.46, 16.92, | 26 |
| | 19.74, 21.44, 22.08, 22.44, 22.74, 23.04, 23.58, | |
| | 23.90, 25.56, 25.88, 26.66, 27.02, 28.00, 29.46, | |
| | 31.30, 31.76, 32.32, 34.18, 34.64, 35.28, 35.74, | |
| | 36.42 | |
| 50 | 7.26, 10.54, 12.84, 13.54, 13.84, 14.60, 15.54, | 27 |
| | 16.36, 17.54, 18.08, 19.82, 20.26, 21.66, 22.18, | |
| | 22.62, 24.48, 25.12, 25.70, 27.92, 28.44, 29.60, | |
| | 31.06, 32.18, 34.88, 36.42, 37.14, 37.56, 39.36, | |
| | 39.60 | |
| 51 | 11.72, 13.28, 14.34, 16.62, 21.24, 21.66, 21.78, | 28 |
| | 22.54, 22.82, 23.68, 24.46, 25.50, 25.78, 26.22, | |
| | 26.54, 27.78, 28.20, 29.02, 31.48, 31.96, 34.16, | |
| | 34.94 | |
| 56 | 7.12, 11.02, 11.78, 13.52, 14.06, 14.36, 15.10, | 29 |
| | 16.70, 19.10, 21.04, 21.66, 22.22, 23.74, 24.54, | |
| | 24.72, 25.14, 25.98, 27.38, 28.46, 30.26, 32.10, | |
| | 33.64, 34.42, 36.04 | |
| 66 | 7.26, 12.48, 14.80, 16.44, 22.28, 22.76, 24.14, | 30 |
| | 29.36, 30.56, 34.50 | |
| 67 | 10.68, 11.28, 12.92, 14.52, 15.28, 15.62, 16.84, | 31 |
| | 21.18, 21.52, 21.88, 22.48, 22.72, 22.96, 24.18, | |
| | 24.44, 25.84, 26.08, 26.90, 27.84, 28.14, 28.34, | |
| | 30.92, 31.14, 34.42, 34.92, 36.72, | |
| | 39.62 | |

TABLE 22

| Ref. | Diffraction angles 2θ (°) of main diffraction peaks | |
|---|---|---|
| Ex. No. | (characteristic diffraction peaks are underlined) | FIG. |
| 69 | 7.16, 14.40, 16.72, 20.96, 21.72, 22.14, 22.68, | 32 |
| | 23.86, 24.22, 25.70, 26.66, 27.58, 29.18, 30.56, 31.04, | |
| | 32.28, 32.84, 33.94, 34.62, 36.34 | |
| 73 | 10.74, 14.48, 15.48, 15.78, 16.78, 19.24, 20.64, | 33 |
| | 21.18, 21.64, 22.48, 22.98, 23.28, 24.52, 25.02, | |
| | 25.54, 25.92, 27.20, 28.62, 29.92, 30.30, 30.94, | |
| | 31.32, 31.88, 32.86, 33.60, 34.02 | |
| 76 | 10.48, 11.32, 11.76, 12.08, 16.58, 18.30, 19.68, | 34 |
| | 20.18, 21.16, 22.28, 22.84, 23.74, 24.62, 25.46, | |
| | 25.76, 26.18, 26.74, 27.90, 29.64, 32.88, 33.60 | |
| 77 | 7.38, 12.10, 12.88, 14.88, 16.66, 22.48, 23.04, | 35 |
| | 23.40, 24.42, 24.88, 25.52, 25.98, 27.24, 28.62 | |
| 79 | 9.30, 13.44, 13.96, 14.52, 14.90, 18.64, 19.90, | 36 |
| | 20.50, 21.22, 21.56, 22.46, 23.46, 23.96, 24.68, | |
| | 25.28, 25.54, 26.04, 27.12, 27.74, 28.12, 29.84, | |
| | 30.06, 33.06, 36.10, 36.56, 39.40 | |
| 80 | 8.92, 10.46, 14.82, 15.06, 16.48, 17.98, 18.26, | 37 |
| | 19.58, 21.10, 21.72, 23.40, 24.10, 24.52, 25.66, | |
| | 26.32, 26.88, 27.16, 28.16, 28.54, 29.32, 29.68, | |
| | 30.00, 30.62, 31.02, 31.92, 32.44, 33.28, 33.96, | |
| | 34.98, 37.28, 37.66, 39.10, 39.60 | |
| 81 | 7.12, 10.72, 12.90, 14.32, 15.20, 16.48, 20.98, | 38 |
| | 21.36, 21.62, 21.92, 22.54, 22.80, 24.20, 25.08, | |
| | 25.36, 26.12, 27.54, 27.86, 28.28, 30.54, 31.02, | |
| | 32.38, 32.74, 33.54, 34.08, 34.54, 34.88, 35.94, | |
| | 36.48, 37.88, 39.72 | |
| 82 | 7.22, 13.84, 14.54, 16.16, 16.56, 19.56, 21.02, | 39 |
| | 21.92, 22.34, 23.10, 23.30, 23.56, 24.24, 24.86, | |
| | 25.72, 26.18, 27.30, 27.92, 28.18, 29.08, 31.76, | |
| | 32.64, 33.22, 34.76, 35.42, 36.36, 37.32, 39.18 | |
| 85 | 13.20, 14.00, 15.52, 17.04, 17.58, 18.74, 19.00, | 40 |
| | 19.34, 20.62, 21.04, 21.52, 23.24, 23.60, 24.20, | |
| | 25.48, 26.84, 27.38, 29.90, 30.82, 33.92 | |
| 87 | 6.32, 8.56, 13.94, 15.76, 16.38, 17.30, 18.72, | 41 |
| | 21.64, 22.38, 23.28, 24.12, 25.06, 25.26, 25.88, | |
| | 26.46, 27.62, 30.16, 32.84, 33.18, | |
| | 35.22 | |

### Reference Experimental Example 1

### Uric Acid Transport Inhibitory Test Using Human URAT1-Expressing Cells

Human URAT1 full length cDNA was subcloned to expression vector pcDNA3.1 and human URAT1 gene was transfected into human embryonic kidney derived cell line (HEK293 cells) by liposome method using Lipofectamine2000. Simultaneously, HEK293 cells transfected with expression vector pcDNA3.1 alone (hereinafter mock cells) were also produced. HEK293 cells expressing human URAT1 gene or mock cells were selected with geneticin resistance as an index. The functional expression of human URAT1 gene was confirmed by a method similar to the following method, using transport of uric acid labeled with ¹⁴C into the cells as an index.

Human URAT1 expressing HEK293 cells or mock cells were cultured in Dulbecco's modified Eagle's MEM medium (high glucose) containing 10% fetal bovine serum, 0.5 mg/mL geneticin sulfate, 100 units/mL penicillin and 100 μg/mL streptomycin under the conditions of 37° C. and 5% CO₂ in an incubator. The cells were plated in a 96 well plate (poly-D-Lysine coated) at 1×10⁵ cells/well and the following uric acid transport inhibitory test was performed 24 hr later. This test was performed at room temperature.

After the medium was removed by aspiration from each well, the cells were washed once with Hank's Balanced Salt Solution (HBSS) and preincubated with HBSS (100 mL/well) for 5 min. HBSS was aspirated and an assay buffer (wherein NaCl in the above-mentioned HBSS had been substituted with Na-gluconate) containing various concentrations of the Example compound and a radioactive ligand (uric acid labeled with 14 C; final concentration 50 mM) was added to each well at 50 mL/well and an uptake reaction was carried out for 5 min. After the reaction, the cells were washed twice with ice-cold HBSS (150 mL/well), and Microscin ti TM 20 (PerkinElmer) was added at 50 mL/well. The cells were lysed by stirring and the radioactivity of each well was measured in a liquid scintillation counter (TOP COUNT, Packard).

The uric acid transport rate (%) of the Reference Example compound at each concentration was calculated relative to the radioactivity (difference in radioactivity between human URAT1 expressing HEK293 cells and mock cells without addition of Reference Example compound (DMSO addition)) showing URAT1 specific uric acid transport as 100%, and the concentration (IC₅₀) of the Reference Example compound necessary for inhibiting the uric acid transport rate by 50% was determined. The results are shown in Table 23-Table 24. In Table-Table, “+++” means IC₅₀ value of less than 100 nM, “++” means IC₅₀ value of 100 nM to less than 1000 nM, and “+” means IC₅₀ value of 1000 nM to less than 3000 nM.

TABLE 23

| Ref. | |
|---|---|
| Ex. No. | hURAT1 |
| 1 | +++ |
| 2 | +++ |
| 3 | +++ |
| 4 | +++ |
| 5 | +++ |
| 6 | ++ |
| 7 | ++ |
| 8 | ++ |
| 9 | + |
| 10 | ++ |
| 11 | +++ |
| 12 | +++ |
| 13 | ++ |
| 14 | +++ |
| 15 | +++ |
| 16 | +++ |
| 17 | +++ |
| 18 | ++ |
| 19 | ++ |
| 20 | ++ |
| 21 | ++ |
| 22 | + |
| 23 | + |
| 24 | + |
| 26 | ++ |
| 27 | ++ |
| 28 | ++ |
| 29 | + |
| 30 | ++ |
| 31 | +++ |
| 33 | ++ |
| 35 | + |
| 36 | ++ |
| 38 | ++ |
| 39 | +++ |
| 40 | ++ |
| 41 | ++ |
| 44 | +++ |

TABLE 24

| Ref. | |
|---|---|
| Ex. No. | hURAT1 |
| 50 | ++ |
| 51 | +++ |
| 52 | ++ |
| 54 | ++ |
| 55 | ++ |
| 56 | ++ |
| 58 | ++ |
| 59 | + |
| 61 | + |
| 64 | + |
| 65 | + |
| 66 | ++ |
| 67 | +++ |
| 68 | ++ |
| 69 | +++ |
| 70 | ++ |
| 71 | + |
| 73 | ++ |
| 74 | ++ |
| 75 | ++ |
| 76 | ++ |
| 77 | ++ |
| 78 | ++ |
| 79 | +++ |
| 80 | +++ |
| 81 | +++ |
| 82 | +++ |
| 83 | ++ |
| 84 | + |
| 85 | +++ |
| 86 | + |
| 87 | ++ |
| 89 | +++ |
| 90 | ++ |
| 91 | + |
| 93 | ++ |
| 94 | ++ |
| 95 | ++ |

### Reference Experimental Example 2

### CYP Inhibitory Test Using Human Liver Microsome

Human liver microsome (20 mg protein/mL, 5 μL, Xenotech LLC, purchased from Lenexa Kans.) was suspended in 100 mM potassium phosphate buffer (70 μL, pH 7.4), mixed with a solution (0.5 μL) of the Example compound dissolved in DMSO and preincubated at 37° C. for 5 min. NADPH producing system coenzyme solution (β-nicotinamide adenine dinucleotide phosphate: 5.2 mM, D-glucose-6-phosphate: 13.2 mM, magnesium chloride: 13.2 mM, glucose-6-phosphate dehydrogenase: 1.8 U/mL) 25 μL, and a model substrate (CYP3A4: midazolam 1 mM, CYP2D6: bufuralol 1 mM, CYP2C9: diclofenac 2 mM) 0.5 μL dissolved in DMSO were added to start the reaction. After incubation at 37° C. for 10 min., acetonitrile (200 μL) containing an internal standard substance (propranolol 1 μM) was added and the mixture was centrifuged (room temperature, 3000 rpm, 20 min). The amount of the metabolite produced from each model substrate in the supernatant was measured by high performance liquid chromatography/mass spectrometry (LC/MS/MS) and each CYP enzyme activity was determined. The concentration showing 50% inhibition (IC50) was calculated relative to the enzyme activity without addition of Reference Example compound (DMSO 0.5 μL added) as 100%. The results are shown in Table 25-Table 27.

TABLE 25

| Ref. | CYP2C9 inhibition |
|---|---|
| Ex. No. | IC50 (μM) |
| 1 | >50 |
| 2 | 37 |
| 3 | 27 |
| 4 | 9 |
| 5 | 2 |
| 6 | >50 |
| 7 | 4 |
| 8 | 19 |
| 9 | 39 |
| 10 | >50 |
| 11 | 12 |
| 12 | 22 |
| 13 | 18 |
| 14 | 16 |
| 15 | 6 |
| 16 | 10 |
| 17 | 33 |
| 18 | 9 |
| 19 | >50 |
| 20 | 13 |
| 21 | 17 |
| 22 | 1 |
| 23 | 19 |
| 24 | >50 |
| 25 | >50 |
| 26 | 13 |
| 27 | 21 |
| 28 | 24 |
| 29 | >50 |
| 30 | >50 |
| 31 | 20 |
| 32 | 10 |
| 33 | 4 |
| 34 | 11 |
| 35 | 14 |
| 36 | 16 |
| 37 | >50 |
| 38 | 5 |
| 39 | 21 |
| 40 | >50 |

TABLE 26

| Ref. | CYP2C9 inhibition |
|---|---|
| Ex. No. | IC50 (μM) |
| 41 | >50 |
| 42 | >50 |
| 43 | >50 |
| 44 | 31 |
| 45 | >50 |
| 46 | >50 |
| 47 | >50 |
| 48 | >50 |
| 49 | >50 |
| 50 | 15 |
| 51 | 16 |
| 52 | 11 |
| 53 | 6 |
| 54 | 48 |
| 55 | 19 |
| 56 | >50 |
| 57 | >50 |
| 58 | 21 |
| 59 | 8 |
| 60 | >50 |
| 61 | 43 |
| 62 | >50 |
| 63 | >50 |
| 64 | 6 |
| 65 | 14 |
| 66 | 12 |
| 67 | 49 |
| 68 | 27 |
| 69 | 21 |
| 70 | 20 |
| 71 | 15 |
| 72 | 13 |
| 73 | >50 |
| 74 | 33 |
| 75 | >50 |
| 76 | 28 |
| 77 | 18 |
| 78 | 18 |
| 79 | 13 |
| 80 | 5 |

TABLE 27

| Ref. | CYP2C9 inhibition |
|---|---|
| Ex. No. | IC50 (μM) |
| 81 | 13 |
| 82 | 11 |
| 83 | >50 |
| 84 | 15 |
| 85 | 19 |
| 86 | >50 |
| 87 | 31 |
| 88 | >50 |
| 89 | 11 |
| 90 | 21 |
| 91 | >50 |
| 92 | >50 |
| 93 | >50 |
| 94 | 6 |
| 95 | >50 |

As is clear from the above-mentioned Reference Experimental Example 1 (uric acid transport inhibitory test using human URAT1 expression cells), compound [1] has a superior inhibitory action on URAT1 activity. As is clear from Reference Experimental Example 2 (CYP inhibitory test), compound [1] has no or extremely low CYP inhibitory action.

They indicate that compound [1] has an effect of strong suppression of reabsorption of uric acid, and very low fear of side effect since they do not substantially inhibit CYP.

Therefore, the pharmaceutical composition of the present invention comprising compound [1] inhibits reabsorption of uric acid and decreases the blood uric acid level by inhibiting URAT1 activity, whereby affords an agent for the prophylaxis or treatment of pathology showing involvement of uric acid, such as hyperuricemia, gouty tophus, acute gouty arthritis, chronic gouty arthritis, gouty kidney, urolithiasis, renal function disorder, coronary artery disease, ischemic heart disease and the like.

Moreover, as the causal disease of the above-mentioned pathology showing involvement of uric acid, complications or diseases highly possibly complicated, for example, gout arthritis, gouty kidney, urolithiasis, hypertension or hypertensive complications, hyperlipidemia or hyperlipidemic complications, diabetes or diabetic complications, obesity or obesity complications, decreased uric acid excretion secondary hyperuricemia; kidney failure, cardiovascular disorder and cerebrovascular disorder caused by hyperuricemia, and the like can be mentioned. A combined use of an agent for the prophylaxis or treatment of these diseases including hyperuricemia and the pharmaceutical composition of the present invention comprising a pharmaceutically effective amount of compound [1] is effective for the prophylaxis or treatment of these diseases. In addition, a combined use of a pharmaceutical agent that increases the blood uric acid level and the pharmaceutical composition of the present invention comprising a pharmaceutically effective amount of compound [1] is effective for suppressing the increase in the blood uric acid level.

The present invention is specifically explained by referring to the following Reference Examples, Examples and Experimental Examples. However, the present invention is not limited by these Reference Examples, Examples and Experimental Examples. For production of a pharmaceutical composition, the order of mixing and mixing conditions of the additives may vary as appropriate, and the order considered to be reasonable can be employed. When the content of compound [1] per 1 tablet is to be changed, the weight of each additive may vary according to the weight of compound [1].

### Reference Example A

Hydroxypropylmethylcellulose 2910 (100.0 g, 6 mm²/S, TC-5 RW, manufactured by Shin-Etsu Chemical Co., Ltd.) was dissolved in purified water (2400.0 g) to give a binder solution (concentration 4 w/w %).

### Reference Example B

Hydroxypropylmethylcellulose 2910 (100.0 g, 6 mm²/s, TC-5 RW, manufactured by Shin-Etsu Chemical Co., Ltd.) was dissolved in purified water (1566.7 g) to give a binder solution (concentration 6 w/w %).

### Reference Example C

Hydroxypropylmethylcellulose 2910.titanium oxide.macrogol 400 mixture (125.0 g, OPADRY OY-7300, manufactured by Colorcon) was dissolved in purified water (875.0 g) to give a coating solution (solid content concentration 12.5 w/w %).

### Reference Example D

Hydroxypropylmethylcellulose 2910 (36.0 g, 6 mm²/S, TC-5 RW, manufactured by Shin-Etsu Chemical Co., Ltd.) was dissolved in purified water (564.0 g) to give a binder solution (concentration 6 w/w %).

### Reference Example E

Hydroxypropylmethylcellulose 2910 (20.0 g, 6 mm²/s, TC-5 RW, manufactured by Shin-Etsu Chemical Co., Ltd.) was dissolved in purified water (313.3 g) to give a binder solution (concentration 6 w/w %).

### Reference Example F

Hydroxypropylmethylcellulose 2910-titanium oxide-macrogol 400 mixture (125.0 g, OPADRY OY-7300, manufactured by Colorcon) was dissolved in purified water (875.0 g) to give a coating solution (solid content concentration 12.5 w/w %).

### Example 1

### 1 mg Tablet

(3-Chloro-4-hydroxyphenyl)-(2,3-dihydrobenzo[1,4]oxazin-4-yl)-methanone (hereinafter to be referred to as compound A) is micronized in a jet mill (JOM-0101, manufactured by SEISHIN ENTERPRISE CO., LTD., pulverization pressure: 0.7 MPa, supply pressure: 0.7 MPa).

The micronized compound A (25.0 g), D-mannitol (D(−)-mannitol fine powder, manufactured by Merck, 887.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example A, and the mixture is granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.) After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm). Magnesium stearate (from plant source, manufactured by TAIHEI CHEMICAL INDUSTRIAL CO., LTD., 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 1 mg of compound A per tablet.

### Example 2

### 5 mg Tablet

Compound A is micronized in a jet mill (JOM-0101, manufactured by SEISHIN ENTERPRISE CO., LTD., pulverization pressure: 0.7 MPa, supply pressure: 0.7 MPa).

The micronized compound A (125.0 g), D-mannitol (D(−)-mannitol fine powder, manufactured by Merck, 787.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example A, and the mixture is granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.) After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL, manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm). Magnesium stearate (from plant source, 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 5 mg of compound A per tablet.

### Example 3

### 50 mg Tablet

Compound A is micronized in a jet mill (JOM-0101, manufactured by SEISHIN ENTERPRISE CO., LTD., pulverization pressure: 0.7 MPa, supply pressure: 0.7 MPa).

The micronized compound A (1250.0 g), D-mannitol (D(−)-mannitol fine powder, manufactured by Merck, 412.5 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example B, and the mixture is granulated by an agitation granulator (FS-25, manufactured by Fukae Powtec., agitator blade: 400 rpm, chopper blade: 1,200 rpm, granulating time: 30 sec×2 times) to give a granulated powder

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.) After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL, manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm). Magnesium stearate (from plant source, 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 50 mg of compound A per tablet.

### Example 4

### 100 mg Tablet

Compound A is micronized in a jet mill (JOM-0101, manufactured by SEISHIN ENTERPRISE CO., LTD., pulverization pressure: 0.7 MPa, supply pressure: 0.7 MPa).

The micronized compound A (450.0 g), D-mannitol (D(−)-mannitol fine powder, manufactured by Merck, 148.5 g), low-substituted hydroxypropylcellulose (LH-21, manufactured by Shin-Etsu Chemical Co., Ltd., 360.0 g) and light anhydrous silicic acid (Adsolider 101, manufactured by Freund Corporation, 22.5 g) are mixed by an agitation granulator (FM-VG-10, manufactured by POWREX CORPORATION, agitator blade: 400 rpm, chopper blade: 1,200 rpm, mixing time: 30 sec×2 times) to give a powder mixture.

To the obtained powder mixture is added the binder solution obtained in Reference Example D, and the mixture is granulated by an agitation granulator (FM-VG-10, manufactured by POWREX CORPORATION, agitator blade: 400 rpm, chopper blade: 1,200 rpm, granulating time: 30 sec×2 times) to give a granulated powder.

The obtained granulated powder is sized with screen mill (P-02, manufactured by DALTON CO., LTD.) (rotation number: 5,900 rpm, screen diameter: 3000 μm), and dried in a fluidized-bed dryer (MP-01, manufactured by Freund Corporation) (charge air temperature setting: 70° C., drying end-point: exhaustion temperature 38° C.). After the completion of the drying, a sized powder is obtained with a screen mill (P-02, manufactured by DALTON CO., LTD) (rotation number: 5,900 rpm, screen diameter: 1000 μm). Two batches thereof are prepared (sized granulated powder 1).

Separately from the above, the micronized compound A (250.0 g), D-mannitol (D(−)-mannitol fine powder, manufactured by Merck, 82.5 g), low-substituted hydroxypropylcellulose (LH-21, manufactured by Shin-Etsu Chemical Co., Ltd., 200.0 g) and light anhydrous silicic acid (Adsolider 101, manufactured by Freund Corporation, 12.5 g) are mixed with an agitation granulator (FM-VG-10, manufactured by POWREX CORPORATION, agitator blade: 400 rpm, chopper blade: 1,200 rpm, mixing time: 30 sec×2 times) to give a mixed powder.

To the obtained mixed powder is added the binder solution obtained in Reference Example E, and the mixture is granulated with an agitation granulator (FM-VG-10, manufactured by POWREX CORPORATION, agitator blade: 400 rpm, chopper blade: 1,200 rpm, granulation time: 30 sec×2 times) to give a granulated powder.

The obtained granulated powder is sized with screen mill (P-02, manufactured by DALTON CO., LTD.) (rotation number: 5,900 rpm, screen diameter: 3000 μm), and dried with a fluidized-bed dryer (MP-01, manufactured by Freund Corporation, charge air temperature setting: 70° C., drying end-point: exhaustion temperature 38° C.). After the completion of the drying, a sized granulated powder is obtained with screen mill (P-02, manufactured by DALTON CO., LTD) (rotation number: 5,900 rpm, screen diameter: 1000 μm). One batch thereof is prepared (sized granulated powder 2).

To the obtained sized granulated powder (sized granulated powder 1+sized granulated powder 2, 2599.0 g) are added crospovidone (Kollidon CL, manufactured by BASF, 322.0 g) and crystalline cellulose (Ceolus PH-102, manufactured by Asahi Kasei Chemicals, 149.5 g), and they are mixed with a V-TYPE MIXER (V-20, manufactured by TOKUJU CORPORATION, 150 rpm). Magnesium stearate (from plant source, manufactured by TAIHEI CHEMICAL INDUSTRIAL CO., LTD., 34.5 g) is added, and the mixture is further mixed (150 rpm, tableting powder 1).

The tableting powder 1 (1107 g) is compressed by a tableting machine (correct 12HUK, manufactured by KIKUSUI SEISAKUSHO LTD., pestle size: 9 mmφ-13R, tableting speed: 20-30 rpm, desired tablet hardness: 90N) to give a core tablet (thickness 4.1-4.3 mm).

The core tablet is coated by spraying the coating solution obtained in Reference Example F (coating amount: 15 mg, coating end-point: the point where the tablet mass increase d by 15.5-16.5 mg from that after pre-drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 100 mg of compound A per tablet.

### Example 5

### 200 mg Tablet

The tableting powder 1 (1669.7 g) obtained in Example 4 is compressed by a tableting machine (correct 12HUK, manufactured by KIKUSUI SEISAKUSHO LTD., pestle size: major axis 16.4 mm, minor axis 7.5 mm, tableting speed: 20-30 rpm,

desired tablet hardness: 100N) to give a core tablet (thickness 5.1-5.5 mm). The core tablet is coated by spraying the coating solution obtained in Reference Example F (coating amount: 30 mg, coating end-point: point when tablet mass increased by 30.5-31.5 mg from that after previous drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 200 mg of compound A per tablet.

### Example 6

### 1 mg Tablet

(3-Bromo-4-hydroxyphenyl)-(2,3-dihydrobenzo[1,4]oxazin-4-yl)-methanone (hereinafter to be referred to as compound B) is micronized in a jet mill (JOM-0101, manufactured by SEISHIN ENTERPRISE CO., LTD., pulverization pressure: 0.7 MPa, supply pressure: 0.7 MPa).

The micronized compound B (25.0 g), D-mannitol (D(−)-mannitol fine powder, manufactured by Merck, 887.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example A, and the mixture is granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.) After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL, manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm) Magnesium stearate (from plant source, manufactured by TAIHEI CHEMICAL INDUSTRIAL CO., LTD., 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 1 mg of compound B per tablet.

### Example 7

### 5 mg Tablet

Compound B is micronized in a jet mill (JOM-0101, manufactured by SEISHIN ENTERPRISE CO., LTD., pulverization pressure: 0.7 MPa, supply pressure: 0.7 MPa).

The micronized compound B (125.0 g), D-mannitol (D(−)-mannitol fine powder, manufactured by Merck, 787.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example A, and the mixture is granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.) After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL, manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm). Magnesium stearate (from plant source, 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 5 mg of compound B per tablet.

### Example 8

### 50 mg Tablet

Compound B is micronized in a jet mill (JOM-0101, manufactured by SEISHIN ENTERPRISE CO., LTD., pulverization pressure: 0.7 MPa, supply pressure: 0.7 MPa).

The micronized compound B (1250.0 g), D-mannitol (D(−)-mannitol fine powder, manufactured by Merck, 412.5 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) are mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture is added the binder solution obtained in Reference Example B, and the mixture is granulated by an agitation granulator (FS-25, manufactured by Fukae Powtec., agitator blade: 400 rpm, chopper blade: 1,200 rpm, granulating time: 30 sec×2 times) to give a granulated powder.

The obtained granulated powder is sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.). After the completion of the drying, the powder is sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) are added crospovidone (Kollidon CL, manufactured by BASF, 350.0 g) and crystalline cellulose (Ceolus PH-102, manufactured by Asahi Kasei Chemicals, 162.5 g), and the mixture is mixed in a V-type mixer (1 cu. ft. PK Blender, manufactured by Patterson Kelley, 150 rpm). Magnesium stearate (from plant source, 37.5 g) is added, and the mixture is further mixed (150 rpm).

This is compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet is coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 50 mg of compound B per tablet.

### Example 9

### 100 mg Tablet

Compound B is micronized in a jet mill (JOM-0101, manufactured by SEISHIN ENTERPRISE CO., LTD., pulverization pressure: 0.7 MPa, supply pressure: 0.7 MPa).

The micronized compound B (450.0 g), D-mannitol (D(−)-mannitol fine powder, manufactured by Merck, 148.5 g), low-substituted hydroxypropylcellulose (LH-21, manufactured by Shin-Etsu Chemical Co., Ltd., 360.0 g) and light anhydrous silicic acid (Adsolider 101, manufactured by Freund Corporation, 22.5 g) are mixed by an agitation granulator (FM-VG-10, manufactured by POWREX CORPORATION, agitator blade: 400 rpm, chopper blade: 1,200 rpm, mixing time: 30 sec×2 times) to give a powder mixture.

To the obtained powder mixture is added the binder solution obtained in Reference Example D, and the mixture is granulated by an agitation granulator (FM-VG-10, manufactured by POWREX CORPORATION, agitator blade: 400 rpm, chopper blade: 1,200 rpm, granulating time: 30 sec×2 times) to give a granulated powder.

The obtained granulated powder is sized with a screen mill (P-02, manufactured by DALTON CO., LTD.) (rotation number: 5,900 rpm, screen diameter: 3000 μm), and dried using a fluidized-bed dryer (MP-01, manufactured by Freund Corporation, charge air temperature set to: 70° C., drying end-point: exhaustion temperature 38° C.). After the completion of the drying, the powder is sized with a screen mill (P-02, manufactured by DALTON CO., LTD.) to give a sized granulated powder (rotation number: 5,900 rpm, screen diameter: 1000 μm). Two batches thereof are prepared (sized granulated powder 3).

Separately from the above, the micronized compound B (250.0 g), D-mannitol (D(−)-mannitol fine powder, manufactured by Merck, 82.5 g), low-substituted hydroxypropylcellulose (LH-21, manufactured by Shin-Etsu Chemical Co., Ltd., 200.0 g) and light anhydrous silicic acid (Adsolider 101, manufactured by Freund Corporation, 12.5 g) are mixed with an agitation granulator (FM-VG-10, manufactured by POWREX CORPORATION, agitator blade: 400 rpm, chopper blade: 1,200 rpm, mixing time: 30 sec×2 times) to give a mixed powder.

To the obtained powder mixture is added the binder solution obtained in Reference Example E, and the mixture is granulated by an agitation granulator (FM-VG-10, manufactured by POWREX CORPORATION, agitator blade: 400 rpm, chopper blade: 1,200 rpm, granulating time: 30 sec×2 times) to give a granulated powder.

The obtained granulated powder is sized with a screen mill (P-02, manufactured by DALTON CO., LTD.) (rotation number: 5,900 rpm, screen diameter: 3000 μm), and dried using a fluidized-bed dryer (MP-01, manufactured by Freund Corporation, charge air temperature set to: 70° C., drying end-point: exhaustion temperature 38° C.). After the completion of the drying, the powder is sized with a screen mill (P-02, manufactured by DALTON CO., LTD.) to give a sized granulated powder (rotation number: 5,900 rpm, screen diameter: 1000 μm). One batch thereof is prepared (sized granulated powder 4).

To the obtained sized granulated powder (2599.0 g, sized granulated powder 3+sized granulated powder 4) are added crospovidone (Kollidon CL, manufactured by BASF, 322.0 g) and crystalline cellulose (Ceolus PH-102, manufactured by Asahi Kasei Chemicals, 149.5 g), and the mixture is mixed in a V-type mixer (V-20, manufactured by TOKUJU CORPORATION, 150 rpm). Magnesium stearate (from plant source, manufactured by TAIHEI CHEMICAL INDUSTRIAL CO., LTD., 34.5 g) is added, and the mixture is further mixed (150 rpm, tableting powder 2)

The tableting powder 2 (1107 g) is compressed (pestle size: 9 mmφ-13R, tableting speed: 20-30 rpm, desired tablet hardness: 90N) by a tableting machine (correct 12HUK, manufactured by KIKUSUI SEISAKUSHO LTD.) to give a core tablet (thickness 4.1-4.3 mm).

The core tablet is coated by spraying the coating solution obtained in Reference Example F (coating amount: 15 mg, coating end-point: the point where the tablet mass increased by 15.5-16.5 mg from that after pre-drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 100 mg of compound B per tablet.

### Example 10

### 200 mg Tablet

The tableting powder 2 (1699.7 g) obtained in Example 9 is compressed (pestle size: major axis 16.4 mm, minor axis 7.5 mm, tableting speed: 20-30 rpm, desired tablet hardness: 100N) by a tableting machine (correct 12HUK, manufactured by KIKUSUI SEISAKUSHO LTD.) to give a core tablet (thickness 5.1-5.5 mm).

The core tablet is coated by spraying the coating solution obtained in Reference Example F (coating amount: 30 mg, coating end-point: the point where the tablet mass increased by 30.5-31.5 mg from that after pre-drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 200 mg of compound B per tablet.

### Example 11

### 1 mg Tablet

(3,5-Dichloro-4-hydroxyphenyl)-(2,3-dihydrobenzo[1,4]oxazin-4-yl)-methanone (hereinafter to be referred to as compound C) was micronized in a jet mill (JOM-0101, manufactured by SEISHIN ENTERPRISE CO., LTD., pulverization pressure: 0.7 MPa, supply pressure: 0.7 MPa).

The micronized compound C (25.0 g), D-mannitol (D(−)-mannitol fine powder, manufactured by Merck, 887.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) were mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture was added the binder solution obtained in Reference Example A, and the mixture is granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder was sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.). After the completion of the drying, the powder was sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) were added crospovidone (Kollidon CL, manufactured by BASF, 350.0 g) and crystalline cellulose (Ceolus PH-102, manufactured by Asahi Kasei Chemicals, 162.5 g), and the mixture was mixed in a V-type mixer (1 cu. ft. PK Blender, manufactured by Patterson Kelley, 150 rpm). Magnesium stearate (from plant source, manufactured by TAIHEI CHEMICAL INDUSTRIAL CO., LTD., 37.5 g) was added, and the mixture was further mixed (150 rpm).

This was compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet was coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 1 mg of compound C per tablet.

### Example 12

### 5 mg Tablet

Compound C was micronized in a jet mill (JOM-0101, manufactured by SEISHIN ENTERPRISE CO., LTD., pulverization pressure: 0.7 MPa, supply pressure: 0.7 MPa).

The micronized compound C (125.0 g), D-mannitol (D(−)-mannitol fine powder, manufactured by Merck, 787.5 g), crystalline cellulose (Ceolus PH-101, manufactured by Asahi Kasei Chemicals, 750.0 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) were mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture was added the binder solution obtained in Reference Example A, and the mixture was granulated by an agitation granulator (FS-25, manufactured by Fukae Powtec., agitator blade: 400 rpm, chopper blade: 1,200 rpm, granulating time: 60 sec×2 times) to give a granulated powder.

The obtained granulated powder was sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.). After the completion of the drying, the powder was sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) were added crospovidone (Kollidon CL, manufactured by BASF, 350.0 g) and crystalline cellulose (Ceolus PH-102, manufactured by Asahi Kasei Chemicals, 162.5 g), and the mixture was mixed in a V-type mixer (1 cu. ft. PK Blender, manufactured by Patterson Kelley, 150 rpm). Magnesium stearate (from plant source, 37.5 g) was added, and the mixture was further mixed (150 rpm).

This was compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet was coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 5 mg of compound C per tablet.

### Example 13

### 50 mg Tablet

Compound C was micronized in a jet mill (JOM-0101, manufactured by SEISHIN ENTERPRISE CO., LTD., pulverization pressure: 0.7 MPa, supply pressure: 0.7 MPa).

The micronized compound C (125.0 g), D-mannitol (D(−)-mannitol fine powder, manufactured by Merck, 412.5 g), low-substituted hydroxypropylcellulose (LH-21, manufactured by Shin-Etsu Chemical Co., Ltd., 1000.0 g) and light anhydrous silicic acid (Adsolider 101, manufactured by Freund Corporation, 62.5 g) were mixed by an agitation granulator (FS-25, manufactured by Fukae Powtec., agitator blade: 400 rpm, chopper blade: 1,200 rpm, mixing time: 30 sec×2 times) to give a powder mixture.

To the obtained powder mixture was added the binder solution obtained in Reference Example B, and the mixture is granulated by an agitation granulator (FS-25, manufactured by Fukae Powtec., agitator blade: 400 rpm, chopper blade: 1,200 rpm, granulating time: 30 sec×2 times) to give a granulated powder.

The obtained granulated powder was sized with a screen mill (QC-197S, manufactured by Quadro) (impeller: square type, impeller rotation number: 2,000 rpm, screen diameter: 2972 μm), and dried using a fluidized-bed dryer (FLM-15, manufactured by Vector/Freund, charge air temperature set to: 70° C., drying end-point: exhaustion temperature 37° C.). After the completion of the drying, the powder was sized with a screen mill (QC-197S, manufactured by Quadro) to give a sized powder (impeller: round type, impeller rotation number: 2,000 rpm, screen diameter: 1143 μm).

To the obtained sized granulated powder (2825.0 g) were added crospovidone (Kollidon CL, manufactured by BASF, 350.0 g) and crystalline cellulose (Ceolus PH-102, manufactured by Asahi Kasei Chemicals, 162.5 g), and the mixture was mixed in a V-type mixer (1 cu. ft. PK Blender, manufactured by Patterson Kelley, 150 rpm). Magnesium stearate (from plant source, 37.5 g) was added, and the mixture was further mixed (150 rpm).

This was compressed (pestle size: 7 mmφ-10R, tableting speed: 20-30 rpm, desired tablet hardness: 65N) by a tableting machine (XL-100, manufactured by Korsch) to give a core tablet (thickness 3.2-3.4 mm).

The core tablet was coated by spraying the coating solution obtained in Reference Example C (coating amount: 5 mg, coating end-point: the point where the tablet mass increased by 5.5-6.0 mg from that after pre-drying, charge air temperature: 80° C. during coating and 70° C. during post-drying, coating solution spray amount: 10-15 g/min, coating pan rotating speed: 15 rpm) using an automatic coating machine (LDCS-3, manufactured by Vector/Freund) to give a film-coated tablet containing 50 mg of compound C per tablet.

### Example 14

### 100 mg Tablet

Compound C was micronized in a jet mill (JOM-0101, manufactured by SEISHIN ENTERPRISE CO., LTD., pulverization pressure: 0.7 MPa, supply pressure: 0.7 MPa).

The micronized compound C (450.0 g), D-mannitol (D(−)-mannitol fine powder, manufactured by Merck, 148.5 g), low-substituted hydroxypropylcellulose (LH-21, manufactured by Shin-Etsu Chemical Co., Ltd., 360.0 g) and light anhydrous silicic acid (Adsolider 101, manufactured by Freund Corporation, 22.5 g) were mixed by an agitation granulator (FM-VG-10, manufactured by POWREX CORPORATION, agitator blade: 400 rpm, chopper blade: 1,200 rpm, mixing time: 30 sec×2 times) to give a powder mixture.

To the obtained powder mixture was added the binder solution obtained in Reference Example D, and the mixture was granulated by an agitation granulator (FM-VG-10, manufactured by POWREX CORPORATION, agitator blade: 400 rpm, chopper blade: 1,200 rpm, granulating time: 30 sec×2 times) to give a granulated powder.

The obtained granulated powder was sized with screen mill (P-02, manufactured by DALTON CO., LTD.) (rotation number: 5,900 rpm, screen diameter: 3000 μm), and dried in a fluidized-bed dryer (MP-01, manufactured by Freund Corporation) (charge air temperature setting: 70° C., drying end-point: exhaustion temperature 38° C.). After the completion of the drying, a sized granulated powder was obtained with a screen mill (P-02, manufactured by DALTON CO., LTD) (rotation number: 5,900 rpm, screen diameter: 1000 μm). Two batches thereof were prepared (sized granulated powder 5).

Separately from the above, the micronized compound C (250.0 g), D-mannitol (D(−)-mannitol fine powder, manufactured by Merck, 82.5 g), low-substituted hydroxypropylcellulose (LH-21, manufactured by Shin-Etsu Chemical Co., Ltd., 200.0 g) and light anhydrous silicic acid (Adsolider 101, manufactured by Freund Corporation, 12.5 g) were mixed with an agitation granulator (FM-VG-10, manufactured by POWREX CORPORATION, agitator blade: 400 rpm, chopper blade: 1,200 rpm, mixing time: 30 sec×2 times) to give a mixed powder.

To the obtained mixed powder was added the binder solution obtained in Reference Example E, and the mixture was granulated with an agitation granulator (FM-VG-10, manufactured by POWREX CORPORATION, agitator blade: 400 rpm, chopper blade: 1,200 rpm, granulation time: 30 sec×2 times) to give a granulated powder.

The obtained granulated powder was sized with screen mill (P-02, manufactured by DALTON CO., LTD.) (rotation number: 5,900 rpm, screen diameter: 3000 μm), and dried with a fluidized-bed dryer (MP-01, manufactured by Freund Corporation, charge air temperature setting: 70° C., drying end-point: exhaustion temperature 38° C.). After the completion of the drying, a sized powder was obtained with screen mill (P-02, manufactured by DALTON CO., LTD) (rotation number: 5,900 rpm, screen diameter: 1000 μm). One batch thereof was prepared (sized granulated powder 6).

To the obtained sized granulated powder (sized granulated powder 5+sized granulated powder 6, 2599.0 g) were added crospovidone (Kollidon CL, manufactured by BASF, 322.0 g) and crystalline cellulose (Ceolus PH-102, manufactured by Asahi Kasei Chemicals, 149.5 g), and they were mixed with a V-TYPE MIXER (V-20, manufactured by TOKUJU CORPORATION, 150 rpm). Magnesium stearate (from plant source, manufactured by TAIHEI CHEMICAL INDUSTRIAL CO., LTD., 34.5 g) was added, and the mixture was further mixed (150 rpm, tableting powder 3).

The tableting powder 3 (1107 g) was compressed by a tableting machine (correct 12HUK, manufactured by KIKUSUI SEISAKUSHO LTD., pestle size: 9 mmφ-13R, tableting speed: 20-30 rpm, desired tablet hardness: 90N) to give a core tablet (thickness 4.1-4.3 mm).

The core tablet was coated by spraying the coating solution obtained in Reference Example F (coating amount: 15 mg, coating end-point: the point where the tablet mass increased by 15.5-16.5 mg from that after pre-drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 100 mg of compound C per tablet.

### Example 15

### 200 mg Tablet

The tableting powder 3 (1699.7 g) obtained in Example 14 was compressed (pestle size: major axis 16.4 mm, minor axis 7.5 mm, tableting speed: 20-30 rpm, desired tablet hardness: 100N) by a tableting machine (correct 12HUK, manufactured by KIKUSUI SEISAKUSHO LTD.) to give a core tablet (thickness 5.1-5.5 mm).

The core tablet was coated by spraying the coating solution obtained in Reference Example F (coating amount: 30 mg, coating end-point: the point where the tablet mass increased by 30.5-31.5 mg from that after pre-drying, charge air temperature: 75° C., coating solution spray amount: 5-6 g/min, coating pan rotating speed: 18 rpm) using an automatic coating machine (HCT-30, manufactured by Freund Corporation) to give a film-coated tablet containing 200 mg of compound C per tablet.

The compound in Reference Examples 4 to 95 can be also produced a tablet in the same manner as in the above-mentioned Examples 1 to 15.

### Experimental Example 1

### Evaluation of Stability on Excipient Compatibility

Compound A is physically mixed in various additives shown in Table 28 in a mortar (see Table 28 for mixing ratio), and the obtained mixed powder is placed in a glass sample bottle in a layer thickness of about 5 mm.

The glass sample bottle is placed in a desiccator humidity-conditioned to 75% RH (relative humidity) without capping, and the desiccator containing the bottle is preserved for 2 weeks in a constant-temperature equipment set to 60° C.

After the completion of the preservation, a 60 v/v % aqueous acetonitrile solution is added to a concentration of compound A of 0.5 mg/mL, and the mixture is subjected to a dissolution treatment by ultrasonication and a filtration treatment. Each sample is measured by high performance liquid chromatography, and the stability is evaluated based on the changes from the start of preservation. The measurement conditions of high performance liquid chromatography are shown in Table 29.

TABLE 28

| | compounding ratio |
|---|---|
| | (mass ratio) |
| additive | (compound A/additive) |
| calcium silicate | 1/1 |
| lactose | 1/9 |
| crystalline cellulose | 1/9 |
| D-mannitol | 1/9 |
| light anhydrous silicic acid | 1/1 |
| hydroxypropylmethylcellulose 2910 | 1/1 |
| hydroxypropylcellulose | 1/1 |
| low-substituted | 1/9 |
| hydroxypropylcellulose | |
| crospovidone | 1/1 |
| croscarmellose sodium | 1/1 |
| magnesium stearate | 1/1 |
| talc | 1/4 |
| Macrogol 6000 | 1/4 |
| titanium oxide | 1/4 |

TABLE 29

| item | conditions | | |
|---|---|---|---|
| analytical | octadecyl silylated silica gel for liquid | | |
| column | chromatography | | |
| | (150 mm × 4.6 mmφ, 3.5 μm, XTerra MS C₁₈, | | |
| | Waters) | | |
| mobile phase A | distilled water for liquid chromatography | | |
| | containing 0.1 v/v % formic acid | | |
| mobile phase B | acetonitrile for liquid chromatography | | |
| | containing 0.1 v/v % formic acid | | |
| | Time | mobile phase | mobile phase |
| | (min) | A | B |
| mobile phase | 0 | 90 | 10 |
| composition | 10 | 60 | 40 |
| control | 20 | 60 | 40 |
| | 25 | 5 | 95 |
| | 38 | 5 | 95 |
| | 39 | 90 | 10 |
| | 48 | 90 | 10 |

| | |
|---|---|
| column | 40° C. |
| temperature | |
| flow-rate | 0.9 mL/min |
| detection | ultraviolet or visible light |
| method | |
| sample | 20 μL (0.5 mg/mL, distilled water for liquid |
| injection | chromatography containing 60 v/v % |
| volume | acetonitrile for liquid chromatography) |
| analysis | 40 min |
| time | |

By not using a basic additive (particularly, calcium silicate) for making a preparation from compound A and various additives, a preparation capable of ensuring the time-course stability of compound A during preservation can be provided.

### Experimental Example 2

### Dissolution Test 1

The dissolution property of the tablet prepared according to the formulation shown in Table 30 is evaluated by a dissolution test (see Table 31 for dissolution test conditions).

### Preparation of Tablet

A, B and any one of C is gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 30

| | | amount of | |
|---|---|---|---|
| | | formulation | mass ratio |
| | | (mg/1 | in tablet |
| | | tablet) | (w/w %) |
| A | Compound A | 25.0 | 20.0 |
| B | D-mannitol | 58.5 | 46.8 |
| C | low-substituted | 25.0 | 20.0 |
| | hydroxypropylcellulose | | |
| | carmellose | | |
| | carmellose calcium | | |
| | carboxymethyl starch sodium | | |
| | crystalline cellulose | | |
| D | hydroxypropylmethylcellulose 2910 | 3.0 | 2.4 |
| E | croscarmellose sodium | 12.5 | 10.0 |
| F | magnesium stearate | 1.0 | 0.80 |

| | | |
|---|---|---|
| mass (mg) of 1 tablet | 125.0 | 100.0 |

TABLE 31

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound A per 125 mg |
| amount | tablet), n = 3 |
| test method | Paddle Method (described in Japanese |
| | Pharmacopoeia 14th edition) |
| | rotation speed condition: 50 rpm |
| dissolution | 450 mL of second fluid* described in the |
| medium | above-mentioned Pharmacopoeia, containing 0.5 w/w % |
| | polysorbate 80. |
| | temperature conditions: 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding low-substituted hydroxypropylcellulose, a preparation permitting rapid dissolution of compound A can be provided.

### Experimental Example 3

### Dissolution Test 2

The dissolution property of the tablet prepared according to the formulation shown in Table 32 is evaluated by a dissolution test (see Table 33 for dissolution test conditions).

### Preparation of Tablet

A, B and C are gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 32

| | | amount of formulation (mg/1 | | | |
|---|---|---|---|---|---|
| | | tablet) | | | |
| | | (mass ratio in tablet (w/w %)) | | | |
| A | compound A | 50.0 | 50.0 | 50.0 | 50.0 |
| | | (33.8) | (39.1) | (36.2) | (31.6) |
| B | D-mannitol | 25.0 | 25.0 | 25.0 | 25.0 |
| | | (16.9) | (19.5) | (18.1) | (15.8) |
| C | low-substituted | 40.0 | 20.0 | 30.0 | 50.0 |
| | hydroxypropylcellulose | (27.0) | (15.6) | (21.7) | (31.6) |
| D | hydroxy- | 6.0 | 6.0 | 6.0 | 6.0 |
| | propylmethylcellulose 2910 | (4.0) | (4.7) | (4.4) | (3.8) |
| E | crospovidone | 25.0 | 25.0 | 25.0 | 25.0 |
| | | (16.9) | (19.5) | (18.1) | (15.8) |
| F | magnesium stearate | 2.0 | 2.0 | 2.0 | 2.0 |
| | | (1.4) | (1.6) | (1.5) | (1.3) |

| | | | | |
|---|---|---|---|---|
| mass (mg) of 1 tablet | 148.0 | 128.0 | 138.0 | 158.0 |

TABLE 33

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (50 mg of compound A in 1 tablet |
| amount | (128, 138, 148 or 158 mg)), n = 3 |
| test method | Paddle Method (described in Japanese |
| | Pharmacopoeia 14th edition) |
| | rotation speed: 50 rpm |
| dissolution | 900 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding a suitable amount of low-substituted hydroxypropylcellulose, a preparation permitting rapid dissolution of compound A while suppressing the amount of addition of low-substituted hydroxypropylcellulose can be provided.

### Experimental Example 4

### Dissolution Test 3

The dissolution property of the tablet prepared according to the formulation shown in Table 34 is evaluated by a dissolution test (see Table 35 for dissolution test conditions).

### Preparation of Tablet

A, B and C are gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with any one of E and then F. The obtained tableting powder is compressed by pressurization (desired value; 100N) in a screw type universal testing machine (SC-50CJ, manufactured by JT TOHSHI CO. LTD.) to give a tablet.

TABLE 34

| | | amount of | |
|---|---|---|---|
| | | formulation | mass ratio |
| | | (mg/1 | in tablet |
| | | tablet) | (w/w %) |
| A | compound A | 25.0 | 20.0 |
| B | D-mannitol | 58.5 | 46.8 |
| C | low-substituted | 25.0 | 20.0 |
| | hydroxypropylcellulose | | |
| D | hydroxypropylmethylcellulose | 3.0 | 2.4 |
| | 2910 | | |
| E | crospovidone | 12.5 | 10.0 |
| | croscarmellose sodium | | |
| | low-substituted | | |
| | hydroxypropylcellulose | | |
| | carboxymethyl starch sodium | | |
| F | magnesium stearate | 1.0 | 0.8 |

| | | |
|---|---|---|
| mass (mg) of 1 tablet | 125.0 | 100.0 |

TABLE 35

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound A in 125 mg |
| amount | tablet), n = 3 |
| test method | Paddle Method (described in Japanese |
| | Pharmacopoeia 14th edition) |
| | rotation speed: 50 rpm |
| dissolution | 450 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding crospovidone, a preparation permitting rapid dissolution of compound A can be provided.

### Experimental Example 5

### Dissolution Test 4

The dissolution property of the tablet prepared according to the formulation (Table 36) shown in the following table is evaluated by a dissolution test (see Table 37 for dissolution test conditions).

### Preparation of Tablet

A, B and C is gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 36

| | | amount of formulation (mg/1 | | | |
|---|---|---|---|---|---|
| | | tablet) | | | |
| | | (ratio of mass in tablet | | | |
| | | (w/w %)) | | | |
| A | compound A | 50.0 | 50.0 | 50.0 | 50.0 |
| | | (36.6) | (33.8) | (38.6) | (40.7) |
| B | D-mannitol | 25.0 | 25.0 | 25.0 | 25.0 |
| | | (18.3) | (16.9) | (19.3) | (20.3) |
| C | low-substituted | 40.0 | 40.0 | 40.0 | 40.0 |
| | hydroxypropylcellulose | (29.3) | (27.0) | (30.9) | (32.5) |
| D | hydroxypropylmethylcellulose | 6.0 | 6.0 | 6.0 | 6.0 |
| | 2910 | (4.4) | (4.0) | (4.6) | (4.9) |
| E | crospovidone | 13.7 | 25.0 | 6.5 | 0.0 |
| | | (10.0) | (16.9) | (5.0) | (0.0) |
| F | magnesium stearate | 2.0 | 2.0 | 2.0 | 2.0 |
| | | (1.5) | (1.4) | (1.5) | (1.6) |

| | | | | |
|---|---|---|---|---|
| mass (mg) of 1 tablet | 136.7 | 148.0 | 129.5 | 123.0 |

TABLE 37

| item | conditions |
|---|---|
| apparatus | NTR-VS6P/UV-160, or NTR-6100/UV-1600 |
| | (Toyama Sangyo Co., Ltd./SHIMADZU CORPORATION) |
| feeding | 1 tablet (50 mg of compound A in 1 tablet (123, |
| amount | 129.5, 136.7 or 148 mg)), n = 3 |
| test | Paddle Method (described in Japanese |
| method | Pharmacopoeia 14th edition) |
| | rotation speed: 50 rpm |
| dissolution | 900 mL of second fluid* described in the above- |
| medium | mentioned Pharmacopoeia, containing 0.5 w/w % |
| | polysorbate 80. temperature conditions: 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding a suitable amount of crospovidone, a preparation permitting rapid dissolution of compound A while suppressing the amount of addition of crospovidone can be provided.

### Experimental Example 6

### Evaluation of Stability on Excipient Compatibility

Compound B is physically mixed in various additives shown in Table 38 in a mortar (see Table 38 for mixing ratio), and the obtained mixed powder is placed in a glass sample bottle in a layer thickness of about 5 mm.

The glass sample bottle is placed in a desiccator humidity-conditioned to 75% RH (relative humidity) without capping, and the desiccator containing the bottle is preserved for 2 weeks in a constant-temperature equipment set to 60° C.

After the completion of the preservation, a 60 v/v % aqueous acetonitrile solution is added to a concentration of compound B of 0.5 mg/mL, and the mixture is subjected to a dissolution treatment by ultrasonication and a filtration treatment. Each sample is measured by high performance liquid chromatography, and the stability is evaluated based on the changes from the start of preservation. The measurement conditions of high performance liquid chromatography are shown in Table 39.

TABLE 38

| | compounding |
|---|---|
| | ratio (mass |
| | ratio) |
| | (compound |
| additive | B/additive) |
| calcium silicate | 1/1 |
| lactose | 1/9 |
| crystalline cellulose | 1/9 |
| D-mannitol | 1/9 |
| light anhydrous silicic acid | 1/1 |
| hydroxypropylmethylcellulose | 1/1 |
| 2910 | |
| hydroxypropylcellulose | 1/1 |
| low-substituted | 1/9 |
| hydroxypropylcellulose | |
| crospovidone | 1/1 |
| croscarmellose sodium | 1/1 |
| magnesium stearate | 1/1 |
| talc | 1/4 |
| macrogol6000 | 1/4 |
| titanium oxide | 1/4 |

TABLE 39

| | |
|---|---|
| item | conditions |

| analytical | octadecyl silylated silica gel for liquid | | |
|---|---|---|---|
| column | chromatography | | |
| | (150 mm × 4.6 mmφ, 3.5 μm, XTerra MS C₁₈, | | |
| | Waters) | | |
| mobile phase A | distilled water for liquid chromatography | | |
| | containing 0.1 v/v % formic acid | | |
| mobile phase B | acetonitrile for liquid chromatography | | |
| | containing 0.1 v/v % formic acid | | |
| | Time | mobile phase | mobile phase |
| | (min) | A | B |
| mobile phase | 0 | 90 | 10 |
| composition | 10 | 60 | 40 |
| control | 20 | 60 | 40 |
| | 25 | 5 | 95 |
| | 38 | 5 | 95 |
| | 39 | 90 | 10 |
| | 48 | 90 | 10 |

| | |
|---|---|
| column | 40° C. |
| temperature | |
| flow-rate | 0.9 mL/min |
| detection | ultraviolet or visible light |
| method | |
| sample | 20 μL (0.5 mg/mL, distilled water for liquid |
| injection | chromatography containing 60 v/v % |
| volume | acetonitrile for liquid chromatography) |
| analysis | 40 min |
| time | |

By not using a basic additive (particularly, calcium silicate) for making a preparation from compound B and various additives, a preparation capable of ensuring the time-course stability of compound B during preservation can be provided.

### Experimental Example 7

### Dissolution Test 5

The dissolution property of the tablet prepared according to the formulation shown in Table 40 is evaluated by a dissolution test (see Table 41 for dissolution test conditions).

### Preparation of Tablet

A, B and any one of C is gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 40

| | | amount of | |
|---|---|---|---|
| | | formulation | Mass ratio |
| | | (mg/1 | in tablet |
| | | tablet) | (w/w %) |
| A | compound B | 25.0 | 20.0 |
| B | D-mannitol | 58.5 | 46.8 |
| C | low-substituted | 25.0 | 20.0 |
| | hydroxypropylcellulose | | |
| | carmellose | | |
| | carmellose calcium | | |
| | carboxymethyl starch sodium | | |
| | crystalline cellulose | | |
| D | Hydroxypropylmethylcellulose 2910 | 3.0 | 2.4 |
| E | croscarmellose sodium | 12.5 | 10.0 |
| F | magnesium stearate | 1.0 | 0.80 |

| | | |
|---|---|---|
| mass (mg) of 1 tablet | 125.0 | 100.0 |

TABLE 41

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound B in 1 tablet |
| amount | (125 mg)), n = 3 |
| test | Paddle Method (described in Japanese |
| method | Pharmacopoeia 14th edition) |
| | rotation speed: 50 rpm |
| dissolution | 450 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding low-substituted hydroxypropylcellulose, a preparation permitting rapid dissolution of compound B can be provided.

### Experimental Example 8

### Dissolution Test 6

The dissolution property of the tablet prepared according to the formulation shown in Table 42 is evaluated by a dissolution test (see Table 43 for dissolution test conditions).

### Preparation of Tablet

A, B and C are gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 42

| | | amount of formulation (mg/1 | | | |
|---|---|---|---|---|---|
| | | tablet) | | | |
| | | (mass ratio in tablet | | | |
| | | (w/w %)) | | | |
| A | compound B | 50.0 | 50.0 | 50.0 | 50.0 |
| | | (33.8) | (39.1) | (36.2) | (31.6) |
| B | D-mannitol | 25.0 | 25.0 | 25.0 | 25.0 |
| | | (16.9) | (19.5) | (18.1) | (15.8) |
| C | low-substituted | 40.0 | 20.0 | 30.0 | 50.0 |
| | hydroxypropylcellulose | (27.0) | (15.6) | (21.7) | (31.6) |
| D | Hydroxypropylmethyl- | 6.0 | 6.0 | 6.0 | 6.0 |
| | cellulose 2910 | (4.0) | (4.7) | (4.4) | (3.8) |
| E | crospovidone | 25.0 | 25.0 | 25.0 | 25.0 |
| | | (16.9) | (19.5) | (18.1) | (15.8) |
| F | magnesium stearate | 2.0 | 2.0 | 2.0 | 2.0 |
| | | (1.4) | (1.6) | (1.5) | (1.3) |

| | | | | |
|---|---|---|---|---|
| mass (mg) of 1 tablet | 148.0 | 128.0 | 138.0 | 158.0 |

TABLE 43

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (50 mg of compound B in 1 tablet |
| amount | (128, 138, 148 or 158 mg)), n = 3 |
| test | Paddle Method (described in Japanese |
| method | Pharmacopoeia 14th edition) |
| | rotation speed: 50 rpm |
| dissolution | 900 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding a suitable amount of low-substituted hydroxypropylcellulose, a preparation permitting rapid dissolution of compound B while suppressing the amount of addition of low-substituted hydroxypropylcellulose can be provided.

### Experimental Example 9

### Dissolution Test 7

The dissolution property of the tablet prepared according to the formulation shown in Table 44 is evaluated by a dissolution test (see Table 45 for dissolution test conditions).

### Preparation of Tablet

A, B and C are gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with any one of E and then F. The obtained tableting powder is compressed by pressurization (desired value; 100N) in a screw type universal testing machine (SC-50CJ, manufactured by JT TOHSHI CO. LTD.) to give a tablet.

TABLE 44

| | | amount of | |
|---|---|---|---|
| | | formulation | mass ratio |
| | | (mg/1 | in tablet |
| | | tablet) | (w/w %) |
| A | compound B | 25.0 | 20.0 |
| B | D-mannitol | 58.5 | 46.8 |
| C | low-substituted | 25.0 | 20.0 |
| | hydroxypropylcellulose | | |
| D | Hydroxypropylmethylcellulose | 3.0 | 2.4 |
| | 2910 | | |
| E | crospovidone | 12.5 | 10.0 |
| | croscarmellose sodium | | |
| | low-substituted | | |
| | hydroxypropylcellulose | | |
| | carboxymethyl starch sodium | | |
| F | magnesium stearate | 1.0 | 0.8 |

| | | |
|---|---|---|
| mass (mg) of 1 tablet | 125.0 | 100.0 |

TABLE 45

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound B in 1 tablet |
| amount | (125 mg)), n = 3 |
| test | Paddle Method (described in Japanese |
| method | Pharmacopoeia 14th edition) |
| | rotation speed: 50 rpm |
| dissolution | 450 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding crospovidone, a preparation permitting rapid dissolution of compound B can be provided.

### Experimental Example 10

### Dissolution Test 8

The dissolution property of the tablet prepared according to the formulation (Table 46) shown in the following table is evaluated by a dissolution test (see Table 47 for dissolution test conditions).

### Preparation of Tablet

A, B and C are gently mixed in a mortar, and a solution of D adjusted to a concentration of 5 w/w % with purified water in advance is added thereto to conduct granulation. The obtained granulated powder is passed through a sieve (sieve mesh size 710 μm), and dried at 60° C. for 2 hr. After the completion of the drying, the powder is sized with a 500 μm sieve, and mixed successively with E and F. The obtained tableting powder is compressed by pressurization (desired value; 100N) in a screw type universal testing machine (SC-50CJ, manufactured by JT TOHSHI CO. LTD.) to give a tablet.

TABLE 46

| | | amount of formulation (mg/1 | | | |
|---|---|---|---|---|---|
| | | tablet) | | | |
| | | (mass ratio in tablet | | | |
| | | (w/w %)) | | | |
| A | compound B | 50.0 | 50.0 | 50.0 | 50.0 |
| | | (36.6) | (33.8) | (38.6) | (40.7) |
| B | D-mannitol | 25.0 | 25.0 | 25.0 | 25.0 |
| | | (18.3) | (16.9) | (19.3) | (20.3) |
| C | low-substituted | 40.0 | 40.0 | 40.0 | 40.0 |
| | hydroxypropylcellulose | (29.3) | (27.0) | (30.9) | (32.5) |
| D | Hydroxy- | 6.0 | 6.0 | 6.0 | 6.0 |
| | propylmethylcellulose 2910 | (4.4) | (4.0) | (4.6) | (4.9) |
| E | crospovidone | 13.7 | 25.0 | 6.5 | 0.0 |
| | | (10.0) | (16.9) | (5.0) | (0.0) |
| F | magnesium stearate | 2.0 | 2.0 | 2.0 | 2.0 |
| | | (1.5) | (1.4) | (1.5) | (1.6) |

| | | | | |
|---|---|---|---|---|
| mass (mg) of 1 tablet | 136.7 | 148.0 | 129.5 | 123.0 |

TABLE 47

| item | conditions |
|---|---|
| apparatus | NTR-VS6P/UV-160, or NTR-6100/UV-1600 |
| | (Toyama Sangyo Co., Ltd./SHIMADZU CORPORATION) |
| feeding | 1 tablet (50 mg of compound B in 1 tablet (123, |
| amount | 129.5, 136.7 or 148 mg)), n = 3 |
| test | Paddle Method (described in Japanese |
| method | Pharmacopoeia 14th edition) |
| | rotation speed: 50 rpm |
| dissolution | 900 mL of second fluid* described in the above- |
| medium | mentioned Pharmacopoeia, containing 0.5 w/w % |
| | polysorbate 80. temperature conditions: 37° C. |
| optical | 10 mm |
| length | |
| measurement | ultraviolet or visible light |
| wavelength | |

*buffer (pH 6.8) prepared from potassium dihydrogen phosphate and sodium hydroxide

By adding a suitable amount of crospovidone, a preparation permitting rapid dissolution of compound B while suppressing the amount of addition of crospovidone can be provided.

### Experimental Example 11

### Evaluation of Stability on Excipient Compatibility

Compound C was physically mixed in various additives shown in Table 48 in a mortar (see Table 48 for mixing ratio), and the obtained mixed powder was placed in a glass sample bottle in a layer thickness of about 5 mm.

The glass sample bottle was placed in a desiccator humidity-conditioned to 75% RH (relative humidity) without capping, and the desiccator containing the bottle was preserved for 2 weeks in a constant-temperature equipment set to 60° C.

After the completion of the preservation, a 60 v/v % aqueous acetonitrile solution was added to a concentration of compound C of 0.5 mg/mL, and the mixture was subjected to a dissolution treatment by ultrasonication and a filtration treatment. Each sample was measured by high performance liquid chromatography, and the stability was evaluated based on the changes from the start of preservation. The measurement conditions of high performance liquid chromatography are shown in Table 49.

In Table 48, the area percentage of the decomposition product shows the ratio of the peak area of the decomposition product to the sum of the areas of all the peaks detected by high performance liquid chromatography.

TABLE 48

| | compounding | area |
|---|---|---|
| | ratio (mass | percentage |
| | ratio) | (%) of |
| | (compound | decomposition |
| additive | C/additive) | product |
| calcium silicate | 1/1 | 0.19 |
| lactose | 1/9 | N/A |
| crystalline cellulose | 1/9 | N/A |
| D-mannitol | 1/9 | N/A |
| light anhydrous silicic acid | 1/1 | N/A |
| hydroxypropylmethylcellulose | 1/1 | N/A |
| 2910 | | |
| hydroxypropylcellulose | 1/1 | N/A |
| low-substituted | 1/9 | N/A |
| hydroxypropylcellulose | | |
| crospovidone | 1/1 | N/A |
| croscarmellose sodium | 1/1 | N/A |
| magnesium stearate | 1/1 | N/A |
| talc | 1/4 | N/A |
| macrogol6000 | 1/4 | N/A |
| titanium oxide | 1/4 | N/A |

N/A: not appearance

TABLE 49

| item | conditions | | |
|---|---|---|---|
| analytical | octadecyl silylated silica gel for liquid | | |
| column | chromatography | | |
| | (150 mm × 4.6 mmφ, 3.5 μm, XTerra MS C₁₈, Waters) | | |
| mobile phase A | distilled water for liquid chromatography | | |
| | containing 0.1 v/v % formic acid | | |
| mobile phase B | acetonitrile for liquid chromatography | | |
| | containing 0.1 v/v % formic acid | | |
| | Time (min) | mobile phase A | mobile phase B |
| mobile phase | 0 | 90 | 10 |
| composition | 10 | 60 | 40 |
| control | 20 | 60 | 40 |
| | 25 | 5 | 95 |
| | 38 | 5 | 95 |
| | 39 | 90 | 10 |
| | 48 | 90 | 10 |

| | |
|---|---|
| column | 40° C. |
| temperature | |
| flow-rate | 0.9 mL/min |
| | (dissolution time of compound C; about 21 min) |
| detection | UV; 220 nm, 284 nm |
| method | |
| sample | 20 μL (0.5 mg/mL, distilled water for liquid |
| injection | chromatography containing 60 v/v % acetonitrile |
| volume | for liquid chromatography) |
| analysis | 40 min |
| time | |

From Table 48, it has been clarified that

(1) compound C shows good stability when compounded with an additive other than calcium silicate, and
(2) compound C shows a novel peak considered to derive from the decomposition product of compound C when calcium silicate is added.

From the foregoing, a preparation capable of ensuring the time-course stability of compound C during preservation can be provided by not using a basic additive (particularly, calcium silicate) for making a preparation from compound C and various additives.

### Experimental Example 12

### Dissolution Test 9

The dissolution property of the tablet prepared according to the formulation shown in Table 50 was evaluated by a dissolution test (see Table 51 for dissolution test conditions). The results of the dissolution test are shown in Table 52.

### Preparation of Tablet

A, B and any one of C was gently mixed in a mortar, and a solution of D adjusted to a concentration of 5 w/w % with purified water in advance was added thereto to conduct granulation. The obtained granulated powder was passed through a sieve (sieve mesh size 710 μm), and dried at 60° C. for 2 hr. After the completion of the drying, the powder was sized with a 500 μm sieve, and mixed successively with E and F. The obtained tableting powder was compressed by pressurization (desired value; 100N) in a screw type universal testing machine (SC-50CJ, manufactured by JT TOHSHI CO. LTD.) to give a tablet.

TABLE 50

| | | amount of | |
|---|---|---|---|
| | | formulation | mass ratio |
| | | (mg/1 | in tablet |
| | | tablet) | (w/w %) |
| A | compound C | 25.0 | 20.0 |
| B | D-mannitol | 58.5 | 46.8 |
| C | low-substituted | 25.0 | 20.0 |
| | hydroxypropylcellulose | | |
| | carmellose | | |
| | carmellose calcium | | |
| | carboxymethyl starch sodium | | |
| | crystalline cellulose | | |
| D | Hydroxypropylmethylcellulose 2910 | 3.0 | 2.4 |
| E | croscarmellose sodium | 12.5 | 10.0 |
| F | magnesium stearate | 1.0 | 0.80 |

| | | |
|---|---|---|
| mass (mg) of 1 tablet | 125.0 | 100.0 |

TABLE 51

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound C in 1 tablet |
| amount | (125 mg)), n = 3 |
| test | Paddle Method (described in Japanese |
| method | Pharmacopoeia 14th edition) |
| | rotation speed: 50 rpm |
| dissolution | 450 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | 287 nm, 500 nm |
| wavelength | |
| λmax | 1.7752 |

*buffer (pH 6.8) prepared from potassium dihydrogen phosphate and sodium hydroxide

TABLE 52

| | D₁₀* | D₂₀* | D₃₀* |
|---|---|---|---|
| low-substituted | 62.62 | 94.95 | 98.04 |
| hydroxypropylcellulose | | | |
| carmellose | 58.86 | 87.28 | 97.03 |
| carmellose calcium | 26.52 | 80.50 | 90.04 |
| carboxymethyl starch sodium | 39.50 | 87.86 | 95.78 |
| crystalline cellulose | 33.24 | 61.99 | 72.90 |

*Dₓₓ shows a dissolution ratio (%) at XX minutes.

As shown in Table 52, the most rapid dissolution was found in 0-30 min with the formulation containing low-substituted hydroxypropylcellulose.

From the foregoing, a preparation permitting rapid dissolution of compound C can be provided by adding low-substituted hydroxypropylcellulose.

### Experimental Example 13

### Dissolution Test 10

The dissolution property of the tablet prepared according to the formulation shown in Table 53 was evaluated by a dissolution test (see Table 54 for dissolution test conditions). The results of the dissolution test are shown in Table 55.

### Preparation of Tablet

A, B and C were gently mixed in a mortar, and a solution of D adjusted to a concentration of 5 w/w % with purified water in advance was added thereto to conduct granulation. The obtained granulated powder was passed through a sieve (sieve mesh size 710 μm), and dried at 60° C. for 2 hr. After the completion of the drying, the powder was sized with a 500 μm sieve, and mixed successively with E and F. The obtained tableting powder was compressed by pressurization (desired value; 100N) in a screw type universal testing machine (SC-50CJ, manufactured by JT TOHSHI CO. LTD.) to give a tablet.

TABLE 53

| | | amount of formulation (mg/1 | | | |
|---|---|---|---|---|---|
| | | tablet) | | | |
| | | (mass ratio in tablet | | | |
| | | (w/w %)) | | | |
| A | compound C | 50.0 | 50.0 | 50.0 | 50.0 |
| | | (33.8) | (39.1) | (36.2) | (31.6) |
| B | D-mannitol | 25.0 | 25.0 | 25.0 | 25.0 |
| | | (16.9) | (19.5) | (18.1) | (15.8) |
| C | low-substituted | 40.0 | 20.0 | 30.0 | 50.0 |
| | hydroxypropylcellulose | (27.0) | (15.6) | (21.7) | (31.6) |
| D | Hydroxy- | 6.0 | 6.0 | 6.0 | 6.0 |
| | propylmethylcellulose 2910 | (4.0) | (4.7) | (4.4) | (3.8) |
| E | crospovidone | 25.0 | 25.0 | 25.0 | 25.0 |
| | | (16.9) | (19.5) | (18.1) | (15.8) |
| F | magnesium stearate | 2.0 | 2.0 | 2.0 | 2.0 |
| | | (1.4) | (1.6) | (1.5) | (1.3) |

| | | | | |
|---|---|---|---|---|
| mass (mg) of 1 tablet | 148.0 | 128.0 | 138.0 | 158.0 |

TABLE 54

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (50 mg of compound C in 1 tablet |
| amount | (128, 138, 148 or 158 mg)), n = 3 |
| test | Paddle Method (described in Japanese |
| method | Pharmacopoeia 14th edition) |
| | rotation speed: 50 rpm |
| dissolution | 900 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | 287 nm, 500 nm |
| wavelength | |
| λmax | 1.7752 |

*buffer (pH 6.8) prepared from potassium dihydrogen phosphate and sodium hydroxide

TABLE 55

| low-substituted | | | |
|---|---|---|---|
| hydroxypropylcellulose | | | |
| addition amount (mg) | D₁₀* | D₂₀* | D₃₀* |
| 40.0 | 78.45 | 95.54 | 98.24 |
| 20.0 | 65.36 | 88.70 | 94.68 |
| 30.0 | 64.46 | 89.31 | 95.07 |
| 50.0 | 83.43 | 97.53 | 99.44 |

*Dₓₓ shows a dissolution ratio (%) at XX minutes.

As shown in Table 55, a clearly rapid dissolution was observed in 0-30 min with the formulation containing 40.0 mg of low-substituted hydroxypropylcellulose, as compared to the formulations containing 20.0 mg or 30.0 mg thereof. Even when compared to the formulation containing 50.0 mg thereof, which is a 10 mg higher amount, almost equal dissolution property was observed.

From the foregoing, a preparation permitting rapid dissolution of compound C while suppressing the amount of addition of low-substituted hydroxypropylcellulose can be provided by adding low-substituted hydroxypropylcellulose (40 mg, mass ratio in tablet 27.0 w/w %).

### Experimental Example 14

### Dissolution Test 11

The dissolution property of the tablet prepared according to the formulation shown in Table 56 was evaluated by a dissolution test (see Table 57 for dissolution test conditions). The results of the dissolution test are shown in Table 58.

### Preparation of Tablet

A, B and C were gently mixed in a mortar, and a solution of D adjusted to a concentration of 5 w/w % with purified water in advance was added thereto to conduct granulation. The obtained granulated powder was passed through a sieve (sieve mesh size 710 μm), and dried at 60° C. for 2 hr. After the completion of the drying, the powder was sized with a 500 μm sieve, and mixed successively with any one of E and then F. The obtained tableting powder was compressed by pressurization (desired value; 100N) in a screw type universal testing machine (SC-50CJ, manufactured by JT TOHSHI CO. LTD.) to give a tablet.

TABLE 56

| | | amount of | |
|---|---|---|---|
| | | formulation | mass ratio |
| | | (mg/1 | in tablet |
| | | tablet) | (w/w %) |
| A | compound C | 25.0 | 20.0 |
| B | D-mannitol | 58.5 | 46.8 |
| C | low-substituted | 25.0 | 20.0 |
| | hydroxypropylcellulose | | |
| D | Hydroxypropylmethylcellulose | 3.0 | 2.4 |
| | 2910 | | |
| E | crospovidone | 12.5 | 10.0 |
| | croscarmellose sodium | | |
| | low-substituted | | |
| | hydroxypropylcellulose | | |
| | carboxymethyl starch sodium | | |
| F | magnesium stearate | 1.0 | 0.8 |

| | | |
|---|---|---|
| mass (mg) of 1 tablet | 125.0 | 100.0 |

TABLE 57

| item | conditions |
|---|---|
| apparatus | NTR-VS6P/UV-160, or NTR-6100/UV-1600 |
| | (Toyama Sangyo Co., Ltd./SHIMADZU |
| | CORPORATION) |
| feeding | 1 tablet (25 mg of compound C in 1 tablet |
| amount | (125 mg)), n = 3 |
| test | Paddle Method (described in Japanese |
| method | Pharmacopoeia 14th edition) |
| | rotation speed: 50 rpm |
| dissolution | 450 mL of second fluid* described in the |
| medium | above-mentioned Pharmacopoeia, containing 0.5 w/w % |
| | polysorbate 80. temperature conditions: |
| | 37° C. |
| optical | 10 mm |
| length | |
| measurement | 287 nm, 500 nm |
| wavelength | |
| λmax | 1.7752 |

*buffer (pH 6.8) prepared from potassium dihydrogen phosphate and sodium hydroxide

TABLE 58

| | D₁₀* | D₂₀* | D₃₀* |
|---|---|---|---|
| crospovidone | 81.72 | 93.38 | 95.20 |
| croscarmellose sodium | 49.78 | 90.22 | 95.33 |
| low-substituted | 60.80 | 92.16 | 95.09 |
| hydroxypropylcellulose | | | |
| carboxymethyl starch sodium | 64.20 | 90.47 | 94.28 |

*Dₓₓ shows a dissolution ratio (%) at XX minutes.

As shown in Table 58, the most rapid dissolution of compound C was found in 0-30 min with the formulation containing crospovidone.

From the foregoing, a preparation permitting rapid dissolution of compound C can be provided by adding crospovidone.

### Experimental Example 15

### Dissolution Test 12

The dissolution property of the tablet prepared according to the formulation (Table 59) shown in the following table was evaluated by a dissolution test (see Table 60 for dissolution test conditions). The results of the dissolution test are shown in Table 61.

### Preparation of Tablet

A, B and C were gently mixed in a mortar, and a solution of D adjusted to a concentration of 5 w/w % with purified water in advance was added thereto to conduct granulation. The obtained granulated powder was passed through a sieve (sieve mesh size 710 μm), and dried at 60° C. for 2 hr. After the completion of the drying, the powder was sized with a 500 μm sieve, and mixed successively with E and F. The obtained tableting powder was compressed by pressurization (desired value; 100N) in a screw type universal testing machine (SC-50CJ, manufactured by JT TOHSHI CO. LTD.) to give a tablet.

TABLE 59

| | | amount of formulation (mg/1 | | | |
|---|---|---|---|---|---|
| | | tablet) | | | |
| | | (mass ratio in tablet | | | |
| | | (w/w %)) | | | |
| A | compound C | 50.0 | 50.0 | 50.0 | 50.0 |
| | | (36.6) | (33.8) | (38.6) | (40.7) |
| B | D-mannitol | 25.0 | 25.0 | 25.0 | 25.0 |
| | | (18.3) | (16.9) | (19.3) | (20.3) |
| C | low-substituted | 40.0 | 40.0 | 40.0 | 40.0 |
| | hydroxypropylcellulose | (29.3) | (27.0) | (30.9) | (32.5) |
| D | Hydroxy- | 6.0 | 6.0 | 6.0 | 6.0 |
| | propylmethylcellulose 2910 | (4.4) | (4.0) | (4.6) | (4.9) |
| E | crospovidone | 13.7 | 25.0 | 6.5 | 0.0 |
| | | (10.0) | (16.9) | (5.0) | (0.0) |
| F | magnesium stearate | 2.0 | 2.0 | 2.0 | 2.0 |
| | | (1.5) | (1.4) | (1 5) | (1.6) |

| | | | | |
|---|---|---|---|---|
| mass (mg) of 1 tablet | 136.7 | 148.0 | 129.5 | 123.0 |

TABLE 60

| item | conditions |
|---|---|
| apparatus | NTR-VS6P/UV-160, or NTR-6100/UV-1600 |
| | (Toyama Sangyo Co., Ltd./SHIMADZU CORPORATION) |
| feeding | 1 tablet (50 mg of compound C in 1 tablet (123, |
| amount | 129.5, 136.7 or 148 mg)), n = 3 |
| test | Paddle Method (described in Japanese |
| method | Pharmacopoeia 14th edition) |
| | rotation speed: 50 rpm |
| dissolution | 900 mL of second fluid* described in the above- |
| medium | mentioned Pharmacopoeia, containing 0.5 w/w % |
| | polysorbate 80. temperature conditions: 37° C. |
| optical | 10 mm |
| length | |
| measurement | 287 nm, 500 nm |
| wavelength | |
| λmax | 1.7752 |

*buffer (pH 6.8) prepared from potassium dihydrogen phosphate and sodium hydroxide

TABLE 61

| crospovidone addition | | | |
|---|---|---|---|
| amount (mg) | D₁₀* | D₂₀* | D₃₀* |
| 13.7 | 88.19 | 98.08 | 99.03 |
| 25.0 | 88.95 | 97.61 | 98.26 |
| 6.5 | 80.96 | 95.41 | 96.88 |
| 0.0 | 72.39 | 94.40 | 97.45 |

*Dₓₓ shows a dissolution ratio (%) at XX minutes.

As shown in Table 61, the formulation containing 13.7 mg of crospovidone showed clearly rapid dissolution in 0-30 min as compared to the formulation containing 6.5 mg of crospovidone and the formulation free of crospovidone. Even when compared to the formulation containing 25.0 mg thereof, which is close to a double dose, almost equal dissolution property was observed.

From the foregoing, a preparation permitting rapid dissolution of compound C while suppressing the amount of addition of crospovidone can be provided by adding crospovidone (13.7 mg, mass ratio in tablet 10.0 w/w %).

### INDUSTRIAL APPLICABILITY

The pharmaceutical composition of the present invention is superior in the time-course stability and dissolution property (disintegratability) of the effective ingredient and useful for the prophylaxis or treatment of pathology showing involvement of uric acid, such as hyperuricemia, gouty tophus, acute gouty arthritis, chronic gouty arthritis, gouty kidney, urolithiasis, renal function disorder, coronary artery disease, ischemic heart disease and the like, and the like.

This application is based on a patent application No. 2006/147440 filed in Japan and a patent application No. 60/810,008 filed in USA the contents of which are hereby incorporated by reference.

1. A pharmaceutical composition comprising a nitrogen-containing fused ring compound represented by the following formula [1] or a pharmaceutically acceptable salt thereof, and one or more pharmaceutically acceptable additives, wherein the nitrogen-containing fused ring compound or a pharmaceutically acceptable salt thereof is not in contact with a basic additive: 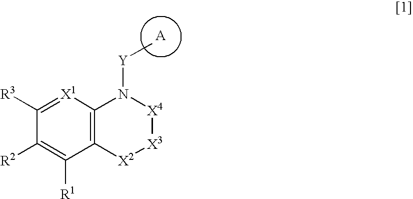
wherein
R¹, R² and R³ are the same or different and each is
1) a hydrogen atom, or
2) a group selected from group A below, or
3) R¹ and R² may form, together with the carbon atoms they are bonded to, a saturated or unsaturated carbon ring having 3 to 14 carbon atoms optionally substituted by one or more, the same or different substituents selected from group A below, or
4) R² and R³ may form, together with the carbon atoms they are bonded to, a saturated or unsaturated carbon ring having 3 to 14 carbon atoms optionally substituted by one or more, the same or different substituents selected from group A below;
Y is
1) —CO—,
2) —CS—, or
3) —S(═O)₂—;
X¹ is
1) a nitrogen atom, or
2) CR⁴ wherein R⁴ is
(a) a hydrogen atom, or
(b) a group selected from group A below, or
(c) R³ and R⁴ may form, together with the carbon atoms they are bonded to, a saturated or unsaturated carbon ring having 3 to 14 carbon atoms optionally substituted by one or more, the same or different substituents selected from group A below;
X² is
1) an oxygen atom,
2) —N(R⁵)— wherein R⁵ is
(a) a hydrogen atom, or
(b) a C₁₋₆ alkyl group optionally substituted by one or more, the same or different substituents selected from group B below,
3) —N(COR⁶)— wherein R⁶ is
(a) a hydroxyl group,
(b) a C₁₋₆ alkyl group optionally substituted by one or more, the same or different substituents selected from group B below,
(c) a C₁₋₆ alkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
(d) a saturated or unsaturated carbon ring group having 3 to 14 carbon atoms optionally substituted by one or more, the same or different substituents selected from group A below,
(e) a cycloalkylalkoxy group optionally substituted by one or more, the same or different substituents selected from group A below,
(f) an aralkyl group optionally substituted by one or more, the same or different substituents selected from group A below, or
(g) an aralkoxy group optionally substituted by one or more, the same or different substituents selected from group A below,
4) —N(S(═O)₂R⁶)— wherein R⁶ is as defined above,
5) —N(CONR⁷R⁸)— wherein R⁷ and R⁸ are the same or different and each is
(a) a hydrogen atom, or
(b) a C₁₋₆ alkyl group optionally substituted by one or more, the same or different substituents selected from group B below, or
(c) R⁷ and R⁸ may form, together with the nitrogen atom they are bonded to, a monocyclic nitrogen-containing saturated heterocycle optionally substituted by one or more, the same or different substituents selected from group A below,
6) a sulfur atom,
7) —S(═O)—,
8) —S(═O)₂—, or
9) —CR⁹R¹⁰— wherein R⁹ and R¹⁰ are the same or different and each is
(a) a hydrogen atom, or
(b) a C₁₋₆ alkyl group optionally substituted by one or more, the same or different substituents selected from group B below, or
(c) R⁹ and R¹⁰ may in combination form an oxo group;
—X³—X⁴— is
—(CR¹¹R¹²)n- wherein n is an integer of 1 to 3, and R¹¹ and R¹² each in the number of n are the same or different and each is
(a) a hydrogen atom, or
(b) a C₁₋₆ alkyl group optionally substituted by one or more, the same or different substituents selected from group B below, or
(c) R¹¹ and R¹² bonded to a single carbon atom may in combination form an oxo group, or
(d) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms optionally substituted by one or more, the same or different substituents selected from group A below; and
ring A is
1) a saturated or unsaturated carbon ring group having 3 to 14 carbon atoms optionally substituted by one or more, the same or different substituents selected from group A below, or
2) a saturated or unsaturated heterocyclic group containing at least one heteroatom selected from a nitrogen atom, an oxygen atom and a sulfur atom, optionally substituted by one or more, the same or different substituents selected from group A below,
[group A]
1) a halogen atom,
2) —OR¹³ wherein R¹³ is
(a) a hydrogen atom,
(b) a C₁₋₆ alkyl group optionally substituted by one or more, the same or different substituents selected from group B below, or
(c) —COR¹⁴ wherein R¹⁴ is
a) a hydrogen atom,
b) a hydroxyl group,
c) a C₁₋₆ alkyl group optionally substituted by one or more, the same or different substituents selected from group B below,
d) a C₁₋₆ alkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
e) a saturated or unsaturated carbon ring group having 3 to 14 carbon atoms optionally substituted by one or more, the same or different substituents selected from (i) and (ii):
(i) a substituent selected from group B below,
(ii) a C₁₋₆ alkyl group optionally substituted by one or more, the same or different substituents selected from group B below,
f) a cycloalkylalkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
g) an aralkyl group optionally substituted by one or more, the same or different substituents selected from (i) and (ii):
(i) a substituent selected from group B below,
(ii) a C₁₋₆ alkyl group optionally substituted by one or more, the same or different substituents selected from group B below, or
h) an aralkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
3) a C₁₋₆ alkyl group optionally substituted by one or more, the same or different substituents selected from group B below,
4) a cycloalkylalkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
5) an aralkyl group optionally substituted by one or more, the same or different substituents selected from (a) and (b):
(a) a substituent selected from group B below,
(b) a C₁₋₆ alkyl group optionally substituted by one or more, the same or different substituents selected from group B below,
6) an aralkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
7) —COR¹⁴ wherein R¹⁴ is as defined above,
8) —NR¹⁵R¹⁶ wherein R¹⁵ and R¹⁶ are the same or different and each is
(a) a hydrogen atom, or
(b) a C₁₋₆ alkyl group optionally substituted by one or more, the same or different substituents selected from group B below, or
(c) R¹⁵ and R¹⁶ may form, together with the nitrogen atom they are bonded to, a monocyclic nitrogen-containing saturated heterocycle optionally substituted by one or more, the same or different substituents selected from (i) and (ii):
(i) a substituent selected from group B below,
(ii) a C₁₋₆ alkyl group optionally substituted by one or more, the same or different substituents selected from group B below,
9) —CONR¹⁵R¹⁶ wherein R¹⁵ and R¹⁶ are as defined above,
10) —NR¹⁷COR¹⁴ wherein R¹⁴ is as defined above, and R¹⁷ is
(a) a hydrogen atom, or
(b) a C₁₋₆ alkyl group optionally substituted by one or more, the same or different substituents selected from group B below,
11) —NR¹⁷S(═O)₂R¹⁴ wherein R¹⁴ and R¹⁷ are as defined above,
12) —NR¹⁷CONR¹⁵R¹⁶ wherein R¹⁵, R¹⁶ and R¹⁷ are as defined above,
13) —SR¹³ wherein R¹³ is as defined above,
14) —S(═O)R¹⁴ wherein R¹⁴ is as defined above,
15) —S(═O)₂R¹⁴ wherein R¹⁴ is as defined above,
16) —S(═O)₂NR¹⁵R¹⁶ wherein R¹⁵ and R¹⁶ are as defined above,
17) a saturated or unsaturated carbon ring group having 3 to 14 carbon atoms optionally substituted by one or more, the same or different substituents selected from (a) and (b):
(a) a substituent selected from group B below,
(b) a C₁₋₆ alkyl group optionally substituted by one or more, the same or different substituents selected from group B below,
18) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B below,
(b) a C₁₋₆ alkyl group optionally substituted by one or more, the same or different substituents selected from group B below,
19) an aryloxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
20) a cyano group, and
21) a nitro group,
[group B]
1) a halogen atom,
2) a hydroxyl group,
3) a C₁₋₆ alkoxy group,
4) —NR¹⁸R¹⁹ wherein R¹⁸ and R¹⁹ are the same or different and each is
(a) a hydrogen atom, or
(b) a C₁₋₆ alkyl group, or
(c) R¹⁸ and R¹⁹ may form, together with the nitrogen atom they are bonded to, a monocyclic nitrogen-containing saturated heterocycle,
5) —CONR¹⁸R¹⁹ wherein R¹⁸ and R¹⁹ are as defined above,
6) —COR²⁰ wherein R²⁰ is
(a) a hydrogen atom,
(b) a hydroxyl group,
(c) a C₁₋₆ alkyl group, or
(d) a C₁₋₆ alkoxy group,
7) —NR²¹COR²⁰ wherein R²⁰ is as defined above, and R²¹ is
(a) a hydrogen atom, or
(b) a C₁₋₆ alkyl group,
8) —NR²¹CONR¹⁸R¹⁹ wherein R¹⁸, R¹⁹ and R²¹ are as defined above,
9) —NR²¹S(═O)₂R²² wherein R²¹ is as defined above, and R²² is a C₁₋₆ alkyl group, and
10) —S(═O)₂R²² wherein R²² is as defined above, wherein the C₁₋₆ alkyl group and C₁₋₆ alkoxy group in 3) to 10) above are optionally further substituted by one or more, the same or different substituents selected from
1′) a halogen atom,
2′) a hydroxyl group,
3′) a C₁₋₆ alkoxy group,
4′) —NR¹⁸′R¹⁹′ wherein R¹⁸′ and R¹⁹′ are the same or different and each is
(a) a hydrogen atom, or
(b) a C₁₋₆ alkyl group, or
(c) R¹⁸′ and R¹⁹′ may form, together with the nitrogen atom they are bonded to, a monocyclic nitrogen-containing saturated heterocycle,
5′) —CONR¹⁸′R¹⁹′ wherein R¹⁸′ and R¹⁹′ are as defined above,
6′) —COR²⁰′ wherein R²⁰′ is
(a) a hydrogen atom,
(b) a hydroxyl group,
(c) a C₁₋₆ alkyl group, or
(d) a C₁₋₆ alkoxy group,
7′) —NR²¹′COR²⁰′ wherein R²⁰′ is as defined above, and R²¹′ is
(a) a hydrogen atom, or
(b) a C₁₋₆ alkyl group,
8′) —NR²¹′CONR¹⁸′R¹⁹′ wherein R¹⁸′, R¹⁹′ and R²¹′ are as defined above,
9′) —NR²¹′S(═O)₂R²²′ wherein R²¹′ is as defined above, and R²²′ is a C₁₋₆ alkyl group, and
10′) —S(═O)₂R²²′ wherein R²²′ is as defined above,
and the monocyclic nitrogen-containing saturated heterocycle in 4), 5) and 8) above are optionally further substituted by one or more substituents selected from a C₁₋₆ alkyl group and 1′) to 10′) above.

2. A pharmaceutical composition comprising a nitrogen-containing fused ring compound represented by the following formula [1] or a pharmaceutically acceptable salt thereof, and one or more pharmaceutically acceptable additives, wherein the additives are free of a basic additive, or comprise less than 1 part by weight of a basic additive per 1 part by weight of the nitrogen-containing fused ring compound represented by the following formula [1] or a pharmaceutically acceptable salt thereof: 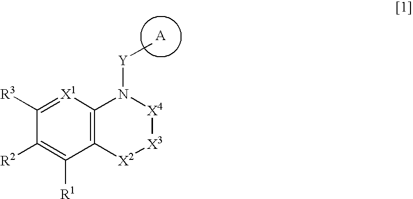
wherein each symbol is as defined in claim 1.

3. The pharmaceutical composition of claim 1, wherein all of the additives are not basic additives.

4. The pharmaceutical composition of claim 1, wherein the basic additive is selected from an alkali metal hydroxide, an alkaline earth metal hydroxide, an alkali metal carbonate, an alkaline earth metal carbonate, an alkali metal hydrogencarbonate, an alkaline earth metal hydrogencarbonate, an alkali metal silicate and an alkaline earth metal silicate.

5. The pharmaceutical composition of claim 1, wherein the basic additive is selected from an alkali metal silicate and an alkaline earth metal silicate.

6. The pharmaceutical composition of claim 1, wherein the basic additive is calcium silicate.

7. A pharmaceutical composition comprising a nitrogen-containing fused ring compound represented by the following formula [1] or a pharmaceutically acceptable salt thereof, and one or more pharmaceutically acceptable additives selected from a group consisting of an acidic additive and a neutral additive, which dose not comprise a basic additive or comprises less than 1 part by weight of a basic additive per 1 part by weight of the nitrogen-containing fused ring compound represented by the following formula [1] or a pharmaceutically acceptable salt thereof: 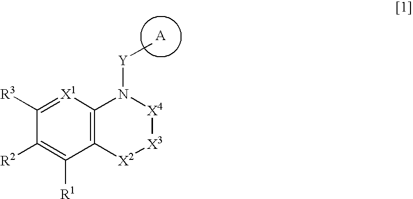
wherein each symbol is as defined in claim 1.

8. A pharmaceutical composition comprising a nitrogen-containing fused ring compound represented by the following formula [1] or a pharmaceutically acceptable salt thereof, and one or more pharmaceutically acceptable additives, wherein all of the additives are selected from an acidic additive and a neutral additive: 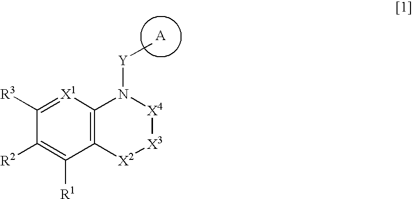
wherein each symbol is as defined in claim 1.

9. The pharmaceutical composition of claim 1, wherein the additive to be contained is one or more selected from a group consisting of D-mannitol, crystalline cellulose, low-substituted hydroxypropylcellulose, light anhydrous silicic acid, crospovidone, magnesium stearate, lactose, corn starch, sodium croscarmellose, carmellose, sodium carmellose, calcium carmellose, sodium carboxymethylstarch, hydroxypropylcellulose, hydroxypropylmethylcellulose, povidone, titanium oxide and macrogol.

10. The pharmaceutical composition of claim 1, which comprises low-substituted hydroxypropylcellulose.

11. The pharmaceutical composition of claim 1, which comprises crospovidone.

12. The pharmaceutical composition of claim 1, which comprises low-substituted hydroxypropylcellulose and crospovidone.

13. The pharmaceutical composition of claim 1, which comprises D-mannitol, crystalline cellulose, low-substituted hydroxypropylcellulose, light anhydrous silicic acid, hydroxypropylmethylcellulose, crospovidone and magnesium stearate.

14. A pharmaceutical composition comprising a nitrogen-containing fused ring compound represented by the following formula [1] or a pharmaceutically acceptable salt thereof, and low-substituted hydroxypropylcellulose: 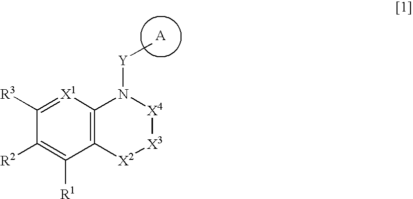
wherein each symbol is as defined in claim 1.

15. A pharmaceutical composition comprising a nitrogen-containing fused ring compound represented by the following formula [1] or a pharmaceutically acceptable salt thereof, and crospovidone: 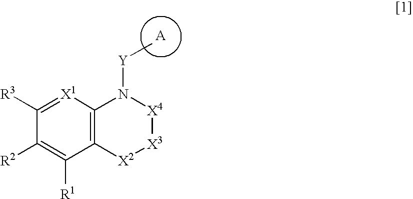
wherein each symbol is as defined in claim 1.

16. A pharmaceutical composition comprising a nitrogen-containing fused ring compound represented by the following formula [1] or a pharmaceutically acceptable salt thereof, and low-substituted hydroxypropylcellulose and crospovidone: 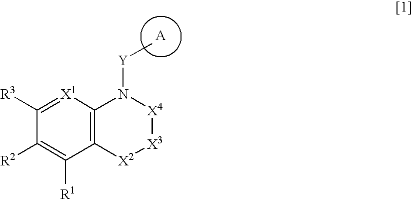
wherein each symbol is as defined in claim 1.

17. The pharmaceutical composition of claim 1, which is in the form of a tablet.

18. The pharmaceutical composition of claim 17, which is coated with a coating agent.

19. The pharmaceutical composition of claim 18, wherein the coating agent comprises hydroxypropylmethylcellulose, titanium oxide and macrogol.

20. The pharmaceutical composition of claim 1, which is a URAT1 activity inhibitor.

21. The pharmaceutical composition of claim 1, which is an agent for decreasing a blood uric acid value.

22. The pharmaceutical composition of claim 1, which is an agent for the prophylaxis or treatment of pathology with involvement of uric acid.

23. The pharmaceutical composition of claim 22, wherein the pathology with involvement of uric acid is hyperuricemia, gouty tophus, acute gouty arthritis, chronic gouty arthritis, gouty kidney, urolithiasis, renal function disorder, coronary artery disease or ischemic heart disease.

24. The pharmaceutical composition of claim 1, which does not substantially inhibit CYP.

25. The pharmaceutical composition of claim 1, wherein the nitrogen-containing fused ring compound represented by the formula [1] is a nitrogen-containing fused ring compound represented by the following formula [2]: 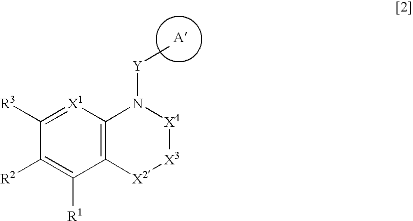
wherein R¹, R², R³, Y, X¹, X³ and X⁴ are as defined in claim 1,
ring A′ is
1) a saturated or unsaturated carbon ring group having 3 to 14 carbon atoms optionally substituted by one or more, the same or different substituents selected from the group C below, or
2) a saturated or unsaturated heterocyclic group containing at least one heteroatom selected from a nitrogen atom, an oxygen atom and a sulfur atom, optionally substituted by one or more, the same or different substituents selected from the group C below,
the ring A′ is substituted by at least one —OR¹³′ wherein R¹³′ is as defined in the group C below;
X²′: is
1) an oxygen atom,
2) —N(R⁵)— wherein R⁵ is
(a) a hydrogen atom, or
(b) a C₁₋₆ alkyl group optionally substituted by one or more, the same or different substituents selected from group B below,
3) —N(COR⁶)— wherein R⁶ is
(a) a hydroxyl group,
(b) a C₁₋₆ alkyl group optionally substituted by one or more, the same or different substituents selected from group B below,
(c) a C₁₋₆ alkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
(d) a saturated or unsaturated carbon ring group having 3 to 14 carbon atoms optionally substituted by one or more, the same or different substituents selected from group A below,
(e) a cycloalkylalkoxy group optionally substituted by one or more, the same or different substituents selected from group A below,
(f) an aralkyl group optionally substituted by one or more, the same or different substituents selected from group A below, or
(g) an aralkoxy group optionally substituted by one or more, the same or different substituents selected from group A below,
4) —N(S(═O)₂R⁶)— wherein R⁶ is as defined above,
5) —N(CONR⁷R⁸)— wherein R⁷ and R⁸ are the same or different and each is
(a) a hydrogen atom, or
(b) a C₁₋₆ alkyl group optionally substituted by one or more, the same or different substituents selected from group B below, or
(c) R⁷ and R⁸ may form, together with the nitrogen atom they are bonded to, a monocyclic nitrogen-containing saturated heterocycle optionally substituted by one or more, the same or different substituents selected from group A below,
6) a sulfur atom,
7) —S(═O)—,
8) —S(═O)₂—, or
9) —CH₂—,
(provided that when X²′ is —CH₂—,
then —X³—X⁴— should be
—(CR¹¹R¹²)n- wherein n is an integer of 1 to 3, and R¹¹ and R¹² each in the number of n are the same or different and each is
(a) a hydrogen atom, or
(b) R¹¹ and R¹² bonded to a single carbon atom may in combination form an oxo group, or
(c) two of R¹¹ and R¹² each in the number of n, which are bonded to a single carbon atom or two adjacent carbon atoms, may form, together with the carbon atom(s), a saturated or unsaturated carbon ring having 3 to 14 carbon atoms optionally substituted by one or more, the same or different substituents selected from group A below;
R¹³′ should be a hydrogen atom; and
ring A′ should be further substituted by at least one a halogen atom;
provided that when both R¹¹ and R¹² are hydrogen atoms, and n is 2, then all of R¹, R² and R³ should be hydrogen atoms),
[group A]
1) a halogen atom,
2) —OR¹³ wherein R¹³ is
(a) a hydrogen atom,
(b) a C₁₋₆ alkyl group optionally substituted by one or more, the same or different substituents selected from group B below, or
(c) —COR¹⁴ wherein R¹⁴ is
a) a hydrogen atom,
b) a hydroxyl group,
c) a C₁₋₆ alkyl group optionally substituted by one or more, the same or different substituents selected from group B below,
d) a C₁₋₆ alkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
e) a saturated or unsaturated carbon ring group having 3 to 14 carbon atoms optionally substituted by one or more, the same or different substituents selected from (i) and (ii):
(i) a substituent selected from group B below,
(ii) a C₁₋₆ alkyl group optionally substituted by one or more, the same or different substituents selected from group B below,
f) a cycloalkylalkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
g) an aralkyl group optionally substituted by one or more, the same or different substituents selected from (i) and (ii):
(i) a substituent selected from group B below,
(ii) a C₁₋₆ alkyl group optionally substituted by one or more, the same or different substituents selected from group B below, or
h) an aralkoxy group optionally substituted by one or more, the same or different substituents selected from (i) and (ii):
(i) a substituent selected from group B below,
(ii) a C₁₋₆ alkyl group optionally substituted by one or more, the same or different substituents selected from group B below,
3) a C₁₋₆ alkyl group optionally substituted by one or more, the same or different substituents selected from group B below,
4) a cycloalkylalkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
5) an aralkyl group optionally substituted by one or more, the same or different substituents selected from (a) and (b):
(a) a substituent selected from group B below,
(b) a C₁₋₆ alkyl group optionally substituted by one or more, the same or different substituents selected from group B below,
6) an aralkoxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
7) —COR¹⁴ wherein R¹⁴ is as defined above,
8) —NR¹⁵R¹⁶ wherein R¹⁵ and R¹⁶ are the same or different and each is
(a) a hydrogen atom, or
(b) a C₁₋₆ alkyl group optionally substituted by one or more, the same or different substituents selected from group B below, or
(c) R¹⁵ and R¹⁶ may form, together with the nitrogen atom they are bonded to, a monocyclic nitrogen-containing saturated heterocycle optionally substituted by one or more, the same or different substituents selected from (i) and (ii):
(i) a substituent selected from group B below,
(ii) a C₁₋₆ alkyl group optionally substituted by one or more, the same or different substituents selected from group B below,
9) —CONR¹⁵R¹⁶ wherein R¹⁵ and R¹⁶ are as defined above,
10) —NR¹⁷COR¹⁴ wherein R¹⁴ is as defined above, and R¹⁷ is
(a) a hydrogen atom, or
(b) a C₁₋₆ alkyl group optionally substituted by one or more, the same or different substituents selected from group B below,
11) —NR¹⁷S(═O)₂R¹⁴ wherein R¹⁴ and R¹⁷ are as defined above,
12) —NR¹⁷CONR¹⁵R¹⁶ wherein R¹⁵, R¹⁶ and R¹⁷ are as defined above,
13) —SR¹³ wherein R¹³ is as defined above,
14) —S(═O)R¹⁴ wherein R¹⁴ is as defined above,
15) —S(═O)₂R¹⁴ wherein R¹⁴ is as defined above,
16) —S(═O)₂NR¹⁵R¹⁶ wherein R¹⁵ and R¹⁶ are as defined above,
17) a saturated or unsaturated carbon ring group having 3 to 14 carbon atoms optionally substituted by one or more, the same or different substituents selected from (a) and (b):
(a) a substituent selected from group B below,
(b) a C₁₋₆ alkyl group optionally substituted by one or more, the same or different substituents selected from group B below,
18) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B below,
(b) a C₁₋₆ alkyl group optionally substituted by one or more, the same or different substituents selected from group B below,
19) an aryloxy group optionally substituted by one or more, the same or different substituents selected from (a) and (b):
(a) a substituent selected from group B below,
(b) a C₁₋₆ alkyl group optionally substituted by one or more, the same or different substituents selected from group B below,
20) a cyano group, and
21) a nitro group,
[group B]
1) a halogen atom,
2) a hydroxyl group,
3) a C₁₋₆ alkoxy group,
4) —NR¹⁸R¹⁹ wherein R¹⁸ and R¹⁹ are the same or different and each is
(a) a hydrogen atom, or
(b) a C₁₋₆ alkyl group, or
(c) R¹⁸ and R¹⁹ may form, together with the nitrogen atom they are bonded to, a monocyclic nitrogen-containing saturated heterocycle,
5) —CONR¹⁸R¹⁹ wherein R¹⁸ and R¹⁹ are as defined above,
6) —COR²⁰ wherein R²⁰ is
(a) a hydrogen atom,
(b) a hydroxyl group,
(c) a C₁₋₆ alkyl group, or
(d) a C₁₋₆ alkoxy group,
7) —NR²¹COR²⁰ wherein R²⁰ is as defined above, and R²¹ is
(a) a hydrogen atom, or
(b) a C₁₋₆ alkyl group,
8) —NR²¹CONR¹⁸R¹⁹ wherein R¹⁸, R¹⁹ and R²¹ are as defined above,
9) —NR²¹S(═O)₂R²² wherein R²¹ is as defined above, and R²² is a C₁₋₆ alkyl group, and
10) —S(═O)₂R²² wherein R²² is as defined above,
wherein the C₁₋₆ alkyl group and C₁₋₆ alkoxy group in 3) to 10) above are optionally further substituted by one or more, the same or different substituents selected from
1′) a halogen atom,
2′) a hydroxyl group,
3′) a C₁₋₆ alkoxy group,
4′) —NR¹⁸′R¹⁹′ wherein R¹⁸′ and R¹⁹′ are the same or different and each is
(a) a hydrogen atom, or
(b) a C₁₋₆ alkyl group, or
(c) R¹⁸′ and R¹⁹′ may form, together with the nitrogen atom they are bonded to, a monocyclic nitrogen-containing saturated heterocycle,
5′) —CONR¹⁸′R¹⁹′ wherein R¹⁸′ and R¹⁹′ are as defined above,
6′) —COR²⁰′ wherein R²⁰′ is
(a) a hydrogen atom,
(b) a hydroxyl group,
(c) a C₁₋₆ alkyl group, or
(d) a C₁₋₆ alkoxy group,
7′) —NR²¹′COR²⁰′ wherein R²⁰′ is as defined above, and R²¹′ is
(a) a hydrogen atom, or
(b) a C₁₋₆ alkyl group,
8′) —NR²¹′CONR¹⁸′R¹⁹′ wherein R¹⁸′, R¹⁹′ and R²¹′ are as defined above,
9′) —NR²¹′S(═O)₂R²²′ wherein R²¹′ is as defined above, and R²²′ is a C₁₋₆ alkyl group, and
10′) —S(═O)₂R²²′ wherein R²²′ is as defined above,
and the monocyclic nitrogen-containing saturated heterocycle in 4), 5) and 8) above are optionally further substituted by one or more substituents selected from a C₁₋₆ alkyl group and 1′) to 10′) above.
[group C]
1) a halogen atom,
2) —OR¹³′ wherein R¹³′ is
(a) a hydrogen atom,
(b) a C₁₋₆ alkyl group optionally substituted by one or more, the same or different substituents selected from group B above, or
(c) —COR¹⁴′ wherein R¹⁴′ is
a) a hydrogen atom,
b) a hydroxyl group,
c) a C₁₋₆ alkyl group optionally substituted by one or more, the same or different substituents selected from group B above,
d) a C₁₋₆ alkoxy group optionally substituted by one or more, the same or different substituents selected from (i) and (ii):
(i) a substituent selected from group B above,
(ii) a C₁₋₆ alkyl group optionally substituted by one or more, the same or different substituents selected from group B above,
e) a saturated or unsaturated carbon ring group having 3 to 14 carbon atoms optionally substituted by one or more, the same or different substituents selected from (i) and (ii):
(i) a substituent selected from group B above,
(ii) a C₁₋₆ alkyl group optionally substituted by one or more, the same or different substituents selected from group B above,
f) a cycloalkylalkoxy group optionally substituted by one or more, the same or different substituents selected from (i) and (ii):
(i) a substituent selected from group B above,
(ii) a C₁₋₆ alkyl group optionally substituted by one or more, the same or different substituents selected from group B above,
g) an aralkyl group optionally substituted by one or more, the same or different substituents selected from (i) and (ii):
(i) a substituent selected from group B above,
(ii) a C₁₋₆ alkyl group optionally substituted by one or more, the same or different substituents selected from group B above, or
h) an aralkoxy group optionally substituted by one or more, the same or different substituents selected from (i) and (ii):
(i) a substituent selected from group B above,
(ii) a C₁₋₆ alkyl group optionally substituted by one or more, the same or different substituents selected from group B above,
3) a C₁₋₆ alkyl group optionally substituted by one or more, the same or different substituents selected from group B above,
4) a cycloalkylalkoxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
5) an aralkyl group optionally substituted by one or more, the same or different substituents selected from (a) and (b):
(a) a substituent selected from group B above,
(b) a C₁₋₆ alkyl group optionally substituted by one or more, the same or different substituents selected from group B above,
6) an aralkoxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
7) —COR¹⁴′ wherein R¹⁴′ is as defined above,
8) —NR¹⁵′R¹⁶′ wherein R¹⁵′ and R¹⁶′ are the same or different and each is
(a) a hydrogen atom, or
(b) a C₁₋₆ alkyl group optionally substituted by one or more, the same or different substituents selected from group B above, or
(c) R¹⁵′ and R¹⁶′ may form, together with the nitrogen atom they are bonded to, a monocyclic nitrogen-containing saturated heterocycle optionally substituted by one or more, the same or different substituents selected from (i) and (ii):
(i) a substituent selected from group B above,
(ii) a C₁₋₆ alkyl group optionally substituted by one or more, the same or different substituents selected from group B above,
9) —NR¹⁷′COR¹⁴′ wherein R¹⁴′ is as defined above, and R¹⁷′ is
(a) a hydrogen atom, or
(b) a C₁₋₆ alkyl group optionally substituted by one or more, the same or different substituents selected from group B above,
10) —NR¹⁷′S(═O)₂R¹⁴′ wherein R¹⁴′ and R¹⁷′ are as defined above,
11) —NR¹⁷′CONR¹⁵′R¹⁶′ wherein R¹⁵′, R¹⁶′ and R¹⁷′ are as defined above,
12) —SR¹³′ wherein R¹³′ is as defined above,
13) —S(═O)R¹⁴′ wherein R¹⁴′ is as defined above,
14) —S(═O)₂R¹⁴′ wherein R¹⁴′ is as defined above,
15) —S(═O)₂NR¹⁵′R¹⁶′ wherein R¹⁵′ and R¹⁶′ are as defined above,
16) a saturated or unsaturated carbon ring group having 3 to 14 carbon atoms optionally substituted by one or more, the same or different substituents selected from (a) and (b):
(a) a substituent selected from group B above,
(b) a C₁₋₆ alkyl group optionally substituted by one or more, the same or different substituents selected from group B above,
17) a saturated or unsaturated heterocyclic group containing at least one heteroatom selected from a nitrogen atom, an oxygen atom and a sulfur atom, optionally substituted by one or more, the same or different substituents selected from (a) and (b):
(a) a substituent selected from group B above,
(b) a C₁₋₆ alkyl group optionally substituted by one or more, the same or different substituents selected from group B above,
18) an aryloxy group optionally substituted by one or more, the same or different substituents selected from (a) and (b):
(a) a substituent selected from group B above,
(b) a C₁₋₆ alkyl group optionally substituted by one or more, the same or different substituents selected from group B above,
19) a cyano group, and
20) a nitro group.

26. The pharmaceutical composition of claim 1, wherein the nitrogen-containing fused ring compound represented by the formula [1] is (3-chloro-4-hydroxyphenyl)-(2,3-dihydrobenzo[1,4]oxazin-4-yl)-methanone.

27. The pharmaceutical composition of claim 1, wherein the nitrogen-containing fused ring compound represented by the formula [1] is (3-bromo-4-hydroxyphenyl)-(2,3-dihydrobenzo[1,4]oxazin-4-yl)-methanone.

28. The pharmaceutical composition of claim 1, wherein the nitrogen-containing fused ring compound represented by the formula [1] is (3,5-dichloro-4-hydroxyphenyl)-(2,3-dihydrobenzo[1,4]oxazin-4-yl)-methanone.

29. A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the nitrogen-containing fused ring compound or a pharmaceutically acceptable salt thereof is not in contact with a basic additive: 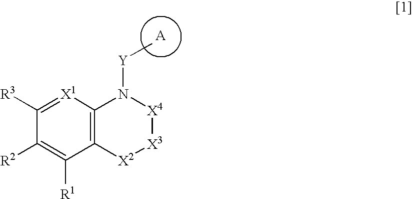
wherein each symbol is as defined in claim 1.

30. A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the additives are free of a basic additive, or comprise less than 1 part by weight of a basic additive per 1 part by weight of the nitrogen-containing fused ring compound or a pharmaceutically acceptable salt thereof: 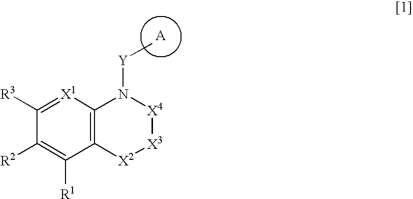
wherein each symbol is as defined in claim 1.

31. A method of stabilizing a nitrogen-containing fused ring compound represented by the following formula [1] or a pharmaceutically acceptable salt thereof, comprising adding one or more pharmaceutically acceptable additives selected from a group consisting of an acidic additive and a neutral additive to the nitrogen-containing fused ring compound or a pharmaceutically acceptable salt thereof, wherein the additives are free of a basic additive, or comprise less than 1 part by weight of a basic additive per 1 part by weight of the nitrogen-containing fused ring compound or a pharmaceutically acceptable salt thereof: 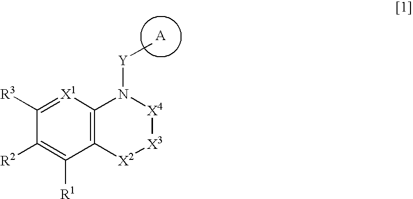
wherein each symbol is as defined in claim 1.

32. A method of stabilizing a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein all of the additives are selected from a group consisting of an acidic additive and a neutral additive: 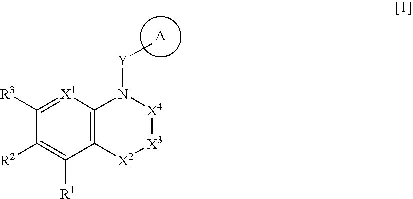
wherein each symbol is as defined in claim 1.

33. A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the nitrogen-containing fused ring compound or a pharmaceutically acceptable salt thereof is not in contact with a basic additive: 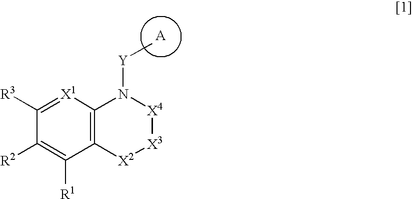
wherein each symbol is as defined in claim 1.

34. A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein the additives are free of a basic additive, or comprise less than 1 part by weight of a basic additive per 1 part by weight of the nitrogen-containing fused ring compound or a pharmaceutically acceptable salt thereof: 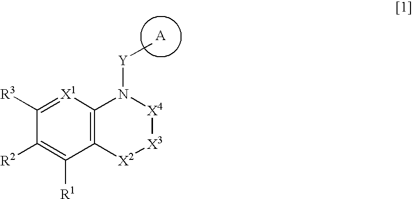
wherein each symbol is as defined in claim 1.

35. A method of preserving a nitrogen-containing fused ring compound represented by the following formula [1] or a pharmaceutically acceptable salt thereof, comprising adding one or more pharmaceutically acceptable additives selected from a group consisting of an acidic additive and a neutral additive to the nitrogen-containing fused ring compound or a pharmaceutically acceptable salt thereof,
wherein the additives are free of a basic additive, or comprise less than 1 part by weight of a basic additive per 1 part by weight of the nitrogen-containing fused ring compound or a pharmaceutically acceptable salt thereof: 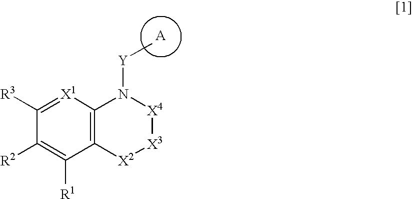
wherein each symbol is as defined in claim 1.

36. A method of preserving a nitrogen-containing fused ring compound represented by the following formula [1] or a pharmaceutically acceptable salt thereof, comprising adding one or more pharmaceutically acceptable additives to the nitrogen-containing fused ring compound or a pharmaceutically acceptable salt thereof, wherein all of the additives are selected from a group consisting of an acidic additive and a neutral additive: 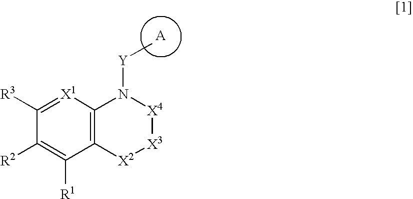
wherein each symbol is as defined in claim 1.
